(19) United States
(12) Patent Application Publication
(10) Pub. No.: US 2010/0179139 A1
(43) Pub. Date: Jul. 15, 2010

(54) PYRROLOPYRIDINE COMPOUNDS, PROCESS FOR THEIR PREPARATION, AND THEIR USE AS MEDICAMENTS

(76) Inventors: Paul Bamborough, Hertfordshire (GB); Michael David Barker, Hertfordshire (GB); Sebastien Andre Campos, Hertfordshire (GB); Richard Peter Charles Cousins, Hertfordshire (GB); Paul Faulder, Hertfordshire (GB); Heather Hobbs, Hertfordshire (GB); Duncan Stuart Holmes, Hertfordshire (GB); Michael John Johnston, Hertfordshire (GB); John Liddle, Hertfordshire (GB); Jeremy John Payne, Hertfordshire (GB); John Martin Pritchard, Hartfordshire (GB); Caroline Whitworth, Hertfordshire (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/602,619

(22) PCT Filed: May 29, 2008
(86) PCT No.: PCT/EP2008/056597

§ 371 Date: Dec. 1, 2009

(30) Foreign Application Priority Data
Jun. 1, 2007 (GB) 0710528.1

(51) Int. Cl. A61K 31/437; C07D 471/04; A61K 31/506; A61K 31/541; A61P 11/02; A61P 11/06; A61P 11/00; A61P 29/00; A61P 19/10; A61P 31/18; A61P 25/00; A61P 3/10; A61P 9/10; A61P 25/28; A61P 35/00

(52) U.S. Cl. 514/228.2; 546/113; 514/300; 544/298; 514/269; 544/58.2

(57) ABSTRACT

The invention is directed to certain novel compounds. Specifically, the invention is directed to compounds according to formula (I):

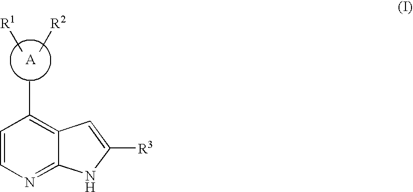

and salts thereof.

The compounds of the invention are inhibitors of kinase activity, in particular IKK2 activity.

### FIELD OF THE INVENTION

The invention is directed to certain novel compounds which are inhibitors of kinase activity. More specifically, the compounds are IKK2 inhibitors. Compounds which are IKK2 inhibitors may be useful in the treatment of disorders associated with inappropriate IKK2 (also known as IKKβ) activity, in particular in the treatment and prevention of disorders mediated by IKK2 mechanisms including inflammatory and tissue repair disorders. Such disorders include rheumatoid arthritis, COPD (chronic obstructive pulmonary disease), asthma and rhinitis.

### BACKGROUND OF THE INVENTION

An important large family of enzymes is the protein kinase enzyme family. Currently, there are about 500 different known protein kinases. However, because three to four percent of the human genome is a code for the formation of protein kinases, there may be many thousands of distinct and separate kinases in the human body. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The serine/threonine kinases (PSTK) include cyclic AMP- and cyclic GMP-dependent protein kinases, calcium and phospholipid dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also in progress to identify modulators of tyrosine kinases as well.

Nuclear factor κB (NF-κB) represents a family of closely related dimeric transcription factor complexes composed of various combinations of the ReI/NF-κB family of polypeptides. The family consists of five individual gene products in mammals, ReIA (p65), NF-κB1 (p50/p105), NF-κB2 (p52/p100), c-ReI, and ReIB, all of which can form hetero- or homo-dimers. These proteins share a highly homologous 300 amino acid “ReI homology domain” which contains the DNA binding and dimerization domains. The NFkBs also carry a nuclear localisation sequence near the C-terminus of the ReI homology domain which is important in the transport of NF-κB from the cytoplasm to the nucleus. In addition, p65 and cReI possess potent transactivation domains at their C-terminal ends.

The activity of NF-κB is regulated by its interaction with a member of the inhibitor IκB family of proteins. This interaction effectively blocks the nuclear localization sequence on the NF-κB proteins, thus preventing migration of the dimer to the nucleus. A wide variety of stimuli activate NF-κB through what are likely to be multiple signal transduction pathways. Included are bacterial products (LPS), some viruses (HIV-1, HTLV-1), inflammatory cytokines (TNFα, IL-1), environmental and oxidative stress and DNA damaging agents. Apparently common to all stimuli however, is the phosphorylation and subsequent degradation of IκB. IκBα and β for example, are phosphorylated on two N-terminal serines by the recently identified IκB kinases (IKK-α and IKK-β), whilst NF-κB2, which carries an IkB-like C terminal region is phosphorylated on N and C terminal serines by IKK-α. IKK-β is also known as IKK2 and it's now widely accepted that it is essential for rapid NFkB activation in response to pro-inflammatory stimuli. IKK2 is an example of a serine/threonine kinase. Site-directed mutagenesis studies indicate that these phosphorylations are critical for the subsequent activation of NF-κB in that once phosphorylated the protein is flagged for degradation via the ubiquitin-proteasome pathway. Free from IκB, the active NF-κB complexes are able to translocate to the nucleus where they bind in a selective manner to preferred gene-specific enhancer sequences. Included in the genes regulated by NF-κB are a number of cytokines and chemokines, cell adhesion molecules, acute phase proteins, immunoregulatory proteins, eicosanoid metabolizing enzymes and anti-apoptotic genes.

It is well-known that NF-κB plays a key role in the regulated expression of a large number of pro-inflammatory mediators including cytokines such as TNF, IL-1β, IL-6 and IL-8, cell adhesion molecules, such as ICAM and VCAM, and inducible nitric oxide synthase (iNOS). Such mediators are known to play a role in the recruitment of leukocytes at sites of inflammation and in the case of iNOS, may lead to organ destruction in some inflammatory and autoimmune diseases.

The importance of NF-κB in inflammatory disorders is further strengthened by studies of airway inflammation including asthma, in which NF-κB has been shown to be activated. This activation may underlie the increased cytokine production and leukocyte infiltration characteristic of these disorders. In addition, inhaled steroids are known to reduce airway hyperresponsiveness and suppress the inflammatory response in asthmatic airways. In light of the recent findings with regard to glucocorticoid inhibition of NF-κB, one may speculate that these effects are mediated through an inhibition of NF-κB.

Further evidence for a role of NF-κB in inflammatory disorders comes from studies of rheumatoid synovium. Although NF-κB is normally present as an inactive cytoplasmic complex, recent immunohistochemical studies have indicated that NF-κB is present in the nuclei, and hence active, in the cells comprising rheumatoid synovium. Furthermore, NF-κB has been shown to be activated in human synovial cells in response to stimulation with TNF-α or IL-β. Such a distribution may be the underlying mechanism for the increased cytokine and eicosanoid production characteristic of this tissue. See Roshak, A. K., et al., J. Biol. Chem., 271, 31496-31501 (1996). Expression of IKK-β has been shown in synoviocytes of rheumatoid arthritis patients and gene transfer studies have demonstrated the central role of IKK-β in stimulated inflammatory mediator production in these cells. See Aupperele, K. R., et al., J. Immunology, 1999., 163:427-433 and Aupperele, K. R., et al., J. Immunology, 2001, 166:2705-11. More recently, the intra-articular administration of a wild type IKK-β adenoviral construct was shown to cause paw swelling while intra-articular administration of dominant-negative IKKβ inhibited adjuvant-induced arthritis in rat. See Tak, P. P., et al., Arthritis and Rheumatism, 2001, 44:1897-1907.

The NF-κB/ReI and IκB proteins are also likely to play a key role in neoplastic transformation and metastasis. Family members are associated with cell transformation in vitro and in vivo as a result of over expression, gene amplification, gene rearrangements or translocations. In addition, rearrangement and/or amplification of the genes encoding these proteins are seen in 20-25% of certain human lymphoid tumors. Further, NF-κB is activated by oncogenic ras, the most common defect in human tumors and blockade of NF-κB activation inhibits ras mediated cell transformation. In addition, a role for NF-κB in the regulation of apoptosis has been reported strengthening the role of this transcription factor in the regulation of tumor cell proliferation. TNF, ionizing radiation and DNA damaging agents have all been shown to activate NF-κB which in turn leads to the upregulated expression of several anti-apoptotic proteins. Conversely, inhibition of NF-κB has been shown to enhance apoptotic-killing by these agents in several tumor cell types. As this likely represents a major mechanism of tumor cell resistance to chemotherapy, inhibitors of NF-κB activation may be useful chemotherapeutic agents as either single agents or adjunct therapy. Recent reports have implicated NF-κB as an inhibitor of skeletal cell differentiation as well as a regulator of cytokine-induced muscle wasting (Guttridge, D. C., et al., Science, 2000, 289: 2363-2365) further supporting the potential of NFκB inhibitors as novel cancer therapies.

Several NF-κB and IKK inhibitors are described in Wahl, C., et al., J. Clin. Invest. 101(5), 1163-1174 (1998); Sullivan, R. W., et al., J. Med. Chem., 41, 413-419 (1998); Pierce, J. W., et al., J. Biol. Chem. 272, 21096-21103 (1997); and Coish, P. D. G., et al., Expert Opin. Ther. Patents, 2006, vol 16(1) 1-12.

The marine natural product hymenialdisine is known to inhibit NF-κB. See Roshak, A., et al., JPET, 283, 955-961 (1997)); and Breton, J. J., and Chabot-Fletcher, M. C., JPET, 282, 459-466 (1997).

Attempts have been made to prepare compounds that inhibit IKK2 activity and a number of such compounds have been disclosed in the art. However, in view of the number of pathological responses that are mediated by IKK2, there remains a continuing need for inhibitors of IKK2 which can be used in the treatment of a variety of conditions.

The present inventors have discovered novel compounds which are inhibitors of kinase activity, in particular IKK2 activity. Compounds which are IKK2 inhibitors may be useful in the treatment of disorders associated with inappropriate kinase activity, in particular inappropriate IKK2 activity, for example in the treatment and prevention of disorders mediated by IKK2 mechanisms. Such disorders include inflammatory and tissue repair disorders (including rheumatoid arthritis, inflammatory bowel disease, COPD (chronic obstructive pulmonary disease), asthma and rhinitis), fibrotic diseases, osteoarthritis, osteoporosis, dermatosis (including psoriasis, atopic dermatitis and ultraviolet radiation (UV)-induced skin damage), autoimmune diseases (including Sjogren's syndrome, systemic lupus erythematosus, multiple sclerosis, psoriatic arthritis, alkylosing spondylitis, tissue and organ rejection), Alzheimer's disease, stroke, atherosclerosis, restonosis, diabetes, glomerulonephritis, cancer (including Hodgkin's disease), cachexia, inflammation associated with infection and certain viral infections (including acquired immune deficiency syndrome (AIDS)), adult respiratory distress syndrome, and Ataxia Telangiestasia.

In one embodiment, the compounds may show selectivity for IKK2 over other kinases.

### SUMMARY OF THE INVENTION

The invention is directed to certain novel compounds. Specifically, the invention is directed to compounds of formula (I):

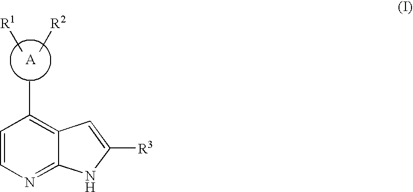

wherein R¹, R² and R³ are as defined below, and salts thereof (hereinafter “compounds of the invention”).

The compounds of the invention are inhibitors of IKK2 activity. Compounds which are IKK2 inhibitors may be useful in the treatment of disorders associated with inappropriate IKK2 (also known as IKKβ) activity, such as rheumatoid arthritis, COPD (chronic obstructive pulmonary disease), asthma and rhinitis (including seasonal rhinitis, allergic rhinitis such as seasonal rhinitis (for example hayfever) or perennial rhinitis, and non-allergic rhinitis or vasomotor rhinitis). Accordingly, the invention is further directed to pharmaceutical compositions comprising a compound of formula (I) or a pharmaceutically acceptable salt thereof. The invention is still further directed to methods of inhibiting IKK2 activity and treatment of disorders associated therewith using a compound of formula (I) or a pharmaceutically acceptable salt thereof, or a pharmaceutical composition comprising a compound of formula (I) or a pharmaceutically acceptable salt thereof.

### DETAILED DESCRIPTION OF THE INVENTION

In one embodiment, the invention is directed to compounds of formula (I):

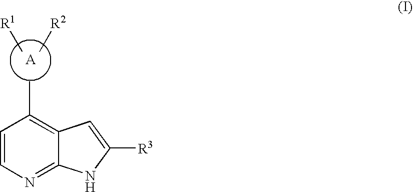

wherein
A is a 5- or 6-membered heteroaryl ring containing one heteroatom selected from nitrogen and sulphur, and optionally one further heteroatom which is nitrogen;
R¹ is hydrogen or methyl;

### R² is —SO₂NR⁴R⁵;

R³ is —CR⁶R⁷R⁸, cyclopropyl or piperidinyl;
R⁴ is hydrogen or methyl,
R⁵ is C₁₋₆alkyl optionally substituted by one or two substituents independently selected from —OH and —NH₂, or a 5- or 6-membered heterocyclyl containing one heteroatom selected from oxygen, nitrogen and S(O)ₐ, or
R⁴ and R⁵ are linked to form a 5- or 6-membered heterocyclyl optionally containing one further heteroatom selected from oxygen, nitrogen and S(O)_{b};
R⁶, R⁷ and R⁸ are each independently hydrogen, fluorine or methyl;
a and b are each independently an integer selected from 0 to 2;
and salts thereof.

In one embodiment, A is thienyl, thiazolyl, pyridinyl or pyrimidinyl. In another embodiment, A is thienyl, for example

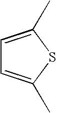

In a further embodiment, A is pyridinyl, for example

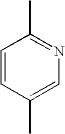

In one embodiment, R¹ is hydrogen.

In one embodiment, R³ is —CR⁶R⁷R⁸. In another embodiment, R³ is cyclopropyl. In a further embodiment, R³ is piperidinyl, for example 3-piperidinyl.

In one embodiment, R⁴ is hydrogen.

In one embodiment, R⁵ is C₁₋₆alkyl optionally substituted by one or two substituents independently selected from —OH and —NH₂. In a further embodiment, R⁵ is C₁₋₆alkyl substituted by one —OH.

In one embodiment, R⁵ is a 5- or 6-membered heterocyclyl containing one heteroatom selected from oxygen, nitrogen and S(O)ₐ.

In one embodiment, R⁴ and R⁵ are linked to form a 5- or 6-membered heterocyclyl optionally containing one further heteroatom which is S(O)_{b}.

In one embodiment, R⁶, R⁷ and R⁸ are each hydrogen. In another embodiment, R⁶, R⁷ and R⁸ are each fluorine. In another embodiment, R⁶, R⁷ and R⁸ are each methyl. In another embodiment, R⁶ and R⁷ are each fluorine and R⁸ is hydrogen. In a further embodiment, R⁶ and R⁷ are each methyl and R⁸ is hydrogen.

In one embodiment, a is 0. In a further embodiment, a is 2.

In one embodiment, b is 2.

It is to be understood that the present invention covers all combinations of substituent groups described hereinabove.

Compounds of the invention include the compounds of Examples 1 to 144 and salts thereof.

In one embodiment, the compound of the invention is:
N-(2-hydroxyethyl)-5-(2-methyl-1H-pyrrolo[2,3-b]pyridin-4-yl)-2-thiophenesulfonamide;
N-(1,1-dioxidotetrahydro-2H-thiopyran-4-yl)-5-(2-methyl-1H-pyrrolo[2,3-b]pyridin-4-yl)-2-thiophenesulfonamide;
N-(2-aminoethyl)-5-(2-methyl-1H-pyrrolo[2,3-b]pyridin-4-yl)-2-thiophenesulfonamide;
N-(2-hydroxyethyl)-5-[2-(3-piperidinyl)-1H-pyrrolo[2,3-b]pyridin-4-yl]-2-thiophenesulfonamide;
N-(2-aminoethyl)-5-[2-(3-piperidinyl)-1H-pyrrolo[2,3-b]pyridin-4-yl]-2-thiophenesulfonamide;
N-(1,1-dioxidotetrahydro-3-thienyl)-5-[2-(3-piperidinyl)-1H-pyrrolo[2,3-b]pyridin-4-yl]-2-thiophenesulfonamide;
N-(1,1-dioxidotetrahydro-2H-thiopyran-4-yl)-5-[2-(3-piperidinyl)-1H-pyrrolo[2,3-b]pyridin-4-yl]-2-thiophenesulfonamide;
N-(1,1-dioxidotetrahydro-2H-thiopyran-4-yl)-6-(2-methyl-1H-pyrrolo[2,3-b]pyridin-4-yl)-3-pyridinesulfonamide;
2-methyl-4-[5-(1-pyrrolidinylsulfonyl)-2-pyridinyl]-1H-pyrrolo[2,3-b]pyridine;
N-(1,1-dioxidotetrahydro-2H-thiopyran-4-yl)-5-(2-methyl-1H-pyrrolo[2,3-b]pyridin-4-yl)-2-pyridinesulfonamide;
2-methyl-4-[6-(1-pyrrolidinylsulfonyl)-3-pyridinyl]-1H-pyrrolo[2,3-b]pyridine;
N-(1,1-dioxidotetrahydro-2H-thiopyran-4-yl)-2-methyl-5-[2-(trifluoromethyl)-1H-pyrrolo[2,3-b]pyridin-4-yl]-3-thiophenesulfonamide;
N-(1,1-dioxidotetrahydro-2H-thiopyran-4-yl)-4-methyl-2-[2-(trifluoromethyl)-1H-pyrrolo[2,3-b]pyridin-4-yl]-1,3-thiazole-5-sulfonamide;
N-(1,1-dioxidotetrahydro-2H-thiopyran-4-yl)-2-(2-methyl-1H-pyrrolo[2,3-b]pyridin-4-yl)-5-pyrimidinesulfonamide;
6-[2-(difluoromethyl)-1H-pyrrolo[2,3-b]pyridin-4-yl]-N-(1,1-dioxidotetrahydro-2H-thiopyran-4-yl)-3-pyridinesulfonamide;
6-[2-(difluoromethyl)-1H-pyrrolo[2,3-b]pyridin-4-yl]-N-4-piperidinyl-3-pyridinesulfonamide;
2-(difluoromethyl)-4-{5-[(1,1-dioxido-4-thiomorpholinyl)sulfonyl]-2-pyridinyl}-1H-pyrrolo[2,3-b]pyridine;
2-(difluoromethyl)-4-{6-[(1,1-dioxido-4-thiomorpholinyl)sulfonyl]-3-pyridinyl}-1H-pyrrolo[2,3-b]pyridine;
5-[2-(difluoromethyl)-1H-pyrrolo[2,3-b]pyridin-4-yl]-N-4-piperidinyl-2-pyridinesulfonamide;
6-[2-(difluoromethyl)-1H-pyrrolo[2,3-b]pyridin-4-yl]-N-(1,1-dioxidotetrahydro-2H-thiopyran-4-yl)-N-methyl-3-pyridinesulfonamide;
5-[2-(difluoromethyl)-1H-pyrrolo[2,3-b]pyridin-4-yl]-N-(1,1-dioxidotetrahydro-2H-thiopyran-4-yl)-N-methyl-2-pyridinesulfonamide;
5-[2-(difluoromethyl)-1H-pyrrolo[2,3-b]pyridin-4-yl]-N-(1,1-dioxidotetrahydro-2H-thiopyran-4-yl)-2-pyridinesulfonamide;
N-(1,1-dioxidotetrahydro-2H-thiopyran-4-yl)-6-(2-methyl-1H-pyrrolo[2,3-b]pyridin-4-yl)-3-pyridinesulfonamide;
N-(2-hydroxyethyl)-6-(2-methyl-1H-pyrrolo[2,3-b]pyridin-4-yl)-3-pyridinesulfonamide;
6-(2-methyl-1H-pyrrolo[2,3-b]pyridin-4-yl)-N-4-piperidinyl-3-pyridinesulfonamide;
5-(2-methyl-1H-pyrrolo[2,3-b]pyridin-4-yl)-N-4-piperidinyl-2-pyridinesulfonamide;
N-(1,1-dioxidotetrahydro-2H-thiopyran-4-yl)-N-methyl-6-(2-methyl-1H-pyrrolo[2,3-b]pyridin-4-yl)-3-pyridinesulfonamide;
N-(1,1-dioxidotetrahydro-2H-thiopyran-4-yl)-N-methyl-5-(2-methyl-1H-pyrrolo[2,3-b]pyridin-4-yl)-2-pyridinesulfonamide;
N-(2,3-dihydroxypropyl)-5-(2-methyl-1H-pyrrolo[2,3-b]pyridin-4-yl)-2-pyridinesulfonamide;
N-(2,3-dihydroxypropyl)-5-(2-methyl-1H-pyrrolo[2,3-b]pyridin-4-yl)-2-pyridinesulfonamide;
N-(1,1-dioxidotetrahydro-3-thienyl)-5-(2-methyl-1H-pyrrolo[2,3-b]pyridin-4-yl)-2-thiophenesulfonamide;
2-(1,1-dimethylethyl)-4-[6-(1-pyrrolidinylsulfonyl)-3-pyridinyl]-1H-pyrrolo[2,3-b]pyridine;
N-(2-aminoethyl)-6-[2-(trifluoromethyl)-1H-pyrrolo[2,3-b]pyridin-4-yl]-3-pyridinesulfonamide;
N-(2-aminoethyl)-6-[2-(trifluoromethyl)-1H-pyrrolo[2,3-b]pyridin-4-yl]-3-pyridinesulfonamide trifluoroacetate;
N-(1,1-dioxidotetrahydro-2H-thiopyran-4-yl)-6-[2-(trifluoromethyl)-1H-pyrrolo[2,3-b]pyridin-4-yl]-3-pyridinesulfonamide;
N-4-piperidinyl-5-[2-(trifluoromethyl)-1H-pyrrolo[2,3-b]pyridin-4-yl]-2-pyridinesulfonamide;
N-(1,1-dioxidotetrahydro-2H-thiopyran-4-yl)-6-[2-(trifluoromethyl)-1H-pyrrolo[2,3-b]pyridin-4-yl]-3-pyridinesulfonamide;
4-{6-[(1,1-dioxido-4-thiomorpholinyl)sulfonyl]-3-pyridinyl}-2-(trifluoromethyl)-1H-pyrrolo[2,3-b]pyridine;
N-(2-hydroxyethyl)-5-[2-(trifluoromethyl)-1H-pyrrolo[2,3-b]pyridin-4-yl]-2-pyridinesulfonamide;
N-(2,3-dihydroxypropyl)-5-[2-(trifluoromethyl)-1H-pyrrolo[2,3-b]pyridin-4-yl]-2-pyridinesulfonamide;
6-[2-(1,1-dimethylethyl)-1H-pyrrolo[2,3-b]pyridin-4-yl]-N-(1,1-dioxidotetrahydro-2H-thiopyran-4-yl)-3-pyridinesulfonamide;
6-[2-(1,1-dimethylethyl)-1H-pyrrolo[2,3-b]pyridin-4-yl]-N-(2-hydroxyethyl)-3-pyridinesulfonamide;
2-(1,1-dimethylethyl)-4-{5-[(1,1-dioxido-4-thiomorpholinyl)sulfonyl]-2-pyridinyl}-1H-pyrrolo[2,3-b]pyridine;
6-[2-(1,1-dimethylethyl)-1H-pyrrolo[2,3-b]pyridin-4-yl]-N-(1,1-dioxidotetrahydro-3-thienyl)-3-pyridinesulfonamide;
5-[2-(1,1-dimethylethyl)-1H-pyrrolo[2,3-b]pyridin-4-yl]-N-4-piperidinyl-2-pyridinesulfonamide;
6-[2-(1,1-dimethylethyl)-1H-pyrrolo[2,3-b]pyridin-4-yl]-N-(1,1-dioxidotetrahydro-2H-thiopyran-4-yl)-N-methyl-3-pyridinesulfonamide;
5-[2-(1,1-dimethylethyl)-1H-pyrrolo[2,3-b]pyridin-4-yl]-N-(1,1-dioxidotetrahydro-2H-thiopyran-4-yl)-N-methyl-2-pyridinesulfonamide;
5-[2-(1,1-dimethylethyl)-1H-pyrrolo[2,3-b]pyridin-4-yl]-N-(2-hydroxyethyl)-2-pyridinesulfonamide;
N-(2,3-dihydroxypropyl)-5-[2-(1,1-dimethylethyl)-1H-pyrrolo[2,3-b]pyridin-4-yl]-2-pyridinesulfonamide;
N-(2-hydroxyethyl)-6-[2-(trifluoromethyl)-1H-pyrrolo[2,3-b]pyridin-4-yl]-3-pyridinesulfonamide;
N-4-piperidinyl-6-[2-(trifluoromethyl)-1H-pyrrolo[2,3-b]pyridin-4-yl]-3-pyridinesulfonamide;
N-(1,1-dioxidotetrahydro-3-thienyl)-6-[2-(trifluoromethyl)-1H-pyrrolo[2,3-b]pyridin-4-yl]-3-pyridinesulfonamide;
4-{6-[(1,1-dioxido-4-thiomorpholinyl)sulfonyl]-3-pyridinyl}-2-(trifluoromethyl)-1H-pyrrolo[2,3-b]pyridine;
N-(1,1-dioxidotetrahydro-2H-thiopyran-4-yl)-6-[2-(1-methylethyl)-1H-pyrrolo[2,3-b]pyridin-4-yl]-3-pyridinesulfonamide;
2-(1-methylethyl)-4-[6-(1-pyrrolidinylsulfonyl)-3-pyridinyl]-1H-pyrrolo[2,3-b]pyridine;
N-(2-hydroxyethyl)-6-[2-(1-methylethyl)-1H-pyrrolo[2,3-b]pyridin-4-yl]-3-pyridinesulfonamide;
4-{5-[(1,1-dioxido-4-thiomorpholinyl)sulfonyl]-2-pyridinyl}-2-(1-methylethyl)-1H-pyrrolo[2,3-b]pyridine;
N-(2-aminoethyl)-6-[2-(1-methylethyl)-1H-pyrrolo[2,3-b]pyridin-4-yl]-3-pyridinesulfonamide;
6-[2-(1-methylethyl)-1H-pyrrolo[2,3-b]pyridin-4-yl]-N-4-piperidinyl-3-pyridinesulfonamide;
N-(1,1-dioxidotetrahydro-3-thienyl)-6-[2-(1-methylethyl)-1H-pyrrolo[2,3-b]pyridin-4-yl]-3-pyridinesulfonamide;
N-(1,1-dioxidotetrahydro-2H-thiopyran-4-yl)-5-[2-(1-methylethyl)-1H-pyrrolo[2,3-b]pyridin-4-yl]-2-pyridinesulfonamide;
4-{6-[(1,1-dioxido-4-thiomorpholinyl)sulfonyl]-3-pyridinyl}-2-(1-methylethyl)-1H-pyrrolo[2,3-b]pyridine;
5-[2-(1-methylethyl)-1H-pyrrolo[2,3-b]pyridin-4-yl]-N-4-piperidinyl-2-pyridinesulfonamide;
N-(1,1-dioxidotetrahydro-2H-thiopyran-4-yl)-N-methyl-6-[2-(1-methylethyl)-1H-pyrrolo[2,3-b]pyridin-4-yl]-3-pyridinesulfonamide;
N-(1,1-dioxidotetrahydro-2H-thiopyran-4-yl)-N-methyl-5-[2-(1-methylethyl)-1H-pyrrolo[2,3-b]pyridin-4-yl]-2-pyridinesulfonamide;
N-(2-hydroxyethyl)-5-[2-(1-methylethyl)-1H-pyrrolo[2,3-b]pyridin-4-yl]-2-pyridinesulfonamide;
N-(2-aminoethyl)-6-[2-(1,1-dimethylethyl)-1H-pyrrolo[2,3-b]pyridin-4-yl]-3-pyridinesulfonamide;
6-[2-(1,1-dimethylethyl)-1H-pyrrolo[2,3-b]pyridin-4-yl]-N-4-piperidinyl-3-pyridinesulfonamide;
2-(1,1-dimethylethyl)-4-{6-[(1,1-dioxido-4-thiomorpholinyl)sulfonyl]-3-pyridinyl}-1H-pyrrolo[2,3-b]pyridine;
2-(1,1-dimethylethyl)-4-[6-(1-pyrrolidinylsulfonyl)-3-pyridinyl]-1H-pyrrolo[2,3-b]pyridine;
2-(1,1-dimethylethyl)-4-[6-(1-pyrrolidinylsulfonyl)-3-pyridinyl]-1H-pyrrolo[2,3-b]pyridine hydrochloride;
2-(1,1-dimethylethyl)-4-[6-(1-pyrrolidinylsulfonyl)-3-pyridinyl]-1H-pyrrolo[2,3-b]pyridine sulfate;
N-(2-aminoethyl)-5-[2-(difluoromethyl)-1H-pyrrolo[2,3-b]pyridin-4-yl]-2-thiophenesulfonamide;
5-[2-(difluoromethyl)-1H-pyrrolo[2,3-b]pyridin-4-yl]-N-(1,1-dioxidotetrahydro-3-thienyl)-2-thiophenesulfonamide;
5-[2-(difluoromethyl)-1H-pyrrolo[2,3-b]pyridin-4-yl]-N-(1,1-dioxidotetrahydro-2H-thiopyran-4-yl)-2-thiophenesulfonamide;
N-(2-hydroxy-2-methylpropyl)-5-[2-(trifluoromethyl)-1H-pyrrolo[2,3-b]pyridin-4-yl]-2-thiophenesulfonamide;
N-(2-hydroxyethyl)-5-[2-(trifluoromethyl)-1H-pyrrolo[2,3-b]pyridin-4-yl]-2-thiophenesulfonamide;
N-(tetrahydro-2H-pyran-4-yl)-5-[2-(trifluoromethyl)-1H-pyrrolo[2,3-b]pyridin-4-yl]-2-thiophenesulfonamide;
N-(2,3-dihydroxypropyl)-5-[2-(trifluoromethyl)-1H-pyrrolo[2,3-b]pyridin-4-yl]-2-thiophenesulfonamide;
N-(2-aminoethyl)-5-[2-(trifluoromethyl)-1H-pyrrolo[2,3-b]pyridin-4-yl]-2-thiophenesulfonamide;
N-4-piperidinyl-5-[2-(trifluoromethyl)-1H-pyrrolo[2,3-b]pyridin-4-yl]-2-thiophenesulfonamide;
N-(tetrahydro-2H-thiopyran-4-yl)-6-[2-(trifluoromethyl)-1H-pyrrolo[2,3-b]pyridin-4-yl]-3-pyridinesulfonamide;
N-(2-aminoethyl)-6-[2-(trifluoromethyl)-1H-pyrrolo[2,3-b]pyridin-4-yl]-3-pyridinesulfonamide;
5-(2-cyclopropyl-1H-pyrrolo[2,3-b]pyridin-4-yl)-N-4-piperidinyl-2-thiophenesulfonamide;
5-(2-cyclopropyl-1H-pyrrolo[2,3-b]pyridin-4-yl)-N-(2-hydroxyethyl)-2-thiophenesulfonamide;
5-(2-cyclopropyl-1H-pyrrolo[2,3-b]pyridin-4-yl)-N-(2-hydroxy-2-methylpropyl)-2-thiophenesulfonamide;
N-(2-aminoethyl)-5-(2-cyclopropyl-1H-pyrrolo[2,3-b]pyridin-4-yl)-2-thiophenesulfonamide;
5-(2-cyclopropyl-1H-pyrrolo[2,3-b]pyridin-4-yl)-N-(2,3-dihydroxypropyl)-2-thiophenesulfonamide;
5-(2-cyclopropyl-1H-pyrrolo[2,3-b]pyridin-4-yl)-N-(1,1-dioxidotetrahydro-2H-thiopyran-4-yl)-2-thiophenesulfonamide;
5-(2-cyclopropyl-1H-pyrrolo[2,3-b]pyridin-4-yl)-N-(tetrahydro-2H-pyran-4-yl)-2-thiophenesulfonamide;
N-(2-hydroxy-1,1-dimethylethyl)-5-[2-(trifluoromethyl)-1H-pyrrolo[2,3-b]pyridin-4-yl]-2-thiophenesulfonamide;
N-(2-hydroxy-2-methylpropyl)-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(1R)-2-hydroxy-1-methylethyl]-2-thiophenesulfonamide;
N-[(2S)-2-hydroxypropyl]-5-[2-(trifluoromethyl)-1H-pyrrolo[2,3-b]pyridin-4-yl]-2-thiophenesulfonamide;
N-[(1R)-2-hydroxy-1-methylethyl]-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(2S)-2-hydroxypropyl]-2-thiophenesulfonamide;
N-[(2R)-2-hydroxypropyl]-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(1S)-2-hydroxy-1-methylethyl]-2-thiophenesulfonamide;
N-[(1S)-2-hydroxy-1-methylethyl]-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(2R)-2-hydroxypropyl]-2-thiophenesulfonamide;
N-(2-hydroxy-1,1-dimethylethyl)-5-(2-methyl-1H-pyrrolo[2,3-b]pyridin-4-yl)-2-thiophenesulfonamide;
N-[(1R)-2-hydroxy-1-methylethyl]-5-(2-methyl-1H-pyrrolo[2,3-b]pyridin-4-yl)-2-thiophenesulfonamide;
N-[(2S)-2-hydroxypropyl]-5-(2-methyl-1H-pyrrolo[2,3-b]pyridin-4-yl)-2-thiophenesulfonamide;
N-[(2R)-2-hydroxypropyl]-5-(2-methyl-1H-pyrrolo[2,3-b]pyridin-4-yl)-2-thiophenesulfonamide;
N-(2-hydroxy-2-methylpropyl)-5-(2-methyl-1H-pyrrolo[2,3-b]pyridin-4-yl)-2-thiophenesulfonamide;
5-(2-cyclopropyl-1H-pyrrolo[2,3-b]pyridin-4-yl)-N-(2-hydroxy-1,1-dimethylethyl)-2-thiophenesulfonamide;
N-(2-aminoethyl)-6-[2-(1,1-dimethylethyl)-1H-pyrrolo[2,3-b]pyridin-4-yl]-3-pyridinesulfonamide;
5-(2-cyclopropyl-1H-pyrrolo[2,3-b]pyridin-4-yl)-N-(2-hydroxy-2-methylpropyl)-2-pyridinesulfonamide;
5-(2-cyclopropyl-1H-pyrrolo[2,3-b]pyridin-4-yl)-N-(2-hydroxy-1,1-dimethylethyl)-2-pyridinesulfonamide;
N-(2-hydroxy-1,1-dimethylethyl)-5-[2-(trifluoromethyl)-1H-pyrrolo[2,3-b]pyridin-4-yl]-2-pyridinesulfonamide;
6-(2-cyclopropyl-1H-pyrrolo[2,3-b]pyridin-4-yl)-N-(2-hydroxy-2-methylpropyl)-3-pyridinesulfonamide;
N-(2-hydroxy-2-methylpropyl)-6-(2-methyl-1H-pyrrolo[2,3-b]pyridin-4-yl)-3-pyridinesulfonamide;
6-(2-cyclopropyl-1H-pyrrolo[2,3-b]pyridin-4-yl)-N-(2-hydroxy-1,1-dimethylethyl)-3-pyridinesulfonamide;
N-(2-hydroxy-1,1-dimethylethyl)-6-[2-(trifluoromethyl)-1H-pyrrolo[2,3-b]pyridin-4-yl]-3-pyridinesulfonamide;
N-[(1S)-2-hydroxy-1-methylethyl]-6-[2-(trifluoromethyl)-1H-pyrrolo[2,3-b]pyridin-4-yl]-3-pyridinesulfonamide;
6-(2-cyclopropyl-1H-pyrrolo[2,3-b]pyridin-4-yl)-N-[(2R)-2-hydroxypropyl]-3-pyridinesulfonamide;
N-[(1R)-2-hydroxy-1-methylethyl]-6-[2-(trifluoromethyl)-1H-pyrrolo[2,3-b]pyridin-4-yl]-3-pyridinesulfonamide;
N-[(1S)-2-hydroxy-1-methylethyl]-6-(2-methyl-1H-pyrrolo[2,3-b]pyridin-4-yl)-3-pyridinesulfonamide;
N-[(2S)-2-hydroxypropyl]-6-[2-(trifluoromethyl)-1H-pyrrolo[2,3-b]pyridin-4-yl]-3-pyridinesulfonamide;
6-(2-cyclopropyl-1H-pyrrolo[2,3-b]pyridin-4-yl)-N-[(2S)-2-hydroxypropyl]-3-pyridinesulfonamide;
N-[(2R)-2-hydroxypropyl]-6-[2-(trifluoromethyl)-1H-pyrrolo[2,3-b]pyridin-4-yl]-3-pyridinesulfonamide;
N-[(2R)-2-hydroxypropyl]-6-(2-methyl-1H-pyrrolo[2,3-b]pyridin-4-yl)-3-pyridinesulfonamide;
6-(2-cyclopropyl-1H-pyrrolo[2,3-b]pyridin-4-yl)-N-[(1R)-2-hydroxy-1-methylethyl]-3-pyridinesulfonamide;
N-[(2S)-2-hydroxypropyl]-6-(2-methyl-1H-pyrrolo[2,3-b]pyridin-4-yl)-3-pyridinesulfonamide;
6-(2-cyclopropyl-1H-pyrrolo[2,3-b]pyridin-4-yl)-N-[(1S)-2-hydroxy-1-methylethyl]-3-pyridinesulfonamide;
N-[(1R)-2-hydroxy-1-methylethyl]-6-(2-methyl-1H-pyrrolo[2,3-b]pyridin-4-yl)-3-pyridinesulfonamide;
N-(2-hydroxy-1,1-dimethylethyl)-5-(2-methyl-1H-pyrrolo[2,3-b]pyridin-4-yl)-2-pyridinesulfonamide;
5-(2-cyclopropyl-1H-pyrrolo[2,3-b]pyridin-4-yl)-N-[(2R)-2-hydroxypropyl]-2-pyridinesulfonamide;
N-[(1S)-2-hydroxy-1-methylethyl]-5-(2-methyl-1H-pyrrolo[2,3-b]pyridin-4-yl)-2-pyridinesulfonamide;
5-(2-cyclopropyl-1H-pyrrolo[2,3-b]pyridin-4-yl)-N-[(1R)-2-hydroxy-1-methylethyl]-2-pyridinesulfonamide;
N-[(2R)-2-hydroxypropyl]-5-[2-(trifluoromethyl)-1H-pyrrolo[2,3-b]pyridin-4-yl]-2-pyridinesulfonamide;
N-[(2R)-2-hydroxypropyl]-5-(2-methyl-1H-pyrrolo[2,3-b]pyridin-4-yl)-2-pyridinesulfonamide;
N-[(2S)-2-hydroxypropyl]-5-[2-(trifluoromethyl)-1H-pyrrolo[2,3-b]pyridin-4-yl]-2-pyridinesulfonamide;
5-(2-cyclopropyl-1H-pyrrolo[2,3-b]pyridin-4-yl)-N-[(1S)-2-hydroxy-1-methylethyl]-2-pyridinesulfonamide;
N-[(1R)-2-hydroxy-1-methylethyl]-5-(2-methyl-1H-pyrrolo[2,3-b]pyridin-4-yl)-2-pyridinesulfonamide;
5-(2-cyclopropyl-1H-pyrrolo[2,3-b]pyridin-4-yl)-N-[(2S)-2-hydroxypropyl]-2-pyridinesulfonamide;
N-(2-hydroxy-1,1-dimethylethyl)-6-(2-methyl-1H-pyrrolo[2,3-b]pyridin-4-yl)-3-pyridinesulfonamide;
N-[(1S)-2-hydroxy-1-methylethyl]-5-[2-(trifluoromethyl)-1H-pyrrolo[2,3-b]pyridin-4-yl]-2-pyridinesulfonamide;
N-[(2S)-2-hydroxypropyl]-5-(2-methyl-1H-pyrrolo[2,3-b]pyridin-4-yl)-2-pyridinesulfonamide;
N-[(1R)-2-hydroxy-1-methylethyl]-5-[2-(trifluoromethyl)-1H-pyrrolo[2,3-b]pyridin-4-yl]-2-pyridinesulfonamide;
5-(2-cyclopropyl-1H-pyrrolo[2,3-b]pyridin-4-yl)-N-(2-hydroxy-1,1-dimethylpropyl)-2-pyridinesulfonamide;
5-(2-cyclopropyl-1H-pyrrolo[2,3-b]pyridin-4-yl)-N-(2-hydroxy-1,2-dimethylpropyl)-2-pyridinesulfonamide;
5-(2-cyclopropyl-1H-pyrrolo[2,3-b]pyridin-4-yl)-N-[(1R)-1-(hydroxymethyl)propyl]-2-pyridinesulfonamide;
N-(2-hydroxy-2-methylpropyl)-5-(2-methyl-1H-pyrrolo[2,3-b]pyridin-4-yl)-2-pyridinesulfonamide;
   or a salt thereof.

In another embodiment, the compound of the invention is:
N-(2-hydroxyethyl)-5-(2-methyl-1H-pyrrolo[2,3-b]pyridin-4-yl)-2-thiophenesulfonamide;
N-(1,1-dioxidotetrahydro-2H-thiopyran-4-yl)-5-(2-methyl-1H-pyrrolo[2,3-b]pyridin-4-yl)-2-thiophenesulfonamide;
N-(2-aminoethyl)-5-(2-methyl-1H-pyrrolo[2,3-b]pyridin-4-yl)-2-thiophenesulfonamide;
N-(2-hydroxyethyl)-5-[2-(3-piperidinyl)-1H-pyrrolo[2,3-b]pyridin-4-yl]-2-thiophenesulfonamide;
N-(2-aminoethyl)-5-[2-(3-piperidinyl)-1H-pyrrolo[2,3-b]pyridin-4-yl]-2-thiophenesulfonamide;
N-(1,1-dioxidotetrahydro-3-thienyl)-5-[2-(3-piperidinyl)-1H-pyrrolo[2,3-b]pyridin-4-yl]-2-thiophenesulfonamide;
N-(1,1-dioxidotetrahydro-2H-thiopyran-4-yl)-5-[2-(3-piperidinyl)-1H-pyrrolo[2,3-b]pyridin-4-yl]-2-thiophenesulfonamide;
N-(1,1-dioxidotetrahydro-2H-thiopyran-4-yl)-6-(2-methyl-1H-pyrrolo[2,3-b]pyridin-4-yl)-3-pyridinesulfonamide;
2-methyl-4-[5-(1-pyrrolidinylsulfonyl)-2-pyridinyl]-1H-pyrrolo[2,3-b]pyridine;
N-(1,1-dioxidotetrahydro-2H-thiopyran-4-yl)-5-(2-methyl-1H-pyrrolo[2,3-b]pyridin-4-yl)-2-pyridinesulfonamide;
2-methyl-4-[6-(1-pyrrolidinylsulfonyl)-3-pyridinyl]-1H-pyrrolo[2,3-b]pyridine;
N-(1,1-dioxidotetrahydro-2H-thiopyran-4-yl)-2-methyl-5-[2-(trifluoromethyl)-1H-pyrrolo[2,3-b]pyridin-4-yl]-3-thiophenesulfonamide;
N-(1,1-dioxidotetrahydro-2H-thiopyran-4-yl)-4-methyl-2-[2-(trifluoromethyl)-1H-pyrrolo[2,3-b]pyridin-4-yl]-1,3-thiazole-5-sulfonamide;
N-(1,1-dioxidotetrahydro-2H-thiopyran-4-yl)-2-(2-methyl-1H-pyrrolo[2,3-b]pyridin-4-yl)-5-pyrimidinesulfonamide;
6-[2-(difluoromethyl)-1H-pyrrolo[2,3-b]pyridin-4-yl]-N-(1,1-dioxidotetrahydro-2H-thiopyran-4-yl)-3-pyridinesulfonamide;
6-[2-(difluoromethyl)-1H-pyrrolo[2,3-b]pyridin-4-yl]-N-4-piperidinyl-3-pyridinesulfonamide;
2-(difluoromethyl)-4-{5-[(1,1-dioxido-4-thiomorpholinyl)sulfonyl]-2-pyridinyl}-1H-pyrrolo[2,3-b]pyridine;
2-(difluoromethyl)-4-{6-[(1,1-dioxido-4-thiomorpholinyl)sulfonyl]-3-pyridinyl}-1H-pyrrolo[2,3-b]pyridine;
5-[2-(difluoromethyl)-1H-pyrrolo[2,3-b]pyridin-4-yl]-N-4-piperidinyl-2-pyridinesulfonamide;
6-[2-(difluoromethyl)-1H-pyrrolo[2,3-b]pyridin-4-yl]-N-(1,1-d ioxidotetrahydro-2H-thiopyran-4-yl)-N-methyl-3-pyridinesulfonamide;
5-[2-(difluoromethyl)-1H-pyrrolo[2,3-b]pyridin-4-yl]-N-(1,1-dioxidotetrahydro-2H-thiopyran-4-yl)-N-methyl-2-pyridinesulfonamide;
5-[2-(difluoromethyl)-1H-pyrrolo[2,3-b]pyridin-4-yl]-N-(1,1-dioxidotetrahydro-2H-thiopyran-4-yl)-2-pyridinesulfonamide;
N-(1,1-dioxidotetrahydro-2H-thiopyran-4-yl)-6-(2-methyl-1H-pyrrolo[2,3-b]pyridin-4-yl)-3-pyridinesulfonamide;
N-(2-hydroxyethyl)-6-(2-methyl-1H-pyrrolo[2,3-b]pyridin-4-yl)-3-pyridinesulfonamide;
6-(2-methyl-1H-pyrrolo[2,3-b]pyridin-4-yl)-N-4-piperidinyl-3-pyridinesulfonamide;
5-(2-methyl-1H-pyrrolo[2,3-b]pyridin-4-yl)-N-4-piperidinyl-2-pyridinesulfonamide;
N-(1,1-dioxidotetrahydro-2H-thiopyran-4-yl)-N-methyl-6-(2-methyl-1H-pyrrolo[2,3-b]pyridin-4-yl)-3-pyridinesulfonamide;
N-(1,1-dioxidotetrahydro-2H-thiopyran-4-yl)-N-methyl-5-(2-methyl-1H-pyrrolo[2,3-b]pyridin-4-yl)-2-pyridinesulfonamide;
N-(2,3-dihydroxypropyl)-5-(2-methyl-1H-pyrrolo[2,3-b]pyridin-4-yl)-2-pyridinesulfonamide;
N-(2,3-dihydroxypropyl)-5-(2-methyl-1H-pyrrolo[2,3-b]pyridin-4-yl)-2-pyridinesulfonamide;
N-(1,1-dioxidotetrahydro-3-thienyl)-5-(2-methyl-1H-pyrrolo[2,3-b]pyridin-4-yl)-2-thiophenesulfonamide;
2-(1,1-dimethylethyl)-4-[6-(1-pyrrolidinylsulfonyl)-3-pyridinyl]-1H-pyrrolo[2,3-b]pyridine;
N-(2-aminoethyl)-6-[2-(trifluoromethyl)-1H-pyrrolo[2,3-b]pyridin-4-yl]-3-pyridinesulfonamide;
N-(2-aminoethyl)-6-[2-(trifluoromethyl)-1H-pyrrolo[2,3-b]pyridin-4-yl]-3-pyridinesulfonamide trifluoroacetate;
N-(1,1-dioxidotetrahydro-2H-thiopyran-4-yl)-6-[2-(trifluoromethyl)-1H-pyrrolo[2,3-b]pyridin-4-yl]-3-pyridinesulfonamide;
N-4-piperidinyl-5-[2-(trifluoromethyl)-1H-pyrrolo[2,3-b]pyridin-4-yl]-2-pyridinesulfonamide;
N-(1,1-dioxidotetrahydro-2H-thiopyran-4-yl)-6-[2-(trifluoromethyl)-1H-pyrrolo[2,3-b]pyridin-4-yl]-3-pyridinesulfonamide;
4-{6-[(1,1-dioxido-4-thiomorpholinyl)sulfonyl]-3-pyridinyl}-2-(trifluoromethyl)-1H-pyrrolo[2,3-b]pyridine;
N-(2-hydroxyethyl)-5-[2-(trifluoromethyl)-1H-pyrrolo[2,3-b]pyridin-4-yl]-2-pyridinesulfonamide;
N-(2,3-dihydroxypropyl)-5-[2-(trifluoromethyl)-1H-pyrrolo[2,3-b]pyridin-4-yl]-2-pyridinesulfonamide;
6-[2-(1,1-dimethylethyl)-1H-pyrrolo[2,3-b]pyridin-4-yl]-N-(1,1-dioxidotetrahydro-2H-thiopyran-4-yl)-3-pyridinesulfonamide;
6-[2-(1,1-dimethylethyl)-1H-pyrrolo[2,3-b]pyridin-4-yl]-N-(2-hydroxyethyl)-3-pyridinesulfonamide;
2-(1,1-dimethylethyl)-4-{5-[(1,1-dioxido-4-thiomorpholinyl)sulfonyl]-2-pyridinyl}-1H-pyrrolo[2,3-b]pyridine;
6-[2-(1,1-dimethylethyl)-1H-pyrrolo[2,3-b]pyridin-4-yl]-N-(1,1-dioxidotetrahydro-3-thienyl)-3-pyridinesulfonamide;
5-[2-(1,1-dimethylethyl)-1H-pyrrolo[2,3-b]pyridin-4-yl]-N-4-piperidinyl-2-pyridinesulfonamide;
6-[2-(1,1-dimethylethyl)-1H-pyrrolo[2,3-b]pyridin-4-yl]-N-(1,1-dioxidotetrahydro-2H-thiopyran-4-yl)-N-methyl-3-pyridinesulfonamide;
5-[2-(1,1-dimethylethyl)-1H-pyrrolo[2,3-b]pyridin-4-yl]-N-(1,1-dioxidotetrahydro-2H-thiopyran-4-yl)-N-methyl-2-pyridinesulfonamide;
5-[2-(1,1-dimethylethyl)-1H-pyrrolo[2,3-b]pyridin-4-yl]-N-(2-hydroxyethyl)-2-pyridinesulfonamide;
N-(2,3-dihydroxypropyl)-5-[2-(1,1-dimethylethyl)-1H-pyrrolo[2,3-b]pyridin-4-yl]-2-pyridinesulfonamide;
N-(2-hydroxyethyl)-6-[2-(trifluoromethyl)-1H-pyrrolo[2,3-b]pyridin-4-yl]-3-pyridinesulfonamide;
N-4-piperidinyl-6-[2-(trifluoromethyl)-1H-pyrrolo[2,3-b]pyridin-4-yl]-3-pyridinesulfonamide;
N-(1,1-dioxidotetrahydro-3-thienyl)-6-[2-(trifluoromethyl)-1H-pyrrolo[2,3-b]pyridin-4-yl]-3-pyridinesulfonamide;
4-{6-[(1,1-dioxido-4-thiomorpholinyl)sulfonyl]-3-pyridinyl}-2-(trifluoromethyl)-1H-pyrrolo[2,3-b]pyridine;
N-(1,1-dioxidotetrahydro-2H-thiopyran-4-yl)-6-[2-(1-methylethyl)-1H-pyrrolo[2,3-b]pyridin-4-yl]-3-pyridinesulfonamide;
2-(1-methylethyl)-4-[6-(1-pyrrolidinylsulfonyl)-3-pyridinyl]-1H-pyrrolo[2,3-b]pyridine;
N-(2-hydroxyethyl)-6-[2-(1-methylethyl)-1H-pyrrolo[2,3-b]pyridin-4-yl]-3-pyridinesulfonamide;
4-{5-[(1,1-dioxido-4-thiomorpholinyl)sulfonyl]-2-pyridinyl}-2-(1-methylethyl)-1H-pyrrolo[2,3-b]pyridine;
N-(2-aminoethyl)-6-[2-(1-methylethyl)-1H-pyrrolo[2,3-b]pyridin-4-yl]-3-pyridinesulfonamide;
6-[2-(1-methylethyl)-1H-pyrrolo[2,3-b]pyridin-4-yl]-N-4-piperidinyl-3-pyridinesulfonamide;
N-(1,1-dioxidotetrahydro-3-thienyl)-6-[2-(1-methylethyl)-1H-pyrrolo[2,3-b]pyridin-4-yl]-3-pyridinesulfonamide;
N-(1,1-dioxidotetrahydro-2H-thiopyran-4-yl)-5-[2-(1-methylethyl)-1H-pyrrolo[2,3-b]pyridin-4-yl]-2-pyridinesulfonamide;
4-{6-[(1,1-dioxido-4-thiomorpholinyl)sulfonyl]-3-pyridinyl}-2-(1-methylethyl)-1H-pyrrolo[2,3-b]pyridine;
5-[2-(1-methylethyl)-1H-pyrrolo[2,3-b]pyridin-4-yl]-N-4-piperidinyl-2-pyridinesulfonamide;
N-(1,1-dioxidotetrahydro-2H-thiopyran-4-yl)-N-methyl-6-[2-(1-methylethyl)-1H-pyrrolo[2,3-b]pyridin-4-yl]-3-pyridinesulfonamide;
N-(1,1-dioxidotetrahydro-2H-thiopyran-4-yl)-N-methyl-5-[2-(1-methylethyl)-1H-pyrrolo[2,3-b]pyridin-4-yl]-2-pyridinesulfonamide;
N-(2-hydroxyethyl)-5-[2-(1-methylethyl)-1H-pyrrolo[2,3-b]pyridin-4-yl]-2-pyridinesulfonamide;
N-(2-aminoethyl)-6-[2-(1,1-dimethylethyl)-1H-pyrrolo[2,3-b]pyridin-4-yl]-3-pyridinesulfonamide;
6-[2-(1,1-dimethylethyl)-1H-pyrrolo[2,3-b]pyridin-4-yl]-N-4-piperidinyl-3-pyridinesulfonamide;
2-(1,1-dimethylethyl)-4-{6-[(1,1-dioxido-4-thiomorpholinyl)sulfonyl]-3-pyridinyl}-1H-pyrrolo[2,3-b]pyridine;
2-(1,1-dimethylethyl)-4-[6-(1-pyrrolidinylsulfonyl)-3-pyridinyl]-1H-pyrrolo[2,3-b]pyridine;
2-(1,1-dimethylethyl)-4-[6-(1-pyrrolidinylsulfonyl)-3-pyridinyl]-1H-pyrrolo[2,3-b]pyridine hydrochloride;
2-(1,1-dimethylethyl)-4-[6-(1-pyrrolidinylsulfonyl)-3-pyridinyl]-1H-pyrrolo[2,3-b]pyridine sulfate;
N-(2-aminoethyl)-5-[2-(difluoromethyl)-1H-pyrrolo[2,3-b]pyridin-4-yl]-2-thiophenesulfonamide;
5-[2-(difluoromethyl)-1H-pyrrolo[2,3-b]pyridin-4-yl]-N-(1,1-dioxidotetrahydro-3-thienyl)-2-thiophenesulfonamide;
5-[2-(difluoromethyl)-1H-pyrrolo[2,3-b]pyridin-4-yl]-N-(1,1-dioxidotetrahydro-2H-thiopyran-4-yl)-2-thiophenesulfonamide;
N-(2-hydroxy-2-methylpropyl)-5-[2-(trifluoromethyl)-1H-pyrrolo[2,3-b]pyridin-4-yl]-2-thiophenesulfonamide;
N-(2-hydroxyethyl)-5-[2-(trifluoromethyl)-1H-pyrrolo[2,3-b]pyridin-4-yl]-2-thiophenesulfonamide;
N-(tetrahydro-2H-pyran-4-yl)-5-[2-(trifluoromethyl)-1H-pyrrolo[2,3-b]pyridin-4-yl]-2-thiophenesulfonamide;
N-(2,3-dihydroxypropyl)-5-[2-(trifluoromethyl)-1H-pyrrolo[2,3-b]pyridin-4-yl]-2-thiophenesulfonamide;
N-(2-aminoethyl)-5-[2-(trifluoromethyl)-1H-pyrrolo[2,3-b]pyridin-4-yl]-2-thiophenesulfonamide;
N-4-piperidinyl-5-[2-(trifluoromethyl)-1H-pyrrolo[2,3-b]pyridin-4-yl]-2-thiophenesulfonamide;
N-(tetrahydro-2H-thiopyran-4-yl)-6-[2-(trifluoromethyl)-1H-pyrrolo[2,3-b]pyridin-4-yl]-3-pyridinesulfonamide;
N-(2-aminoethyl)-6-[2-(trifluoromethyl)-1H-pyrrolo[2,3-b]pyridin-4-yl]-3-pyridinesulfonamide;
5-(2-cyclopropyl-1H-pyrrolo[2,3-b]pyridin-4-yl)-N-4-piperidinyl-2-thiophenesulfonamide;
5-(2-cyclopropyl-1H-pyrrolo[2,3-b]pyridin-4-yl)-N-(2-hydroxyethyl)-2-thiophenesulfonamide;
5-(2-cyclopropyl-1H-pyrrolo[2,3-b]pyridin-4-yl)-N-(2-hydroxy-2-methylpropyl)-2-thiophenesulfonamide;
N-(2-aminoethyl)-5-(2-cyclopropyl-1H-pyrrolo[2,3-b]pyridin-4-yl)-2-thiophenesulfonamide;
5-(2-cyclopropyl-1H-pyrrolo[2,3-b]pyridin-4-yl)-N-(2,3-dihydroxypropyl)-2-thiophenesulfonamide;
5-(2-cyclopropyl-1H-pyrrolo[2,3-b]pyridin-4-yl)-N-(1,1-dioxidotetrahydro-2H-thiopyran-4-yl)-2-thiophenesulfonamide;
5-(2-cyclopropyl-1H-pyrrolo[2,3-b]pyridin-4-yl)-N-(tetrahydro-2H-pyran-4-yl)-2-thiophenesulfonamide;
N-(2-hydroxy-1,1-dimethylethyl)-5-[2-(trifluoromethyl)-1H-pyrrolo[2,3-b]pyridin-4-yl]-2-thiophenesulfonamide;
N-(2-hydroxy-2-methylpropyl)-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(1R)-2-hydroxy-1-methylethyl]-2-thiophenesulfonamide;
N-[(2S)-2-hydroxypropyl]-5-[2-(trifluoromethyl)-1H-pyrrolo[2,3-b]pyridin-4-yl]-2-thiophenesulfonamide;
N-[(1R)-2-hydroxy-1-methylethyl]-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(2S)-2-hydroxypropyl]-2-thiophenesulfonamide;
N-[(2R)-2-hydroxypropyl]-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(1S)-2-hydroxy-1-methylethyl]-2-thiophenesulfonamide;
N-[(1S)-2-hydroxy-1-methylethyl]-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(2R)-2-hydroxypropyl]-2-thiophenesulfonamide;
N-(2-hydroxy-1,1-dimethylethyl)-5-(2-methyl-1H-pyrrolo[2,3-b]pyridin-4-yl)-2-thiophenesulfonamide;
N-[(1R)-2-hydroxy-1-methylethyl]-5-(2-methyl-1H-pyrrolo[2,3-b]pyridin-4-yl)-2-thiophenesulfonamide;
N-[(2S)-2-hydroxypropyl]-5-(2-methyl-1H-pyrrolo[2,3-b]pyridin-4-yl)-2-thiophenesulfonamide;
N-[(2R)-2-hydroxypropyl]-5-(2-methyl-1H-pyrrolo[2,3-b]pyridin-4-yl)-2-thiophenesulfonamide;
N-(2-hydroxy-2-methylpropyl)-5-(2-methyl-1H-pyrrolo[2,3-b]pyridin-4-yl)-2-thiophenesulfonamide;
5-(2-cyclopropyl-1H-pyrrolo[2,3-b]pyridin-4-yl)-N-(2-hydroxy-1,1-dimethylethyl)-2-thiophenesulfonamide;
N-(2-aminoethyl)-6-[2-(1,1-dimethylethyl)-1H-pyrrolo[2,3-b]pyridin-4-yl]-3-pyridinesulfonamide;
5-(2-cyclopropyl-1H-pyrrolo[2,3-b]pyridin-4-yl)-N-(2-hydroxy-2-methylpropyl)-2-pyridinesulfonamide;
5-(2-cyclopropyl-1H-pyrrolo[2,3-b]pyridin-4-yl)-N-(2-hydroxy-1,1-dimethylethyl)-2-pyridinesulfonamide;
N-(2-hydroxy-1,1-dimethylethyl)-5-[2-(trifluoromethyl)-1H-pyrrolo[2,3-b]pyridin-4-yl]-2-pyridinesulfonamide;
6-(2-cyclopropyl-1H-pyrrolo[2,3-b]pyridin-4-yl)-N-(2-hydroxy-2-methylpropyl)-3-pyridinesulfonamide;
N-(2-hydroxy-2-methylpropyl)-6-(2-methyl-1H-pyrrolo[2,3-b]pyridin-4-yl)-3-pyridinesulfonamide;
6-(2-cyclopropyl-1H-pyrrolo[2,3-b]pyridin-4-yl)-N-(2-hydroxy-1,1-dimethylethyl)-3-pyridinesulfonamide;
N-(2-hydroxy-1,1-dimethylethyl)-6-[2-(trifluoromethyl)-1H-pyrrolo[2,3-b]pyridin-4-yl]-3-pyridinesulfonamide;
N-[(1S)-2-hydroxy-1-methylethyl]-6-[2-(trifluoromethyl)-1H-pyrrolo[2,3-b]pyridin-4-yl]-3-pyridinesulfonamide;
6-(2-cyclopropyl-1H-pyrrolo[2,3-b]pyridin-4-yl)-N-[(2R)-2-hydroxypropyl]-3-pyridinesulfonamide;
N-[(1R)-2-hydroxy-1-methylethyl]-6-[2-(trifluoromethyl)-1H-pyrrolo[2,3-b]pyridin-4-yl]-3-pyridinesulfonamide;
N-[(1S)-2-hydroxy-1-methylethyl]-6-(2-methyl-1H-pyrrolo[2,3-b]pyridin-4-yl)-3-pyridinesulfonamide;
N-[(2S)-2-hydroxypropyl]-6-[2-(trifluoromethyl)-1H-pyrrolo[2,3-b]pyridin-4-yl]-3-pyridinesulfonamide;
6-(2-cyclopropyl-1H-pyrrolo[2,3-b]pyridin-4-yl)-N-[(2S)-2-hydroxypropyl]-3-pyridinesulfonamide;
N-[(2R)-2-hydroxypropyl]-6-[2-(trifluoromethyl)-1H-pyrrolo[2,3-b]pyridin-4-yl]-3-pyridinesulfonamide;
N-[(2R)-2-hydroxypropyl]-6-(2-methyl-1H-pyrrolo[2,3-b]pyridin-4-yl)-3-pyridinesulfonamide;
6-(2-cyclopropyl-1H-pyrrolo[2,3-b]pyridin-4-yl)-N-[(1R)-2-hydroxy-1-methylethyl]-3-pyridinesulfonamide;
N-[(2S)-2-hydroxypropyl]-6-(2-methyl-1H-pyrrolo[2,3-b]pyridin-4-yl)-3-pyridinesulfonamide;
6-(2-cyclopropyl-1H-pyrrolo[2,3-b]pyridin-4-yl)-N-[(1S)-2-hydroxy-1-methylethyl]-3-pyridinesulfonamide;
N-[(1R)-2-hydroxy-1-methylethyl]-6-(2-methyl-1H-pyrrolo[2,3-b]pyridin-4-yl)-3-pyridinesulfonamide;
N-(2-hydroxy-1,1-dimethylethyl)-5-(2-methyl-1H-pyrrolo[2,3-b]pyridin-4-yl)-2-pyridinesulfonamide;
5-(2-cyclopropyl-1H-pyrrolo[2,3-b]pyridin-4-yl)-N-[(2R)-2-hydroxypropyl]-2-pyridinesulfonamide;
N-[(1S)-2-hydroxy-1-methylethyl]-5-(2-methyl-1H-pyrrolo[2,3-b]pyridin-4-yl)-2-pyridinesulfonamide;
5-(2-cyclopropyl-1H-pyrrolo[2,3-b]pyridin-4-yl)-N-[(1R)-2-hydroxy-1-methylethyl]-2-pyridinesulfonamide;
N-[(2R)-2-hydroxypropyl]-5-[2-(trifluoromethyl)-1H-pyrrolo[2,3-b]pyridin-4-yl]-2-pyridinesulfonamide;
N-[(2R)-2-hydroxypropyl]-5-(2-methyl-1H-pyrrolo[2,3-b]pyridin-4-yl)-2-pyridinesulfonamide;
N-[(2S)-2-hydroxypropyl]-5-[2-(trifluoromethyl)-1H-pyrrolo[2,3-b]pyridin-4-yl]-2-pyridinesulfonamide;
5-(2-cyclopropyl-1H-pyrrolo[2,3-b]pyridin-4-yl)-N-[(1S)-2-hydroxy-1-methylethyl]-2-pyridinesulfonamide;
N-[(1R)-2-hydroxy-1-methylethyl]-5-(2-methyl-1H-pyrrolo[2,3-b]pyridin-4-yl)-2-pyridinesulfonamide;
5-(2-cyclopropyl-1H-pyrrolo[2,3-b]pyridin-4-yl)-N-[(2S)-2-hydroxypropyl]-2-pyridinesulfonamide;
N-(2-hydroxy-1,1-dimethylethyl)-6-(2-methyl-1H-pyrrolo[2,3-b]pyridin-4-yl)-3-pyridinesulfonamide;
N-[(1S)-2-hydroxy-1-methylethyl]-5-[2-(trifluoromethyl)-1H-pyrrolo[2,3-b]pyridin-4-yl]-2-pyridinesulfonamide;
N-[(2S)-2-hydroxypropyl]-5-(2-methyl-1H-pyrrolo[2,3-b]pyridin-4-yl)-2-pyridinesulfonamide;
N-[(1R)-2-hydroxy-1-methylethyl]-5-[2-(trifluoromethyl)-1H-pyrrolo[2,3-b]pyridin-4-yl]-2-pyridinesulfonamide; or a salt thereof.

In another embodiment, the compound of the invention is:
N-(2-hydroxyethyl)-5-(2-methyl-1H-pyrrolo[2,3-b]pyridin-4-yl)-2-thiophenesulfonamide;
N-(1,1-dioxidotetrahydro-2H-thiopyran-4-yl)-5-(2-methyl-1H-pyrrolo[2,3-b]pyridin-4-yl)-2-thiophenesulfonamide;
N-(2-aminoethyl)-5-(2-methyl-1H-pyrrolo[2,3-b]pyridin-4-yl)-2-thiophenesulfonamide;
N-(2-hydroxyethyl)-5-[2-(3-piperidinyl)-1H-pyrrolo[2,3-b]pyridin-4-yl]-2-thiophenesulfonamide;
N-(2-aminoethyl)-5-[2-(3-piperidinyl)-1H-pyrrolo[2,3-b]pyridin-4-yl]-2-thiophenesulfonamide;
N-(1,1-dioxidotetrahydro-3-thienyl)-5-[2-(3-piperidinyl)-1H-pyrrolo[2,3-b]pyridin-4-yl]-2-thiophenesulfonamide;
N-(1,1-dioxidotetrahydro-2H-thiopyran-4-yl)-5-[2-(3-piperidinyl)-1H-pyrrolo[2,3-b]pyridin-4-yl]-2-thiophenesulfonamide;
N-(1,1-dioxidotetrahydro-2H-thiopyran-4-yl)-6-(2-methyl-1H-pyrrolo[2,3-b]pyridin-4-yl)-3-pyridinesulfonamide;
2-Methyl-4-[5-(1-pyrrolidinylsulfonyl)-2-pyridinyl]-1H-pyrrolo[2,3-b]pyridine;
N-(1,1-dioxidotetrahydro-2H-thiopyran-4-yl)-5-(2-methyl-1H-pyrrolo[2,3-b]pyridin-4-yl)-2-pyridinesulfonamide;
2-methyl-4-[6-(1-pyrrolidinylsulfonyl)-3-pyridinyl]-1H-pyrrolo[2,3-b]pyridine;
N-(1,1-dioxidotetrahydro-2H-thiopyran-4-yl)-2-methyl-5-[2-(trifluoromethyl)-1H-pyrrolo[2,3-b]pyridin-4-yl]-3-thiophenesulfonamide;
N-(1,1-dioxidotetrahydro-2H-thiopyran-4-yl)-4-methyl-2-[2-(trifluoromethyl)-1H-pyrrolo[2,3-b]pyridin-4-yl]-1,3-thiazole-5-sulfonamide;
N-(1,1-dioxidotetrahydro-2H-thiopyran-4-yl)-2-(2-methyl-1H-pyrrolo[2,3-b]pyridin-4-yl)-5-pyrimidinesulfonamide;
6-[2-(difluoromethyl)-1H-pyrrolo[2,3-b]pyridin-4-yl]-N-(1,1-dioxidotetrahydro-2H-thiopyran-4-yl)-3-pyridinesulfonamide;
6-[2-(difluoromethyl)-1H-pyrrolo[2,3-b]pyridin-4-yl]-N-4-piperidinyl-3-pyridinesulfonamide;
2-(difluoromethyl)-4-{5-[(1,1-dioxido-4-thiomorpholinyl)sulfonyl]-2-pyridinyl}-1H-pyrrolo[2,3-b]pyridine;
2-(difluoromethyl)-4-{6-[(1,1-dioxido-4-thiomorpholinyl)sulfonyl]-3-pyridinyl}-1H-pyrrolo[2,3-b]pyridine;
5-[2-(difluoromethyl)-1H-pyrrolo[2,3-b]pyridin-4-yl]-N-4-piperidinyl-2-pyridinesulfonamide;
6-[2-(difluoromethyl)-1H-pyrrolo[2,3-b]pyridin-4-yl]-N-(1,1-dioxidotetrahydro-2H-thiopyran-4-yl)-N-methyl-3-pyridinesulfonamide;
5-[2-(difluoromethyl)-1H-pyrrolo[2,3-b]pyridin-4-yl]-N-(1,1-dioxidotetrahydro-2H-thiopyran-4-yl)-N-methyl-2-pyridinesulfonamide;
5-[2-(difluoromethyl)-1H-pyrrolo[2,3-b]pyridin-4-yl]-N-(1,1-dioxidotetrahydro-2H-thiopyran-4-yl)-2-pyridinesulfonamide;
N-(1,1-dioxidotetrahydro-2H-thiopyran-4-yl)-6-(2-methyl-1H-pyrrolo[2,3-b]pyridin-4-yl)-3-pyridinesulfonamide;
N-(2-hydroxyethyl)-6-(2-methyl-1H-pyrrolo[2,3-b]pyridin-4-yl)-3-pyridinesulfonamide;
6-(2-methyl-1H-pyrrolo[2,3-b]pyridin-4-yl)-N-4-piperidinyl-3-pyridinesulfonamide;
5-(2-methyl-1H-pyrrolo[2,3-b]pyridin-4-yl)-N-4-piperidinyl-2-pyridinesulfonamide;
N-(1,1-dioxidotetrahydro-2H-thiopyran-4-yl)-N-methyl-6-(2-methyl-1H-pyrrolo[2,3-b]pyridin-4-yl)-3-pyridinesulfonamide;
N-(1,1-dioxidotetrahydro-2H-thiopyran-4-yl)-N-methyl-5-(2-methyl-1H-pyrrolo[2,3-b]pyridin-4-yl)-2-pyridinesulfonamide;
N-(2,3-dihydroxypropyl)-5-(2-methyl-1H-pyrrolo[2,3-b]pyridin-4-yl)-2-pyridinesulfonamide;
N-(2,3-dihydroxypropyl)-5-(2-methyl-1H-pyrrolo[2,3-b]pyridin-4-yl)-2-pyridinesulfonamide;
N-(1,1-dioxidotetrahydro-3-thienyl)-5-(2-methyl-1H-pyrrolo[2,3-b]pyridin-4-yl)-2-thiophenesulfonamide;
2-(1,1-dimethylethyl)-4-[6-(1-pyrrolidinylsulfonyl)-3-pyridinyl]-1H-pyrrolo[2,3-b]pyridine;
N-(2-aminoethyl)-6-[2-(trifluoromethyl)-1H-pyrrolo[2,3-b]pyridin-4-yl]-3-pyridinesulfonamide;
N-(2-aminoethyl)-6-[2-(trifluoromethyl)-1H-pyrrolo[2,3-b]pyridin-4-yl]-3-pyridinesulfonamide trifluoroacetate;
N-(1,1-dioxidotetrahydro-2H-thiopyran-4-yl)-6-[2-(trifluoromethyl)-1H-pyrrolo[2,3-b]pyridin-4-yl]-3-pyridinesulfonamide;
N-4-piperidinyl-5-[2-(trifluoromethyl)-1H-pyrrolo[2,3-b]pyridin-4-yl]-2-pyridinesulfonamide;
N-(1,1-dioxidotetrahydro-2H-thiopyran-4-yl)-6-[2-(trifluoromethyl)-1H-pyrrolo[2,3-b]pyridin-4-yl]-3-pyridinesulfonamide;
4-{6-[(1,1-d ioxido-4-thiomorpholinyl)sulfonyl]-3-pyridinyl}-2-(trifluoromethyl)-1H-pyrrolo[2,3-b]pyridine;
N-(2-hydroxyethyl)-5-[2-(trifluoromethyl)-1H-pyrrolo[2,3-b]pyridin-4-yl]-2-pyridinesulfonamide;
N-(2,3-dihydroxypropyl)-5-[2-(trifluoromethyl)-1H-pyrrolo[2,3-b]pyridin-4-yl]-2-pyridinesulfonamide;
6-[2-(1,1-dimethylethyl)-1H-pyrrolo[2,3-b]pyridin-4-yl]-N-(1,1-dioxidotetrahydro-2H-thiopyran-4-yl)-3-pyridinesulfonamide;
6-[2-(1,1-dimethylethyl)-1H-pyrrolo[2,3-b]pyridin-4-yl]-N-(2-hydroxyethyl)-3-pyridinesulfonamide;
2-(1,1-dimethylethyl)-4-{5-[(1,1-dioxido-4-thiomorpholinyl)sulfonyl]-2-pyridinyl}-1H-pyrrolo[2,3-b]pyridine;
6-[2-(1,1-dimethylethyl)-1H-pyrrolo[2,3-b]pyridin-4-yl]-N-(1,1-dioxidotetrahydro-3-thienyl)-3-pyridinesulfonamide;
5-[2-(1,1-dimethylethyl)-1H-pyrrolo[2,3-b]pyridin-4-yl]-N-4-piperidinyl-2-pyridinesulfonamide;
6-[2-(1,1-dimethylethyl)-1H-pyrrolo[2,3-b]pyridin-4-yl]-N-(1,1-dioxidotetrahydro-2H-thiopyran-4-yl)-N-methyl-3-pyridinesulfonamide;
5-[2-(1,1-dimethylethyl)-1H-pyrrolo[2,3-b]pyridin-4-yl]-N-(1,1-dioxidotetrahydro-2H-thiopyran-4-yl)-N-methyl-2-pyridinesulfonamide;
5-[2-(1,1-dimethylethyl)-1H-pyrrolo[2,3-b]pyridin-4-yl]-N-(2-hydroxyethyl)-2-pyridinesulfonamide;
N-(2,3-dihydroxypropyl)-5-[2-(1,1-dimethylethyl)-1H-pyrrolo[2,3-b]pyridin-4-yl]-2-pyridinesulfonamide;
N-(2-hydroxyethyl)-6-[2-(trifluoromethyl)-1H-pyrrolo[2,3-b]pyridin-4-yl]-3-pyridinesulfonamide;
N-4-piperidinyl-6-[2-(trifluoromethyl)-1H-pyrrolo[2,3-b]pyridin-4-yl]-3-pyridinesulfonamide;
N-(1,1-dioxidotetrahydro-3-thienyl)-6-[2-(trifluoromethyl)-1H-pyrrolo[2,3-b]pyridin-4-yl]-3-pyridinesulfonamide;
4-{6-[(1,1-d ioxido-4-thiomorpholinyl)sulfonyl]-3-pyridinyl}-2-(trifluoromethyl)-1H-pyrrolo[2,3-b]pyridine;
N-(1,1-dioxidotetrahydro-2H-thiopyran-4-yl)-6-[2-(1-methylethyl)-1H-pyrrolo[2,3-b]pyridin-4-yl]-3-pyridinesulfonamide;
2-(1-methylethyl)-4-[6-(1-pyrrolidinylsulfonyl)-3-pyridinyl]-1H-pyrrolo[2,3-b]pyridine;
N-(2-hydroxyethyl)-6-[2-(1-methylethyl)-1H-pyrrolo[2,3-b]pyridin-4-yl]-3-pyridinesulfonamide;
4-{5-[(1,1-dioxido-4-thiomorpholinyl)sulfonyl]-2-pyridinyl}-2-(1-methylethyl)-1H-pyrrolo[2,3-b]pyridine;
N-(2-aminoethyl)-6-[2-(1-methylethyl)-1H-pyrrolo[2,3-b]pyridin-4-yl]-3-pyridinesulfonamide;
6-[2-(1-methylethyl)-1H-pyrrolo[2,3-b]pyridin-4-yl]-N-4-piperidinyl-3-pyridinesulfonamide;
N-(1,1-dioxidotetrahydro-3-thienyl)-6-[2-(1-methylethyl)-1H-pyrrolo[2,3-b]pyridin-4-yl]-3-pyridinesulfonamide;
N-(1,1-dioxidotetrahydro-2H-thiopyran-4-yl)-5-[2-(1-methylethyl)-1H-pyrrolo[2,3-b]pyridin-4-yl]-2-pyridinesulfonamide;
4-{6-[(1,1-dioxido-4-thiomorpholinyl)sulfonyl]-3-pyridinyl}-2-(1-methylethyl)-1H-pyrrolo[2,3-b]pyridine;
5-[2-(1-methylethyl)-1H-pyrrolo[2,3-b]pyridin-4-yl]-N-4-piperidinyl-2-pyridinesulfonamide;
N-(1,1-dioxidotetrahydro-2H-thiopyran-4-yl)-N-methyl-6-[2-(1-methylethyl)-1H-pyrrolo[2,3-b]pyridin-4-yl]-3-pyridinesulfonamide;
N-(1,1-dioxidotetrahydro-2H-thiopyran-4-yl)-N-methyl-5-[2-(1-methylethyl)-1H-pyrrolo[2,3-b]pyridin-4-yl]-2-pyridinesulfonamide;
N-(2-hydroxyethyl)-5-[2-(1-methylethyl)-1H-pyrrolo[2,3-b]pyridin-4-yl]-2-pyridinesulfonamide;
N-(2-aminoethyl)-6-[2-(1,1-dimethylethyl)-1H-pyrrolo[2,3-b]pyridin-4-yl]-3-pyridinesulfonamide;
6-[2-(1,1-dimethylethyl)-1H-pyrrolo[2,3-b]pyridin-4-yl]-N-4-piperidinyl-3-pyridinesulfonamide;
2-(1,1-dimethylethyl)-4-{6-[(1,1-dioxido-4-thiomorpholinyl)sulfonyl]-3-pyridinyl}-1H-pyrrolo[2,3-b]pyridine;
2-(1,1-dimethylethyl)-4-[6-(1-pyrrolidinylsulfonyl)-3-pyridinyl]-1H-pyrrolo[2,3-b]pyridine;
2-(1,1-dimethylethyl)-4-[6-(1-pyrrolidinylsulfonyl)-3-pyridinyl]-1H-pyrrolo[2,3-b]pyridine hydrochloride;
2-(1,1-dimethylethyl)-4-[6-(1-pyrrolidinylsulfonyl)-3-pyridinyl]-1H-pyrrolo[2,3-b]pyridine sulfate;
N-(2-aminoethyl)-5-[2-(difluoromethyl)-1H-pyrrolo[2,3-b]pyridin-4-yl]-2-thiophenesulfonamide;
5-[2-(difluoromethyl)-1H-pyrrolo[2,3-b]pyridin-4-yl]-N-(1,1-dioxidotetrahydro-3-thienyl)-2-thiophenesulfonamide;
5-[2-(difluoromethyl)-1H-pyrrolo[2,3-b]pyridin-4-yl]-N-(1,1-dioxidotetrahydro-2H-thiopyran-4-yl)-2-thiophenesulfonamide;
N-(2-hydroxy-2-methylpropyl)-5-[2-(trifluoromethyl)-1H-pyrrolo[2,3-b]pyridin-4-yl]-2-thiophenesulfonamide;
N-(2-hydroxyethyl)-5-[2-(trifluoromethyl)-1H-pyrrolo[2,3-b]pyridin-4-yl]-2-thiophenesulfonamide;
N-(tetrahydro-2H-pyran-4-yl)-5-[2-(trifluoromethyl)-1H-pyrrolo[2,3-b]pyridin-4-yl]-2-thiophenesulfonamide;
N-(2,3-dihydroxypropyl)-5-[2-(trifluoromethyl)-1H-pyrrolo[2,3-b]pyridin-4-yl]-2-thiophenesulfonamide;
N-(2-aminoethyl)-5-[2-(trifluoromethyl)-1H-pyrrolo[2,3-b]pyridin-4-yl]-2-thiophenesulfonamide;
N-4-piperidinyl-5-[2-(trifluoromethyl)-1H-pyrrolo[2,3-b]pyridin-4-yl]-2-thiophenesulfonamide;
N-(tetrahydro-2H-thiopyran-4-yl)-6-[2-(trifluoromethyl)-1H-pyrrolo[2,3-b]pyridin-4-yl]-3-pyridinesulfonamide;
N-(2-aminoethyl)-6-[2-(trifluoromethyl)-1H-pyrrolo[2,3-b]pyridin-4-yl]-3-pyridinesulfonamide;
   or a salt thereof.

In a further embodiment, the compound of the invention is:
5-(2-cyclopropyl-1H-pyrrolo[2,3-b]pyridin-4-yl)-N-(2-hydroxyethyl)-2-thiophenesulfonamide;
5-(2-cyclopropyl-1H-pyrrolo[2,3-b]pyridin-4-yl)-N-(2-hydroxy-1,1-dimethylethyl)-2-pyridinesulfonamide;
   or a salt thereof.

### TERMS AND DEFINITIONS

“Alkyl” refers to a saturated hydrocarbon chain having the specified number of member atoms. For example, C₁₋₆alkyl refers to an alkyl group having from 1 to 6 member atoms, for example 1 to 4 members. Alkyl groups may be optionally substituted with one or more substituents as defined herein. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl. In one embodiment, alkyl is ethyl. In a further embodiment, alkyl is t-butyl.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” refers to products whose enantiomeric excess is 99% ee or greater.

“Half-life” (or “half-lives”) refers to the time required for half of a quantity of a substance to be converted to another chemically distinct specie in vitro or in vivo.

“Heteroaryl”, unless otherwise defined, refers to an aromatic ring containing from 1 to 4 heteroatoms as member atoms in the ring. Heteroaryl groups containing more than one heteroatom may contain different heteroatoms. Heteroaryl groups may be optionally substituted with one or more substituents as defined herein. Heteroaryl groups are monocyclic ring systems or are fused, spiro, or bridged bicyclic ring systems. Monocyclic heteroaryl rings have 5 or 6 member atoms. Bicyclic heteroaryl rings have from 7 to 11 member atoms. Bicyclic heteroaryl rings include those rings wherein phenyl and a monocyclic heterocycloalkyl ring are attached forming a fused, spiro, or bridged bicyclic ring system, and those rings wherein a monocyclic heteroaryl ring and a monocyclic cycloalkyl, cycloalkenyl, heterocycloalkyl, or heteroaryl ring are attached forming a fused, spiro, or bridged bicyclic ring system. Heteroaryl includes pyrrolyl, pyrazolyl, imidazolyl, oxazolyl, isoxazolyl, thiazolyl, isothiazolyl, furanyl, furazanyl, thienyl, triazolyl, pyridinyl, pyrimidinyl, pyridazinyl, pyrazinyl, triazinyl, tetrazinyl, indolyl, isoindolyl, indolizinyl, indazolyl, purinyl, quinolinyl, isoquinolinyl, quinoxalinyl, quinazolinyl, pteridinyl, cinnolinyl, benzimidazolyl, benopyranyl, benzoxazolyl, benzofuranyl, isobenzofuranyl, benzothiazolyl, benzothienyl, furopyridinyl, and napthyridinyl. For example, 5-membered heteroaryl groups having from 1 to 4 nitrogen atoms include pyrrolyl, pyrazolyl, imidazolyl, triazolyl (including 1,2,3-triazolyl and 1,2,4-triazolyl) and tetrazolyl.

“Heteroatom”, unless otherwise defined, refers to a nitrogen, sulphur, or oxygen atom. Where indicated, a sulphur heteroatom may be optionally substituted with one or two oxo groups.

“Heterocyclyl”, unless otherwise defined, refers to a saturated or unsaturated ring containing from 1 to 4 heteroatoms as member atoms in the ring. However, heterocyclyl rings are not aromatic. Heterocyclyl groups containing more than one heteroatom may contain different heteroatoms. Heterocyclyl groups may be optionally substituted with one or more substituents as defined herein. Heterocyclyl groups are monocyclic ring systems having from 4 to 7 member atoms. In certain embodiments, heterocyclyl is saturated. In other embodiments, heterocyclyl is unsaturated but not aromatic. Heterocyclyl includes pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, homopiperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, and azetidinyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as alkyl, may be unsubstituted or substituted with one or more substituents as defined herein.

“Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. Suitable substituents are defined herein for each substituted or optionally substituted group.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

Ac (acetyl);
Aq (aqueous);
ATP (adenosine triphosphate);
BOC (tert-butyloxycarbonyl);
BSA (bovine serum albumin);
Bu (butyl);
nBu (n-butyl);
CHAPS (3[(3-Cholamidopropyl)dimethylammonio]-propanesulfonic acid);
DCM (dichloromethane);
DIPEA (diisopropylethylamine);

### DMF (N,N-dimethylformamide);

DMSO (dimethylsulfoxide);
dppf (1,1′-bis(diphenylphosphino)ferrocene);
DTT (1,4-dithiothreitol);
EDTA (ethylenediaminetetraacetic acid);
Et (ethyl);
EtOAc (ethyl acetate);
g (grams);
HPLC (high pressure liquid chromatography);
H (hours);

### Hz (Hertz);

IMS (industrial methylated spirits);
IPA (isopropyl alcohol);
L (liters);
LDA (lithium diisopropylamide);
M (molar);
MCPBA (meta-chloroperbenzoic acid);
MDAP (mass directed autopreparative HPLC);
Me (methyl);
MeOH (methanol);
mg (milligrams);
MHz (megahertz);
Min (minutes);
ml or mL (milliliters);
μl or μL (microliters);
mM (millimolar);
mmol (millimoles);
mol (moles);
Ph (phenyl);
ⁱPr (isopropyl);
rt (retention time);
SPE (solid phase extraction);
TBAF (tetra-n-butylammonium fluoride);
TEA (triethylamine);
TFA (trifluoroacetic acid);
THF (tetrahydrofuran);
TLC (thin layer chromatography);
TMAB (tetramethylammoniumbromide); and
Ts (tosyl or p-toluenesulfonyl).

All references to ether are to diethyl ether and brine refers to a saturated aqueous solution of NaCl.

Included within the scope of the “compounds of the invention” are all solvates, hydrates, complexes, polymorphs, prodrugs, radiolabelled derivatives, stereoisomers and optical isomers of the compounds of formula (I) and salts thereof.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and EtOAc, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The invention also includes isotopically-labelled compounds, which are identical to the compounds of formula (I) and salts thereof, but for the fact that one or more atoms are replaced by an atom having an atomic mass or mass number different from the atomic mass or mass number most commonly found in nature. Examples of isotopes that can be incorporated into the compounds of the invention include isotopes of hydrogen, carbon, nitrogen, oxygen and fluorine, such as 3H, 11C, 14C and 18F.

The compounds according to formula (I) may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in formula (I), or in any chemical structure illustrated herein, is not specified the structure is intended to encompass any stereoisomer and all mixtures thereof. Thus, compounds according to formula (I) containing one or more chiral centers may be used as racemic mixtures, enantiomerically enriched mixtures, or as enantiomerically pure individual stereoisomers.

Individual stereoisomers of a compound according to formula (I) which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds according to formula (I) may also contain centers of geometric asymmetry. Where the stereochemistry of a center of geometric asymmetry present in formula (I), or in any chemical structure illustrated herein, is not specified, the structure is intended to encompass the trans (E) geometric isomer, the cis (Z) geometric isomer, and all mixtures thereof. Likewise, all tautomeric forms are also included in formula (I) whether such tautomers exist in equilibrium or predominately in one form.

It is to be understood that the references herein to compounds of formula (I) and salts thereof covers the compounds of formula (I) as the free base or as salts thereof, for example as a pharmaceutically acceptable salt thereof.

The skilled artisan will appreciate that pharmaceutically acceptable salts of the compounds according to formula (I) may be prepared. Indeed, in certain embodiments of the invention, pharmaceutically acceptable salts of the compounds according to formula (I) may be preferred over the respective free base or free acid because such salts impart greater stability or solubility to the molecule thereby facilitating formulation into a dosage form. Accordingly, the invention is further directed to compounds of formula (I) and pharmaceutically acceptable salts thereof.

As used herein, the term “pharmaceutically acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

Salts and solvates having non-pharmaceutically acceptable counter-ions or associated solvents are within the scope of the present invention, for example, for use as intermediates in the preparation of other compounds of formula (I) and their pharmaceutically acceptable salts. Thus one embodiment of the invention embraces compounds of formula (I) and salts thereof.

In certain embodiments, compounds according to formula (I) may contain an acidic functional group. Suitable pharmaceutically-acceptable salts include salts of such acidic functional groups. Representative salts include pharmaceutically acceptable metal salts such as sodium, potassium, lithium, calcium, magnesium, aluminum, and zinc salts; carbonates and bicarbonates of a pharmaceutically acceptable metal cation such as sodium, potassium, lithium, calcium, magnesium, aluminum, and zinc; pharmaceutically acceptable organic primary, secondary, and tertiary amines including aliphatic amines, aromatic amines, aliphatic diamines, and hydroxy alkylamines such as methylamine, ethylamine, 2-hydroxyethylamine, diethylamine, TEA, ethylenediamine, ethanolamine, diethanolamine, and cyclohexylamine.

In certain embodiments, compounds according to formula (I) may contain a basic functional group and are therefore capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

### Compound Preparation

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Examples section.

In one embodiment of the invention, the compounds of formula (I), and salts thereof, may be prepared by a process comprising reacting a compound of formula (II)

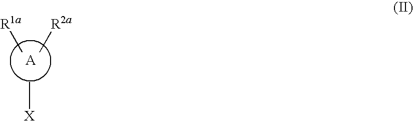

wherein R^{1a} and R^{2a} are R¹ and R² as defined above or groups convertible to R¹ and R², and X is a leaving group such as chlorine or bromine, with a compound of formula (IIIA) or (IIIB)

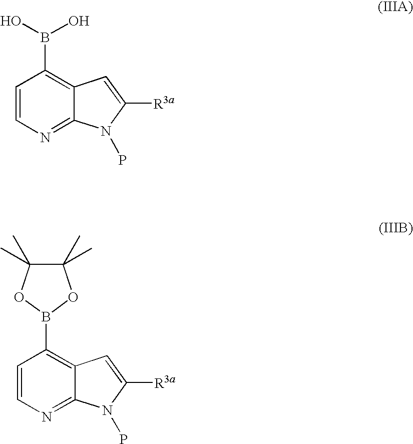

wherein P is hydrogen or a protecting group and R^{3a} is R³ as defined above or a group convertible to R³, in the presence of a catalyst, for example a palladium (II) complex.

The above processes may be followed, if required, by subjecting the resulting compound to one or more of the following operations:

i) removal of the protecting group P,
ii) conversion of R^{1a} to R¹,
iii) conversion of R^{2a} to R²,
iv) conversion of R^{3a} to R³; and
v) conversion of the resultant compound of formula (I) into a salt thereof.

A comprehensive discussion of the ways in which groups may be protected and methods for cleaving the resulting protected derivatives is given by for example T. W. Greene and P. G. M Wuts in Protective Groups in Organic Synthesis 2^{nd} ed., John Wiley & Son, Inc., 1991 and by P. J. Kocienski in Protecting Groups, Georg Thieme Verlag, 1994 which are incorporated herein by reference. Examples of suitable protecting groups, P, include phenylsulfone and 4-methylphenylsulfone.

Such protecting groups may be removed under basic conditions, for example using sodium hydroxide or potassium hydroxide.

In a further embodiment of the invention, the compounds of formula (I), and salts thereof, may be prepared by conversion of one compound of formula (I) into another compound of formula (I).

Suitable functional group transformations for converting one compound of formula (I) into another compound of formula (I), or converting R^{1a} to R¹, R^{2a} to R² or R^{3a} to R³, are well known in the art and are described in, for instance, Comprehensive Heterocyclic Chemistry II, eds. A. R. Katritzky, C. W. Rees and E. F. V. Scriven (Pergamon Press, 1996), Comprehensive Organic Functional Group Transformations, eds. A. R. Katritzky, O. Meth-Cohn and C. W. Rees (Elsevier Science Ltd., Oxford, 1995), Comprehensive Organic Chemistry, eds. D. Barton and W. D. Ollis (Pergamon Press, Oxford, 1979), and Comprehensive Organic Transformations, R. C. Larock (VCH Publishers Inc., New York, 1989).

Compounds of formula (II) may be prepared from compounds of formula (IV)

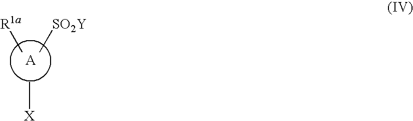

wherein R^{1a} and X are as defined above and Y is a leaving group, for example chlorine or activated imidazole, by reaction with the required amine under suitable conditions, for example in the presence of a hindered organic base, for example TEA or DIPEA, and in an inert solvent, for example DCM. Sulfonyl chloride groups may be obtained from sulfonic acids using a chlorinating reagent such as thionyl chloride. Activated imidazole groups may be obtained from sulphonyl chlorides and imidazole followed by a subsequent alkylation with an alkylating agent such as methyltriflate.

Compounds of formula (IIIA) or (IIIB) may be prepared from compounds of formula (V)

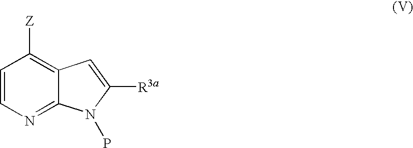

wherein R^{3a} and P are as defined above and Z is halogen, for example bromine, by reaction with triisopropylborate or bis(pinacolato)diboron in the presence of a catalyst, for example palladium (II) compex.

Compounds of formula (IIIA) may also be prepared from compounds of formula (V) wherein R^{3a} is defined above, P is hydrogen and Z is halogen, for example bromine, by reaction with sodium hydride, butyl lithium and triisopropylborate.

Compounds of formula (V) wherein P is a protecting group may be obtained from compounds of formula (VI)

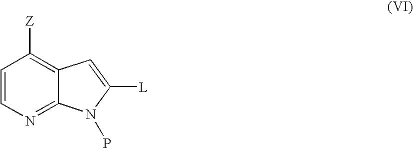

wherein P is a protecting group, Z is as defined above and L is a leaving group such as a halogen, for example iodine, by reaction with a Grignard reagent, for example isopropylmagnesium chloride, and subsequent reaction with a suitable electrophile.

Compounds of formula (V) wherein P is a protecting group may also be obtained from compounds of formula (VII)

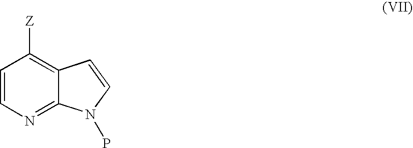

wherein P is a protecting group and Z is as defined above, via generation of an anion at the 2-position with a strong hindered base, for example lithum di-isopropylamide, at low temperature, for example −78° C., and subsequent reaction with an electrophile such as an alkyl halide, for example methyl iodide, or a formylating reagent, for example dimethylformamide.

Alternatively, compounds of formula (V) may be obtained from compounds of formula (VIII)

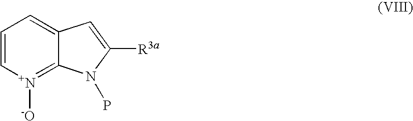

wherein P and R^{3a} are as defined above, in the presence of a suitable halogenating reagent, for example tetrabutylammonium bromide, with methanesulfonic anhydride in a suitable solvent, for example dimethylformamide.

Compounds of formula (VIII) may be obtained by reacting compounds of formula (IX)

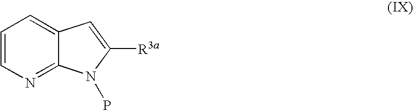

wherein R^{3a} is as defined above, with a suitable oxidising agent, for example meta-chloroperbenzoic acid, in a suitable solvent, for example EtOAc.

Compounds of formula (IX) may be prepared by reacting a compound of formula (X)

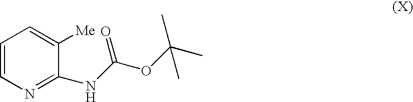

with a suitable strong base, for example butyl lithium, at low temperature, for example −4 to 0° C., in an inert solvent, for example THF, and subsequently reacting with a N,N-dimethylamide or N-methyl-N-methoxy (Weinreb) amide at low temperature, for example 0 to 10° C. The preparation of compounds of formula (IX) may be completed by acidification with a strong mineral acid, for example hydrochloric acid, at low temperature, for example 0 to 5° C., followed by heating at an elevated temperature, for example 50 to 90° C.

Compounds of the invention and intermediates useful in the preparation of the compounds of the invention can be prepared, for example, according to Schemes 1 to 23 below:

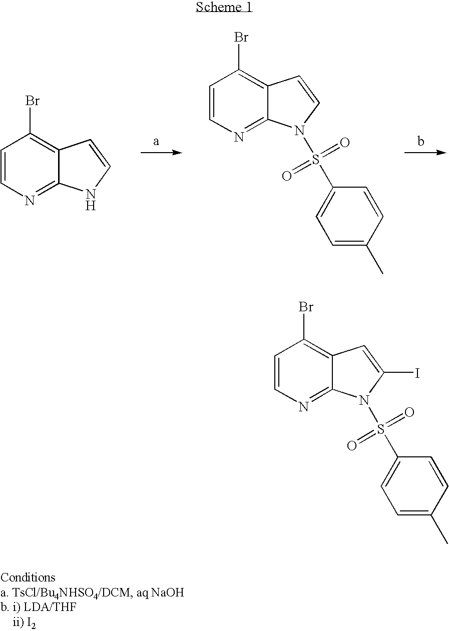

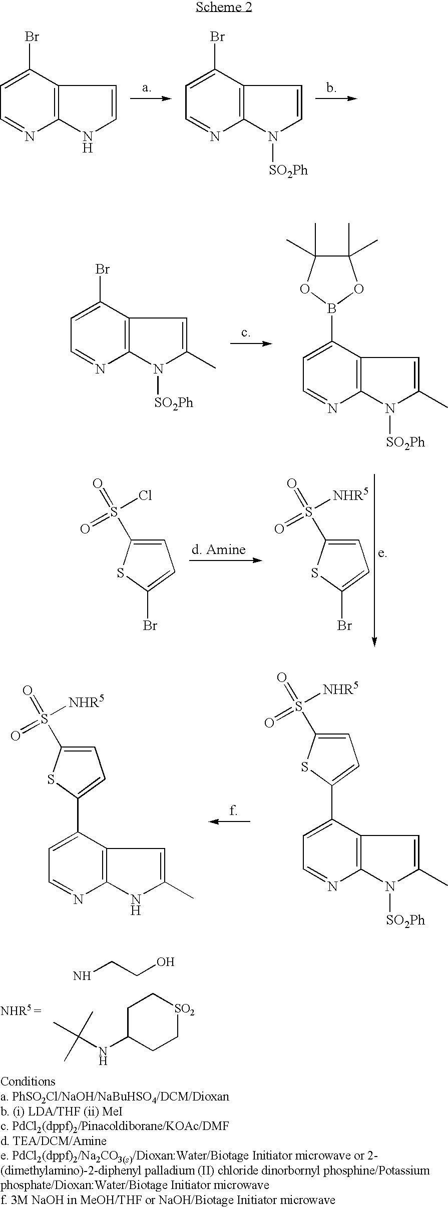

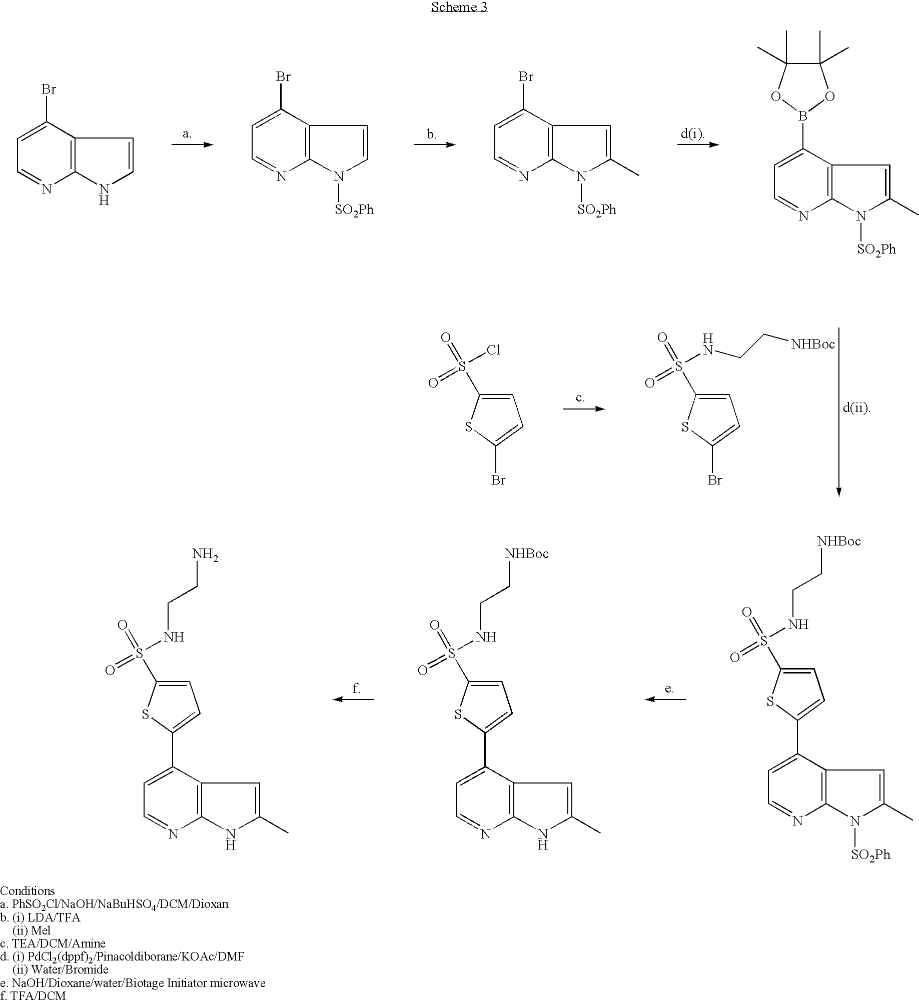

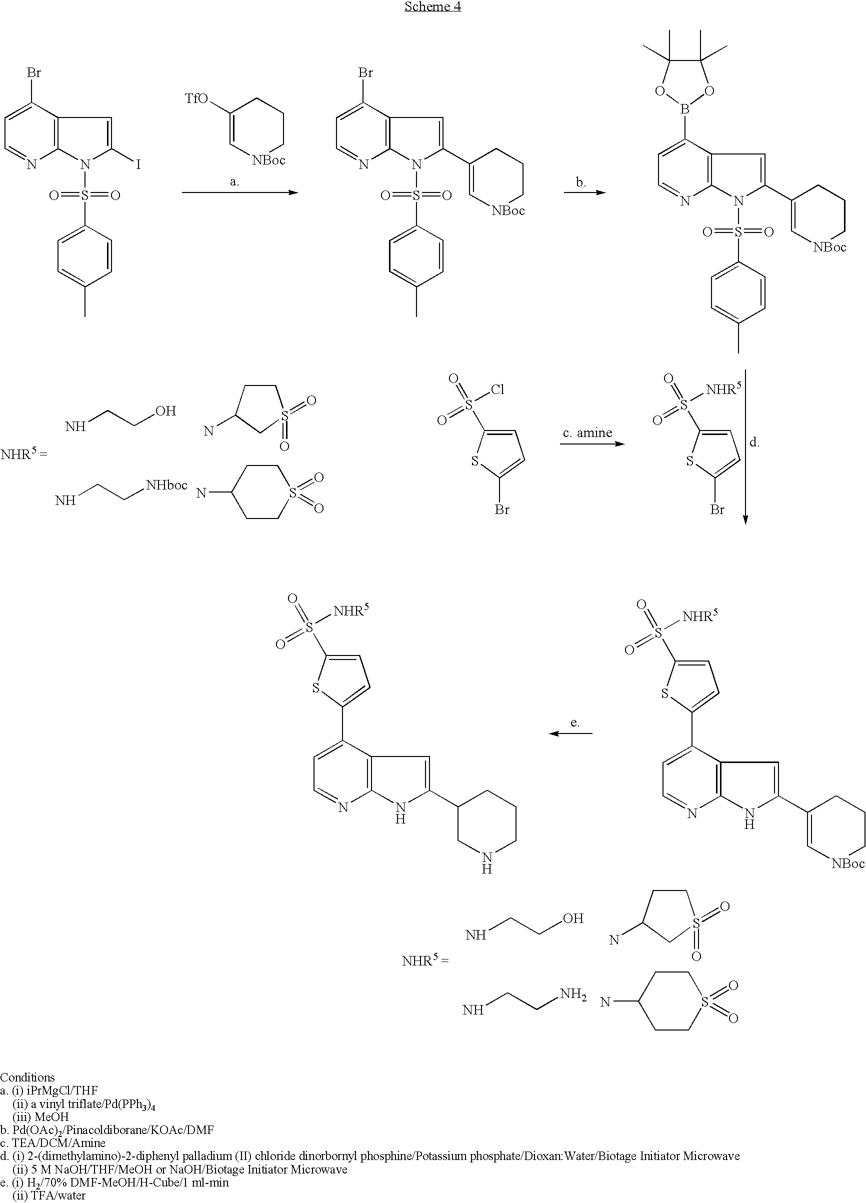

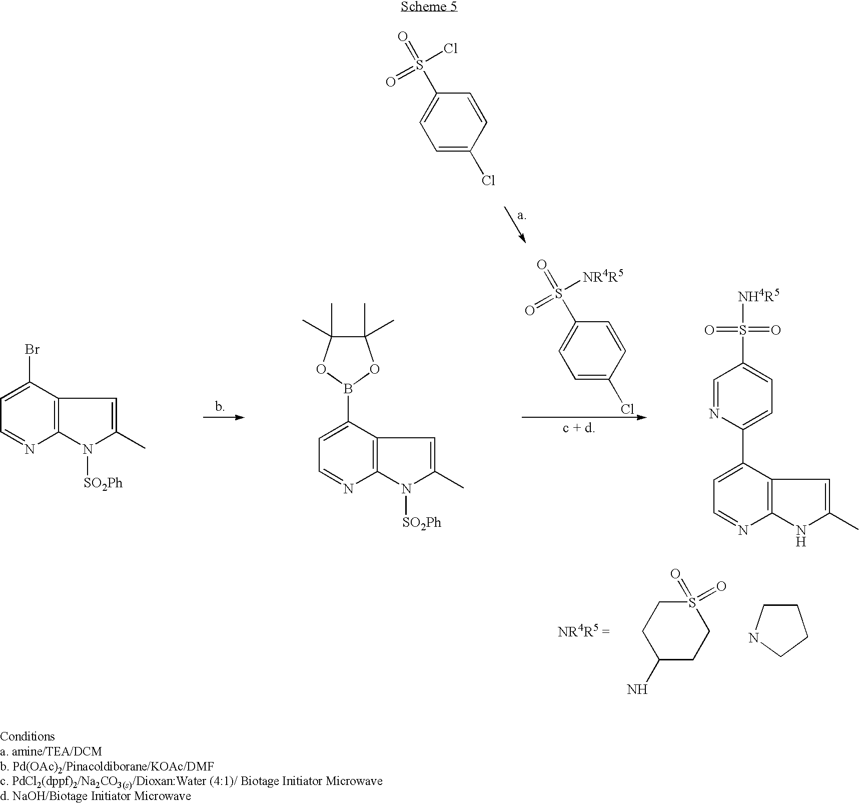

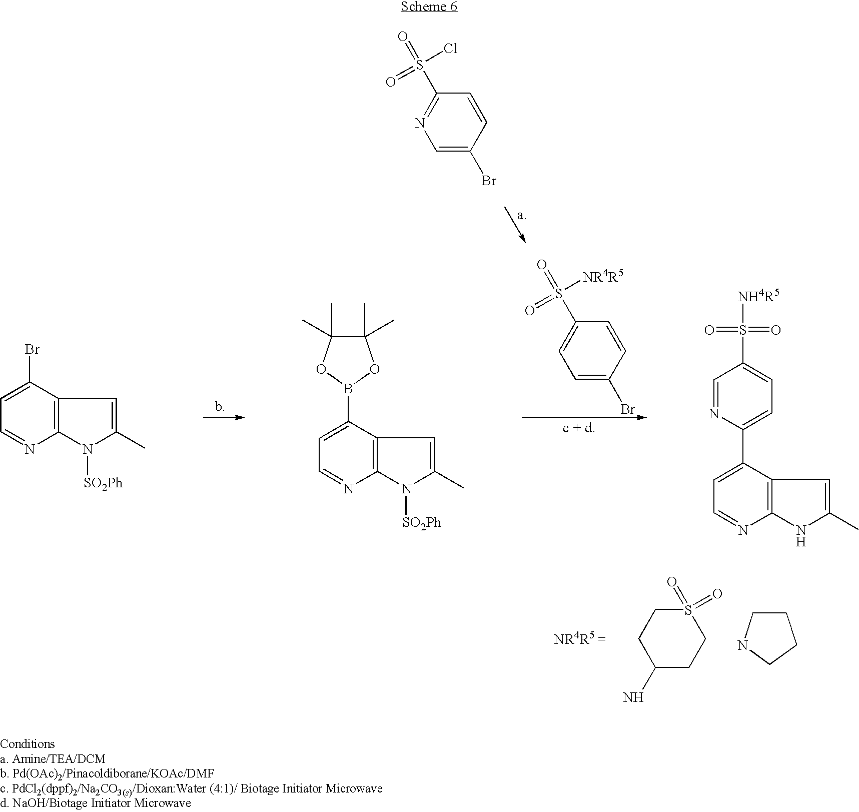

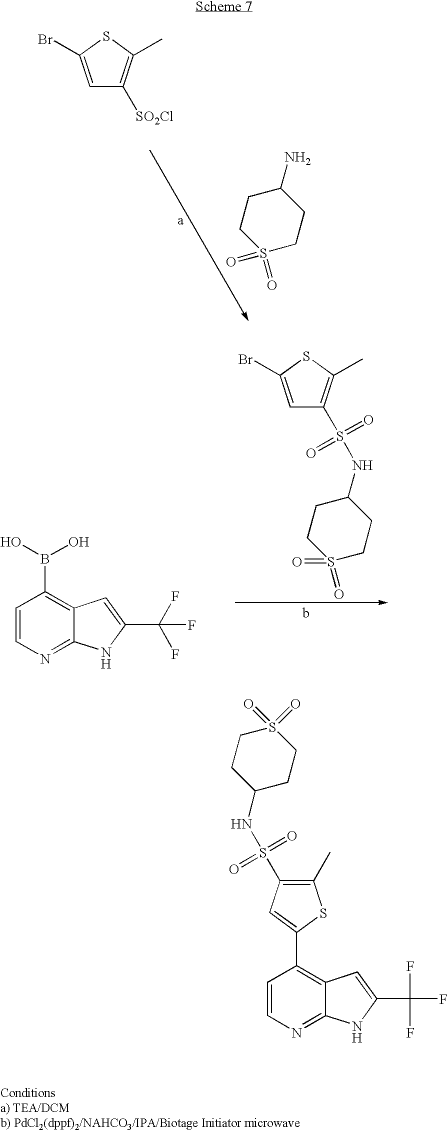

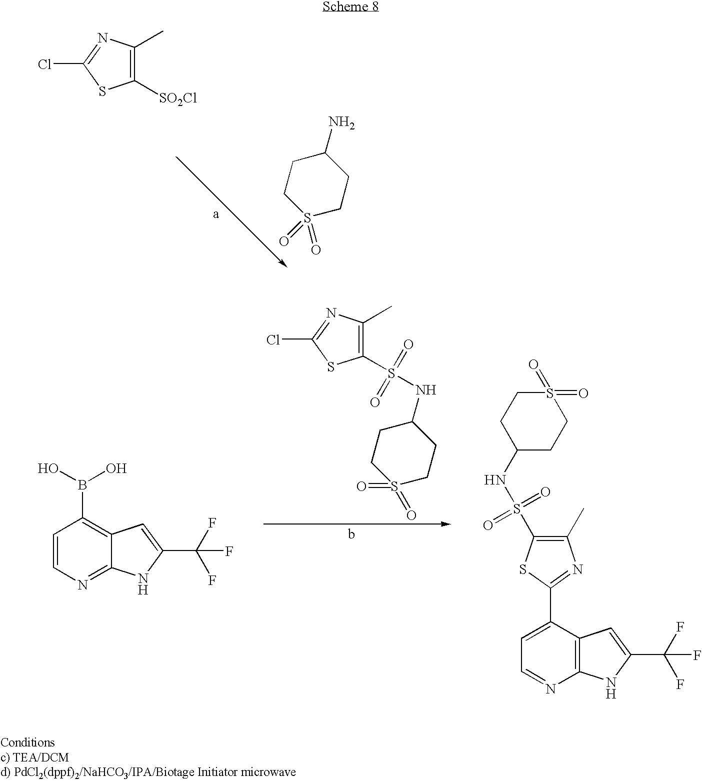

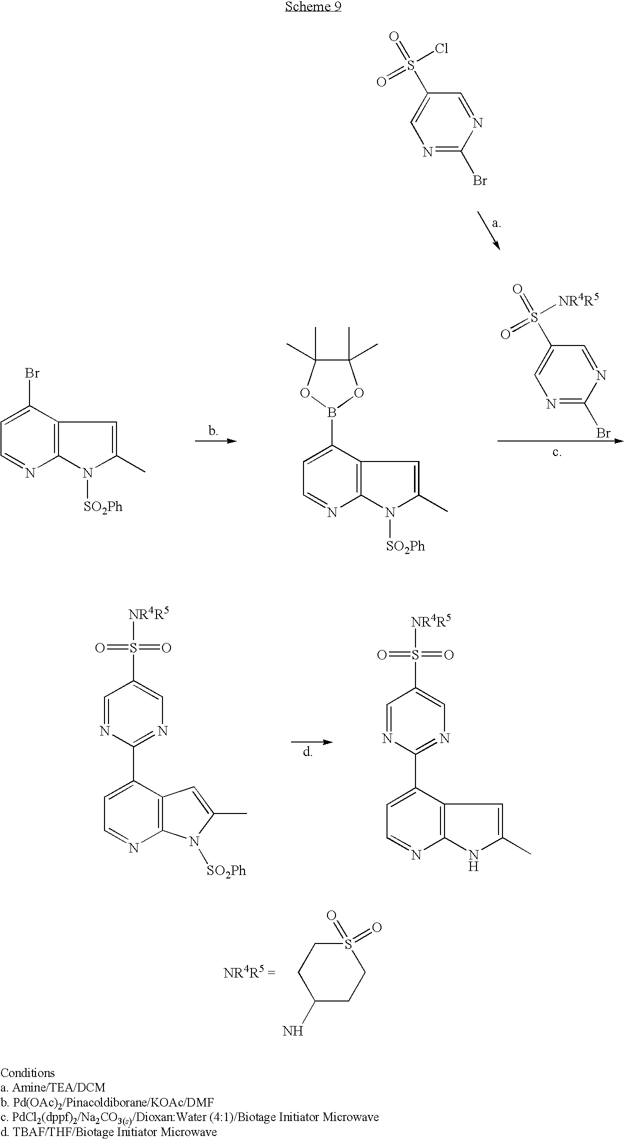

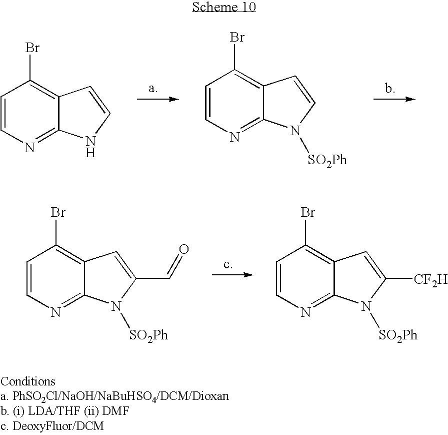

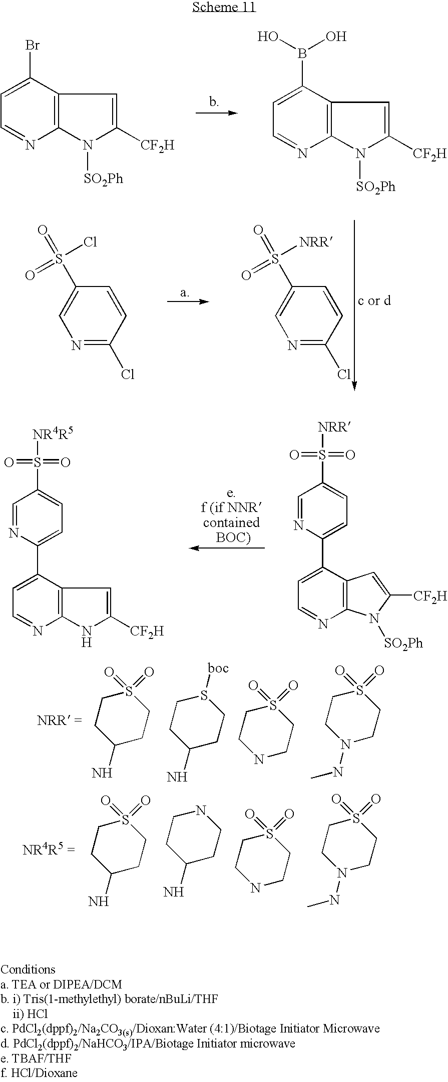

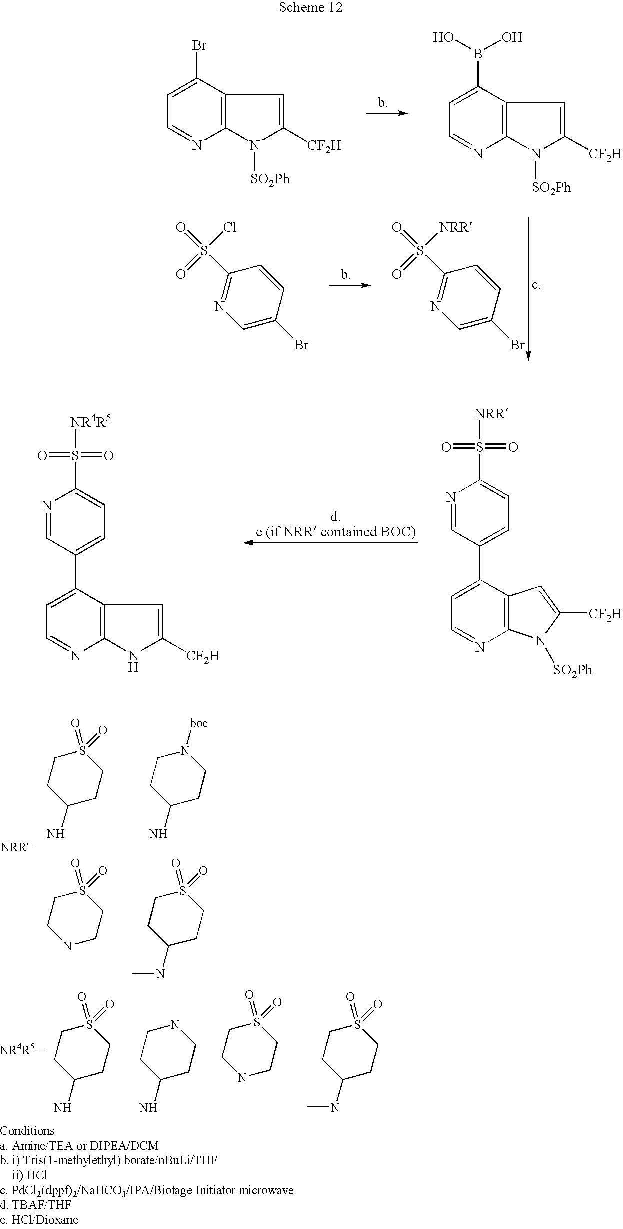

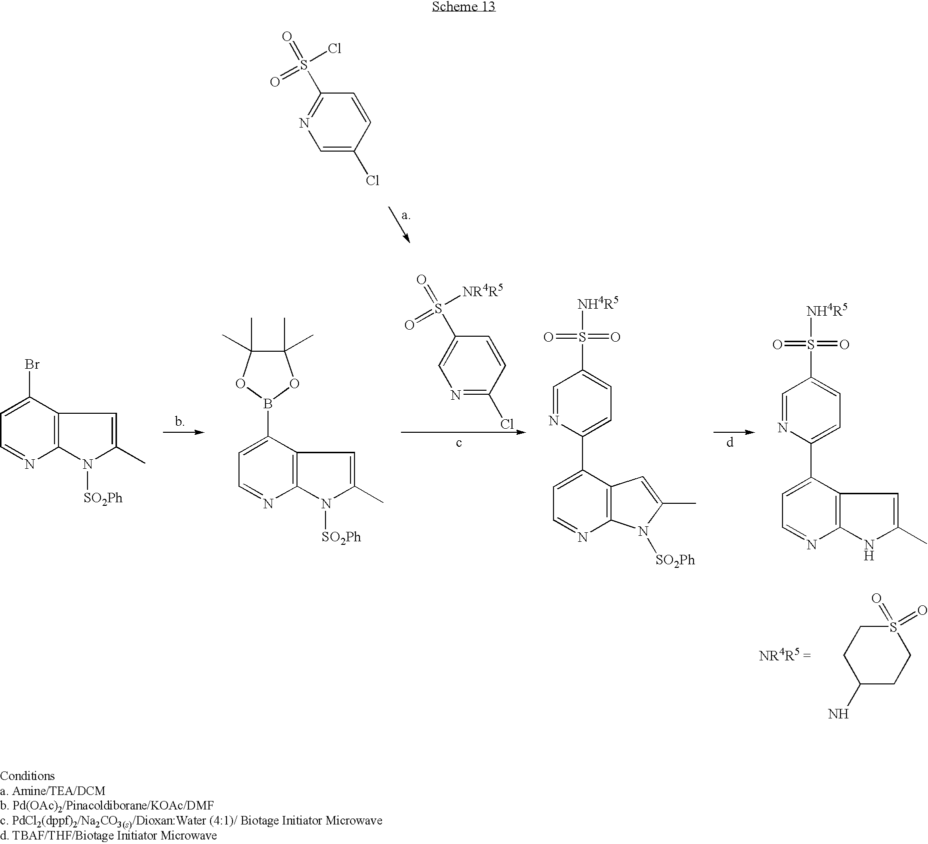

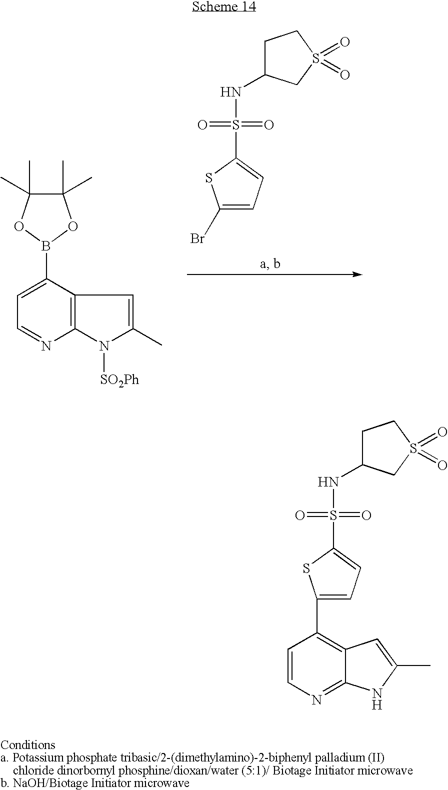

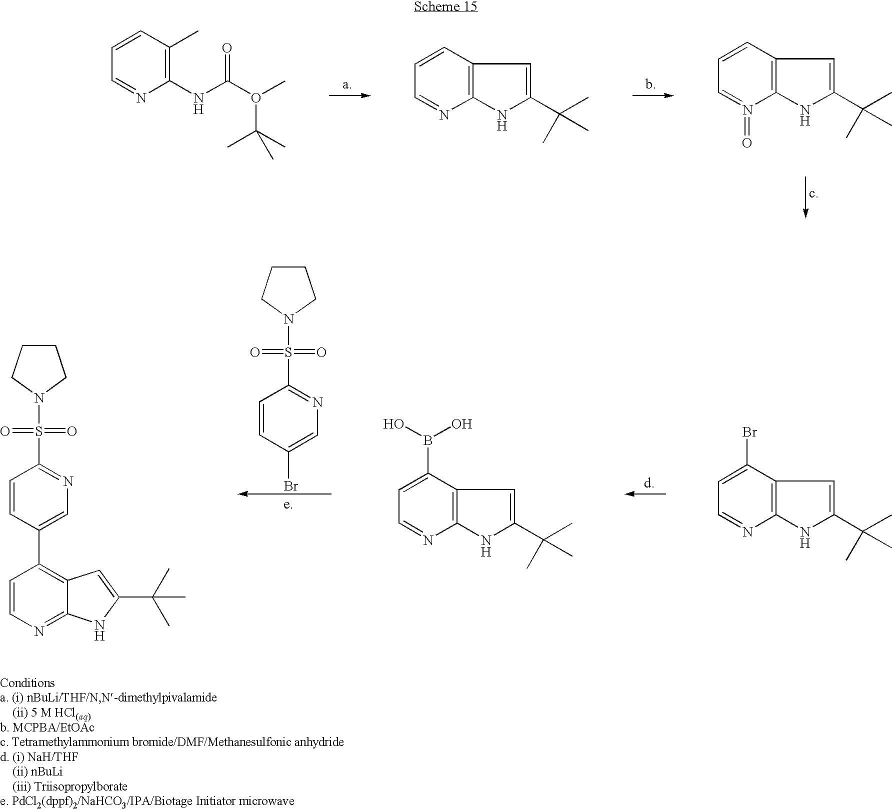

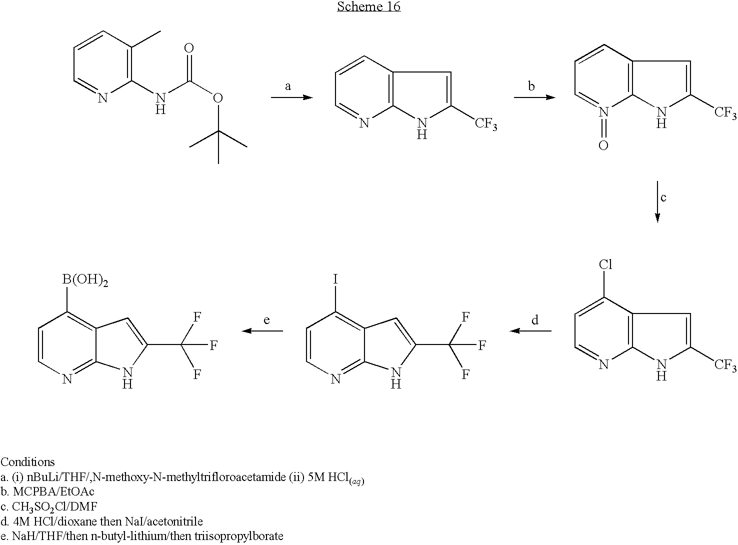

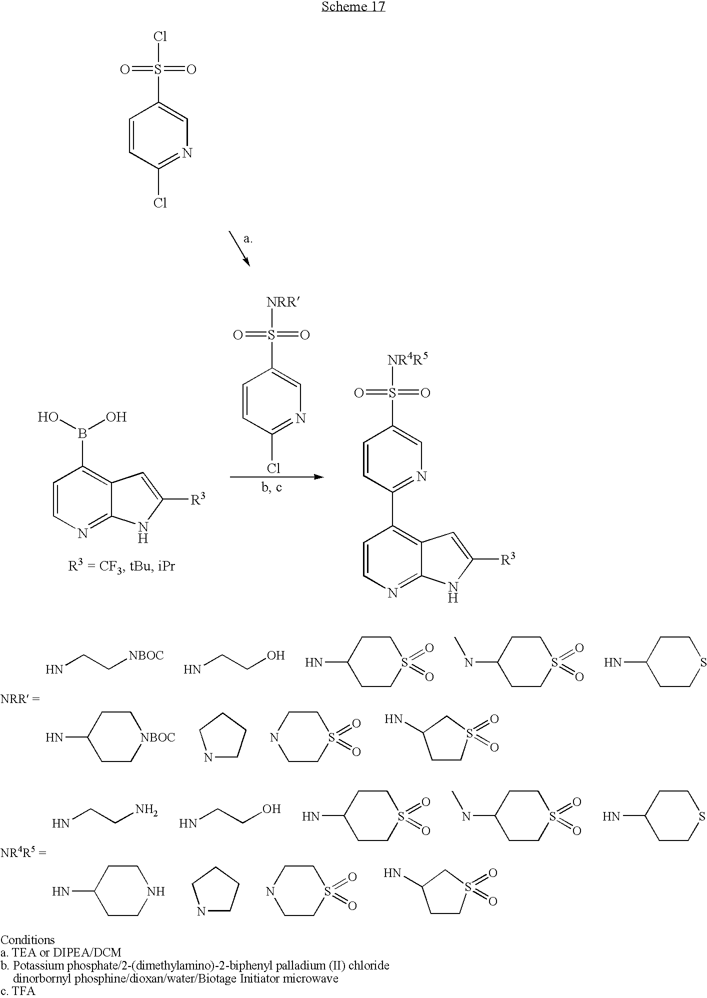

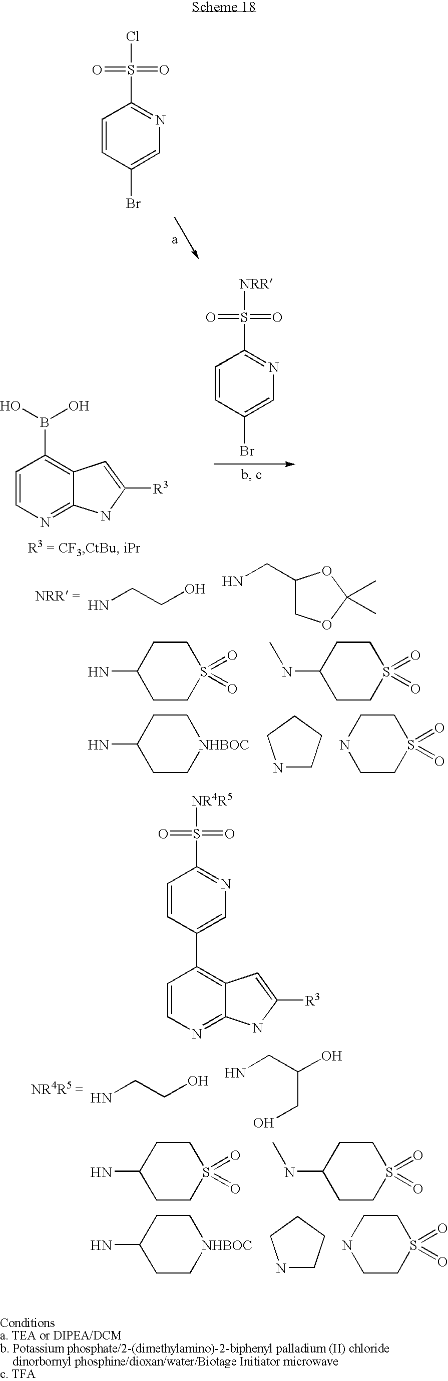

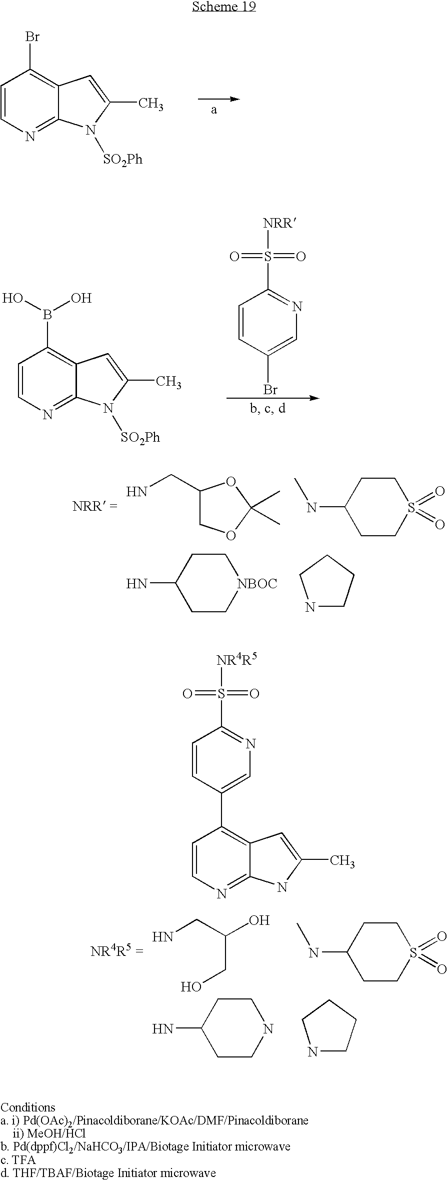

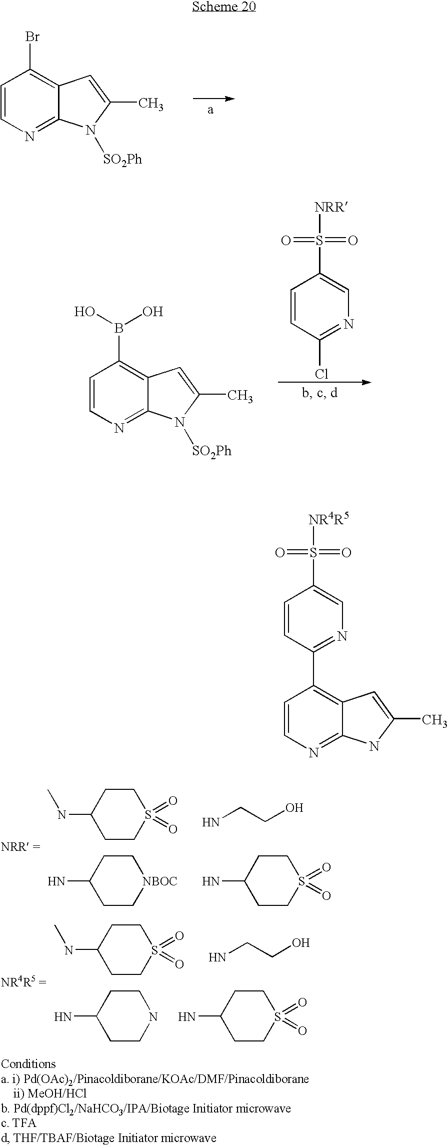

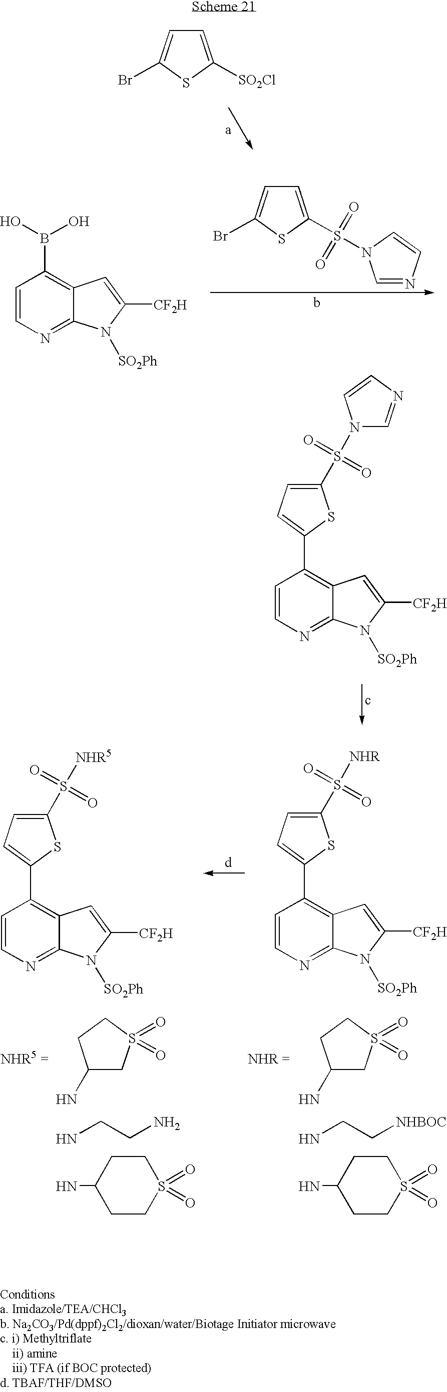

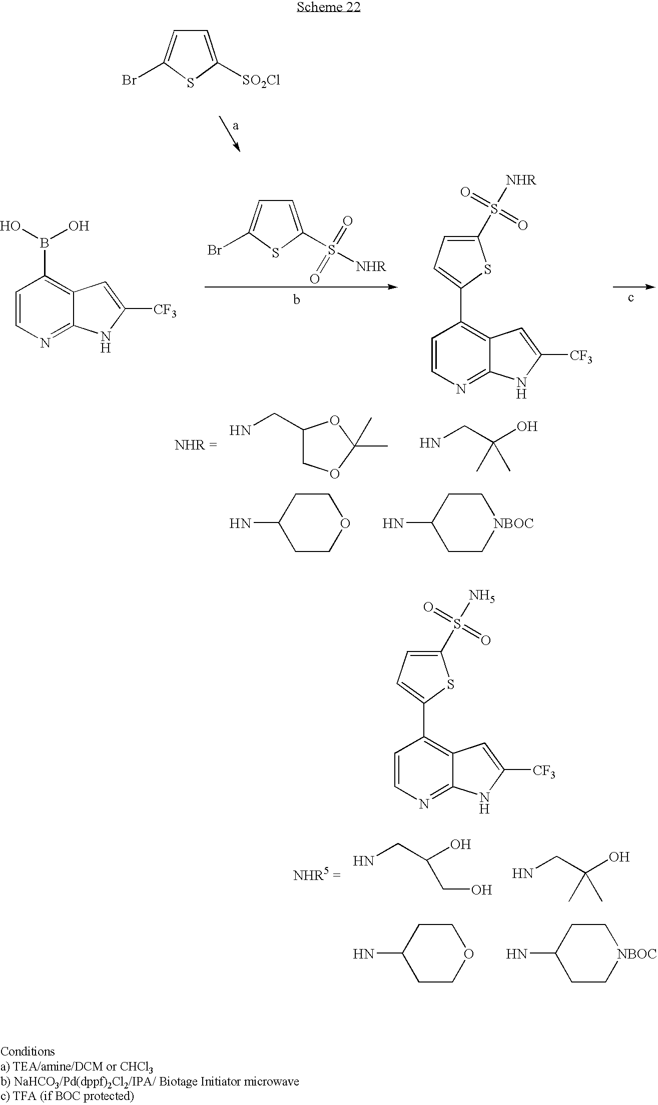

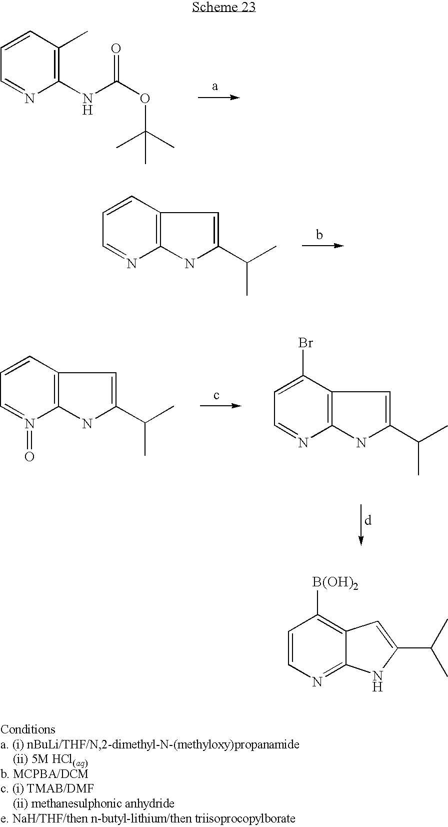

### Methods of Use

The compounds of the invention are inhibitors of IKK2. Compounds which are inhibitors of IKK2 may be useful in the treatment of disorders wherein the underlying pathology is (at least in part) attributable to inappropriate IKK2 (also known as IKKβ) activity such as rheumatoid arthritis, inflammatory bowel disease, COPD (chronic obstructive pulmonary disease), asthma and rhinitis. “Inappropriate IKK2 activity” refers to any IKK2 activity that deviates from the normal IKK2 activity expected in a particular patient. Inappropriate IKK2 activity may take the form of, for instance, an abnormal increase in activity, or an aberration in the timing and or control of IKK2 activity. Such inappropriate activity may result then, for example, from overexpression or mutation of the protein kinase leading to inappropriate or uncontrolled activation. Accordingly, in another aspect the invention is directed to methods of treating such disorders.

Such disorders include inflammatory and tissue repair disorders (including rheumatoid arthritis, inflammatory bowel disease, COPD (chronic obstructive pulmonary disease), asthma and rhinitis), fibrotic diseases, osteoarthritis, osteoporosis, dermatosis (including psoriasis, atopic dermatitis and ultraviolet radiation (UV)-induced skin damage), autoimmune diseases (including Sjogren's syndrome, systemic lupus eythematosus, multiple sclerosis, psoriatic arthritis, alkylosing spondylitis, tissue and organ rejection), Alzheimer's disease, stroke, atherosclerosis, restonosis, diabetes, glomerulonephritis, cancer (including Hodgkin's disease), cachexia, inflammation associated with infection and certain viral infections (including acquired immune deficiency syndrome (AIDS)), adult respiratory distress syndrome, and Ataxia Telangiestasia.

The methods of treatment of the invention comprise administering a safe and effective amount of a compound of formula (I) or a pharmaceutically acceptable salt thereof to a patient in need thereof. Individual embodiments of the invention include methods of treating any one of the above-mentioned disorders by administering a safe and effective amount of a compound of formula (I) or a pharmaceutically acceptable salt thereof to a patient in need thereof.

As used herein, “treat” in reference to a disorder means: (1) to ameliorate or prevent the disorder or one or more of the biological manifestations of the disorder, (2) to interfere with (a) one or more points in the biological cascade that leads to or is responsible for the disorder or (b) one or more of the biological manifestations of the disorder, (3) to alleviate one or more of the symptoms or effects associated with the disorder, or (4) to slow the progression of the disorder or one or more of the biological manifestations of the disorder.

As indicated above, “treatment” of a disorder includes prevention of the disorder. The skilled artisan will appreciate that “prevention” is not an absolute term. In medicine, “prevention” is understood to refer to the prophylactic administration of a drug to substantially diminish the likelihood or severity of a disorder or biological manifestation thereof, or to delay the onset of such disorder or biological manifestation thereof.

As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the disorder being treated; the severity of the disorder being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human (including adults and children) or other animal.

The compounds of formula (I) or pharmaceutically acceptable salts thereof may be administered by any suitable route of administration, including both systemic administration and topical administration. Systemic administration includes oral administration, parenteral administration, transdermal administration and rectal administration. Parenteral administration refers to routes of administration other than enteral or transdermal, and is typically by injection or infusion. Parenteral administration includes intravenous, intramuscular, and subcutaneous injection or infusion. Topical administration includes application to the skin as well as intraocular, otic, intravaginal, inhaled and intranasal administration. Inhalation refers to administration into the patient's lungs whether inhaled through the mouth or through the nasal passages. In one embodiment, the compounds of formula (I) or pharmaceutically acceptable salts thereof may be administered orally. In another embodiment, the compounds of formula (I) or pharmaceutically acceptable salts thereof may be administered by inhalation. In a further embodiment, the compounds of formula (I) or pharmaceutically acceptable salts thereof may be administered intranasally.

The compounds of formula (I) or pharmaceutically acceptable salts thereof may be administered once or according to a dosing regimen wherein a number of doses are administered at varying intervals of time for a given period of time. For example, doses may be administered one, two, three, or four times per day. In one embodiment, a dose is administered once per day. In a further embodiment, a dose is administered twice per day. Doses may be administered until the desired therapeutic effect is achieved or indefinitely to maintain the desired therapeutic effect. Suitable dosing regimens for a compound of formula (I) or a pharmaceutically acceptable salt thereof depend on the pharmacokinetic properties of that compound, such as absorption, distribution, and half-life, which can be determined by the skilled artisan. In addition, suitable dosing regimens, including the duration such regimens are administered, for a compound of formula (I) or a pharmaceutically acceptable salt thereof depend on the disorder being treated, the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical daily dosages for oral administration range from 0.001 mg to 50 mg per kg of total body weight, for example from 1 mg to 10 mg per kg of total body weight. For example, daily dosages for oral administration may be from 0.5 mg to 2 g per patient, such as 10 mg to 1 g per patient.

Additionally, the compounds of formula (I) may be administered as prodrugs. As used herein, a “prodrug” of a compound of formula (I) is a functional derivative of the compound which, upon administration to a patient, eventually liberates the compound of formula (I) in vivo. Administration of a compound of formula (I) as a prodrug may enable the skilled artisan to do one or more of the following: (a) modify the onset of the compound in vivo; (b) modify the duration of action of the compound in vivo; (c) modify the transportation or distribution of the compound in vivo; (d) modify the solubility of the compound in vivo; and (e) overcome or overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

The invention thus provides a method of treating a disorder mediated by inappropriate IKK2 activity comprising administering a safe and effective amount of a compound of formula (I) or a pharmaceutically acceptable salt thereof to a patient in need thereof.

In one embodiment, the disorder mediated by inappropriate IKK2 activity is selected from the group consisting of inflammatory and tissue repair disorders (including rheumatoid arthritis, inflammatory bowel disease, COPD (chronic obstructive pulmonary disease), asthma and rhinitis), fibrotic diseases, osteoarthritis, osteoporosis, dermatosis (including psoriasis, atopic dermatitis and ultraviolet radiation (UV)-induced skin damage), autoimmune diseases (including Sjogren's syndrome, systemic lupus eythematosus, multiple sclerosis, psoriatic arthritis, alkylosing spondylitis, tissue and organ rejection), Alzheimer's disease, stroke, atherosclerosis, restonosis, diabetes, glomerulonephritis, cancer (including Hodgkin's disease), cachexia, inflammation associated with infection and certain viral infections (including acquired immune deficiency syndrome (AIDS)), adult respiratory distress syndrome, and Ataxia Telangiestasia.

In another embodiment, the disorder mediated by inappropriate IKK2 activity is selected from the group consisting of: rheumatoid arthritis, inflammatory bowel disease, COPD (chronic obstructive pulmonary disease), asthma, rhinitis, osteoarthritis, osteoporosis, psoriasis, atopic dermatitis, ultraviolet radiation (UV)-induced skin damage, systemic lupus eythematosus, multiple sclerosis, psoriatic arthritis, alkylosing spondylitis, tissue rejection, organ rejection, Alzheimer's disease, stroke, atherosclerosis, restonosis, diabetes, glomerulonephritis, Hodgkin's disease, cachexia, inflammation associated with infection and certain viral infections, including acquired immune deficiency syndrome (AIDS), adult respiratory distress syndrome, and Ataxia Telangiestasia.

In another embodiment, the disorder mediated by inappropriate IKK2 activity is an inflammatory or tissue repair disorder. In another aspect, the disorder mediated by inappropriate IKK2 activity is rheumatoid arthritis, COPD, asthma or rhinitis. In another aspect, the disorder mediated by inappropriate IKK2 activity is rheumatoid arthritis. In another aspect, the disorder mediated by inappropriate IKK2 activity is COPD. In another aspect, the disorder mediated by inappropriate IKK2 activity is asthma. In a further aspect, the disorder mediated by inappropriate IKK2 activity is rhinitis (including seasonal rhinitis, allergic rhinitis and vasomotor rhinitis).

In another embodiment, the disorder mediated by inappropriate IKK2 activity is an autoimmune disease. In another embodiment, the disorder mediated by inappropriate IKK2 activity is Sjogren's syndrome, systemic lupus eythematosus, multiple sclerosis, psoriatic arthritis, or alkylosing spondylitis. In a further embodiment, the disorder mediated by inappropriate IKK2 activity is systemic lupus eythematosus, multiple sclerosis, psoriatic arthritis, or alkylosing spondylitis.

In another embodiment, the disorder mediated by inappropriate IKK2 activity is selected from the group consisting of Alzheimer's disease, stroke, atherosclerosis, restenosis, diabetes, glomerulonephritis, osteoarthritis, osteoporosis, and Ataxia Telangiestasia.

In another embodiment, the disorder mediated by inappropriate IKK2 activity is cancer or cachexia. In a further embodiment, the disorder mediated by inappropriate IKK2 activity is cancer.

The term “rhinitis” is used herein to refer to all types of rhinitis including allergic rhinitis such as seasonal rhinitis (for example hayfever) or perennial rhinitis, and non-allergic rhinitis or vasomotor rhinitis.

The invention also provides a compound of formula (I) or a pharmaceutically acceptable salt thereof for use in medical therapy, and particularly in the treatment of disorders mediated by IKK2 activity. Thus, in a further aspect, the invention is directed to the use of a compound of formula (I) or a pharmaceutically acceptable salt thereof in the manufacture of a medicament for use in the treatment of a disorder characterized by inappropriate IKK2 activity.

Particular disorders characterised by inappropriate IKK2 activity include inflammatory and tissue repair disorders, particularly rheumatoid arthritis, inflammatory bowel disease, COPD (chronic obstructive pulmonary disease), asthma and rhinitis; osteoarthritis, osteoporosis and fibrotic diseases; dermatosis, including psoriasis, atopic dermatitis and ultraviolet radiation (UV)-induced skin damage; autoimmune diseases including systemic lupus eythematosus, multiple sclerosis, psoriatic arthritis, alkylosing spondylitis, tissue and organ rejection, Alzheimer's disease, stroke, atherosclerosis, restenosis, diabetes, glomerulonephritis, cancer, including Hodgkin's disease, cachexia, inflammation associated with infection and certain viral infections, including acquired immune deficiency syndrome (AIDS), adult respiratory distress syndrome, and Ataxia Telangiestasia as a result of inhibition of the protein kinase IKK2.

### Compositions

The compounds of formula (I) or a pharmaceutically acceptable salts thereof will normally, but not necessarily, be formulated into pharmaceutical compositions prior to administration to a patient. Accordingly, in another aspect the invention is directed to pharmaceutical compositions comprising a compound of formula (I) or a pharmaceutically acceptable salt thereof and one or more pharmaceutically-acceptable excipients.

The pharmaceutical compositions of the invention may be prepared and packaged in bulk form wherein a safe and effective amount of a compound of formula (I) or a pharmaceutically acceptable salt thereof can be extracted and then given to the patient such as with powders or syrups. Alternatively, the pharmaceutical compositions of the invention may be prepared and packaged in unit dosage form wherein each physically discrete unit contains a safe and effective amount of a compound of formula (I) or a pharmaceutically acceptable salt thereof. When prepared in unit dosage form, the pharmaceutical compositions of the invention typically may contain, for example, from 0.5 mg to 1 g, or from 1 mg to 700 mg, or from 5 mg to 100 mg of a compound of formula (I) or a pharmaceutically acceptable salt thereof.

The pharmaceutical compositions of the invention typically contain one compound of formula (I) or pharmaceutically acceptable salt thereof. However, in certain embodiments, the pharmaceutical compositions of the invention contain more than one compound of formula (I) or pharmaceutically acceptable salt thereof. For example, in certain embodiments the pharmaceutical compositions of the invention contain two compounds of formula (I) or pharmaceutically acceptable salts thereof.

In addition, the pharmaceutical compositions of the invention may optionally further comprise one or more additional pharmaceutically active compounds.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when comingled such that interactions which would substantially reduce the efficacy of the compound of formula (I) or pharmaceutically acceptable salt thereof when administered to a patient and interactions which would result in pharmaceutical compositions that are not pharmaceutically acceptable are avoided. In addition, each excipient must of course be of sufficiently high purity to render it pharmaceutically-acceptable.

The compound of formula (I) or pharmaceutically acceptable salt thereof and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of formula (I) or pharmaceutically acceptable salts thereof once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

Accordingly, in another aspect the invention is directed to process for the preparation of a pharmaceutical composition comprising a compound of formula (I) or a pharmaceutically acceptable salt thereof and one or more pharmaceutically-acceptable excipients which comprises mixing the ingredients. A pharmaceutical composition comprising a compound of formula (I) or a pharmaceutically acceptable salt thereof may be prepared by, for example, admixture at ambient temperature and atmospheric pressure.

In one embodiment, the compounds of formula (I) or pharmaceutically acceptable salts thereof will be formulated for oral administration. In another embodiment, the compounds of formula (I) or pharmaceutically acceptable salts thereof will be formulated for inhaled administration. In a further embodiment, the compounds of formula (I) or pharmaceutically acceptable salts thereof will be formulated for intranasal administration.

In one aspect, the invention is directed to a solid oral dosage form such as a tablet or capsule comprising a safe and effective amount of a compound of formula (I) or a pharmaceutically acceptable salt thereof and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmellose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compounds of formula (I) or pharmaceutically acceptable salts thereof may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds of formula (I) or pharmaceutically acceptable salts thereof may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

In another aspect, the invention is directed to a liquid oral dosage form. Oral liquids such as solution, syrups and elixirs can be prepared in dosage unit form so that a given quantity contains a predetermined amount of a compound of formula (I) or a pharmaceutically acceptable salt thereof. Syrups can be prepared by dissolving a compound of formula (I) or a pharmaceutically acceptable salt thereof in a suitably flavored aqueous solution, while elixirs are prepared through the use of a non-toxic alcoholic vehicle. Suspensions can be formulated by dispersing a compound of formula (I) or a pharmaceutically acceptable salt thereof in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to a dosage form adapted for administration to a patient by inhalation. For example, as a dry powder, an aerosol, a suspension, or a solution composition.

Dry powder compositions for delivery to the lung by inhalation typically comprise a compound of formula (I) or a pharmaceutically acceptable salt thereof as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The finely divided powder may be prepared by, for example, micronisation and milling. Generally, the size-reduced (e.g. micronised) compound can be defined by a D₅₀ value of about 1 to about 10 microns (for example as measured using laser diffraction).

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of a compound of formula (I) or a pharmaceutically acceptable salt thereof.

Aerosols may be formed by suspending or dissolving a compound of formula (I) or a pharmaceutically acceptable salt thereof in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of formula (I) or a pharmaceutically acceptable salt thereof will typically be administered to a patient via a metered dose inhaler (MDI). Such devices are known to those skilled in the art.

The aerosol may contain additional pharmaceutically-acceptable excipients typically used with MDIs such as surfactants, lubricants, cosolvents and other excipients to improve the physical stability of the formulation, to improve valve performance, to improve solubility, or to improve taste.

There is thus provided as a further aspect of the invention a pharmaceutical aerosol formulation comprising a compound of formula (I) or a pharmaceutically acceptable salt thereof and a fluorocarbon or hydrogen-containing chlorofluorocarbon as propellant, optionally in combination with a surfactant and/or a cosolvent.

According to another aspect of the invention, there is provided a pharmaceutical aerosol formulation wherein the propellant is selected from 1,1,1,2-tetrafluoroethane, 1,1,1,2,3,3,3-heptafluoro-n-propane and mixtures thereof.

The formulations of the invention may be buffered by the addition of suitable buffering agents.

Capsules and cartridges for use in an inhaler or insufflator, of for example gelatine, may be formulated containing a powder mix for inhalation of a compound of formula (I) or a pharmaceutically acceptable salt thereof and a suitable powder base such as lactose or starch. Each capsule or cartridge may generally contain from 20 μg to 10 mg of the compound of formula (I) or a pharmaceutically acceptable salt thereof. Alternatively, the compound of formula (I) or a pharmaceutically acceptable salt thereof may be presented without excipients such as lactose.

The proportion of the active compound of formula (I) or pharmaceutically acceptable salt thereof in the local compositions according to the invention depends on the precise type of formulation to be prepared but will generally be within the range of from 0.001 to 10% by weight. Generally, for most types of preparations, the proportion used will be within the range of from 0.005 to 1%, for example from 0.01 to 0.5%. However, in powders for inhalation or insufflation the proportion used will normally be within the range of from 0.1 to 5%.

Aerosol formulations are preferably arranged so that each metered dose or “puff” of aerosol contains from 20 μg to 10 mg, preferably from 20 μg to 2000 μg, more preferably from about 20 μg to 500 μg of a compound of formula (I). Administration may be once daily or several times daily, for example 2, 3, 4 or 8 times, giving for example 1, 2 or 3 doses each time. The overall daily dose with an aerosol will be within the range from 100 μg to 10 mg, preferably from 200 μg to 2000 μg. The overall daily dose and the metered dose delivered by capsules and cartridges in an inhaler or insufflator will generally be double that delivered with aerosol formulations.

In the case of suspension aerosol formulations, the particle size of the particulate (e.g., micronised) drug should be such as to permit inhalation of substantially all the drug into the lungs upon administration of the aerosol formulation and will thus be less than 100 microns, desirably less than 20 microns, and in particular in the range of from 1 to 10 microns, such as from 1 to 5 microns, more preferably from 2 to 3 microns.

The formulations of the invention may be prepared by dispersal or dissolution of the medicament and a compound of formula (I) or a pharmaceutically acceptable salt thereof in the selected propellant in an appropriate container, for example, with the aid of sonication or a high-shear mixer. The process is desirably carried out under controlled humidity conditions.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The stability of the suspension aerosol formulations according to the invention may be measured by conventional techniques, for example, by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above.

The term “metered dose inhaler” or MDI means a unit comprising a can, a secured cap covering the can and a formulation metering valve situated in the cap. MDI system includes a suitable channeling device. Suitable channeling devices comprise for example, a valve actuator and a cylindrical or cone-like passage through which medicament may be delivered from the filled canister via the metering valve to the nose or mouth of a patient such as a mouthpiece actuator.

MDI canisters generally comprise a container capable of withstanding the vapour pressure of the propellant used such as a plastic or plastic-coated glass bottle or preferably a metal can, for example, aluminium or an alloy thereof which may optionally be anodised, lacquer-coated and/or plastic-coated (for example incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDIs taught herein may be prepared by methods of the art (e.g. see Byron, above and WO96/32099). Preferably the canister is fitted with a cap assembly, wherein a drug-metering valve is situated in the cap, and said cap is crimped in place.

In one embodiment of the invention the metallic internal surface of the can is coated with a fluoropolymer, more preferably blended with a non-fluoropolymer. In another embodiment of the invention the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, bromobutyl, EPDM, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK357) and 3M-Neotechnic Ltd., UK (e.g. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; and 6,679,374, as well as dose counter units such as, but not limited to, those described in U.S. Pat. Nos. 6,360,739 and 6,431,168.

Conventional bulk manufacturing methods and machinery well known to those skilled in the art of pharmaceutical aerosol manufacture may be employed for the preparation of large-scale batches for the commercial production of filled canisters. Thus, for example, in one bulk manufacturing method for preparing suspension aerosol formulations a metering valve is crimped onto an aluminium can to form an empty canister. The particulate medicament is added to a charge vessel and liquefied propellant together with the optional excipients is pressure filled through the charge vessel into a manufacturing vessel. The drug suspension is mixed before recirculation to a filling machine and an aliquot of the drug suspension is then filled through the metering valve into the canister. In one example bulk manufacturing method for preparing solution aerosol formulations a metering valve is crimped onto an aluminium can to form an empty canister. The liquefied propellant together with the optional excipients and the dissolved medicament is pressure filled through the charge vessel into a manufacturing vessel.

In an alternative process, an aliquot of the liquefied formulation is added to an open canister under conditions which are sufficiently cold to ensure the formulation does not vaporise, and then a metering valve crimped onto the canister.

Typically, in batches prepared for pharmaceutical use, each filled canister is check-weighed, coded with a batch number and packed into a tray for storage before release testing.

Suspensions and solutions comprising a compound of formula (I) or a pharmaceutically acceptable salt thereof may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically-acceptable excipients may be added to the suspension or solution. The compound of formula (I) or pharmaceutically acceptable salt thereof may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of formula (I) or pharmaceutically acceptable salt thereof. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

In a further aspect, the invention is directed to a dosage form adapted for intranasal administration.

Formulations for administration to the nose may include pressurised aerosol formulations and aqueous formulations administered to the nose by pressurised pump. Formulations which are non-pressurised and adapted to be administered topically to the nasal cavity are of particular interest. Suitable formulations contain water as the diluent or carrier for this purpose. Aqueous formulations for administration to the lung or nose may be provided with conventional excipients such as buffering agents, tonicity modifying agents and the like. Aqueous formulations may also be administered to the nose by nebulisation.

The compounds of formula (I) or pharmaceutically acceptable salts thereof may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO05/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. In one embodiment, the fluid dispenser is of the general type illustrated in FIGS. 30-40 of WO05/044354.

Pharmaceutical compositions adapted for intranasal administration wherein the carrier is a solid include a coarse powder having a particle size for example in the range 20 to 500 microns which is administered by rapid inhalation through the nasal passage from a container of the powder held close up to the nose. Suitable compositions wherein the carrier is a liquid, for administration as a nasal spray or as nasal drops, include aqueous or oil solutions of the compound of formula (I) or pharmaceutically acceptable salt thereof.

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

Topical preparations may be administered by one or more applications per day to the affected area; over skin areas occlusive dressings may advantageously be used. Continuous or prolonged delivery may be achieved by an adhesive reservoir system.

For treatments of the eye or other external tissues, for example mouth and skin, the compositions may be applied as a topical ointment or cream. When formulated in an ointment, the compound of formula (I) or a pharmaceutically acceptable salt thereof may be employed with either a paraffinic or a water-miscible ointment base. Alternatively, the compound of formula (I) or pharmaceutically acceptable salt thereof may be formulated in a cream with an oil-in-water cream base or a water-in-oil base.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compound and pharmaceutical formulations according to the invention may be used in combination with or include one or more other therapeutic agents, for example selected from anti-inflammatory agents, anticholinergic agents (particularly an M₁/M₂/M₃ receptor antagonist), β₂-adrenoreceptor agonists, antiinfective agents, such as antibiotics or antivirals, or antihistamines. The invention thus provides, in a further aspect, a combination comprising a compound of formula (I) or a pharmaceutically acceptable salt thereof together with one or more other therapeutically active agents, for example selected from an anti-inflammatory agent, such as a corticosteroid or an NSAID, an anticholinergic agent, a β₂-adrenoreceptor agonist, an antiinfective agent, such as an antibiotic or an antiviral, or an antihistamine. One embodiment of the invention encompasses combinations comprising a compound of formula (I) or a pharmaceutically acceptable salt thereof together with a β₂-adrenoreceptor agonist, and/or an anticholinergic, and/or a PDE-4 inhibitor, and/or an antihistamine.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimise the activity and/or stability and/or physical characteristics, such as solubility, of the therapeutic ingredient. It will be clear also that, where appropriate, the therapeutic ingredients may be used in optically pure form.

In one embodiment, the invention encompasses a combination comprising a compound of formula (I) or a pharmaceutically acceptable salt thereof together with a β₂-adrenoreceptor agonist.

Examples of β₂-adrenoreceptor agonists include salmeterol (which may be a racemate or a single enantiomer such as the R-enantiomer), salbutamol (which may be a racemate or a single enantiomer such as the R-enantiomer), formoterol (which may be a racemate or a single duastereomer such as the R,R-diastereomer), salmefamol, fenoterol carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. In one embodiment, the β₂-adrenoreceptor agonists are long-acting β₂ adrenoreceptor agonists, for example, compounds which provide effective bronchodilation for about 12 hours or longer.

Other β₂-adrenoreceptor agonists include those described in WO 02/066422, WO 02/070490, WO 02/076933, WO 03/024439, WO 03/072539, WO 03/091204, WO 04/016578, WO 2004/022547, WO 2004/037807, WO 2004/037773, WO 2004/037768, WO 2004/039762, WO 2004/039766, WO01/42193 and WO03/042160.

Examples of β₂-adrenoreceptor agonists include:
3-(4-{[6-({(2R)-2-hydroxy-2-[4-hydroxy-3-(hydroxymethyl)phenyl]ethyl}amino)hexyl]oxy}butyl)benzenesulfonamide;
3-(3-{[7-({(2R)-2-hydroxy-2-[4-hydroxy-3-hydroxymethyl)phenyl]ethyl}-amino)heptyl]oxy}propyl)benzenesulfonamide;
4-{(1R)-2-[(6-{2-[(2,6-dichlorobenzyl)oxy]ethoxy}hexyl)amino]-1-hydroxyethyl}-2-(hydroxymethyl)phenol;
4-{(1R)-2-[(6-{-4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;
N-2{2-[4-(3-phenyl-4-methoxyphenyl)aminophenyl]ethyl}-2-hydroxy-2-(8-hydroxy-2(1H)-quinolinon-5-yl)ethylamine; and
5-[(R)-2-(2-{-4-[4-(2-amino-2-methyl-propoxy)-phenylamino]-phenyl}-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Suitable anti-inflammatory agents include corticosteroids. Suitable corticosteroids which may be used in combination with the compounds of formula (I) or pharmaceutically acceptable salts thereof are those oral and inhaled corticosteroids and their pro-drugs which have anti-inflammatory activity. Examples include methyl prednisolone, prednisolone, dexamethasone, fluticasone propionate, 6α,9α-difluoro-1,8-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. Preferred corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester. In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Examples of corticosteroids may include those described in WO2002/088167, WO2002/100879, WO2002/12265, WO2002/12266, WO2005/005451, WO2005/005452, WO2006/072599 and WO2006/072600.

Non-steroidal compounds having glucocorticoid agonism that may possess selectivity for transrepression over transactivation and that may be useful in combination therapy include those covered in the following patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651 and WO03/08277. Further non-steroidal compounds are covered in: WO2006/000401, WO2006/000398 and WO2006/015870.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example montelukast), iNOS inhibitors, tryptase and elastase inhibitors, beta-2 integrin antagonists and adenosine receptor agonists or antagonists (e.g. adenosine 2a agonists), cytokine antagonists (for example chemokine antagonists, such as a CCR3 antagonist) or inhibitors of cytokine synthesis, or 5-lipoxygenase inhibitors. An iNOS (inducible nitric oxide synthase inhibitor) is preferably for oral administration. Examples of iNOS inhibitors include those disclosed in WO93/13055, WO98/30537, WO02/50021, WO95/34534 and WO99/62875. Examples of CCR3 inhibitors include those disclosed in WO02/26722.

In one embodiment, the invention provides the use of the compounds of formula (I) and pharmaceutically acceptable salts thereof in combination with a phosphodiesterase 4 (PDE4) inhibitor, especially in the case of a formulation adapted for inhalation. The PDE4-specific inhibitor useful in this aspect of the invention may be any compound that is known to inhibit the PDE4 enzyme or which is discovered to act as a PDE4 inhibitor, e.g. as an inhibitor of PDE4B and/or PDE4D, not compounds which inhibit other members of the PDE family, such as PDE3 and PDE5, as well as PDE4.

Compounds include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep., 1996; this patent and the compounds it discloses are incorporated herein in full by reference.

Other compounds include AWD-12-281 from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Further compounds are disclosed in the published international patent application WO04/024728 (Glaxo Group Ltd.), WO04/056823 (Glaxo Group Ltd.) and WO04/103998 (Glaxo Group Ltd.) (e.g. Example 399 or 544 disclosed therein). Further compounds are also disclosed in WO2005/058892, WO2005/090348, WO2005/090353, and WO2005/090354, all in the name of Glaxo Group Limited.

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (for example, as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Additional compounds are disclosed in WO 2005/037280, WO 2005/046586 and WO 2005/104745, incorporated herein by reference. The present combinations include, but are not limited to:
(3-endo)-3-(2,2-di-2-thienylethenyl)-8,8-dimethyl-8-azoniabicyclo[3.2.1]octane iodide;
(3-endo)-3-(2-cyano-2,2-diphenylethyl)-8,8-dimethyl-8-azoniabicyclo[3.2.1]octane bromide;
4-[hydroxy(diphenyl)methyl]-1-{2-[(phenylmethyl)oxy]ethyl}-1-azoniabicyclo[2.2.2]octane bromide; and
(1R,5S)-3-(2-cyano-2,2-diphenylethyl)-8-methyl-8-{2-[(phenylmethyl)oxy]ethyl}-8-azoniabicyclo[3.2.1]octane bromide.

Other anticholinergic agents include compounds which are disclosed in U.S. patent application 60/487,981 including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further anticholinergic agents include compounds which are disclosed in U.S. patent application 60/511,009 including, for example:
(endo)-3-(2-methoxy-2,2-di-thiophen-2-yl-ethyl)-8,8-dimethyl-8-azonia-bicyclo[3.2.1]octane iodide;
3-((endo)-8-methyl-8-aza-bicyclo[3.2.1]oct-3-yl)-2,2-diphenyl-propionitrile;
(endo)-8-methyl-3-(2,2,2-triphenyl-ethyl)-8-aza-bicyclo[3.2.1]octane;
3-((endo)-8-methyl-8-aza-bicyclo[3.2.1]oct-3-yl)-2,2-diphenyl-propionamide;
3-((endo)-8-methyl-8-aza-bicyclo[3.2.1]oct-3-yl)-2,2-diphenyl-propionic acid;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
3-((endo)-8-methyl-8-aza-bicyclo[3.2.1]oct-3-yl)-2,2-diphenyl-propan-1-ol;
N-benzyl-3-((endo)-8-methyl-8-aza-bicyclo[3.2.1]oct-3-yl)-2,2-diphenyl-propionamide;
(endo)-3-(2-carbamoyl-2,2-diphenyl-ethyl)-8,8-dimethyl-8-azonia-bicyclo[3.2.1]octane iodide;
1-benzyl-3-[3-((endo)-8-methyl-8-aza-bicyclo[3.2.1]oct-3-yl)-2,2-diphenyl-propyl]-urea;
1-ethyl-3-[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-acetamide;
N-[3-((endo)-8-methyl-8-aza-bicyclo[3.2.1]oct-3-yl)-2,2-diphenyl-propyl]-benzamide;
3-((endo)-8-methyl-8-aza-bicyclo[3.2.1]oct-3-yl)-2,2-di-thiophen-2-yl-propionitrile;
(endo)-3-(2-cyano-2,2-di-thiophen-2-yl-ethyl)-8,8-dimethyl-8-azonia-bicyclo[3.2.1]octane iodide;
N-[3-((endo)-8-methyl-8-aza-bicyclo[3.2.1]oct-3-yl)-2,2-diphenyl-propyl]-benzenesulfonamide;
[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-methanesulfonamide; and/or
(endo)-3-{2,2-diphenyl-3-[(1-phenyl-methanoyl)-amino]-propyl}-8,8-dimethyl-8-azonia-bicyclo[3.2.1]octane bromide.

Further compounds include:
(endo)-3-(2-methoxy-2,2-di-thiophen-2-yl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
(endo)-3-(2-carbamoyl-2,2-diphenyl-ethyl)-8,8-dimethyl-8-azonia-bicyclo[3.2.1]octane iodide;
(endo)-3-(2-cyano-2,2-di-thiophen-2-yl-ethyl)-8,8-dimethyl-8-azonia-bicyclo[3.2.1]octane iodide; and/or
(endo)-3-{2,2-diphenyl-3-[(1-phenyl-methanoyl)-amino]-propyl}-8,8-dimethyl-8-azonia-bicyclo[3.2.1]octane bromide.

In one embodiment the invention provides a combination comprising a compound of formula (I) or a pharmaceutically acceptable salt thereof together with an H1 antagonist. Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I) or a pharmaceutically acceptable salt thereof together with an H3 antagonist (and/or inverse agonist). Examples of H3 antagonists include, for example, those compounds disclosed in WO2004/035556 and in WO2006/045416. Other histamine receptor antagonists which may be used in combination with the compounds of formula (I) or pharmaceutically acceptable salts thereof include antagonists (and/or inverse agonists) of the H4 receptor, for example, the compounds disclosed in Jablonowski et al., J. Med. Chem. 46:3957-3960 (2003).

The invention thus provides, in a further aspect, a combination comprising a compound of formula (I) or a pharmaceutically acceptable salt thereof together with a PDE4 inhibitor.

The invention thus provides, in a further aspect, a combination comprising a compound of formula (I) or a pharmaceutically acceptable salt thereof together with a β₂-adrenoreceptor agonist.

The invention thus provides, in a further aspect, a combination comprising a compound of formula (I) or a pharmaceutically acceptable salt thereof together with a corticosteroid.

The invention thus provides, in a further aspect, a combination comprising a compound of formula (I) or a pharmaceutically acceptable salt thereof together with a non-steroidal GR agonist.

The invention thus provides, in a further aspect, a combination comprising a compound of formula (I) or a pharmaceutically acceptable salt thereof together with an anticholinergic.

The invention thus provides, in a further aspect, a combination comprising a compound of formula (I) or a pharmaceutically acceptable salt thereof together with an antihistamine.

The invention thus provides, in a further aspect, a combination comprising a compound of formula (I) or a pharmaceutically acceptable salt thereof together with a PDE4 inhibitor and a β₂-adrenoreceptor agonist.

The invention thus provides, in a further aspect, a combination comprising a compound of formula (I) or a pharmaceutically acceptable salt thereof together with an anticholinergic and a PDE-4 inhibitor.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The invention thus provides, in a further aspect, a pharmaceutical composition comprising a combination of a compound of formula (I) or a pharmaceutically acceptable salt thereof together with another therapeutically active agent.

The invention thus provides, in a further aspect, a pharmaceutical composition comprising a combination of a compound of formula (I) or a pharmaceutically acceptable salt thereof together with a PDE4 inhibitor.

The invention thus provides, in a further aspect, a pharmaceutical composition comprising a combination of a compound of formula (I) or a pharmaceutically acceptable salt thereof together with a β₂-adrenoreceptor agonist.

The invention thus provides, in a further aspect, a pharmaceutical composition comprising a combination of a compound of formula (I) or a pharmaceutically acceptable salt thereof together with a corticosteroid.

The invention thus provides, in a further aspect, a pharmaceutical composition comprising a combination of a compound of formula (I) or a pharmaceutically acceptable salt thereof together with a non-steroidal GR agonist.

The invention thus provides, in a further aspect, a pharmaceutical composition comprising a combination of a compound of formula (I) or a pharmaceutically acceptable salt thereof together with an anticholinergic.

The invention thus provides, in a further aspect, a pharmaceutical composition comprising a combination of a compound of formula (I) or a pharmaceutically acceptable salt thereof together with an antihistamine.

The invention thus provides, in a further aspect, a pharmaceutical composition comprising a combination of a compound of formula (I) or a pharmaceutically acceptable salt thereof together with a PDE4 inhibitor and a β₂-adrenoreceptor agonist.

The invention thus provides, in a further aspect, a pharmaceutical composition comprising a combination of a compound of formula (I) or a pharmaceutically acceptable salt thereof together with an anticholinergic and a PDE4 inhibitor.

The invention will now be illustrated by way of the following non-limiting examples.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Unless otherwise noted, all starting materials were obtained from commercial suppliers and used without further purification. Unless otherwise indicated, all temperatures are expressed in ° C. (degrees Centigrade). All reactions are conducted under an inert atmosphere at room temperature unless otherwise noted. All references to ether are to diethyl ether; brine refers to a saturated aq. solution of NaCl.

¹H NMR spectra were recorded using a Bruker DPX 400 MHz, referenced to tetramethylsilane.

LC/MS was conducted using either Method A or Method B:

Method A: LC/MS (5 min system) was conducted on a Supelcosil LCABZ+PLUS column (3.3 cm×4.6 mm ID) eluting with 0.1% HCO₂H and 0.01M ammonium acetate in water (solvent A) and 0.05% HCO₂H 5% water in acetonitrile (solvent B), using the following elution gradient 0.0-0.7 min 0% B, 0.7-4.2 min 0-100% B, 4.2-4.6 min 100% B, 4.6-4.8 min 100-0% B at a flow rate of 3 ml/min. The mass spectra were recorded on a Waters ZQ Mass spectrometer using electrospray positive and negative mode (ES+ve and ES−ve)
Method B: LC/MS (2 min system) was conducted on a Acquity HPLC BEH C₁₈ column (5.0 cm×2.1 mm) at 40° C., eluting with 0.1% HCO₂H and 0.01M ammonium acetate in water (solvent A) and 0.05% HCO₂H 5% water in acetonitrile (solvent B), using the following elution gradient 0.0-0.1 min 3% B, 0.1-1.4 min 3-100% B, 1.4-1.9 min 100% B, 1.9-2 min 3% B at a flow rate of 1 ml/min. The mass spectra were recorded on a Waters ZQ Mass spectrometer using electrospray with pos negative switching (ES+ve and ES−ve).

In the LCMS data reported herein, the mass ion was mathematically rounded to the nearest integer.

“Mass directed autoprep”/“MDAP”/“preparative mass directed HPLC” was conducted on a system such as; a Waters FractionLynx system comprising of a Waters 600 pump with extended pump heads, Waters 2700 autosampler, Waters 996 diode array and Gilson 202 fraction collector on a 10 cm 2.54 cm ID ABZ+ column, eluting with either 0.1% formic acid or TFA in water (solvent A) and 0.1% formic or TFA in acetonitrile (solvent B) using the appropriate elution gradient. Mass spectra were recorded on Micromass ZMD mass spectrometer using electrospray positive and negative mode, alternate scans. The software used was MassLynx 3.5 with OpenLynx and FractionLynx optio or using equivalent alternative systems.

“Hydrophobic frits” refers to filtration tubes sold by Whatman. SPE (solid phase extraction, SCX-2 and aminopropyl) refers to the use of cartridges sold by International Sorbent Technology Ltd. The Flashmaster II is an automated multi-user flash chromatography system, available from Argonaut Technologies Ltd., which utilises disposable, normal phase, SPE cartridges (2 g to 100 g). It provides quaternary on-line solvent mixing to enable gradient methods to be run. Samples are queued using the multi-functional open access software, which manages solvents, flow-rates, gradient profile and collection conditions. The system is equipped with a Knauer variable wavelength uv-detector and two Gilson FC204 fraction-collectors enabling automated peak cutting, collection and tracking.

Silica chromatography techniques include either automated (Flashmaster) techniques or manual chromatography on pre-packed cartridges (SPE) or manually-packed flash columns.

Microwave chemistry was typically performed in sealed vessels, irradiating with a suitable microwave reactor system, such as a Biotage Initiator™ Microwave Synthesiser.

When the name of a commercial supplier is given after the name of a compound or a reagent, for instance “compound X (Aldrich)” or “compound X/Aldrich”, this means that compound X is obtainable from a commercial supplier, such as the commercial supplier named. H cubes are hydrogen generators commercially available from, for example, Asynt. For example, 1,1′-bis(diphenylphosphino) ferrocenedichloro palladium(II), complex with dichloromethane may be purchased from Acros, and tetrabutylammonium fluoride (1M solution in tetrahydrofuran) and trifluoroacetic acid may be purchased from Aldrich.

Similarly, when a literature or a patent reference is given after the name of a compound, for instance compound Y (EP 0 123 456), this means that the preparation of the compound is described in the named reference.

The names of the Examples have been obtained from the structures using the compound naming programme “ACD Name Pro 6.02”.

### Intermediate 1

### 4-Bromo-1-[(4-methyl phenyl)sulfonyl]-1H-pyrrolo[2,3-b]pyridine

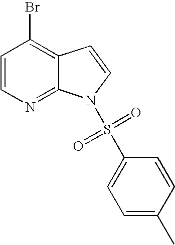

Toluenesulfonyl chloride (219.0 g, 1.126 mol) and tetrabutylammonium hydrogen sulphate (9.8 g, 28.0 mmol) were added to a solution of 4-bromo-1H-pyrrolo[2,3-b]pyridine (which may be prepared as described in Org Letts, 2003, 5, 5023) (111.0 g, 0.563 mol) in DCM (2.8 L). To this was then added dropwise over 30 min a solution of sodium hydroxide (67.56 g, 1.59 mol) in water (281.2 mL), and the resultant reaction mixture was stirred at room temperature for 4 h. Water (562 mL) was then added, the organic phase was separated, washed with water (2×562 mL), dried (Na₂SO₄), and the solvent was removed under reduced pressure. The residue was taken up and heated at reflux in hexane twice (1.24 L) before being allowed to cool to room temperature. The resultant precipitate was then collected by filtration, washed with hexane (2×570 mL), and dried. It was then allowed to form a slurry in 1:1 MeOH/water (304 mL) before once again being filtered off, washed with 1:1 MeOH/water (2×304 mL), and dried under vacuum. The process was repeated a further time, with a slurry being formed in hexane (1.14 L), to give 4-bromo-1-[(4-methylphenyl)sulfonyl]-1H-pyrrolo[2,3-b]pyridine (158.9 g, 80%) as a brown solid.

### Intermediate 2

### 4-Bromo-2-iodo-1-[(4-methylphenyl)sulfonyl]-1H-pyrrolo[2,3-b]pyridine

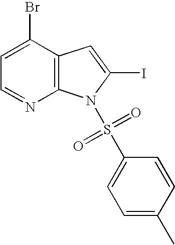

n-Bulithium (63.2 mL of a 2.5 M solution in hexanes, 0.158 mol) was added by syringe to a solution of diisopropylamine (23.8 mL, 0.17 mol) in anhydrous THF (717 mL) at 0° C. and upon complete addition, the mixture was cooled to −78° C. 4-Bromo-1-[(4-methylphenyl)sulfonyl]-1H-pyrrolo[2,3-b]pyridine (52.8 g, 0.15 mol) was added, the resultant reaction mixture was stirred at −78° C. for 1.5 h, and iodine (50.13 g, 0.196 mol) was then added. After being allowed to stir at −78° C. for a further 20 min, the reaction mixture was allowed to warm to −20° C. and was quenched by the addition of saturated NH₄Cl solution (226 mL). Upon reaching room temperature, the organic phase was separated, washed with saturated NH₄Cl solution, dried (Na₂SO₄), and the solvent was removed under reduced pressure to afford the crude product. This was purified by repeated slurrying at 5° C. in MeOH (3×190 mL) to give 4-bromo-2-iodo-1-[(4-methylphenyl)sulfonyl]-1H-pyrrolo[2,3-b]pyridine (50.23 g, 70%) as a brown solid, mp 140-143° C.

### Intermediate 3

### Tetrahydro-4H-thiopyran-4-one oxime

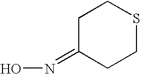

Anhydrous sodium acetate (16.25 g, 18.86 mmol) and hydroxylamine hydrochloride (7.72 g, 10.66 mmol) were added to a solution of 4-oxothiane (10.02 g, 8.2 mmol) in ethanol (100 ml) and water (100 ml). The reaction mixture was stirred and heated at reflux for 56 h. The cooled reaction mixture was diluted with water (200 ml) and extracted with ether (2×150 ml). The combined organic extracts were washed with brine (100 ml), dried (hydrophobic frit) and evaporated to dryness to give the title compound as a colourless solid (12.8 g).

MH+=132, rt=1.56 min

### Intermediate 4

### Tetrahydro-2H-thiopyran-4-amine

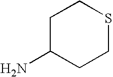

2.3M Lithium aluminium hydride in THF (100 ml) was transferred under nitrogen to a 1 litre 3-necked flask and diluted with dry THF (130 ml) to make a 1M lithium aluminium hydride solution. The solution was stirred under nitrogen whilst a solution of tetrahydro-4H-thiopyran-4-one oxime (12.8 g, 97.6 mmol) in dry THF (90 ml) was added drop wise. The addition was exothermic and was done slowly whilst cooling the reaction in a water bath so that the reaction stayed at or below 25° C. Addition was over 2 h. After the addition the mixture was stirred at ambient temperature for a further 0.5 to 1 hour and cautiously warmed to reflux. Stirring at reflux was continued overnight. The reaction was allowed to cool, diluted by addition of dry THF (130 ml) and cooled to below 10° C. in an ice/water bath. It was quenched by slowly adding water (10 ml) keeping the temperature below 20° C. (very exothermic and mixture became quite thick). Aq. sodium hydroxide solution (15% w/v, 10 ml) was added slowly drop wise keeping the temperature below 20° C. Water (26 ml) was added drop wise. The mixture was stirred for a while longer then the solid was filtered off washing the flask and solid with THF. The solvent was removed and the remaining yellow oil was dried under vacuum overnight to give the title compound as a colourless solid (6.3 g).

MH+=118, rt=0.32 min

### Intermediate 5

### 1,1-Dimethylethyl tetrahydro-2H-thiopyran-4-ylcarbamate

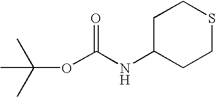

Tetrahydro-2H-thiopyran-4-amine (5.8 g, 49.5 mmol) was dissolved in dioxan (55 ml). Aq. 2M sodium hydroxide was added. Di-tert-buty dicarbonate (21.6 g, 99 mmol) was added portion wise keeping the temperature below 30° C. (ice/water bath). The last portion was washed in with a little dioxan. The mixture was stirred for a further 2 h at ambient temperature. It was diluted with water (100 ml) and extracted with EtOAc (3×200 ml). The combined EtOAc extracts were washed with water (100 ml), brine (100 ml), dried (MgSO₄), filtered, evaporated and dried (high vacuum) to give crude material (18 g). This was dissolved in DCM (60 ml) and applied to a 9 cm diameter glass column. The column was packed in 9:1 cyclohexane:EtOAc. It was eluted with 9:1 cyclohexane:EtOAc. Product containing fractions were pooled and evaporated to give the title compound (9.5 g). MH+=218, rt=2.81 min

### Intermediate 6

### 1,1-Dimethylethyl (1,1-dioxidotetrahydro-2H-thiopyran-4-yl)carbamate

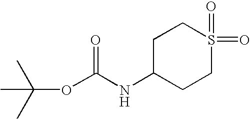

A solution of 1,1-dimethylethyl tetrahydro-2H-thiopyran-4-ylcarbamate (9.5 g, 44 mmol) in methanol (300 ml) was stirred and cooled to 0-5° C. in an ice/IMS bath. A solution of oxone (43.6 g, 70.9 mmol) in water (300 ml) was added drop wise over 2 h keeping the temperature below 10° C. After the addition was complete, water was added to the cooling bath. The mixture was stirred overnight whilst slowly warming to ambient temperature. The mixture was poured, with stirring, onto stirred aq. potassium carbonate solution (10% w/v, 650 ml). Water (200 ml) was added and the mixture extracted with EtOAc (3×500 ml). The combined EtOAc extracts were washed with water (500 ml), brine (300 ml), dried (MgSO₄), filtered, evaporated and dried (high vacuum) to give the title compound as a white solid (9.9 g).

ELSD MH+250, MNH₄+=267 seen at rt=2.14 min

### Intermediate 7

### (1,1-Dioxidotetrahydro-2H-thiopyran-4-yl)amine hydrochloride

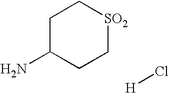

1,1-Dimethylethyl (1,1-dioxidotetrahydro-2H-thiopyran-4-yl)carbamate (9.9 g, 40 mmol) was dissolved in 1,4 dioxan (210 ml) and the solution stirred under nitrogen. 5M Aq. HCl (105 ml, 525 mmol) was added dropwise keeping the temperature below 25° C. (ice/water bath). Stirring was continued at ambient temperature overnight. The solvent was removed in vacuo. The residue was evaporated down again from dioxan to azeotrope off the water and HCl. The residual white powder was dried (high vacuum) and overnight in the vac oven (40° C.) to give the title compound (7.2 g). MH+=150, rt=0.31 min

### Intermediate 8

### 4-Bromo-1-(phenylsulfonyl)-1H-pyrrolo[2,3-b]pyridine

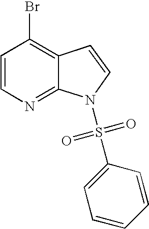

4-Bromo-1H-pyrrolo[2,3-b]pyridine (which may be prepared as described in Org Letts, 2003, 5, 5023) (15.0 g, 76 mmol) was suspended in DCM (300 ml) and 1,4-dioxane (100 ml) and 50% aq. sodium hydroxide (23 ml) was added followed by NaBu₄HSO₄ solution (7.5 ml). The mixture was vigorously stirred and cooled in an ice bath whilst benzene sulfonyl chloride (14.6 ml, 115 mmol) was added dropwise. The reaction mixture was left stirring vigorously for 5 days. It was evaporated to dryness and the resulting solid was washed with water (100 ml) followed by methanol (50 ml). The yellow solid was dried in vacuo. The title compound was obtained as a pale yellow solid (23.5 g, 92%).

MH⁺337/339, rt=3.35 min

### Intermediate 9

### 4-Bromo-2-methyl-1-(phenylsulfonyl)-1H-pyrrolo[2,3-b]pyridine

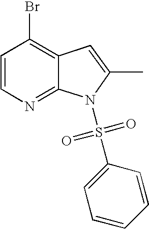

To the solution of 4-bromo-1-(phenylsulfonyl)-1H-pyrrolo[2,3-b]pyridine (9.4 g, 28.0 mmol) in dry THF (100 ml) stirred at −35° C. was added 2M LDA in heptane/THF/ethylbenzene (28.0 ml, 56.0 mmol) and the reaction was stirred at −35° C. for 30 min. Methyl iodide (10.5 ml, 168.0 mmol) was added drop wise to the solution and the mixture was allowed to warm to room temperature over 2 h. The reaction was quenched with aqueous ammonium chloride solution (100 ml) and extracted with EtOAc (3×60 ml). The combined organic layers were dried (phase separator) and concentrated in vacuo. Purification was by FlashMaster on silica gel (2×70 g) using EtOAc-cyclohexane (0-100% gradient) to give the title compound as a white solid (7.85 g, 80%).

MH+351/353, rt=3.45 min

### Intermediate 10

### 2-Methyl-1-(phenylsulfonyl)-4-(4,4,5,5-tetramethyl-1,3,2-dioxaborolan-2-yl)-1H-pyrrolo[2,3-b]pyridine

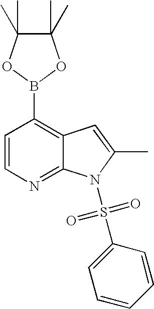

4-Bromo-2-methyl-1-(phenylsulfonyl)-1H-pyrrolo[2,3-b]pyridine (0.500 g, 1.42 mmol) was stirred at room temperature in dry DMF (10 ml) and the solution was degassed for 10 min.

4,4,4′,4′,5,5,5′,5′-Octamethyl-2,2′-bi-1,3,2-dioxaborolane (0.723 g, 2.85 mmol), bis(diphenylphosphino)ferrocene palladium II chloride (0.058 g, 0.07 mmol) and potassium acetate (0.447 g, 4.56 mmol) were added and the reaction stirred at 80° C. under nitrogen for 4 h. The reaction mixture was cooled to room temperature and concentrated in vacuo. The residue was partitioned between DCM (100 ml), water (50 ml) and saturated citric acid (50 ml). The organic layer was washed with water (50 ml), dried (hydrophobic frit) and concentrated in vacuo. The residue was purified by Flashmaster (Silica, 50 g) using 0-100% EtOAc-cyclohexane. The desired fractions were combined and concentrated in vacuo to give the title compound as a white solid (0.362 g, 64%).

MH+=399, rt=3.69 min

### Intermediate 11

### 5-Bromo-N-(2-hydroxyethyl)-2-thiophenesulfonamide

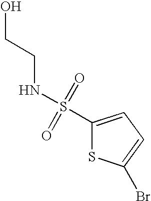

Ethanolamine (12.69 ml, 210.28 mmol), TEA (79.94 ml, 573.5 mmol) and anhydrous DCM (200 ml) were combined and cooled to 0° C. 5-Bromothiophene-2-sulfonyl chloride (50 g, 191.17 mmol) in DCM (50 ml) was added slowly and the reaction mixture stirred overnight under nitrogen. The reaction mixture was concentrated and diluted with EtOAc (750 ml). The organic phase was washed with 1M sodium hydroxide (250 ml), sodium hydrogen carbonate, brine, dried (MgSO₄), filtered and concentrated to give the title compound as a cream solid (26.68 g, 48%).

MH+=288, M-H+=284/286, rt=2.37 min

### Intermediate 12

### 5-Bromo-N-(1,1-dioxidotetrahydro-2H-thiopyran-4-yl)-2-thiophenesulfonamide

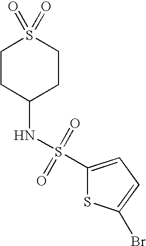

(1,1-Dioxidotetrahydro-2H-thiopyran-4-yl)amine hydrochloride (3.856 g, 21 mmol) and TEA (12.97 ml, 95 mmol) were stirred at room temperature in DCM (25 ml) under nitrogen for 2 h. A solution of 5-bromothiophene-2-sulfonyl chloride (5 g, 19 mmol) in DCM (25 ml) was added slowly to the mixture and stirring was continued for 20 h. DCM (25 ml), citric acid (100 ml) and EtOAc (25 ml) were added. The organic phases were washed with saturated sodium hydrogen carbonate (100 ml), water (100 ml) and brine (100 ml), dried (hydrophobic frit) and concentrated in vacuo to give the title compound (3.4 g, 48%). M-H+=374, rt=2.57 min

### Intermediate 13

### N-(1,1-Dioxidotetrahydro-2H-thiopyran-4-yl)-5-[2-methyl-1-(phenylsulfonyl)-1H-pyrrolo[2,3-b]pyridin-4-yl]-2-thiophenesulfonamide

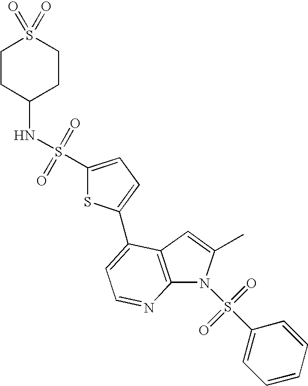

A mixture of 2-methyl-1-(phenylsulfonyl)-4-(4,4,5,5-tetramethyl-1,3,2-dioxaborolan-2-yl)-1H-pyrrolo[2,3-b]pyridine (0.260 g, 0.653 mmol), 5-bromo-N-(1,1-dioxidotetrahydro-2H-thiopyran-4-yl)-2-thiophenesulfonamide (0.366 g, 0.979 mmol), bis(diphenylphosphino)ferrocene palladium II chloride (0.053 g, 0.065 mmol) and sodium carbonate (0.207 g, 1.959 mmol) in dioxan (3.5 ml) and water (0.7 ml) were heated at 150° C. for 30 min in the Biotage Initiator microwave. The dark solution was partitioned between DCM:EtOAc (1:1, 25 ml) and water:saturated citric acid (1:1, 25 ml). The organic phases were combined, dried (hydrophobic frit) and reduced in vacuo. Purification was by FlashMaster on silica gel eluting with 0-100% EtOAc-cyclohexane. The desired fraction was concentrated in vacuo to give the title compound (0.159 g, 43%).

MH+=566, rt=1.1 min

### Intermediate 14

### 1,1-Dimethylethyl (2-{[(5-bromo-2-thienyl)sulfonyl]amino}ethyl)carbamate

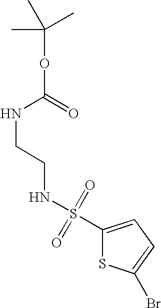

1,1-Dimethylethyl (2-aminoethyl)carbamate (33.29 ml, 210.28 mmol) and TEA (79.94 ml, 573.5 mmol) were dissolved in DCM (250 ml) and 5-bromothiophene-2-sulfonyl chloride (50 g, 191.17 mmol) in DCM (250 ml) was added. The reaction was exothermic. The reaction was stirred at room temperature overnight. The reaction mixture was diluted with 0.5M hydrochloric acid (500 ml). The organic phase was washed with water and brine, dried (MgSO₄), filtered and concentrated to give the title compound as a cream solid (70.73 g, 96%).

MH+385/387 rt=3.11 min

### Intermediate 15

### 1,1-Dimethylethyl{2-[({5-[2-methyl-1-(phenylsulfonyl)-1H-pyrrolo[2,3-b]pyridin-4-yl]-2-thienyl}sulfonyl)amino]ethyl}carbamate

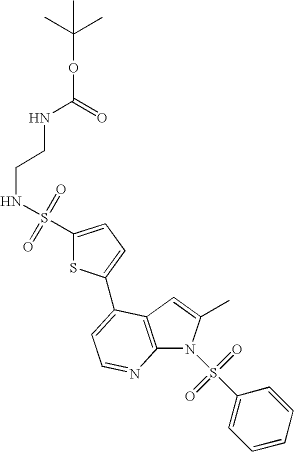

4-Bromo-2-methyl-1-(phenylsulfonyl)-1H-pyrrolo[2,3-b]pyridine (0.250 g, 0.712 mmol), 4,4,4′,4′,5,5,5′,5′-octamethyl-2,2′-bi-1,3,2-dioxaborolane (0.902 g, 3.55 mmol), bis(diphenylphosphino)ferrocene palladium II chloride (0.058 g, 0.07 mmol) and potassium acetate (0.348 g, 3.55 mmol) were stirred at room temperature under nitrogen in dry DMF (7.5 ml) for 4 h and at 80° C. for 2 h. Water (1.5 ml) was added and the reaction heated at 80° C. for 10 min. 1,1-Dimethylethyl (2-{[(5-bromo-2-thienyl)sulfonyl]amino}ethyl)carbamate (0.409 g, 1.068 mmol) was added and the mixture heated at 80° C. for 2 h. The reaction was partitioned between DCM (50 ml) and water (50 ml). The aqueous phase was extracted with DCM (15 ml). The combined organic phases were dried (hydrophobic frit), concentrated in vacuo and purified by FlashMaster II on silica gel eluting with 0-100% EtOAc-cyclohexane. The desired fractions were concentrated in vacuo to give the title compound as a yellow solid (0.187 g, 45%).

MH+=577, rt=1.23 min

### Intermediate 16

### 1,1-Dimethylethyl[2-({[5-(2-methyl-1H-pyrrolo[2,3-b]pyridin-4-yl)-2-thienyl]sulfonyl}amino)ethyl]carbamate trifluoroacetate

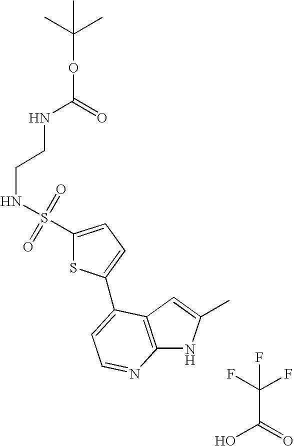

A mixture of 1,1-dimethylethyl{2-[({5-[2-methyl-1-(phenylsulfonyl)-1H-pyrrolo[2,3-b]pyridin-4-yl]-2-thienyl}sulfonyl)amino]ethyl}carbamate (0.180 g, 0.313 mmol) and 10M sodium hydroxide in water (0.5 ml) in dioxan/water (5:1, 3.5 ml:0.7 ml) were heated in the Biotage Initiator microwave at 150° C. for 10 min. The reaction mixture was partitioned between EtOAc (20 ml), water (10 ml) and saturated citric acid was added to pH6. The aqueous phase was extracted with EtOAc (10 ml). The combined organic extracts were dried (hydrophobic frit), concentrated in vacuo and purified by MDAP. The desired fractions were combined and concentrated in vacuo to give the title compound as a yellow glass (0.092 g, 53%, TFA salt).

MH+=437, rt=1.01 min

### Intermediate 17

### 1,1-Dimethylethyl 5-{4-bromo-1-[(4-methylphenyl)sulfonyl]-1H-pyrrolo[2,3-b]pyridin-2-yl}-3,4-dihydro-1(2H)-pyridinecarboxylate

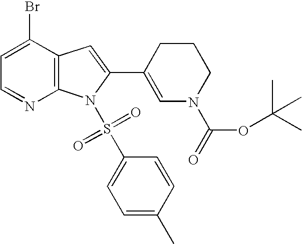

A solution of 4-bromo-2-iodo-1-[(4-methylphenyl)sulfonyl]-1H-pyrrolo[2,3-b]pyridine 3 g, 6.29 mmol) in THF (12.5 ml) at −15° C. was treated drop wise with isopropylmagnesium Grignard solution (2M/THF, 3.77 ml, 7.55 mmol) and stirred between −15° C. and −20° C. for 30 min. 1,1-Dimethylethyl 5-{[(trifluoromethyl)sulfonyl]oxy}-3,4-dihydro-1(2H)-pyridinecarboxylate (which may be prepared as described in Tetrahedron, Vol. 52, No. 27, p9101-9110, 1996) (2.5 g, 7.55 mmol) was added followed by tetrakis(triphenylphosphine)palladium(0) (0.145 g, 0.126 mmol) and the solution was allowed to warm to room temperature. It was heated at reflux for 10 min. The reaction was quenched with MeOH (10 ml), reduced in vacuo and purified by 100 g silica FlashMaster II eluting with 0-100% EtOAc-cyclohexane over 60 min to afford the title compound (3.28 g, 98%).

MH+=532/534, rt=3.92 min

### Intermediate 18

### 1,1-Dimethylethyl 5-[1-[(4-methylphenyl)sulfonyl]-4-(4,4,5,5-tetramethyl-1,3,2-dioxaborolan-2-yl)-1H-pyrrolo[2,3-b]pyridin-2-yl]-3,4-dihydro-1(2H)-pyridinecarboxylate

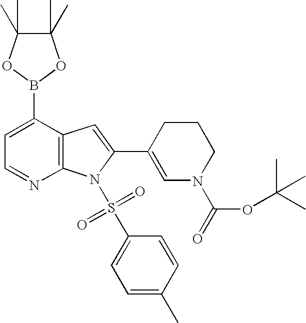

1,1-Dimethylethyl 5-{4-bromo-1-[(4-methylphenyl)sulfonyl]-1H-pyrrolo[2,3-b]pyridin-2-yl}-3,4-dihydro-1(2H)-pyridinecarboxylate (1.5 g, 2.8 mmol) potassium acetate (0.83 g, 8.45 mmol) and palladium acetate (0.034 g, 0.14 mmol) in DMF (30 ml) under nitrogen were treated with bis(pinacolato)diboron (1.72 g, 6.8 mmol) in DMF (6 ml). The reaction was stirred at 40° C. for 20 min and at 50° C. for 2 h. The reaction was reduced in vacuo, diluted with water (100 ml) and extracted with DCM (2×100 ml). The organic layers were dried (phase separator), concentrated in vacuo and the solid triturated with cyclohexane to give impure title compound (1.06 g).

MH+=580, rt=4.09 min

### Intermediate 19

### 1,1-Dimethylethyl 5-[4-(5-{[(2-hydroxyethyl)amino]sulfonyl}-2-thienyl)-1H-pyrrolo[2,3-b]pyridin-2-yl]-3,4-dihydro-1(2H)-pyridinecarboxylate

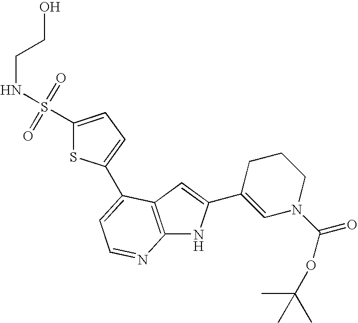

1,1-Dimethylethyl 5-[1-[(4-methylphenyl)sulfonyl]-4-(4,4,5,5-tetramethyl-1,3,2-dioxaborolan-2-yl)-1H-pyrrolo[2,3-b]pyridin-2-yl]-3,4-dihydro-1(2H)-pyridinecarboxylate (0.200 g, 0.34 mmol), 2-(dimethylamino)-2-biphenyl palladium (II) chloride dinorbornyl phosphine (0.010 g), potassium phosphate tribasic (0.220 g, 1.04 mmol) and 5-bromo-N-(2-hydroxyethyl)-2-thiophenesulfonamide (0.119 g, 0.408 mmol) in dioxan (2.5 ml) and water (0.5 ml) were heated in the Biotage Initiator microwave at 140° C. for 40 min. The reaction was poured into water (20 ml) and extracted with DCM (2×25 ml). The combined organic extracts were dried (phase separator) and reduced in vacuo to give (0.350 g) of crude material which was diluted with THF (3 ml) and MeOH (1.5 ml) and treated with 5M sodium hydroxide (0.7 ml). The reaction was stirred for 18 h. The reaction was poured into water (20 ml) and extracted with DCM (2×25 ml). The organic layers were reduced in vacuo (0.300 g) and purified by MDAP to give the title compound (0.035 g, 20%).

MH+=505 rt=3.16 min

### Intermediate 20

### 1,1-Dimethylethyl 5-{4-[5-({[2-({[(1,1-dimethylethyl)oxy]carbonyl}amino)ethyl]amino}sulfonyl)-2-thienyl]-1H-pyrrolo[2,3-b]pyridin-2-yl}-3,4-dihydro-1(2H)-pyridinecarboxylate

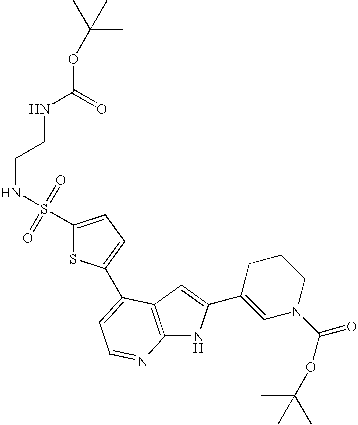

1,1-Dimethylethyl 5-[1-[(4-methylphenyl)sulfonyl]-4-(4,4,5,5-tetramethyl-1,3,2-dioxaborolan-2-yl)-1H-pyrrolo[2,3-b]pyridin-2-yl]-3,4-dihydro-1(2H)-pyridinecarboxylate (0.200 g, 0.34 mmol), 2-(dimethylamino)-2-biphenyl palladium (II) chloride dinorbornyl phosphine (0.010 g), potassium phosphate tribasic (0.220 g, 1.04 mmol) and 1,1-dimethylethyl (2-{[(5-bromo-2-thienyl)sulfonyl]amino}ethyl)carbamate (0.160 g, 0.408 mmol) in dioxan (2.5 ml) and water (0.5 ml) were heated in the Biotage Initiator microwave at 140° C. for 40 min. The reaction was poured into water (20 ml) and extracted with DCM (2×25 ml). The combined organic extracts were dried (phase separator) and reduced in vacuo to give (0.500 g) of crude material which was diluted with THF (3 ml) and MeOH (1.5 ml) and treated with 5M sodium hydroxide (0.7 ml). The reaction was stirred for 18 h. The reaction was poured into water (20 ml) and extracted with DCM (2×25 ml). The organic layers were reduced in vacuo (0.445 g) and purified by MDAP to give the title compound (0.065 g).

MH+=604, rt=3.56 min

### Intermediate 21

### 5-Bromo-N-(1,1-dioxidotetrahydro-3-thienyl)-2-thiophenesulfonamide

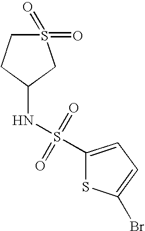

(1,1-Dioxidotetrahydro-3-thienyl)amine hydrochloride (36.1 g, 210.28 mmol), TEA (79.94 ml, 573.50 mmol) and anhydrous DCM (300 ml) were combined followed by slow addition of 5-bromo-2-thiophenesulfonyl chloride (50 g, 191.17 mmol) in DCM (200 ml). The reaction mixture stirred under nitrogen for 5 h. It was diluted with water (500 ml) then 0.5M hydrochloric acid (500 ml). The solid that crashed out was filtered off (25.46 g, 37%). The filtrate layers were separated and the organic layer was washed with brine, dried (MgSO₄), filtered and concentrated to give the title compound as a cream solid (33.31 g, 48%).

M-H+=356, rt=2.55 min

### Intermediate 22

### 1,1-Dimethylethyl 5-[4-(5-{[(1,1-dioxidotetrahydro-3-thienyl)amino]sulfonyl}-2-thienyl)-1H-pyrrolo[2,3-b]pyridin-2-yl]-3,4-dihydro-1(2H)-pyridinecarboxylate

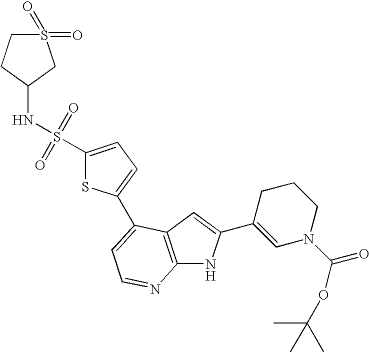

1,1-Dimethylethyl 5-[1-[(4-methylphenyl)sulfonyl]-4-(4,4,5,5-tetramethyl-1,3,2-dioxaborolan-2-yl)-1H-pyrrolo[2,3-b]pyridin-2-yl]-3,4-dihydro-1(2H)-pyridinecarboxylate (0.200 g, 0.34 mmol), 2-(dimethylamino)-2-biphenyl palladium (II) chloride dinorbornyl phosphine (0.010 g), potassium phosphate tribasic (0.220 g, 1.04 mmol) and 5-bromo-N-(1,1-dioxidotetrahydro-3-thienyl)-2-thiophenesulfonamide (0.150 g, 0.408 mmol) in dioxan (2.5 ml) and water (0.5 ml) were heated in the Biotage Initiator microwave at 120° C. for 1 h. The reaction was poured into water (20 ml) and extracted with DCM (2×25 ml). The combined organic extracts were dried (phase separator) and reduced in vacuo to give (0.370 g) of crude material which was diluted with THF (3 ml) and MeOH (1.5 ml) and treated with 5M sodium hydroxide (0.7 ml). The reaction was stirred for 18 h. The reaction was poured into water (20 ml) and extracted with DCM (2×25 ml). The organic layers were reduced in vacuo (0.460 g) and purified by MDAP to give the title compound (0.051 g).

MH+=579, rt=3.33 min

### Intermediate 23

### 1,1-Dimethylethyl 5-[4-(5-{[(1,1-dioxidotetrahydro-2H-thiopyran-4-yl)amino]sulfonyl}-2-thienyl)-1H-pyrrolo[2,3-b]pyridin-2-yl]-3,4-dihydro-1(2H)-pyridinecarboxylate

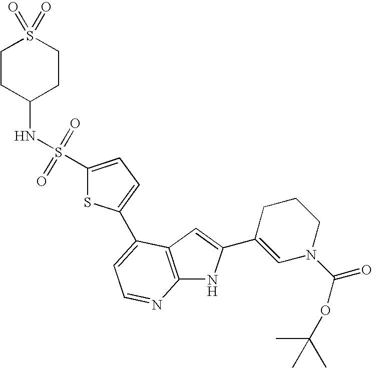

1,1-Dimethylethyl 5-[1-[(4-methylphenyl)sulfonyl]-4-(4,4,5,5-tetramethyl-1,3,2-dioxaborolan-2-yl)-1H-pyrrolo[2,3-b]pyridin-2-yl]-3,4-dihydro-1(2H)-pyridinecarboxylate (0.150 g, 0.26 mmol), 2-(dimethylamino)-2-biphenyl palladium (II) chloride dinorbornyl phosphine (0.0073 g), potassium phosphate tribasic (0.165 g, 0.776 mmol) and 5-bromo-N-(1,1-dioxidotetrahydro-2H-thiopyran-4-yl)-2-thiophenesulfonamide (0.114 g, 0.311 mmol) in dioxan (2 ml) and water (0.4 ml) were heated in the Biotage Initiator microwave at 140° C. for 40 min. Sodium hydroxide (0.07 g) was added and the reaction was heated in the Biotage Initiator microwave at 140° C. for 40 min. The reaction was diluted with water (40 ml), acidified with hydrochloric acid to pH8 and extracted with DCM (2×40 ml). The organic layers were combined, passed through a phase separator and reduced in vacuo. The residue was triturated in MeOH to give the title compound (0.032 g, 21%). The filtrate was reduced in vacuo and triturated in MeOH (10 ml) to give a further batch of the title compound (0.008 g, 5%).

MH+=593, rt=3.25 min

### Intermediate 24

### 6-Chloro-N-(1,1-dioxidotetrahydro-2H-thiopyran-4-yl)-3-pyridinesulfonamide

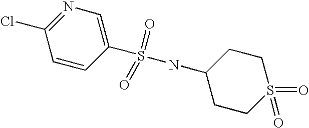

A mixture of 6-chloro-3-pyridinesulfonyl chloride (0.500 g, 2.358 mmol), (1,1-dioxidotetrahydro-2H-thiopyran-4-yl)amine (0.352 g, 2.358 mmol) and TEA (0.657 ml, 4.716 mmol) in anhydrous DCM (10 ml) was stirred at room temperature under nitrogen for 19 h. The mixture was evaporated in vacuo, partitioned between EtOAc (50 ml) and water (25 ml), the layers separated, the aqueous layer extracted with EtOAc (25 ml) and the combined EtOAc extracts were evaporated in vacuo to give the title compound as a white solid (0.705 g, 92%).

M-H+=323/325, rt=2.04 min

### Intermediate 25

### 2-Chloro-5-(1-pyrrolidinylsulfonyl)pyridine

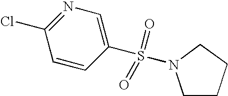

A mixture of 6-chloro-3-pyridinesulfonyl chloride (0.300 g, 1.41 mmol), pyrrolidine (0.210 g, 1.41 mmol) and TEA (0.393 ml, 2.82 mmol) in anhydrous DCM (6 ml) was stirred at room temperature under nitrogen for 18 h. The reaction mixture was washed with water (20 ml) after diluting with DCM (20 ml). The layers were separated and the DCM evaporated in vacuo to give the title compound as a white solid (0.328 g, 95%).

MH+=247/249, rt=2.6 min

### Intermediate 26

### 5-Bromo-N-(1,1-dioxidotetrahydro-2H-thiopyran-4-yl)-2-pyridinesulfonamide

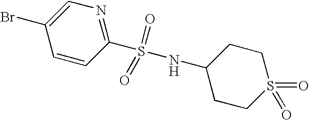

A mixture of 5-bromo-2-pyridinesulfonyl chloride (0.300 g, 1.17 mmol), (1,1-dioxidotetrahydro-2H-thiopyran-4-yl)amine (0.175 g, 1.17 mmol) and TEA (0.326 ml, 2.34 mmol) in anhydrous DCM (6 ml) was stirred at room temperature under nitrogen for 2 h. The reaction mixture was diluted with DCM (20 ml), filtered and washed with water (20 ml) to give the title compound as a white solid (0.220 g).

The DCM extract was separated from the aqueous (hydrophobic frit) and evaporated in vacuo to give the title compound as a white solid (0.107 g).

MH+=371, M-H+367/369, rt=2.17 min

### Intermediate 27

### 5-Bromo-2-(1-pyrrolidinylsulfonyl)pyridine

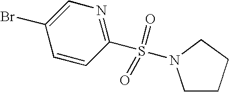

### Method A

A mixture of 5-bromo-2-pyridinesulfonyl chloride (0.300 g, 1.17 mmol), pyrrolidine (0.0976 ml, 1.17 mmol) and TEA (0.326 ml, 2.34 mmol) in anhydrous DCM (6 ml) was stirred at room temperature under nitrogen overnight. The reaction mixture was partitioned between DCM (20 ml) and water (20 ml), the layers separated and the DCM extract evaporated in vacuo to give the title compound as a pale brown solid (0.367 g).

MH+=293, rt=2.74 min

### Method B

To a solution of 5-bromo-2-[(phenylmethyl)thio]pyridine (0.6 g, 2.14 mmol) in anhydrous dichoromethane (25 mL) at 0° C. was added chlorine gas over 20 min. To the reaction was added water (10 mL). The organic was separated and dried (hydrophobic frit), concentration in vacuo gave a yellow oil. This oil was treated with dichloromethane (10 mL), pyrrolidine (0.55 mL, 6.53 mmol) and stirred for 30 min. The reaction was diluted with dichloromethane (15 mL) and washed sequentially with water (25 mL), saturated aqueous sodium carbonate (25 mL), saturated aqueous citric acid (25 mL), dried (hydrophobic frit) and concentrated in vacuo to give a solid (0.6 g). Purification by chromatography (silica 20 g, 0-25% ethylacetate in cyclohexane over 40 min) yielded the title compound as a white solid (339 mg, 63%).

LCMS MH+=291/293, rt=2.77 min

### Intermediate 28

### 5-Bromo-N-(1,1-dioxidotetrahydro-2H-thiopyran-4-yl)-2-methyl-3-thiophenesulfonamide

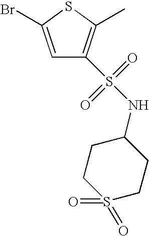

A solution of 5-bromo-2-methyl-3-thiophenesulfonyl chloride (1.32 g, 4.79 mmol) in anhydrous DCM (20 ml) was treated with triethylamine (1.33 ml) then tetrahydro-2H-thiopyran-4-amine 1,1-dioxide (0.714 g, 4.78 mmol) and stirred at room temperature under N₂ for 20 h. The reaction mixture was then diluted with DCM (30 ml), washed with water (50 ml) and the DCM extract evaporated in vacuo, preabsorbed onto florisil and purified by Flashmaster SPE (Silica, 10 g) eluted with 0-100% EtOAc/cyclohexane then 0-20% MeOH/DCM over 20 min. Fractions containing the product were combined and evaporated to give the title compound as a white oily solid (0.091 g).

MH+=388/390, rt=2.84 min

### Intermediate 29

### 2-Chloro-N-(1,1-dioxidotetrahydro-2H-thiopyran-4-yl)-4-methyl-1,3-thiazole-5-sulfonamide

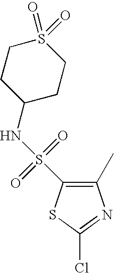

A mixture of 2-chloro-4-methyl-1,3-thiazole-5-sulfonyl chloride (0.2 g, 0.862 mmol), tetrahydro-2H-thiopyran-4-amine 1,1-dioxide (0.129 g, 0.862 mmol), anhydrous DCM (5 ml) and triethylamine (0.24 ml) was stirred at room temperature under N₂ for 2 h, diluted with DCM (10 ml), washed with water (10 ml), then evaporated in vacuo. The residue was dissolved in DCM (10 ml) and washed with 2M HCl (15 ml). The DCM extract was evaporated in vacuo to give the title compound as a yellow oil which solidified to a yellow solid (0.164 g).

MH−=343/345, rt=2.4 min

### Intermediate 30

### N-(1,1-Dioxidotetrahydro-2H-thiopyran-4-yl)-2-[2-methyl-1-(phenylsulfonyl)-1H-pyrrolo[2,3-b]pyridin-4-yl]-5-pyrimidinesulfonamide

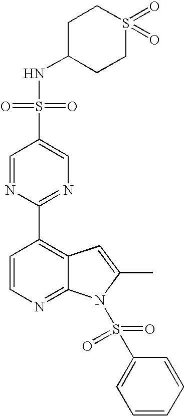

A solution of 4-bromo-2-methyl-1-(phenylsulfonyl)-1H-pyrrolo[2,3-b]pyridine (1.2 g, 3.42 mmol) in anhydrous DCM (60 ml) was de-gassed for 15 min then palladium acetate (0.0384 g, 0.171 mmol) and potassium acetate (1.006 g, 10.26 mmol) were added and the mixture stirred for 5 min under nitrogen at room temperature. 4,4,4′,4′,5,5,5′,5′-Octamethyl-2,2′-bi-1,3,2-dioxaborolane (2.18 g, 8.55 mmol) was added, the reaction was flushed with nitrogen and heated at 65° C. for 6 h, stirred at room temperature overnight then heated for a further 4 h at 65° C. under nitrogen. The reaction mixture was allowed to cool to room temperature then concentrated in vacuo. The residue was partitioned between water (100 ml) and DCM (100 ml). The layers were separated and the aqueous extracted with DCM (2×100 ml). The combined DCM extracts were filtered then evaporated in vacuo. The residue was triturated with cyclohexane, filtered and the filtrate evaporated in vacuo to give 2-methyl-1-(phenylsulfonyl)-4-(4,4,5,5-tetramethyl-1,3,2-dioxaborolan-2-yl)-1H-pyrrolo[2,3-b]pyridine as a yellow oily solid (3.21 g). A mixture of 2-chloro-5-pyrimidinesulfonyl chloride (0.3 g, 1.41 mmol), tetrahydro-2H-thiopyran-4-amine 1,1-dioxide (0.21 g, 1.41 mmol), triethylamine (0.393 ml, 2.82 mmol) in anhydrous DCM (6 ml) was stirred at room temperature under N₂ for 18 h. The reaction mixture was washed with water (20 ml) after diluting with DCM (20 ml) and the layers were separated. The aqueous was extracted with EtOAc (20 ml), washed with 2M HCl and evaporated in vacuo to give 2-chloro-N-(1,1-dioxidotetrahydro-2H-thiopyran-4-yl)-5-pyrimidinesulfonamide (0.038 g). The DCM extract was evaporated in vacuo and purified by Flashmaster SPE (Silica, 10 g) eluted with 0-100% EtOAc/cyclohexane over 20 min. Fractions containing product were combined and evaporated to give 2-chloro-N-(1,1-dioxidotetrahydro-2H-thiopyran-4-yl)-5-pyrimidinesulfonamide (0.078 g). A mixture of 2-methyl-1-(phenylsulfonyl)-4-(4,4,5,5-tetramethyl-1,3,2-dioxaborolan-2-yl)-1H-pyrrolo[2,3-b]pyridine (0.17 g, 0.179 mmol), 2-chloro-N-(1,1-dioxidotetrahydro-2H-thiopyran-4-yl)-5-pyrimidinesulfonamide (0.116 g, 0.358 mmol), sodium carbonate (0.038 g, 0.358 mmol), 1,1′-bis(diphenylphosphino)ferrocenedichloro palladium(II) (0.0073 g, 0.00998 mmol) in dioxane (4 ml) water (0.6 ml) was heated in a sealed tube in the Biotage Initiator microwave at 150° C. for 60 min. The reaction mixture was diluted with methanol, filtered through celite (10 g cartridge) and evaporated in vacuo and purified by MDAP to give the title compound as a white solid (0.037 g).

MH+=562, rt=3.16 min

### Intermediate 31

### 1,1-Dimethylethyl 4-{[(6-chloro-3-pyridinyl)sulfonyl]amino}-1-piperidinecarboxylate

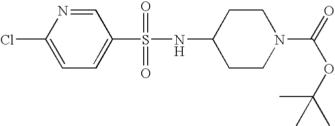

A mixture of 6-chloro-3-pyridinesulfonyl chloride (1 g, 4.716 mmol), 1,1-dimethylethyl 4-amino-1-piperidinecarboxylate (0.944 g, 4.716 mmol), triethylamine (1.31 ml, 9.43 mmol) in anhydrous DCM (15 ml) was stirred at room temperature under N₂ for 4 h. The reaction mixture was then diluted with DCM (50 ml), washed with water (50 ml). The DCM extract was evaporated in vacuo. The residue was dissolved in DCM (50 ml), washed with 2M HCl (50 ml) and the DCM extract evaporated in vacuo to give the title compound as a colourless oil which foamed to a white solid (1.64 g).

MNH4+=393/395, rt=3.13 min

### Intermediate 32

### 1,1-Dimethylethyl 4-[({6-[2-(difluoromethyl)-1-(phenylsulfonyl)-1H-pyrrolo[2,3-b]pyridin-4-yl]-3-pyridinyl}sulfonyl)amino]-1-piperidinecarboxylate

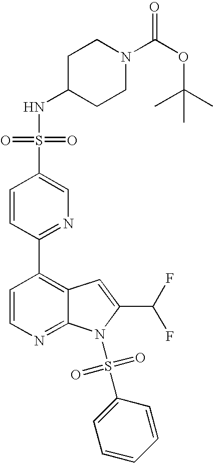

A mixture of 1,1-dimethylethyl 4-{[(6-chloro-3-pyridinyl)sulfonyl]amino}-1-piperidinecarboxylate (0.083 g, 0.22 mmol), [2-(difluoromethyl)-1-(phenylsulfonyl)-1H-pyrrolo[2,3-b]pyridin-4-yl]boronic acid (0.071 g, 0.2 mmol), IPA (1.8 ml), 1,1′-bis(diphenylphosphino)ferrocenedichloro palladium(II) (0.0082 g, 0.011 mmol), 1M sodium bicarbonate (0.6 ml) was degassed for 5 min then heated in a sealed tube in the microwave at 120° C. for 30 min. The reaction mixture was filtered and the filtrate purified by SCX (5 g) eluted with MeOH then 2M NH₃/MeOH. The MeOH fractions were evaporated in vacuo to give the title compound as a brown oil (0.099 g).

MH+=648, rt=3.69 min

### Intermediate 33

### 6-[2-(Difluoromethyl)-1-(phenylsulfonyl)-1H-pyrrolo[2,3-b]pyridin-4-yl]-N-(1,1-dioxidotetrahydro-2H-thiopyran-4-yl)-3-pyridinesulfonamide

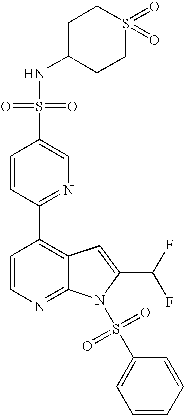

A mixture of [2-(difluoromethyl)-1-(phenylsulfonyl)-1H-pyrrolo[2,3-b]pyridin-4-yl]boronic acid (0.147 g, 0.417 mmol), 6-chloro-N-(1,1-dioxidotetrahydro-2H-thiopyran-4-yl)-3-pyridinesulfonamide (0.1487 g, 0.458 mmol), sodium carbonate (0.088 g, 0.834 mmol), 1,1′-bis(diphenylphosphino)ferrocenedichloro palladium(II) (0.0208 g, 0.028 mmol), dioxane (4 ml), water (1 ml) was heated in a sealed tube in a Biotage Initiator microwave at 150° C. for 1 h. The reaction mixture was then diluted with MeOH and filtered (celite cartridge, 10 g). The filtrate was evaporated in vacuo and purified by MDAP to give the title compound as a light brown solid (0.0217 g).

MH+=597, rt=3.21 min

### Intermediate 34

### 4-[(6-Chloro-3-pyridinyl)sulfonyl]thiomorpholine 1,1-dioxide

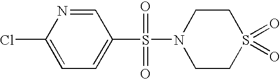

A mixture of 6-chloro-3-pyridinesulfonyl chloride (1 g, 4.716 mmol), thiomorpholine 1,1-dioxide (0.636 g, 4.716 mmol), triethylamine (1.31 ml, 9.433 mmol) in anhydrous DCM (15 ml) were stirred at room temperature under N₂ for 21 h. The reaction mixture was then diluted with DCM (50 ml), washed with water (50 ml), layers separated and the DCM extract evaporated in vacuo to give the title compound as a white solid (0.172 g). The aqueous was filtered to give the title compound as a white solid (1.24 g).

[M+formic acid−H]−=355/357 LCMS rt=2.26 min

### Intermediate 35

### 1,1-Dimethylethyl 4-{[(5-bromo-2-pyridinyl)sulfonyl]amino}-1-piperidinecarboxylate

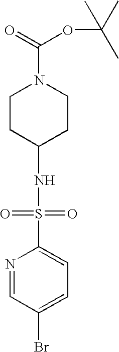

5-Bromo-2-pyridinesulfonyl chloride (1.02 g, 3.97 mmol), 1,1-dimethylethyl 4-amino-1-piperidinecarboxylate (0.97 g, 4.84 mmol) and DIPEA (0.756 g, 5.85 mmol) were dissolved in DCM (20 ml) and the reaction allowed to occur under nitrogen atmosphere for 1.5 h. The mixture was taken into a separating funnel and submitted to an extraction using a saturated solution of sodium carbonate (100 ml) and DCM (500 ml). The organic phase was recovered, passed through a phase separator and the solvent evaporated to give the title compound (1.8512 g).

MH+421/419, rt=3.11 min

### Intermediate 36

### 6-Chloro-N-(1,1-dioxidotetrahydro-2H-thiopyran-4-yl)-N-methyl-3-pyridinesulfonamide

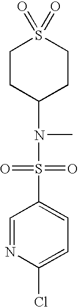

6-Chloro-3-pyridinesulfonyl chloride (0.503 g, 2.37 mmol), N-methyltetrahydro-2H-thiopyran-4-amine 1,1-dioxide (0.467 g, 2.86 mmol) and DIPEA (0.617 ml, 3.53 mmol) were dissolved in DCM (15 ml). The reaction was allowed to occur under a nitrogen atmosphere for 60 min. The mixture was taken into a separating funnel and submitted to an extraction using DCM (150 ml) and a saturated solution of sodium bicarbonate (30 ml). The organic phase was recovered, passed through a phase separator and the solvent evaporated. The residue was dissolved in MeOH (5 ml), absorbed in silica and submitted to column chromatography (Flashmaster, Silica, 50 g) eluted with 0-100% EtOAc/cyclohexane. Fractions containing the product were isolated to give the title compound (0.729 g).

MH+=339/341, rt=2.64 min

### Intermediate 37

### 5-Bromo-N-(1,1-dioxidotetrahydro-2H-thiopyran-4-yl)-N-methyl-2-pyridinesulfonamide

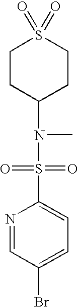

5-Bromo-2-pyridinesulfonyl chloride (0.503 g, 1.96 mmol), N-methyltetrahydro-2H-thiopyran-4-amine 1,1-dioxide (0.384 g, 2.35 mmol) and DIPEA (0.509 ml, 2.92 mmol) were dissolved in DCM (15 ml). The reaction was allowed to occur under nitrogen atmosphere for 60 min. The mixture was taken into a separating funnel and submitted to an extraction using DCM (150 ml) and a saturated solution of sodium carbonate (50 ml). The organic phase was recovered, passed through a phase separator and the solvent evaporated to give the title compound (0.695 g).

MH+=385/383, rt=2.69 min

### Intermediate 38

### N-(1,1-Dioxidotetrahydro-2H-thiopyran-4-yl)-6-[2-methyl-1-(phenylsulfonyl)-1H-pyrrolo[2,3-b]pyridin-4-yl]-3-pyridinesulfonamide

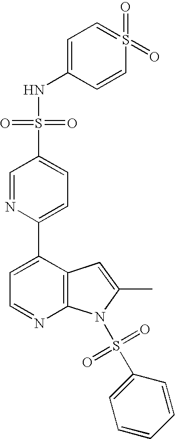

A solution of 4-bromo-2-methyl-1-(phenylsulfonyl)-1H-pyrrolo[2,3-b]pyridine (1.2 g, 3.42 mmol) in anhydrous DCM (60 ml) was de-gassed for 15 min then palladium acetate (0.0384 g, 0.171 mmol) and potassium acetate (1.006 g, 10.26 mmol) were added and the mixture stirred for 5 min under nitrogen at room temperature. 4,4,4′,4′,5,5,5′,5′-Octamethyl-2,2′-bi-1,3,2-dioxaborolane (2.18 g, 8.55 mmol) was added, the reaction was flushed with nitrogen and heated at 65° C. for 6 h, stirred at room temperature overnight then heated for a further 4 h at 65° C. under nitrogen. The reaction mixture was allowed to cool to room temperature then concentrated in vacuo. The residue was partitioned between water (100 ml) and DCM (100 ml). The layers were separated and the aqueous extracted with DCM (2×100 ml). The combined DCM extracts were filtered then evaporated in vacuo. The residue was triturated with cyclohexane, filtered and the filtrate evaporated in vacuo to give 2-methyl-1-(phenylsulfonyl)-4-(4,4,5,5-tetramethyl-1,3,2-dioxaborolan-2-yl)-1H-pyrrolo[2,3-b]pyridine as a yellow oily solid (3.21 g). A mixture of 2-methyl-1-(phenylsulfonyl)-4-(4,4,5,5-tetramethyl-1,3,2-dioxaborolan-2-yl)-1H-pyrrolo[2,3-b]pyridine (0.2 g, 0.212 mmol), 6-chloro-N-(1,1-dioxidotetrahydro-2H-thiopyran-4-yl)-3-pyridinesulfonamide (0.082 g, 0.254 mmol), sodium carbonate (0.045 g, 0.424 mmol), 1,1′-bis(diphenylphosphino)ferrocenedichloro palladium(II) (0.0086 g, 0.0106 mmol) in dioxane (2.4 ml) and water (0.6 ml) was heated in a sealed tube in a Biotage Initiator microwave at 150° C. for 1 h. The reaction mixture was then diluted with MeOH, filtered through celite (10 g cartridge) and evaporated in vacuo. The residue was purified by MDAP to give the title compound as a pale brown solid (0.0274 g).

MH+=561, rt=3.15 min

### Intermediate 39

### 4-(6-Fluoro-3-pyridinyl)-2-(trifluoromethyl)-1H-pyrrolo[2,3-b]pyridine

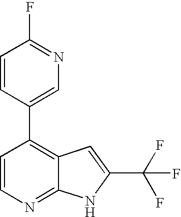

A mixture of (6-fluoro-3-pyridinyl)boronic acid (0.224 g, 1.587 mmol), 4-chloro-2-(trifluoromethyl)-1H-pyrrolo[2,3-b]pyridine (0.350 g, 1.587 mmol), 1M sodium bicarbonate (4.76 ml, 4.76 mmol) and 1,1′-bis(diphenylphosphino)ferrocene-palladium(ii) dichloride dichloromethane complex (0.0648 g, 0.079 mmol) in Isopropanol (14.2 ml) was degassed for 5 min then heated in a sealed tube in a Biotage Initiator microwave at 120° C. for 60 min. The reaction mixture was diluted with MeOH then filtered to give a pale grey solid (0.275 g). 0.225 g of the pale grey solid was partitioned between DCM (30 ml) and water (30 ml), the layers were separated. The aqueous was extracted with EtOAc (30 ml) and the combined DCM and EtOAc extracts were evaporated in vacuo to give the title compound as a light brown solid (0.133 g). The filtrate was evaporated in vacuo, preabsorbed onto florisil and purified by flashmaster SPE (Silica, 20 g), eluted with 0-100% EtOAc/cyclohexane then 0-20% MeOH/DCM over 30 minutes. Fractions containing product were combined and evaporated in vacuo to give the title compound as a white solid (0.050 g).

MH+=282, rt=3.15 min

### Intermediate 40

### 1,1-Dimethylethyl (2-{[(6-chloro-3-pyridinyl)sulfonyl]amino}ethyl)carbamate

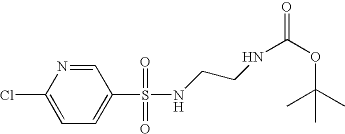

A mixture of 6-chloro-3-pyridinesulfonyl chloride (1 g, 4.716 mmol), 1,1-dimethylethyl (2-aminoethyl)carbamate (0.754 g, 4.716 mmol), triethylamine (1.31 ml, 9.433 mmol) in anhydrous DCM (15 ml) was stirred at room temperature under N₂ for 21 h. The reaction mixture was then diluted with DCM (50 ml), washed with water (50 ml), layers separated and the DCM extract evaporated in vacuo to give the title compound (1.48 g).

MH−=334/336, rt=2.79 min

### Intermediate 41

### 5-Bromo-N-(2-hydroxyethyl)-2-pyridinesulfonamide

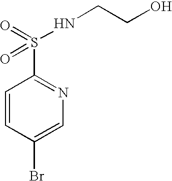

5-Bromo-2-pyridinesulfonyl chloride (1 g, 3.9 mmol), 2-aminoethanol (0.35 ml, 5.9 mmol) and DIPEA (1.3 ml, 7.8 mmol) were mixed together in DCM at room temperature under N₂ for 30 min. The reaction mixture was washed with 30 ml sodium bicarbonate solution, dried on a phase separator and reduced under vacuum to give the title compound (0.838 g).

MH+=281/283, rt=1.91 min

### Intermediate 42

### 4-[(5-Bromo-2-pyridinyl)sulfonyl]thiomorpholine 1,1-dioxide

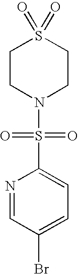

5-Bromo-2-pyridinesulfonyl chloride (1 g, 3.899 mmol), thiomorpholine 1,1-dioxide (0.633 g, 4.678 mmol) and DIPEA (0.756 g, 5.849 mmol) were dissolved in DCM (30 ml) and the reaction occurred at room temperature for 2 h. The mixture was taken into a separating funnel and submitted to an extraction using a saturated solution of sodium carbonate (50 ml) and a mixture of DCM/EtOAc (1:1, 150 ml). The organic phase was recovered, passed through a phase separator and the solvent evaporated. The aqueous phase was extracted with DCM:EtOAc (250 ml) to extract more product. Both the extractions were combined to obtain the title compound (1.278 g).

MH+=356/354, rt=2.35 min

### Intermediate 43

### 5-Bromo-2-pyridinesulfonyl chloride

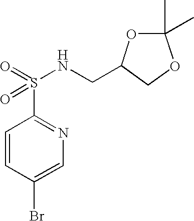

[(2,2-Dimethyl-1,3-dioxolan-4-yl)methyl]amine (0.76 ml, 5.85 mmol), 5-bromo-2-pyridinesulfonyl chloride (1 g, 3.9 mmol), DIPEA (1.3 ml, 7.8 mmol), were stirred together in DCM (20 ml) at room temperature under N₂ for 1 h. The reaction mixture was extracted with sodium bicarbonate (20 ml) dried on a phase separator and reduced under vacuum to give the title compound as a yellow solid (0.629 g)

MH+351/353, rt=2.58 min

### Intermediate 44

### 6-Chloro-N-(2-hydroxyethyl)-3-pyridinesulfonamide

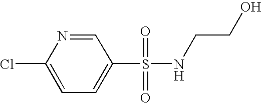

A mixture of 6-chloro-3-pyridinesulfonyl chloride (1 g, 4.716 mmol), 2-aminoethanol (0.284 ml, 4.716 mmol), TEA (1.31 ml, 9.433 mmol) in anhydrous DCM (15 ml) was stirred at room temperature under N₂ for 5 h. The reaction mixture was diluted with DCM (50 ml), washed with water (50 ml) and the layers separated, DCM extract evaporated in vacuo to give a colourless oil which solidified on standing. This was redissolved in DCM (50 ml), washed with 2M HCl (50 ml) and the DCM extract evaporated in vacuo to give the title compound as a white solid (0.272 g). The aqueous was extracted with EtOAc (100 ml) and the EtOAc extract evaporated in vacuo to give the title compound as a white solid (0.545 g).

MH+=237/239, rt=1.85 min

### Intermediate 45

### 6-Chloro-N-(1,1-dioxidotetrahydro-3-thienyl)-3-pyridinesulfonamide

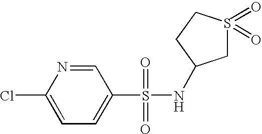

A mixture of 6-chloro-3-pyridinesulfonyl chloride (0.3 g, 1.41 mmol), (1,1-dioxidotetrahydro-3-thienyl)amine (0.19 g, 1.41 mmol), TEA (0.393 ml) in anhydrous DCM (6 ml) was stirred at room temperature under N₂ for 17 h. The mixture was diluted with DCM (20 ml), washed with water (20 ml), the layers were separated and the DCM extract evaporated in vacuo to give the title compound as a white solid (0.358 g).

MH−=309/311, rt=2.11 min

### Intermediate 46

### 4-Bromo-1-(phenylsulfonyl)-1H-pyrrolo[2,3-b]pyridine

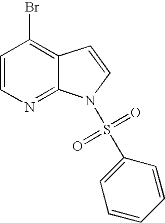

4-Bromo-1H-pyrrolo[2,3-b]pyridine (which may be prepared as described in Org Letts, 2003, 5, 5023) (15.0 g, 76 mmol) was suspended in DCM (300 ml) and 1,4-dioxane (100 ml) and 50% aq. sodium hydroxide (23 ml) was added followed by NaBu₄HSO₄ solution (7.5 ml). The mixture was vigorously stirred and cooled in an ice bath whilst benzene sulfonyl chloride (14.6 ml, 115 mmol) was added dropwise. The reaction mixture was left stirring vigorously for 5 days. It was evaporated to dryness and the resulting solid was washed with water (100 ml) followed by methanol (50 ml). The yellow solid was dried in vacuo. The title compound was obtained as a pale yellow solid (23.5 g, 92%).

MH+337/339, rt=3.35 min

### Intermediate 47

### 4-Bromo-1-(phenylsulfonyl)-1H-pyrrolo[2,3-b]pyridine-2-carbaldehyde

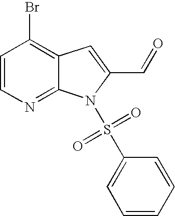

To a solution of 4-bromo-1-(phenylsulfonyl)-1H-pyrrolo[2,3-b]pyridine (3.37 g, 10.0 mmol) in dry THF (50 ml) at −25° C. to −40° C. under nitrogen was added 2M LDA in heptane/THF/ethylbenzene (10 ml, 20.0 mmol). The reaction was stirred at this temperature for 40 min. DMF (2 ml) was added dropwise at −30° C. and allowed to warm up to room temperature. The reaction was poured into 2M aq. HCl, extracted with DCM (3×50 ml) and evaporated to give the crude material which was purified using a silica SPE cartridge (50 g) eluting with DCM. Fractions containing the product were combined and evaporated to give the title compound as a cream foam (0.8 g, 22%).

MH+365/367, rt=3.31 min

### Intermediate 48

### 4-Bromo-2-(difluoromethyl)-1-(phenylsulfonyl)-1H-pyrrolo[2,3-b]pyridine

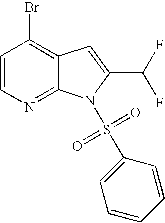

To a solution of 4-bromo-1-(phenylsulfonyl)-1H-pyrrolo[2,3-b]pyridine-2-carbaldehyde (0.420 g, 1.15 mM) in DCM (10 ml) at room temperature was added Deoxyfluor™ (0.64 ml, 3.48 mM). The reaction was stirred for 2 h and left standing overnight. Reaction mixture was poured carefully into aqueous sodium bicarbonate solution. The organic layer was separated and washed with brine (20 ml) and aq. saturated ammonium chloride solution, dried with a hydrophobic frit and solvent was removed in vacuo leaving a yellow solid. Solid was dissolved in DCM (20 ml), washed with HCl (1M, 20 ml×2) and water (20 ml×2), filtered through a phase separator and evaporated to dryness to furnish 4-bromo-2-(difluoromethyl)-1-(phenylsulfonyl)-1H-pyrrolo[2,3-b]pyridine as a yellow solid (0.380 g).

MH+=387/389, rt=3.55 min

### Intermediate 49

### [2-(Difluoromethyl)-1-(phenylsulfonyl)-1H-pyrrolo[2,3-b]pyridin-4-yl]boronic acid

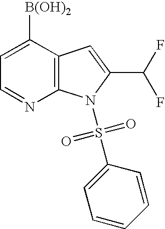

To a solution of 4-bromo-2-(difluoromethyl)-1-(phenylsulfonyl)-1H-pyrrolo[2,3-b]pyridine (2.0 g, 5.17 mmol) in dry THF (50 mL) at −78° C. under nitrogen whilst stirring was added tris(1-methylethyl)borate (1.9 mL, 8.3 mmol) followed by n-butyl lithium (4.1 ml, 1.6M in hexanes, 6.6 mmol) dropwise over 10 min. Stirred at −78° C. for 1 h then allowed to warm up to ambient temperature over 3 h. Cooled to −78° C. and added 2M aqueous hydrochloric acid (100 ml) under nitrogen over 5 min then added 100 ml DCM. Separated organic layer then extracted aqueous layer with ethyl acetate (50 ml). Combined both organic extracts, dried through phase sep. cartridge and evaporated to give a pale brown foam. The foam was triturated with diethyl ether (100 mL). The resulting pale brown solid (900 mg) was dried in vacuo at 60° C. for 2 h.

MH+=353, rt=3.06 min

### Intermediate 50

### 2-(Trifluoromethyl)-1H-pyrrolo[2,3-b]pyridine

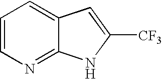

1,1-Dimethylethyl (3-methyl-2-pyridinyl)carbamate (Synthesis, 1996, 7, 877) (5.2 g, 25 mmol) was stirred in dry THF (60 ml) and cooled in an ice bath (ice/salt) to −4° C. The mixture was treated with 2M-Bu lithium in cyclohexane (25 ml, 50 mmol) under nitrogen in a drop wise fashion over 45 min while maintaining the temperature below 0° C. The red suspension was stirred for an hour at −3° C. then treated with N-methoxy-N-methyl trifluoro acetamide (4.71 g, 30 mmol). There was a temperature rise to 20° C. The dark red solution was cooled to 2° C. and then allowed to warm to 10° C. over an hour. The dark orange solution was added to 5M HCl (55 ml) over 30 min at 3° C. The mixture was then heated at 60° C. for an hour. The reaction was heated at 80° C. for a further hour. The phases were separated and the aq. phase was made alkaline with 10M sodium hydroxide. The mixture was extracted with EtOAc (2×50 ml). The organic phase was dried then evaporated to give a pale orange solid which was filtered through silica (70 g) eluting with DCM to DCM:ether 9:1. The main fraction was evaporated to give the title compound as a pale yellow crystalline solid (2.66 g, 57%).

MH+=187, rt=2.73 min

### Intermediate 51

### 2-(Trifluoromethyl)-1H-pyrrolo[2,3-b]pyridine 7-oxide

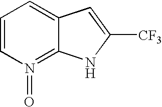

A stirred, cooled (0° C. ice/salt) solution of 2-(trifluoromethyl)-1H-pyrrolo[2,3-b]pyridine (1.86 g, 10 mmol) in EtOAc (35 ml) was treated drop wise with a solution of m-chloroperoxybenzoic acid (2.78 g, 12.2 mmol) in EtOAc (35 ml) over 30 min. The reaction temperature was maintained below 5° C. during the addition. The reaction was warmed to 10° C. over 2 h. The reaction was cooled to 0° C. and treated with an additional portion of MCPBA (0.700 g, 4 mmol) in EtOAc (10 ml). The reaction was warmed to room temperature over 2 h. The solid precipitate was collected to give the title compound as a white solid (0.950 g, 47%).

MH+=203, rt=0.68 min

### Intermediate 52

### 4-Chloro-2-(trifluoromethyl)-1H-pyrrolo[2,3-b]pyridine

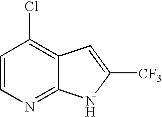

2-(Trifluoromethyl)-1H-pyrrolo[2,3-b]pyridine 7-oxide (1.26 g, 6.25 mmol) was suspended in dimethylformamide (7.5 ml) and heated to 50° C. The mixture was treated with methanesulfonyl chloride (2.5 ml) dropwise. The solid went into solution on addition of the sulphonyl chloride and there was a temperature increase to 60° C. The reaction was heated at 70° C. for 2 h and cooled to room temperature. The reaction was poured into water (50 ml) and neutralised with 10M sodium hydroxide. The solid was collected and dried under air to give (1.24 g). This material was triturated with hot aq. ethanol, collected and was dried in vacuo (50° C.) to afford the title compound as a cream solid (1.1 g, 80%).

MH+=221, rt=3.22 min

### Intermediate 53

### 4-Iodo-2-(trifluoromethyl)-1H-pyrrolo[2,3-b]pyridine

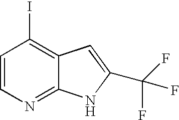

To 4-chloro-2-(trifluoromethyl)-1H-pyrrolo[2,3-b]pyridine (23.92 g, 0.108 mole) dissolved in hot 1,4-dioxane (120 ml, 5 volumes) was added 4M HCl in dioxane (30 ml, 0.119 mole, 1.1 eq). The resulting suspension was cooled to room temperature and the solid collected by filtration washing well with diethyl ether. The solid was then suspended in anhydrous acetonitrile (480 ml, 20 volumes) and then sodium iodide (97.7 g, 0.652 mole, 6 eq) was added. The mixture was then heated to 80° C. and maintained at that temperature overnight. It was then cooled to room temperature and 2M NaOH added till mixture was basic. The layers were then separated and the organic layer was washed with brine, dried over magnesium sulphate, filtered then concentrated in vacuo to a yield the title compound as a brown solid (9.98 g, 29%).

MH+=313, rt=1.19 min

The magnesium sulfate was then suspended in ethyl acetate and the mixture heated to 65° C. The mixture was filtered and the filtrate concentrated in vacuo to yield a second crop of the title compound as a cream solid (15.8 g, 47%).

MH+=313, rt=1.19 min

### Intermediate 54

### [2-(Trifluoromethyl)-1H-pyrrolo[2,3-b]pyridin-4-yl]boronic acid

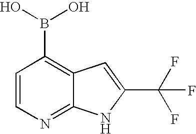

To a degassed solution of 4-iodo-2-(trifluoromethyl)-1H-pyrrolo[2,3-b]pyridine (1.0 g, 3.2 mmol) in anhydrous tetrahydrofuran (20 mL) at 20° C. was added sodium hydride as a 60% dispersion on mineral oil (160 mg 4 mmol) and stirred at 20° C. for 75 min. The mixture was degassed and cooled to −78° C. before addition of n-butyl-lithium 1.5M in hexanes (4.91 mL, 7.36 mmol) over 10 min. Reaction stirred at −78° C. for 20 min. Triisopropylborate (2.26 mL, 9.6 mmol) was added over 5 min. Reaction was warmed to 20° C. over 1.5 h and water (20 mL) added. The aqueous was extracted with ethyl acetate. The aqueous was adjusted to pH=7 (citric acid) and further extracted with ethyl acetate. The combined extracts were dried (hydrophobic frit) and concentrated in vacuo to a yellow solid. Purification by aminopropyl cartridge (10 g, eluent 4M ammonia in methanol) gave the title compound as a pale yellow solid (343 mg, 47%).

MH+=231, rt=0.76 min

### Intermediate 55

### 2-(1,1-Dimethylethyl)-1H-pyrrolo[2,3-b]pyridine

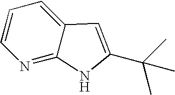

1,1-Dimethylethyl (3-methyl-2-pyridinyl)carbamate (5.2 g, 25 mmol) was suspended in anhydrous THF (60 ml). This suspension was cooled to −5° C. under nitrogen. n-Butyllitium (ca 1.25M in hexanes; 19.5 ml, 24 mmol) was added via dropping funnel until the suspension became red (45 min drop wise). A further portion of n-butyllithium (19.5 ml) was added over 20 min at −5° C. The suspension was stirred for a further 40 min. N,N′ Dimethylpivalamide (3.87 g, 30 mmol) was added in one portion. There was a colour change to bright orange. After 30 min the reaction was allowed to warm to room temperature. After a further 30 min 5M hydrochloric acid (55 ml) was added and the biphasic mixture was heated to 60° C. for 2 h. The organic layer was removed, the aqueous treated with 10M aqueous sodium hydroxide (40 ml) and extracted with EtOAc (2×55 ml). The combined organic extracts were dried and concentrated in vacuo to give (4.35 g). This solid was pre-absorbed onto Florisil and purified by chromatography on silica (100 g) eluting with 0 to 100% EtOAc in DCM over 40 min to give the title compound as a very pale yellow crystalline solid (2.34 g, 54%).

MH+=175, rt=0.99 min

### Intermediate 56

### 2-(1,1-Dimethylethyl)-1H-pyrrolo[2,3-b]pyridine 7-oxide

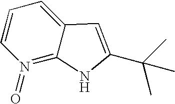

A solution of 2-(1,1-dimethylethyl)-1H-pyrrolo[2,3-b]pyridine (2.34 g, 13.4 mmol) in EtOAc (50 ml) was cooled to 0° C. A solution of MCPBA (5.2 g, 22.8 mmol) in EtOAc (50 ml) was added drop wise over 30 min. Once addition was complete, the reaction was allowed to warm up to 13° C. over 2 h. The reaction was washed sequentially with saturated aqueous sodium hydrogen carbonate (100 ml) followed by saturated sodium metabisulphate (100 ml), dried (MgSO₄), filtered and concentrated in vacuo to give an orange gum. Purification was by chromatography on silica gel (100 g) eluting with 0-25% MeOH in DCM to give impure material as a yellow gum/glass (2.7 g). Trituration with cyclohexane gave the title compound as a white solid (0.572 g, 22%). The remainder was purified again (100 g, silica, 0-25% MeOH/DCM, 60 min) to give a viscous oil (1.729 g) which was triturated in a mixture of water (10 ml) and saturated aqueous potassium carbonate (7 ml) with heating. The solid was filtered and dried in vacuo to give a further batch of the title compound as a very pale yellow solid (0.864 g, 33.5%).

MH+=191, rt=0.84 min

### Intermediate 57

### 4-Bromo-2-(1,1-dimethylethyl)-1H-pyrrolo[2,3-b]pyridine

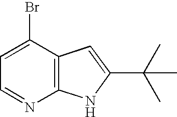

2-(1,1-Dimethylethyl)-1H-pyrrolo[2,3-b]pyridine 7-oxide (11 g, 58 mmol) and tetramethylammonium bromide (13.49 g, 87 mmol) were placed in DMF (78 ml). The mixture was cooled to 0° C. and treated with a portionwise additions of methane sulfonic anhydride (20.2 g, 116 mmol). The reaction was stirred at 5° C. for 1 hour and then allowed to warm to room temperature. The mixture was stirred for a further four hours and the solid collected. (3.4 g).

MH+=255, rt=1.27 min

### Intermediate 58

### 2-(1-Methylethyl)-1H-pyrrolo[2,3-b]pyridine

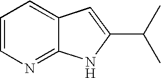

To a stirred suspension of 1,1-dimethylethyl (3-methyl-2-pyridinyl)carbamate (30 g, 144 mmol) in tetrahydrofuran (300 mL) at 0° C. under an atmosphere of nitrogen was added n-butyl-lithium (2.3M, 123 mL) over 90 min. After 30 min at 0° C. N,2-dimethyl-N-(methyloxy)propanamide (22.7 g, 173 mmol) was added. After a further 60 min at 0° C. the reaction was allowed to warm to ambient temperature and was added to 5M aqueous hydrochloric acid (300 mL) and heated to 60° C. for 1.5 h. The organic was separated. The aqueous was basified with 10M aqueous sodium hydroxide and extracted with ethyl acetate. The combined extracts were dried (MgSO₄) and concentrated in vacuo to give the title compound as an orange crystalline solid (22.04 g, 95%).

MH+=161, rt=2.38 min

### Intermediate 59

### 2-(1-Methylethyl)-1H-pyrrolo[2,3-b]pyridine 7-oxide

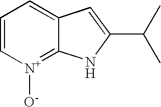

To a stirred solution of 2-(1-methylethyl)-1H-pyrrolo[2,3-b]pyridine (28.59 g, 0.178 mol) in dichloromethane (500 mL) at 0° C. was added a solution of meta-chloroperbenzoic acid (46.25 g, 0.268 mol) in dichloromethane (500 mL). After 1 h 45 min metachloroperbenzoic acid (5 g, 0.03 mol) was added. The reaction was warmed to ambient temperature over a period of 16 h. Metachloroperbenzoic acid (10 g, 0.06 mol) was added and the reaction stirred at 0° C. for 1 h. The reaction was washed with saturated aqueous sodium metabisulphite (600 mL), saturated aqueous potassium carbonate (14 dried (hydrophobic frit) and concentrated in vacuo to a dark red oil. Trituration with diethyl ether and filtration gave title compound (5.8 g, 18.5%). Concentration of the filtrate in vacuo follow by chromatography (silica 200 g, 0 to 50% methanol in dichloromethane) gave an orange solid. Trituration with diethyl ether furnished a second batch of the title compound (3.02 g, 10%).

MH+=177, rt=2.29 min

### Intermediate 60

### 4-Bromo-2-(1-methylethyl)-1H-pyrrolo[2,3-b]pyridine

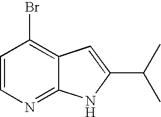

To a stirred solution of 2-(1-methylethyl)-1H-pyrrolo[2,3-b]pyridine 7-oxide (0.75 g, 4.26 mmol) in anhydrous dimethylformamide (7.5 mL) was added tetramethylammoniumbromide [TMAB] (0.94 g, 6.13 mmol) the resultant suspension was cooled to 0° C. The reaction was treated with methane-sulfonic-anhydride (1.48 g, 8.52 mmol) and the reaction was allowed to warm to ambient temperature over 16 h. The reaction was treated further with TMAB (0.33 g, 2.13 mmol) and methane-sulfonic-anhydride (0.37 g, 2.13 mmol). After 1 h the reaction was poured onto water (10 mL) and basified with 10M aqueous sodium hydroxide. The resultant yellow solid was collected by filtration. Purification by chromatography (silica 50 g, 0-25% ethylacetate in cyclohexane) gave the title compound as a white solid (0.498 g, 48%).

MH+=239, rt=3.34 min

### Intermediate 61

### 2-(Difluoromethyl)-4-[5-(1H-imidazol-1-ylsulfonyl)-2-thienyl]-1-(phenylsulfonyl)-1H-pyrrolo[2,3-b]pyridine

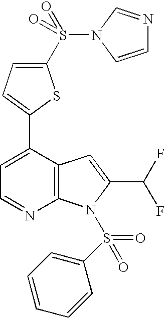

To a degassed suspension of [2-(difluoromethyl)-1-(phenylsulfonyl)-1H-pyrrolo[2,3-b]pyridin-4-yl]boronic acid (400 mg, 1.14 mmol), 1-[(5-bromo-2-thienyl)sulfonyl]-1H-imidazole (400 mg, 1.36 mmol) and sodium carbonate (361 mg, 3.4 mmol) in 4:1 dioxane:water (10 mL) was added 1,1′-Bis(diphenylphosphino)ferrocenedichloro palladium (II) (50 mg, 0.06 mmol) and heated to 120° C. for 30 min (Biotage Initiator microwave). The reaction was diluted with water (50 mL) and extracted with dichloromethane (2×50 mL). The combined extracts were dried (hydrophobic frit) and concentrated in vacuo to a brown oil. Purification by chromatography (silica 50 g, 0-100% Ethylacetate in cyclohexane over 40 min) gave the title compound as a pale orange foam (203 mg).

LCMS MH+=521 rt=1.29 min

### Intermediate 62

### N-(2-Aminoethyl)-5-[2-(difluoromethyl)-1-(phenylsulfonyl)-1H-pyrrolo[2,3-b]pyridin-4-yl]-2-thiophenesulfonamide

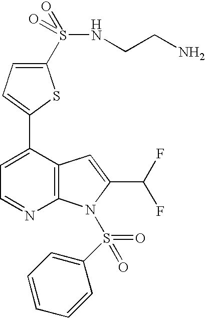

To a solution of 2-(difluoromethyl)-4-[5-(1H-imidazol-1-ylsulfonyl)-2-thienyl]-1-(phenylsulfonyl)-1H-pyrrolo[2,3-b]pyridine (65 mg, 0.125 mmol) in anhydrous tetrahydrofuran (1 mL) at 0° C. was added methyltriflate (164, 0.137 mmol). After 30 min 1,1-dimethylethyl (2-aminoethyl)carbamate (22 μL, 0.137 mmol) was added and warmed to ambient temperature. The reaction was concentrated (nitrogen blowdown) and treated with trifluoroacetic acid (2 mL). After a further 30 min the reaction was concentrated again (nitrogen blowdown). Purification by MDAP gave the title compound (23 mg).

LCMS MH+=513, rt=1.00 min

### Intermediate 63

### 5-[2-(Difluoromethyl)-1-(phenylsulfonyl)-1H-pyrrolo[2,3-b]pyridin-4-yl]-N-(1,1-dioxidotetrahydro-3-thienyl)-2-thiophenesulfonamide

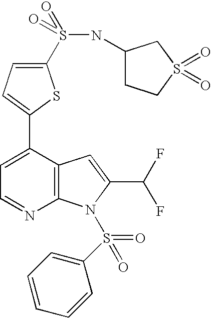

To a solution of 2-(difluoromethyl)-4-[5-(1H-imidazol-1-ylsulfonyl)-2-thienyl]-1-(phenylsulfonyl)-1H-pyrrolo[2,3-b]pyridine (65 mg, 0.125 mmol) in anhydrous tetrahydrofuran (1 mL) at 0° C. was added methyltriflate (16 μL, 0.137 mmol). After 30 min tetrahydro-3-thiophenamine 1,1-dioxide (which is commercially available from, for example, SALOR, 19 mg, 0.137 mmol) was added and warmed to ambient temperature. After 30 min N-Methylpyrollidine (0.5 mL) was added and stirring continued for a further 30 min The reaction was concentrated (nitrogen blowdown). Purification by MDAP gave the title compound (17 mg).

LCMS MH+=588, rt=1.18 min

### Intermediate 64

### 5-[2-(Difluoromethyl)-1-(phenylsulfonyl)-1H-pyrrolo[2,3-b]pyridin-4-yl]-N-(1,1-dioxidotetrahydro-2H-thiopyran-4-yl)-2-thiophenesulfonamide

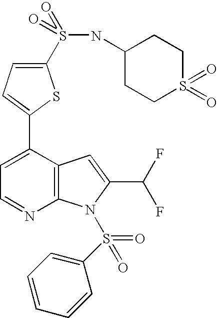

To a solution of 2-(difluoromethyl)-4-[5-(1H-imidazol-1-ylsulfonyl)-2-thienyl]-1-(phenylsulfonyl)-1H-pyrrolo[2,3-b]pyridine (65 mg, 0.125 mmol) in anhydrous tetrahydrofuran (1 mL) at 0° C. was added methyltriflate (16 μL, 0.137 mmol). After 30 min tetrahydro-2H-thiopyran-4-amine 1,1-dioxide (20 mg, 0.137 mmol) was added and warmed to ambient temperature after 30 min the reaction was concentrated (nitrogen blowdown). Purification by MDAP gave the title compound (40 mg).

LCMS MH+=602, rt=1.18 min

### Intermediate 65

### 5-Bromo-N-(2-hydroxy-2-methylpropyl)-2-thiophenesulfonamide

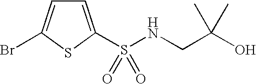

To a stirred solution of 1-amino-2-methyl-2-propanol (0.716 g, 8.03 mmol) and triethylamine (1.17 mL) in chloroform (25 mL) was added 5-bromo-2-thiophenesulfonyl chloride (2.0 g, 7.65 mmol) over 2 min and stirred at ambient temperature for 2 h. The reaction was washed sequentially with water (50 mL), saturated aqueous sodium carbonate (50 mL), saturated aqueous citric acid (50 mL), dried (hydrophobic frit) and concentrated in vacuo to furnish the title compound as white crystals (1.788 g).

LCMS MH+=314/316 rt=0.95 min

### Intermediate 66

### 5-Bromo-N-[(2,2-dimethyl-1,3-dioxolan-4-yl)methyl]-2-thiophenesulfonamide

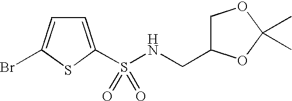

To a stirred solution of [(2,2-dimethyl-1,3-dioxolan-4-yl)methyl]amine (1.04 mL, 8.03 mmol) and triethylamine (1.17 mL) in chloroform (25 mL) was added 5-bromo-2-thiophenesulfonyl chloride (2.0 g, 7.65 mmol) over 2 min and stirred at ambient temperature for 1 h. The reaction was washed sequentially with water (50 mL), saturated aqueous sodium carbonate (50 mL), saturated aqueous citric acid (50 mL), dried (hydrophobic frit) and concentrated in vacuo to furnish the title compound as white crystals (2.546 g).

LCMS MH+=356/358, rt=1.08 min

### Intermediate 67

### 1,1-Dimethylethyl 4-{[(5-bromo-2-thienyl)sulfonyl]amino}-1-piperidinecarboxylate

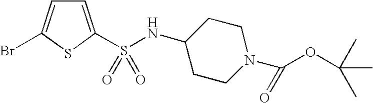

To a stirred solution of 1,1-dimethylethyl 4-amino-1-piperidinecarboxylate (1.6 g, 8.03 mmol) and triethylamine (1.17 mL) in chloroform (25 mL) was added 5-bromo-2-thiophenesulfonyl chloride (2.0 g, 7.65 mmol) over 2 min and stirred at ambient temperature for 1.5 h. The reaction was washed sequentially with water (50 mL), saturated aqueous sodium carbonate (50 mL), saturated aqueous citric acid (50 mL), dried (hydrophobic frit) and concentrated in vacuo, azeotroping with diethyl ether furnished the title compound as a white solid (2.46 g).

LCMS MH+=425/427 rt=3.38 min

### Intermediate 68

### 5-Bromo-N-(tetrahydro-2H-pyran-4-yl)-2-thiophenesulfonamide

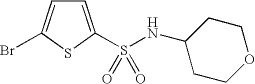

To a stirred solution of tetrahydro-2H-pyran-4-amine (0.84 g, 8.03 mmol) and triethylamine (1.17 mL) in chloroform (25 mL) was added 5-bromo-2-thiophenesulfonyl chloride (2.0 g, 7.65 mmol) over 5 min and stirred at ambient temperature for 24 h. The reaction was washed sequentially with water (50 mL), saturated aqueous sodium carbonate (50 mL), saturated aqueous citric acid (50 mL), dried (hydrophobic frit) and concentrated in vacuo, azeotroping with diethyl ether furnished the title compound as a white solid (2.245 g).

LCMS MH+=326/328, rt=2.78 min

### Intermediate 69

### [2-(1,1-Dimethylethyl)-1H-pyrrolo[2,3-b]pyridin-4-yl]boronic acid

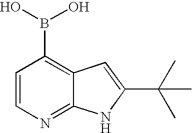

To a degassed solution of 4-bromo-2-(1,1-dimethylethyl)-1H-pyrrolo[2,3-b]pyridine (1.0 g, 3.95 mmol) in anhydrous tetrahydrofuran (25 mL) at 20° C. was added Sodium hydride as a 60% dispersion on mineral oil (190 mg 4.74 mmol) and stirred at 20° C. for 50 min. The mixture was degassed and cooled to −78° C. before addition of n-butyl-lithium 1.5M in hexanes (3.16 mL, 4.74 mmol) over 10 min. Reaction stirred at −78° C. for 1 h. Triisopropylborate (2.57 mL, 11.85 mmol) was added over 5 min. Reaction was warmed to 20° C. over 1 h. and NH₄Cl (20 mL) added. The aqueous was extracted with chloroform. The combined extracts were dried (hydrophobic frit) and concentrated in vacuo to a yellow solid (1.2 g). Purification by aminopropyl cartridge (20 g, eluent 2M ammonia in methanol) gave the title compound as a cream solid (566 mg).

LCMS MH+=219, rt=2.64 min

### Intermediate 70

### [2-(1-Methylethyl)-1H-pyrrolo[2,3-b]pyridin-4-yl]boronic acid

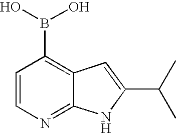

To a degassed solution of 4-bromo-2-(1-methylethyl)-1H-pyrrolo[2,3-b]pyridine (1.57 g, 6.57 mmol) in anhydrous tetrahydrofuran (40 mL) at 20° C. was added Sodium hydride as a 60% dispersion on mineral oil (329 mg 8.21 mmol) and stirred at 20° C. for 50 min. The mixture was degassed and cooled to −78° C. before addition of n-butyl-lithium 1.5M in hexanes (6.57 mL, 9.85 mmol) over 10 min. Reaction stirred at −78° C. for 45 min. Triisopropylborate (4.64 mL, 19.7 mmol) was added over 5 min. Reaction was warmed to 20° C. over 1.5 h and water (50 mL) added. The aqueous was extracted with ethyl acetate. The aqueous was adjusted to pH=7 (citric acid) and further extracted with ethyl acetate. The combined extracts were dried (hydrophobic frit) and concentrated in vacuo to a yellow oil. Purification by aminopropyl cartridge (20 g, eluent 7M ammonia in methanol) gave the title compound as a yellow solid (630 mg).

LCMS MH+=204, rt=0.67 min

### Intermediate 71

### 6-Chloro-N-(tetrahydro-2H-thiopyran-4-yl)-3-pyridinesulfonamide

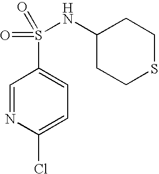

To a stirred solution of tetrahydro-2H-thiopyran-4-amine (1.16 g, 9.9 mmol) and 6-chloro-3-pyridinesulfonyl chloride (2.0 g, 9.43 mmol) in chloroform (25 mL) at ambient temperature was added triethylamine (1.45 mL, 10.3 mmol). After 1 h the reaction was washed sequentially with water (50 mL), saturated aqueous sodium carbonate (50 mL), saturated aqueous citric acid (50 mL), dried (hydrophobic frit) and concentrated in vacuo to furnish the title compound as a yellow solid. (2.46 g).

LCMS MH+=293, rt=1.01 min

### Intermediate 72

### [2-Methyl-1-(phenylsulfonyl)-1H-pyrrolo[2,3-b]pyridin-4-yl]boronic acid

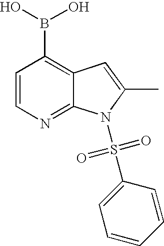

A mixture of 4-bromo-2-methyl-1-(phenylsulfonyl)-1H-pyrrolo[2,3-b]pyridine (10 g, 0.028 mol), palladium acetate (314 mg, 1.4 mmol) and potassium acetate (8.2 g, 0.084 mol) in dry N,N-dimethylformamide (50 mL) was stirred at room temperature under nitrogen. The reaction mixture was degassed and then 4,4,4′,4′,5,5,5′,5′-octamethyl-2,2′-bi-1,3,2-dioxaborolane (18 g, 0.071 mol) in dry N,N-dimethylformamide (50 mL) was slowly added. The reaction mixture was heated at 60° C. for 24 h under nitrogen. 4,4,4′,4′,5,5,5′,5′-octamethyl-2,2′-bi-1,3,2-dioxaborolane (7.2 g, 0.028 mol) was added and the reaction mixture was heated at 80° C. for 4 h under nitrogen. The reaction mixture was cooled down to room temperature and partitioned between dichloromethane (250 mL) and water (200 mL). After separation of the 2 phases, the aqueous layer was extracted with dichloromethane (100 mL). The organic extracts were combined, dried (hydrophobic frit) and concentrated in vacuo. The residue was purified by chromatography on a silica column (2×100 g FlashMaster columns), eluting with a cyclohexane:ethyl acetate gradient (0-100%), to give, after evaporation of the solvents in vacuo a beige solid (19.8 g). The solid was dissolved in Methanol:Dichloromethane (1:1, 10 mL), shared in two and eluted through two ion-exchange column (type SCX, 70 g columns). The columns were washed with methanol:dichloromethane (1:1, 200 mL) and the desired compounds were then released by 2M Ammonia in methanol:dichloromethane (2:1, 250 mL) elution. The desired fractions were combined and concentrated under vacuum to give 6.8 g of a beige solid. The solid was dissolved in methanol (50 mL) and treated with 2M hydrochloric acid (16 mL). The reaction mixture was heated at 45° C. for 2 h. The reaction mixture was cooled down to room temperature and the solvent removed under vacuum. The residue was partitioned between dichloromethane (150 mL) and water (100 mL). The pH of the aqueous phase was adjusted to 6 with a saturated solution of sodium hydrogen carbonate. After separation of the 2 phases, the aqueous layer was extracted with dichloromethane (150 mL). The organic extracts were combined, dried (hydrophobic frit) and concentrated in vacuo to afford the title compound as a light yellow solid (6.3 g).

MH+=317, rt=2.85 min

### Intermediate 73

### 1-[(5-Bromo-2-thienyl)sulfonyl]-1H-imidazole

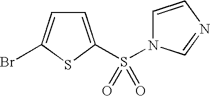

Imidazole (2.6 g, 38.2 mmol) was dissolved in chloroform (100 ml) and treated with triethylamine (5.86 ml). 5-bromo-2-thiophenesulfonyl chloride (10 g, 38.2 mmol) was added portionwise over 10 min. After 2 h the reaction was washed with water (200 ml) and saturated sodium bicarbonate (200 ml), dried (hydrophobic frit) and concentrated in vacuo to give the title compound as a white solid (11.255 g).

MH+=293/295, rt=1.02 min

### Intermediate 74

### 1,1-Dimethylethyl {2-[({6-[2-(trifluoromethyl)-1H-pyrrolo[2,3-b]pyridin-4-yl]-3-pyridinyl}sulfonyl)amino]ethyl}carbamate

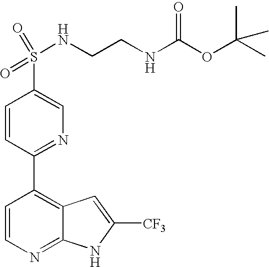

[2-(Trifluoromethyl)-1H-pyrrolo[2,3-b]pyridin-4-yl]boronic acid (140 mg, 0.61 mmol), 1,1-dimethylethyl (2-{[(6-chloro-3-pyridinyl)sulfonyl]amino}ethyl)carbamate (225 mg, 0.67 mmol), and 2-(dimethylamino)-2-biphenyl-palladium II chloride dibnornyl phosphine (5 mg, 0.0089 mmol) were added to a 20 ml microwave vessel followed by potassium phosphate (400 mg, 1.89 mmol), dioxane (8 ml) and water (2 ml). The resulting mixture was heated in a Biotage Initiator microwave whilst being stirred at 120° C. for 30 minutes. Resulting mixture was dissolved in DCM (50 ml) and water (20 ml). Partitioned organic phase and evaporated in vacuo to give the title compound as a yellow solid (600 mg) which was used without further purification.

MH+486, rt=1.10 min.

### Intermediate 75

### 5-Bromo-N-(2-hydroxy-1,1-dimethylpropyl)-2-pyridinesulfonamide

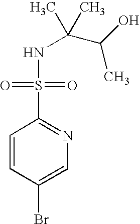

5-Bromo-2-pyridinesulfonyl chloride (1.57 g, 3.962 mmol) was dissolved in anhydrous DCM (35 ml). To 5 ml of this solution (0.566 mmol), a solution of 5-bromo-2-pyridinesulfonyl chloride cooled to 0° C. was added triethylamine (0.157 mL, 1.130 mmol) and 3-amino-3-methyl-2-butanol hydrochloride (79 mg. 0.565 mmol) suspended in anhydrous DCM (2 ml). The reaction mixture was stirred at room temperature overnight then allowed to stand at room temperature for 1 day. The reaction mixture was then diluted with DCM (10 ml), washed with hydrochloric acid (5 ml, 10.00 mmol of 2M aqueous solution) and the layers separated (hydrophobic frit). The DCM extract was evaporated in vacuo. The residue was loaded in dichloromethane and purified by SPE on silica (Si) 5 g eluting with cyclohexane:ethylacetate 10:1, 5:1, 2:1, 1:1, 1:2, 0:1, 10% MeOH/ethylacetate (2 column volumes of each). The appropriate fractions were combined and evaporated in vacuo to give the title compound as a yellow oil which solidified to a yellow solid (70 mg).

MH+=324.96, rt=2.44 min

Similarly prepared were:

### Intermediate 76

### 5-Bromo-N-[(1R)-1-(hydroxymethyl)propyl]-2-pyridinesulfonamide

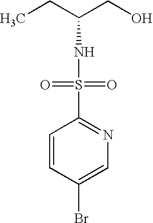

The title compound was obtained as a colourless oil (102 mg).

MH+=308.94/310.97, rt=2.24 min

### Intermediate 77

### 5-Bromo-N-(2-hydroxy-1,2-dimethylpropyl)-2-pyridinesulfonamide

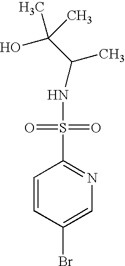

The title compound was obtained as a pale yellow oil (131 mg).

MH+=324.96, rt=2.29 min

### Example 1

### N-(2-Hydroxyethyl)-5-(2-methyl-1H-pyrrolo[2,3-b]pyridin-4-yl)-2-thiophenesulfonamide

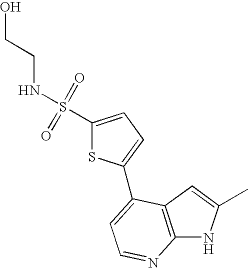

2-Methyl-1-(phenylsulfonyl)-4-(4,4,5,5-tetramethyl-1,3,2-dioxaborolan-2-yl)-1H-pyrrolo[2,3-b]pyridine (0.200 g, 0.5 mmol), 2-(dimethylamino)-2-biphenyl palladium (II) chloride dinorbornyl phosphine (0.014 g, 0.025 mmol), potassium phosphate tribasic (0.320 g, 1.5 mmol) and 5-bromo-N-(2-hydroxyethyl)-2-thiophenesulfonamide (0.286 g, 1 mmol) in dioxan/water (5:1, 5 ml) were heated in the Biotage Initiator microwave at 150° C. for 30 min. Sodium hydroxide (0.085 g, 10 eq) was introduced to the microwave vial and the mixture heated in the Biotage Initiator microwave at 150° C. for 1 h. The mixture was reduced in vacuo, diluted with water (30 ml) and brine (10 ml) and extracted with DCM (3×30 ml). The combined organic extracts were dried (phase separator) and reduced in vacuo and purified by MDAP to afford the title compound as a yellow solid (0.0314 g).

MH+=338, rt=2.63 min

### Example 2

### N-(1,1-Dioxidotetrahydro-2H-thiopyran-4-yl)-5-(2-methyl-1H-pyrrolo[2,3-b]pyridin-4-yl)-2-thiophenesulfonamide

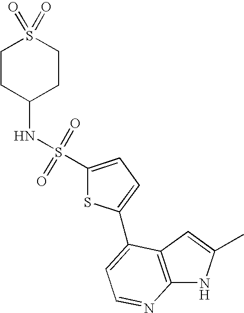

A mixture of N-(1,1-dioxidotetrahydro-2H-thiopyran-4-yl)-5-[2-methyl-1-(phenylsulfonyl)-1H-pyrrolo[2,3-b]pyridin-4-yl]-2-thiophenesulfonamide (0.153 g, 0.271 mmol) and 3M sodium hydroxide in MeOH (0.108 ml, 0.325 mmol) in THF (7 ml) was stirred at room temperature for 4 h. The reaction mixture was quenched with saturated ammonium chloride (10 ml) and extracted with DCM:EtOAc (1:1, 20 ml). The aqueous phase was extracted with DCM:EtOAc (1:1, 15 ml). The combined organic extracts were dried (hydrophobic frit), concentrated in vacuo and the residue purified by MDAP. The desired fractions were combined, concentrated in vacuo and were repurified by MDAP to give the title compound as a yellow solid (0.016 g, 14%).

MH+=426, rt=0.83 min

### Example 3

### N-(2-Aminoethyl)-5-(2-methyl-1H-pyrrolo[2,3-b]pyridin-4-yl)-2-thiophenesulfonamide

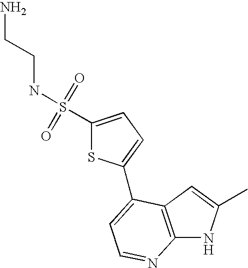

A suspension of 1,1-dimethylethyl[2-({[5-(2-methyl-1H-pyrrolo[2,3-b]pyridin-4-yl)-2-thienyl]sulfonyl}amino)ethyl]carbamate trifluoroacetate (0.090 g, 0.163 mmol) in DCM (5 ml) was treated with TFA (0.125 ml, 1.63 mmol). The reaction mixture was stirred at room temperature overnight. The solvent was removed and the yellow residue was purified by MDAP. The desired fractions were combined and concentrated in vacuo to give a yellow gum (0.058 g). The gum was dissolved in MeOH (2 ml) and eluted through an NH₂ cartridge to remove the TFA. The column was washed with 2 volumes of MeOH and the filtrate concentrated in vacuo to give the title compound as a yellow solid (0.039 g, 71%).

MH+=337, rt=0.66 min

### Example 4

### Formic acid—N-(2-hydroxyethyl)-5-[2-(3-piperidinyl)-1H-pyrrolo[2,3-b]pyridin-4-yl]-2-thiophenesulfonamide (1:1)

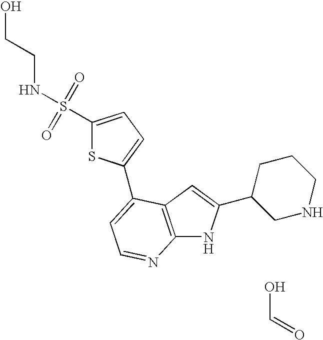

1,1-Dimethylethyl5{4[5({[2({[(1,1-dimethylethyl)oxy]carbonyl}amino)ethyl]amino}sulfonyl)-2-thienyl]-1H-pyrrolo[2,3-b]pyridin-2-yl}-3,4-dihydro-1(2H)-pyridinecarboxylate (0.035 g, 0.069 mmol) in 70% DMF/MeOH (2 ml) was hydrogenated using the H-cube (flow rate=11 ml/min, H₂ full, no heating). 70% DMF/MeOH was used to wash through the products. The reaction mixture was blown down using nitrogen, dissolved in TFA (2 ml) and water (0.2 ml). The reaction was stirred for 40 min, reduced under nitrogen and purified by MDAP to afford the title compound (0.010 g, 32%).

MH+=407, rt=2.08 min

### Example 5

### N-(2-Aminoethyl)-5-[2-(3-piperidinyl)-1H-pyrrolo[2,3-b]pyridin-4-yl]-2-thiophenesulfonamide

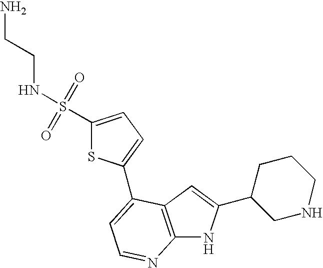

1,1-Dimethylethyl5-{4[-5-({[2-({[(1,1-dimethylethyl)oxy]carbonyl}amino)ethyl]amino}-sulfonyl)-2-thienyl]-1H-pyrrolo[2,3-b]pyridin-2-yl}-3,4-dihydro-1(2Hpyridinecarboxylate (0.065 g, 0.108 mmol) in 70% DMF/MeOH (2 ml) was hydrogenated using the H-cube (flow rate=1 ml/min, H₂ full, no heating). 70% DMF/MeOH was used to wash through the products. The reaction mixture was blown down using nitrogen, dissolved in TFA (2 ml) and water (0.2 ml). The reaction was stirred for 40 min, reduced under nitrogen and purified by MDAP to afford impure material (0.016 g). This material was purified by preparative TLC eluting with 10% ammonia in MeOH. The product was extracted from the silica with 10% ammonia in MeOH and reduced in vacuo to afford the title compound (0.0074 g, 17%).

MH+=406, rt=1.86 min

### Example 6

### Formic acid—N-(1,1-dioxidotetrahydro-3-thienyl)-5-[2-(3-piperidinyl)-1H-pyrrolo[2,3-b]pyridin-4-yl]-2-thiophenesulfonamide (1:1)

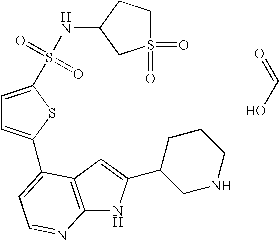

1,1-Dimethylethyl 5-[4-(5-{[(1,1-dioxidotetrahydro-3-thienyl)amino]sulfonyl}-2-thienyl)-1H-pyrrolo[2,3-b]pyridin-2-yl]-3,4-dihydro-1(2H)-pyridinecarboxylate (0.051 g, 0.088 mmol) in 70% DMF/MeOH (3 ml) was hydrogenated using the H-cube (flow rate=1 ml/min, H₂ full, no heating). 70% DMF/MeOH was used to wash through the products. The reaction mixture was blown down using nitrogen, dissolved in TFA (2 ml) and water (0.2 ml). The reaction was stirred for 40 min, reduced under nitrogen and purified by MDAP to afford the title compound (0.029 g, 63%).

MH+=481, rt=2.17 min

### Example 7

### N-(1,1-Dioxidotetrahydro-2H-thiopyran-4-yl)-5-[2-(3-piperidinyl)-1H-pyrrolo[2,3-b]pyridin-4-yl]-2-thiophenesulfonamide

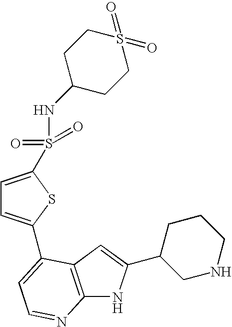

1,1-Dimethylethyl 5-[4-(5-{[(1,1-dioxidotetrahydro-2H-thiopyran-4-yl)amino]sulfonyl}-2-thienyl)-1H-pyrrolo[2,3-b]pyridin-2-yl]-3,4-dihydro-1(2H)-pyridinecarboxylate (0.040 g, 0.067 mmol) in 70% DMF/MeOH (2 ml) was hydrogenated using the H-cube (flow rate=1 ml/min, H₂ full, no heating). 70% DMF/MeOH was used to wash through the products. The first fraction collected was reduced in vacuo and treated with TFA (2.5 ml) and water (0.25 ml). The reaction was stirred for 30 min, reduced in vacuo and purified by MDAP to afford impure compound (0.012 g). This was purified by silica preparative tic (1:1 MeOH:EtOAc+5% ammonia solution) to afford the title compound (0.0048 g, 15%).

MH+=481, rt=2.17 min

### Example 8

### N-(1,1-Dioxidotetrahydro-2H-thiopyran-4-yl)-6-(2-methyl-1H-pyrrolo[2,3-b]pyridin-4-yl)-3-pyridinesulfonamide

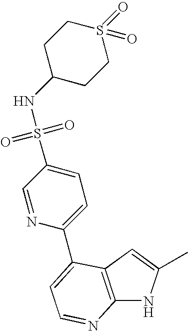

A solution of 4-bromo-2-methyl-1-(phenylsulfonyl)-1H-pyrrolo[2,3-b]pyridine (1.2 g, 3.42 mmol) in anhydrous DCM (60 ml) was de-gassed for 15 min then palladium acetate (0.0384 g, 0.171 mmol) and potassium acetate (1.006 g, 10.26 mmol) were added and the mixture stirred for 5 min under nitrogen at room temperature. 4,4,4′,4′,5,5,5′,5′-Octamethyl-2,2′-bi-1,3,2-dioxaborolane (2.18 g, 8.55 mmol) was added, the reaction was flushed with nitrogen and heated at 65° C. for 6 h, stirred at room temperature overnight then heated for a further 4 h at 65° C. under nitrogen. The reaction mixture was allowed to cool to room temperature then concentrated in vacuo. The residue was partitioned between water (100 ml) and DCM (100 ml). The layers were separated and the aqueous extracted with DCM (2×100 ml). The combined DCM extracts were filtered then evaporated in vacuo. The residue was triturated with cyclohexane, filtered and the filtrate evaporated in vacuo to give 2-methyl-1-(phenylsulfonyl)-4-(4,4,5,5-tetramethyl-1,3,2-dioxaborolan-2-yl)-1H-pyrrolo[2,3-b]pyridine as a yellow oily solid (3.21 g). A mixture of 2-methyl-1-(phenylsulfonyl)-4-(4,4,5,5-tetramethyl-1,3,2-dioxaborolan-2-yl)-1H-pyrrolo[2,3-b]pyridine (0.100 g, 0.106 mmol), 6-chloro-N-(1,1-dioxidotetrahydro-2H-thiopyran-4-yl)-3-pyridinesulfonamide (0.069 g, 0.212 mmol), sodium carbonate (0.023 g, 0.217 mmol) and 1,1′-bis(diphenylphosphino)ferrocene dichloro palladium (II) (0.0049 g, 0.006 mmol) in dioxan (1.2 ml) and water (0.3 ml) was heated at 150° C. for 30 min in the Biotage Initiator microwave. Sodium hydroxide (0.045 g, 1.12 mmol) was added and the mixture heated in the Biotage Initiator microwave at 150° C. for 1 h. The reaction was concentrated in vacuo then partitioned between DCM (15 ml), water (10 ml) and brine (5 ml). The layers were separated (hydrophobic frit) and the aqueous layer extracted with DCM (2×15 ml). The aqueous layer was extracted with EtOAc (2×15 ml). The combined DCM and EtOAc extracts were evaporated in vacuo then purified by MDAP to give the title compound as a yellow solid (0.0037 g).

MH+=421, rt=2.67 min

### Example 9

### 2-Methyl-4-[5-(1-pyrrolidinylsulfonyl)-2-pyridinyl]-1H-pyrrolo[2,3-b]pyridine

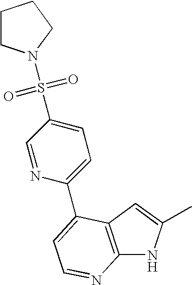

A solution of 4-bromo-2-methyl-1-(phenylsulfonyl)-1H-pyrrolo[2,3-b]pyridine (1.2 g, 3.42 mmol) in anhydrous DCM (60 ml) was de-gassed for 15 min then palladium acetate (0.0384 g, 0.171 mmol) and potassium acetate (1.006 g, 10.26 mmol) were added and the mixture stirred for 5 min under nitrogen at room temperature. 4,4,4′,4′,5,5,5′,5′-Octamethyl-2,2′-bi-1,3,2-dioxaborolane (2.18 g, 8.55 mmol) was added, the reaction was flushed with nitrogen and heated at 65° C. for 6 h, stirred at room temperature overnight then heated for a further 4 h at 65° C. under nitrogen. The reaction mixture was allowed to cool to room temperature then concentrated in vacuo. The residue was partitioned between water (100 ml) and DCM (100 ml). The layers were separated and the aqueous extracted with DCM (2×100 ml). The combined DCM extracts were filtered then evaporated in vacuo. The residue was triturated with cyclohexane, filtered and the filtrate evaporated in vacuo to give 2-methyl-1-(phenylsulfonyl)-4-(4,4,5,5-tetramethyl-1,3,2-dioxaborolan-2-yl)-1H-pyrrolo[2,3-b]pyridine as a yellow oily solid (3.21 g). A mixture of 2-methyl-1-(phenylsulfonyl)-4-(4,4,5,5-tetramethyl-1,3,2-dioxaborolan-2-yl)-1H-pyrrolo[2,3-b]pyridine (0.200 g, 0.212 mmol), 2-chloro-5-(1-pyrrolidinylsulfonyl)pyridine (0.102 g, 0.424 mmol), sodium carbonate (0.045 g, 0.424 mmol) and 1,1′-bis(diphenylphosphino)ferrocene dichloro palladium (II) (0.0086 g, 0.0106 mmol) in dioxan (2.4 ml) and water (0.6 ml) was heated at 150° C. for 30 min in the Biotage Initiator microwave. Sodium hydroxide (0.089 g, 2.22 mmol) was added and the mixture heated in the Biotage Initiator microwave at 150° C. for 1 h. The reaction was diluted with MeOH (10 ml), filtered through celite (10 g cartridge) and evaporated in vacuo. The residue was partitioned between water (10 ml) and DCM (30 ml), the layers were separated and the aqueous extracted with DCM (30 ml). The combined DCM extracts were evaporated in vacuo and purified by MDAP to give the title compound as a light brown solid (0.0164 g).

MH+=343, rt=2.89 min

### Example 10

### N-(1,1-Dioxidotetrahydro-2H-thiopyran-4-yl)-5-(2-methyl-1H-pyrrolo[2,3-b]pyridin-4-yl)-2-pyridinesulfonamide

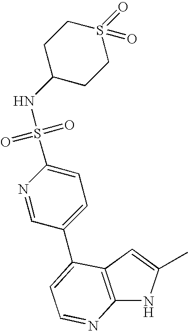

A solution of 4-bromo-2-methyl-1-(phenylsulfonyl)-1H-pyrrolo[2,3-b]pyridine (1.2 g, 3.42 mmol) in anhydrous DCM (60 ml) was de-gassed for 15 min then palladium acetate (0.0384 g, 0.171 mmol) and potassium acetate (1.006 g, 10.26 mmol) were added and the mixture stirred for 5 min under nitrogen at room temperature. 4,4,4′,4′,5,5,5′,5′-Octamethyl-2,2′-bi-1,3,2-dioxaborolane (2.18 g, 8.55 mmol) was added, the reaction was flushed with nitrogen and heated at 65° C. for 6 h, stirred at room temperature overnight then heated for a further 4 h at 65° C. under nitrogen. The reaction mixture was allowed to cool to room temperature then concentrated in vacuo. The residue was partitioned between water (100 ml) and DCM (100 ml). The layers were separated and the aqueous extracted with DCM (2×100 ml). The combined DCM extracts were filtered then evaporated in vacuo. The residue was triturated with cyclohexane, filtered and the filtrate evaporated in vacuo to give 2-methyl-1-(phenylsulfonyl)-4-(4,4,5,5-tetramethyl-1,3,2-dioxaborolan-2-yl)-1H-pyrrolo[2,3-b]pyridine as a yellow oily solid (3.21 g). A mixture of 2-methyl-1-(phenylsulfonyl)-4-(4,4,5,5-tetramethyl-1,3,2-dioxaborolan-2-yl)-1H-pyrrolo[2,3-b]pyridine (0.200 g, 0.212 mmol), 5-bromo-N-(1,1-dioxidotetrahydro-2H-thiopyran-4-yl)-2-pyridinesulfonamide (0.156 g, 0.424 mmol), sodium carbonate (0.045 g, 0.424 mmol) and 1,1′-bis(diphenylphosphino)ferrocene dichloro palladium (II) (0.0086 g, 0.0106 mmol) in dioxan (2.4 ml) and water (0.6 ml) was heated at 150° C. for 30 min in the Biotage Initiator microwave. The reaction was heated with further aliquots of 1,1′-bis(diphenylphosphino)ferrocene dichloro palladium (II) (0.009 g, 0.011 mmol) and sodium carbonate (0.045 g, 0.424 mmol) at 150° C. for 30 min in the Biotage Initiator microwave, then a further 30 min at 150° C. in the Biotage Initiator microwave. Sodium hydroxide (0.085 g-0.090 g, 2.12 mmol-2.25 mmol) was added and the reaction mixture heated in the Biotage Initiator microwave at 150° C. for 1 h. The mixture was diluted with MeOH, filtered through a celite cartridge (10 g cartridge), evaporated in vacuo and partitioned between DCM (30 ml) and water (30 ml). The layers were separated and the aqueous layer extracted with DCM (30 ml) and EtOAc (30 ml). The combined DCM and EtOAc extracts were evaporated in vacuo and purified by MDAP to give the title compound (0.006 g).

MH+=420, rt=2.42 min

### Example 11

### 2-Methyl-4-[6-(1-pyrrolidinylsulfonyl)-3-pyridinyl]-1H-pyrrolo[2,3-b]pyridine

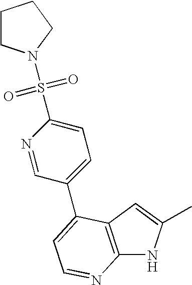

### Method A

A solution of 4-bromo-2-methyl-1-(phenylsulfonyl)-1H-pyrrolo[2,3-b]pyridine (1.2 g, 3.42 mmol) in anhydrous DCM (60 ml) was de-gassed for 15 min then palladium acetate (0.0384 g, 0.171 mmol) and potassium acetate (1.006 g, 10.26 mmol) were added and the mixture stirred for 5 min under nitrogen at room temperature. 4,4,4′,4′,5,5,5′,5′-Octamethyl-2,2′-bi-1,3,2-dioxaborolane (2.18 g, 8.55 mmol) was added, the reaction was flushed with nitrogen and heated at 65° C. for 6 h, stirred at room temperature overnight then heated for a further 4 h at 65° C. under nitrogen. The reaction mixture was allowed to cool to room temperature then concentrated in vacuo. The residue was partitioned between water (100 ml) and DCM (100 ml). The layers were separated and the aqueous extracted with DCM (2×100 ml). The combined DCM extracts were filtered then evaporated in vacuo. The residue was triturated with cyclohexane, filtered and the filtrate evaporated in vacuo to give 2-methyl-1-(phenylsulfonyl)-4-(4,4,5,5-tetramethyl-1,3,2-dioxaborolan-2-yl)-1H-pyrrolo[2,3-b]pyridine as a yellow oily solid (3.21 g). A mixture of 2-methyl-1-(phenylsulfonyl)-4-(4,4,5,5-tetramethyl-1,3,2-dioxaborolan-2-yl)-1H-pyrrolo[2,3-b]pyridine (0.200 g, 0.212 mmol), 5-bromo-2-(1-pyrrolidinylsulfonyl)pyridine (0.123 g, 0.424 mmol), sodium carbonate (0.045 g, 0.424 mmol) and 1,1′-bis(diphenylphosphino)ferrocene dichloro palladium (II) (0.0086 g, 0.0106 mmol) in dioxan (2.4 ml) and water (0.6 ml) was heated at 150° C. for 30 min in the Biotage Initiator microwave. Sodium hydroxide (0.085 g-0.090 g, 2.12 mmol-2.25 mmol) was added and the reaction mixture heated in the Biotage Initiator microwave at 150° C. for 1 h. The mixture was diluted with MeOH, filtered through a celite cartridge (10 g cartridge), evaporated in vacuo and partitioned between DCM (30 ml) and water (30 ml). The layers were separated and the aqueous layer extracted with DCM (30 ml) and EtOAc (30 ml). The combined DCM and EtOAc extracts were evaporated in vacuo and purified by MDAP to give the title compound as a light brown solid (0.026 g).

MH+=343, rt=2.86 min

### Method B

The title compound was also prepared similarly to Example 23.

MH+=343, rt=2.9 min

### Example 12

### N-(1,1-Dioxidotetrahydro-2H-thiopyran-4-yl)-2-methyl-5-[2-(trifluoromethyl)-1H-pyrrolo[2,3-b]pyridin-4-yl]-3-thiophenesulfonamide

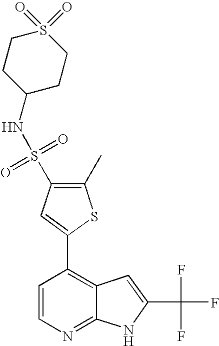

A mixture of [2-(trifluoromethyl)-1H-pyrrolo[2,3-b]pyridin-4-yl]boronic acid (0.047 g, 0.205 mmol), 5-bromo-N-(1,1-dioxidotetrahydro-2H-thiopyran-4-yl)-2-methyl-3-thiophenesulfonamide (0.09 g, 0.23 mmol), isopropylalcohol (2.07 ml), 1M sodium bicarbonate (0.69 ml) 1,1′-bis(diphenylphosphino)ferrocene-palladium (II)dichloride (0.0094 g, 0.0115 mmol) was degassed for 5 min then heated in a sealed tube in a Biotage Initiator microwave at 120° C. for 30 min. The reaction mixture was then diluted with MeOH, filtered, the filtrate evaporated in vacuo. The filtered solid and the evaporated filtrate were purified by MDAP to give the title compound as a white solid. (0.029 g).

MH+=494, rt=3.08 min

### Example 13

### N-(1,1-Dioxidotetrahydro-2H-thiopyran-4-yl)-4-methyl-2-[2-(trifluoromethyl)-1H-pyrrolo[2,3-b]pyridin-4-yl]-1,3-thiazole-5-sulfonamide

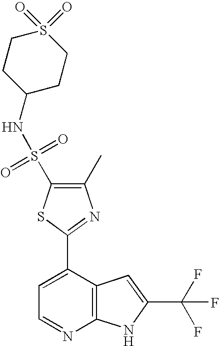

A mixture of 2-chloro-N-(1,1-dioxidotetrahydro-2H-thiopyran-4-yl)-4-methyl-1,3-thiazole-5-sulfonamide (0.069 g, 0.2 mmol), [2-(trifluoromethyl)-1H-pyrrolo[2,3-b]pyridin-4-yl]boronic acid (0.046 g, 0.2 mmol), 1,1′-bis(diphenylphosphino)ferrocenedichloro palladium(II) dichloromethane complex (0.00732 g, 0.01 mmol) and 1M sodium bicarbonate (0.6 ml, 0.6 mmol) in Isopropanol (1.8 ml) was degassed for 5 min then heated in a sealed tube in the Biotage Initiator microwave at 120° C. for 30 min. The reaction mixture was evaporated in vacuo and then purified by MDAP to give the title compound as a white solid (0.023 g).

MH+=495, rt=3.08 min

### Example 14

### N-(1,1-Dioxidotetrahydro-2H-thiopyran-4-yl)-2-(2-methyl-1H-pyrrolo[2,3-b]pyridin-4-yl)-5-pyrimidinesulfonamide

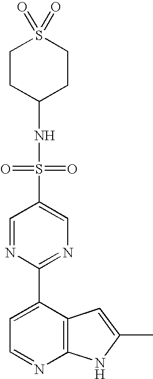

A solution of N-(1,1-dioxidotetrahydro-2H-thiopyran-4-yl)-2-[2-methyl-1-(phenylsulfonyl)-1H-pyrrolo[2,3-b]pyridin-4-yl]-5-pyrimidinesulfonamide (0.035 g, 0.062 mmol), THF (2 ml, anhydrous), TBAF (0.124 ml of 1M solution in THF) was stirred at room temperature for 1.5 h, then at 70° C. for 0.5 h. The reaction mixture was then heated in a sealed tube in a Biotage Initiator microwave at 50° C. for 30 min, then at 70° C. for 60 min. TBAF (0.124 ml of 1M solution in THF) was added and the reaction mixture heated at 70° C. in a sealed tube in the Biotage Initiator microwave for 1 h then at 140° C. in a sealed tube in the Biotage Initiator microwave for 1 h. The reaction mixture was then purified by SCX (5 g) eluted with MeOH then 2M NH₃/MeOH. The ammonia fractions were evaporated in vacuo and the residue triturated with MeOH to give the title compound as a yellow solid (0.016 g). MH+=422, rt=2.58 min

### Example 15

### 6-[2-(Difluoromethyl)-1H-pyrrolo[2,3-b]pyridin-4-yl]-N-(1,1-dioxidotetrahydro-2H-thiopyran-4-yl)-3-pyridinesulfonamide

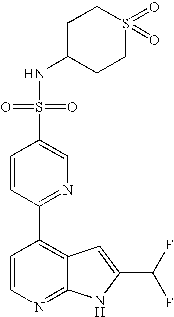

A suspension of 6-[2-(difluoromethyl)-1-(phenylsulfonyl)-1H-pyrrolo[2,3-b]pyridin-4-yl]-N-(1,1-dioxidotetrahydro-2H-thiopyran-4-yl)-3-pyridinesulfonamide (0.0217 g, 0.0364 mmol) in anhydrous THF (2 ml), was treated with TBAF (0.0728 ml of 1M solution in THF), and stirred at room temperature under N₂ for 4 h. The reaction mixture was purified by SCX (2 g) eluted with MeOH and 2M NH₃/MeOH. The ammonia fractions were evaporated by blow down then triturated with MeOH to give the title compound as a brown solid (0.00905 g).

MH+=457, rt=2.72 min

### Example 16

### 6-[2-(Difluoromethyl)-1H-pyrrolo[2,3-b]pyridin-4-yl]-N-4-piperidinyl-3-pyridinesulfonamide

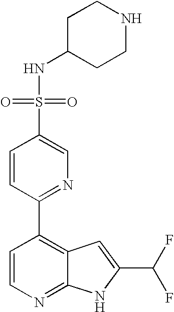

A solution of 1,1-dimethylethyl 4-[({6-[2-(difluoromethyl)-1-(phenylsulfonyl)-1H-pyrrolo[2,3-b]pyridin-4-yl]-3-pyridinyl}sulfonyl)amino]-1-piperidinecarboxylate (0.099 g, 0.15 mmol) in anhydrous THF (4 ml) was treated with TBAF (0.306 ml of 1M solution in THF) and stirred at room temperature under N₂ for 4 h, then the reaction mixture was purified by SCX (5 g) eluted with MeOH, 2M NH₃/MeOH. The ammonia fractions were evaporated in vacuo to give a light brown solid which was treated with MeOH (5 ml) then HCl/Dioxane (4M solution; 0.075 ml) were added and the mixture left to stand at room temperature for 30 min, then HCl/Dioxane (4M solution; 0.075 ml) were added and the mixture left to stand at room temperature overnight. The mixture was evaporated in vacuo, then purified by SCX (2 g) eluted with MeOH, 2M NH₃/MeOH. The ammonia fractions were evaporated in vacuo to give a brown solid which was purified by MDAP to give the title compound as a yellow solid (0.0205 g).

MH+=408, rt=2.72 min

### Example 17

### 2-(Difluoromethyl)-4-{5-[(1,1-dioxido-4-thiomorpholinyl)sulfonyl]-2-pyridinyl}-1H-pyrrolo[2,3-b]pyridine

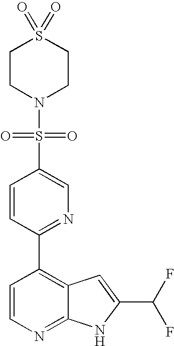

A mixture of [2-(difluoromethyl)-1-(phenylsulfonyl)-1H-pyrrolo[2,3-b]pyridin-4-yl]boronic acid (0.0704 g, 0.2 mmol) 4-[(6-chloro-3-pyridinyl)sulfonyl]thiomorpholine 1,1-dioxide (0.0924 g, 0.22 mmol), 1,1′-bis(diphenylphosphino)ferrocenedichloro palladium(II) (0.0082 g, 0.01 mmol), 0.6 ml of 1M sodium bicarbonate and 1.8 ml IPA was degassed for 5 min then heated in a sealed tube in a Biotage Initiator microwave at 120° C. for 30 min. The reaction mixture was then diluted with MeOH and filtered. The solid was washed with DCM, this contained product so was combined with all the filtrates and evaporated in vacuo. To the residue was added anhydrous THF (4 ml) the TBAF (0.4 ml of 1M solution in THF). The mixture was stirred at room temperature under N₂ for 6 h. The reaction mixture was then diluted with MeOH and purified by SCX (5 g) eluted with MeOH:DMF (1:1,) then 2M NH₃ in MeOH:DMF (1:1). The ammonia fractions were evaporated in vacuo then purified by MDAP to give the title compound as a yellow solid (0.00482 g).

MH+=443, rt=2.79 min

Example 18 was similarly prepared:

### Example 18

### 2-(Difluoromethyl)-4-{6-[(1,1-dioxido-4-thiomorpholinyl)sulfonyl]-3-pyridinyl}-1H-pyrrolo[2,3-b]pyridine

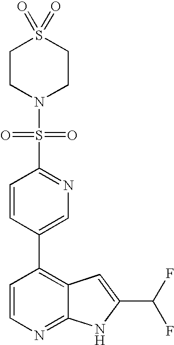

The title compound was obtained as a yellow solid (0.0087 g).

MH+=443, rt=3.22 min

### Example 19

### 5-[2-(Difluoromethyl)-1H-pyrrolo[2,3-b]pyridin-4-yl]-N-4-piperidinyl-2-pyridinesulfonamide

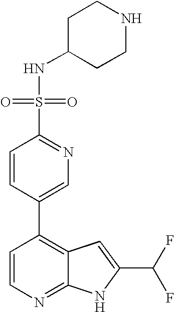

A mixture of [2-(difluoromethyl)-1-(phenylsulfonyl)-1H-pyrrolo[2,3-b]pyridin-4-yl]boronic acid (0.0704 g, 0.2 mmol), 1,1-dimethylethyl 4-{[(5-bromo-2-pyridinyl)sulfonyl]amino}-1-piperidinecarboxylate (0.0924 g, 0.22 mmol), 1,1′-bis(diphenylphosphino)ferrocenedichloro palladium(II) (0.0082 g, 0.01 mmol), 0.6 ml of 1M sodium bicarbonate and 1.8 ml IPA was degassed for 5 min then heated in a sealed tube in a Biotage Initiator microwave at 120° C. for 30 min. The reaction mixture was then diluted with MeOH and filtered. The filtrate was purified by SCX (5 g) eluted with MeOH, 2M NH₃/MeOH. The methanol fractions were evaporated in vacuo. To the residue was added anhydrous THF (4 ml) the TBAF (0.386 ml of 1M solution in THF). The mixture was stirred at room temperature under N₂ for 6 h. The reaction mixtures were then diluted with MeOH and purified by SCX (5 g) eluted with MeOH then 2M NH₃/MeOH. The ammonia fractions were evaporated in vacuo. To the residue was added TFA (2 ml) and the mixture left to stand at room temperature for 2 h then evaporated in vacuo and purified by SCX (2 g) eluted with MeOH, then 2M NH₃/EtOH. The ammonia fractions were evaporated in vacuo then the residue purified by MDAP to give the title compound as a yellow solid (0.0192 g).

MH+=408, rt=2.3 min

### Example 20

### 6-[2-(Difluoromethyl)-1H-pyrrolo[2,3-b]pyridin-4-yl]-N-(1,1-dioxidotetrahydro-2H-thiopyran-4-yl)-N-methyl-3-pyridinesulfonamide

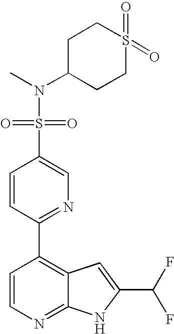

A mixture of [2-(difluoromethyl)-1-(phenylsulfonyl)-1H-pyrrolo[2,3-b]pyridin-4-yl]boronic acid (0.0704 g, 0.2 mmol), 6-chloro-N-(1,1-dioxidotetrahydro-2H-thiopyran-4-yl)-N-methyl-3-pyridinesulfonamide (0.0745 g, 0.22 mmol), 1,1′-bis(diphenylphosphino)ferrocenedichloro palladium(II) (0.0082 g, 0.01 mmol), 0.6 ml of 1M sodium bicarbonate and 1.8 ml IPA was degassed for 5 min then heated in a sealed tube in a Biotage Initiator microwave at 120° C. for 30 min. The reaction mixture was then evaporated by blowdown to give a brown solid. To the brown solid was added anhydrous THF (4 ml), TBAF (0.4 ml of 1M solution in THF) and the mixture stirred at room temperature for 5 h. The reaction mixture was then purified by SCX (5 g) eluted with MeOH, 2M NH₃/EtOH:DMF (1:1). The ammonia fractions were evaporated in vacuo and the residue purified by MDAP to give the title compound as a yellow solid (0.015 g).

MH+=471, rt=2.93 min

### Example 21

### 5-[2-(Difluoromethyl)-1H-pyrrolo[2,3-b]pyridin-4-yl]-N-(1,1-dioxidotetrahydro-2H-thiopyran-4-yl)-N-methyl-2-pyridinesulfonamide

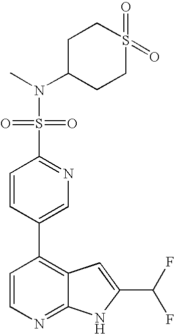

A mixture of [2-(difluoromethyl)-1-(phenylsulfonyl)-1H-pyrrolo[2,3-b]pyridin-4-yl]boronic acid (0.0704 g, 0.2 mmol), 5-bromo-N-(1,1-dioxidotetrahydro-2H-thiopyran-4-yl)-N-methyl-2-pyridinesulfonamide (0.0842 g, 0.22 mmol), 1,1′-bis(diphenylphosphino)ferrocenedichloro palladium(II) (0.0082 g, 0.01 mmol), 0.6 ml of 1M sodium bicarbonate and 1.8 ml IPA was degassed for 5 min then heated in a sealed tube in a Biotage Initiator microwave at 120° C. for 30 min. The reaction mixture was then evaporated by blowdown to give a brown solid. To the brown solid was added anhydrous THF (4 ml), TBAF (0.4 ml of 1M solution in THF) and the mixture stirred at room temperature for 5 h. The reaction mixture was then purified by SCX (5 g) eluted with MeOH, 2M NH₃/EtOH:DMF (1:1). The ammonia fractions were evaporated in vacuo and the residue purified by MDAP to give the title compound as a yellow solid (0.016 g).

MH+=471, rt=2.87 min

### Example 22

### 5-[2-(Difluoromethyl)-1H-pyrrolo[2,3-b]pyridin-4-yl]-N-(1,1-dioxidotetrahydro-2H-thiopyran-4-yl)-2-pyridinesulfonamide

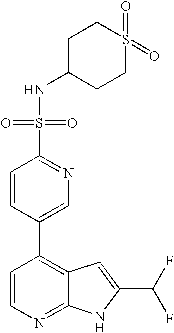

A mixture of [2-(difluoromethyl)-1-(phenylsulfonyl)-1H-pyrrolo[2,3-b]pyridin-4-yl]boronic acid (0.0704 g, 0.2 mmol), 5-bromo-N-(1,1-dioxidotetrahydro-2H-thiopyran-4-yl)-2-pyridinesulfonamide (0.082 g, 0.22 mmol), 1,1′-bis(diphenylphosphino)ferrocenedichloro palladium(II) (0.0082 g, 0.01 mmol), 0.6 ml of 1M sodium bicarbonate and 1.8 ml IPA was degassed for 5 min then heated in a sealed tube in a Biotage Initiator microwave at 120° C. for 30 min. The reaction mixture was then evaporated by blowdown to give a brown solid. To the brown solid was added anhydrous THF (4 ml), TBAF (0.4 ml of 1M solution in THF) and the mixture stirred at room temperature for 5 h. The reaction mixture was then purified by SCX (5 g) eluted with MeOH, 2M NH₃/EtOH, 2M NH₃/EtOH:DMF (1:1). Very little product was present in the ammonia fractions. The MeOH fractions were evaporated in vacuo to recover the starting material, this was then redissolved in anhydrous THF (0.4 ml), treated with TBAF (0.4 ml of 1M solution in THF) and stirred for 3 h. The reaction mixture was purified by SCX (5 g) eluted with MeOH, 2M NH₃/EtOH, 2M NH₃/EtOH:DMF (1:1). The ammonia fractions were evaporated in vacuo and then purified by MDAP to give the title compound as a white solid (0.009 g).

MH+=457, rt=2.6 min

### Example 23

### N-(1,1-Dioxidotetrahydro-2H-thiopyran-4-yl)-6-(2-methyl-1H-pyrrolo[2,3-b]pyridin-4-yl)-3-pyridinesulfonamide

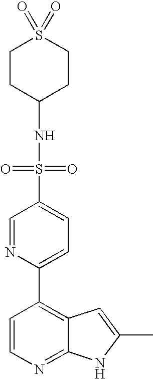

### Method A

A mixture of N-(1,1-dioxidotetrahydro-2H-thiopyran-4-yl)-6-[2-methyl-1-(phenylsulfonyl)-1H-pyrrolo[2,3-b]pyridin-4-yl]-3-pyridinesulfonamide (0.0274 g, 0.0489 mmol), TBAF (0.098 ml of a 1M solution in THF) in anhydrous THF (2 ml) was heated in a sealed in a Biotage Initiator microwave at 140° C. for 1 h. The reaction mixture was then purified by SCX (2 g), eluted with MeOH, 2M NH₃/MeOH. The ammonia fractions were evaporated in vacuo then triterated with MeOH to give the title compound as a yellow solid (0.0156 g).

MH+=421, rt=2.43 min

### Method B

A solution of [2-methyl-1-(phenylsulfonyl)-1H-pyrrolo[2,3-b]pyridin-4-yl]boronic acid in dry isopropanol (900 μL) was added to a microwave vessel containing 4-[(6-chloro-3-pyridinyl)sulfonyl]thiomorpholine 1,1-dioxide (34 mg, 1.1 mmol) and [1,1′-Bis(diphenylphosphino)ferrocene]dichloropalladium (II) complex with DCM 1:1 (0.4 mol %).

A solution of sodium hydrogen carbonate (1M, 300 μL) was added and the reaction mixture heated at 120° C. in the microwave for 30 minutes. The reaction mixture was applied directly to a C18 cartridge (500 mg, pre-equilibrated with acetonitrile) and eluted with 0.1% TFA in acetonitrile (3×1 mL) followed by concentration by blow down. The residue was resuspended in THF (1 mL) and treated with TBAF (1M in THF, 300 μL) and heated at 140° C. in the microwave for 1 h. The residue was concentrated by blowdown and purification by mass directed HPLC gave the title compound.

MH+=421, rt=2.53 min

Similarly prepared were:

| Example | Structure | Name | MH+ | Rt (min) |
|---|---|---|---|---|
| 24 | 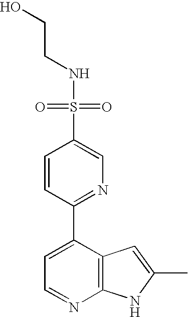 | N-(2-hydroxyethyl)-6-(2-methyl- 1H-pyrrolo[2,3-b]pyridin-4-yl)-3- pyridinesulfonamide | 333 | 2.36 |
| 25 | 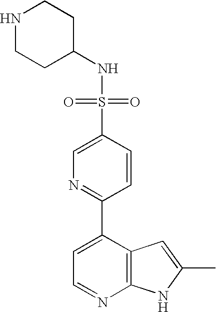 | 6-(2-methyl-1H-pyrrolo[2,3- b]pyridin-4-yl)-N-4-piperidinyl- 3-pyridinesulfonamide | 372 | 2.11 |
| 26 | 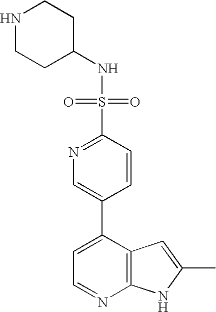 | 5-(2-methyl-1H-pyrrolo[2,3- b]pyridin-4-yl)-N-4-piperidinyl- 2-pyridinesulfonamide | 372 | 2.06 |
| 27 | 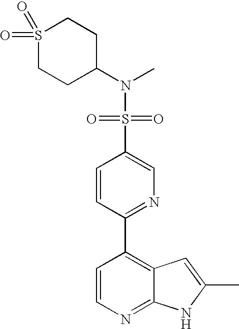 | N-(1,1-dioxidotetrahydro-2H- thiopyran-4-yl)-N-methyl-6-(2- methyl-1H-pyrrolo[2,3- b]pyridin-4-yl)-3- pyridinesulfonamide | 435 | 2.68 |
| 28 | 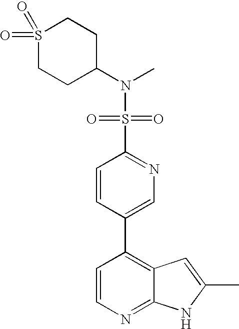 | N-(1,1-dioxidotetrahydro-2H- thiopyran-4-yl)-N-methyl-5-(2- methyl-1H-pyrrolo[2,3- b]pyridin-4-yl)-2- pyridinesulfonamide | 435 | 2.67 |
| 29 | 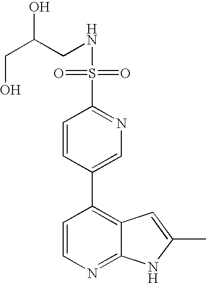 | N-(2,3-dihydroxypropyl)-5-(2- methyl-1H-pyrrolo[2,3- b]pyridin-4-yl)-2- pyridinesulfonamide | 364 | 2.21 |
| 30 | 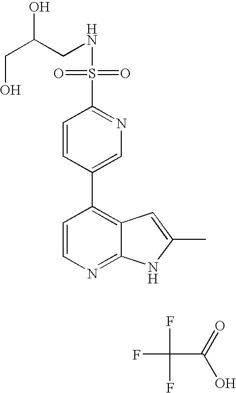 | N-(2,3-dihydroxypropyl)-5-(2- methyl-1H-pyrrolo[2,3- b]pyridin-4-yl)-2- pyridinesulfonamide trifluoroacetate (salt) | 364 | 2.21 |

### Example 31

### N-(1,1-Dioxidotetrahydro-3-thienyl)-5-(2-methyl-1H-pyrrolo[2,3-b]pyridin-4-yl)-2-thiophenesulfonamide

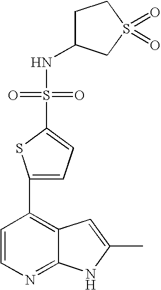

2-Methyl-1-(phenylsulfonyl)-4-(4,4,5,5-tetramethyl-1,3,2-dioxaborolan-2-yl)-1H-pyrrolo[2,3-b]pyridine (0.200 g, 0.5 mmol), 2-(dimethylamino)-2-biphenyl palladium (II) chloride dinorbornyl phosphine (0.014 g, 0.025 mmol), potassium phosphate tribasic (0.320 g, 1.5 mmol) and 5-bromo-N-(1,1-dioxidotetrahydro-3-thienyl)-2-thiophenesulfonamide (0.36 g, 1 mmol) in dioxan/water (5:1, 5 ml) were heated in the Biotage Initiator microwave at 150° C. for 30 min. Sodium hydroxide (0.085 g, 10 eq) was introduced to the microwave vial and the mixture heated in the Biotage Initiator microwave at 150° C. for 1 h. The mixture was reduced in vacuo, diluted with water (30 ml) and brine (10 ml) and extracted with DCM (3×30 ml). The combined organic extracts were dried (phase separator) and reduced in vacuo and purified by MDAP to recover the protected product. To this was added NaOH (0.065 g, 0.01625 mmol), dioxan/water (5:1, 3 ml) and heated in the Biotage Initiator microwave at 120° C. for 1 h. The mixture was reduced in vacuo, diluted with water (30 ml), neutralised with HCl (2M, approx 3 ml) and extracted with EtOAc (3×30 ml). The organics were combined, dried on phase separator reduced in vacuo and purified by MDAP to give the title compound as a yellow solid (0.0061 g).

MH+=412, rt=2.68 min

### Example 32

### 2-(1,1-Dimethylethyl)-4-[6-(1-pyrrolidinylsulfonyl)-3-pyridinyl]-1H-pyrrolo[2,3-b]pyridine

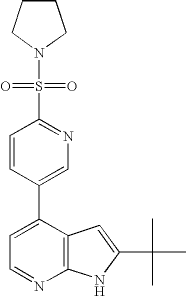

5-Bromo-2-(1-pyrrolidinylsulfonyl)pyridine (0.06739 g, 0.2314 mmol), [2-(1,1-dimethylethyl)-1H-pyrrolo[2,3-b]pyridin-4-yl]boronic acid (0.0555 g, 0.2546 mmol), 1,1′bis(diphenylphosphino)ferrocene dichloropalladium (II) (0.0189 g, 0.02314 mmol), and 1M sodium bicarbonate (0.695 ml) were transferred to a microwave vial (5 ml) The mixture was dissolved in Isopropanol (4 ml) and the reaction heated in a sealed tube in a Biotage Initiator microwave at 120° C. for 30 min. The mixture was taken into a separating funnel and submitted to an extraction using DCM (50 ml) and a saturated solution of sodium bicarbonate (25 ml), The organic phase was recovered, passed through a phase separator and the DCM evaporated. The dry residue was dissolved in DMSO and purified by MDAP. The solvent was then evaporated. The dry residue was dissolved in a mixture of DCM:MeOH (1:1, 10 ml) and passed through an ion exchange cartridge (NH₂, 1 g) preconditioned with the same eluent. The column was then washed with the same mixture (2×10 ml) and the solvent evaporated to give the title compound (0.032 g). MH+=385, rt=3.34 min

### Example 33

### N-(2-Aminoethyl)-6-[2-(trifluoromethyl)-1H-pyrrolo[2,3-b]pyridin-4-yl]-3-pyridinesulfonamide

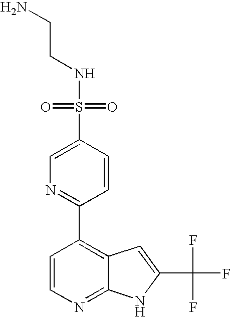

### Method A

[2-(Trifluoromethyl)-1H-pyrrolo[2,3-b]pyridin-4-yl]boronic acid (0.1 g, 0.43 mmol), 1,1-dimethylethyl (2-{[(6-chloro-3-pyridinyl)sulfonyl]amino}ethyl)carbamate (0.175 g, 0.52 mmol), Potassium phosphate (0.275 g, 1.29 mmol), 2′-(dimethylamino)-2-biphenylyl-palladium(ii)-chloride-dinorbornylphosphine complex (0.025 g, 0.043 mmol) were dissolved in dioxane:water (5:1, 5 ml) and heated in a sealed tube in a Biotage Initiator microwave at 120° C. for 30 min. The reaction mixture was extracted with DCM:MeOH (9:1, 20 ml) and water (20 ml), dried on a phase separator and reduced under vacuum to give a yellow solid. The solid was dissolved in DCM and TFA (0.16 ml) was added and left to react. Further TFA (0.32 ml) was added and the reaction continued, by LCMS product present. The mixture was reduced under vacuum, dissolved in DMSO and purified by MDAP. Fractions containing product were combined and reduced by blow down. The residue was dissolved in DCM:MeOH and passed through a NH₂ cartridge. The eluted fractions were reduced by blow down to give the title compound. (0.052 g).

MH+=386, rt=2.44 min

### Method B

The title compound was also prepared similarly to Example 35.

MH+=386, rt=2.3 min

### Example 34

### N-(2-Aminoethyl)-6-[2-(trifluoromethyl)-1H-pyrrolo[2,3-b]pyridin-4-yl]-3-pyridinesulfonamide trifluoroacetate

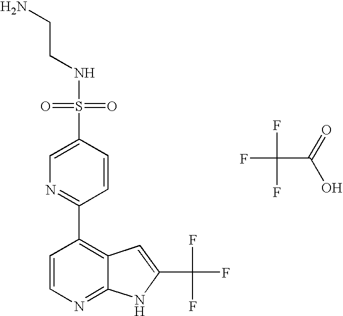

[2-(Trifluoromethyl)-1H-pyrrolo[2,3-b]pyridin-4-yl]boronic acid (0.04 g, 0.17 mmol), 1,1-dimethylethyl (2-{[(6-chloro-3-pyridinyl)sulfonyl]amino}ethyl)carbamate (0.07 g, 0.2 mmol), potassium phosphate (0.036 g, 0.17 mmol), and 2′-(dimethylamino)-2-biphenylyl-palladium(ii)-chloride-dinorbornylphosphine complex (0.011 g, 0.02 mmol) were dissolved in dioxane:water (5:1, 5 ml) and heated in a sealed tube in a Biotage Initiator microwave at 120° C. for 30 min. The reaction mixture was treated with water (5 ml) and extracted with DCM:EtOAc (1:1, 3×10 ml), dried using a phase separator and reduced in vacuo to give a brown oil which was dissolved in DCM (2 ml), TFA (2 ml) was added and the reaction mixture stirred at room temperature under N₂ for approx 1 h 15 min. The reaction mixture was evaporated in vacuo, dissolved in DMSO (2 ml) and purified by MDAP to give the title compound (0.033 g).

MH+=386, rt=2.45 min

### Example 35

### N-(1,1-Dioxidotetrahydro-2H-thiopyran-4-yl)-6-[2-(trifluoromethyl)-1H-pyrrolo[2,3-b]pyridin-4-yl]-3-pyridinesulfonamide

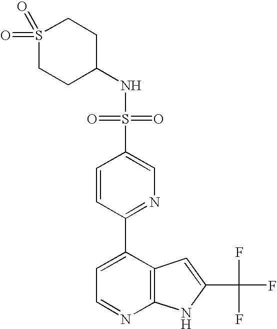

A solution of [2-(trifluoromethyl)-1H-pyrrolo[2,3-b]pyridin-4-yl]boronic acid (21 mg, 0.1 mmol) in dioxan (0.8 mL) was added to a microwave vessel containing 2′(dimethylamino)-2-biphenyl-palladium II chloride dinorbornylphosphine complex (0.25 mg, 0.5 mol %) and 6-chloro-N-(1,1-dioxidotetrahydro-2H-thiopyran-4-yl)-3-pyridinesulfonamide (32 mg, 0.1 mmol), A solution of potassium phosphate (64 mg, 0.3 mmol) in water (200 μl) was added and the reaction mixture heated at 120° C. in the microwave for 30 minutes. The reaction mixture was applied directly to a C18 cartridge (500 mg) and eluted with 0.1% TFA in acetonitrile (3×1 mL). Concentration by blow down followed by purification by mass directed HPLC gave the title compound.

MH+=476, rt=2.89 min

| Example | Structure | Name | MH+ | Rt (min) |
|---|---|---|---|---|
| 36 | 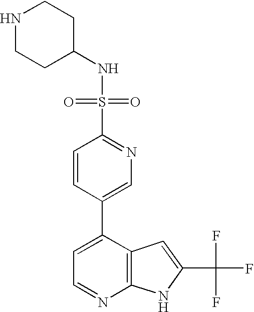 | N-4-piperidinyl-5-[2- (trifluoromethyl)-1H- pyrrolo[2,3-b]pyridin-4-yl]-2- pyridinesulfonamide | 426 | 2.26 |
| 37 | 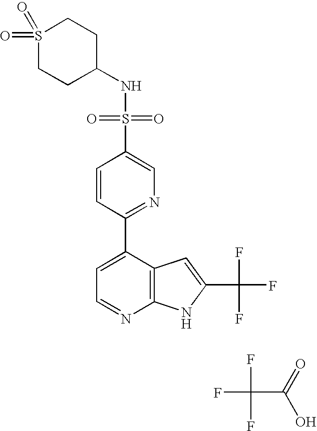 | N-(1,1-dioxidotetrahydro-2H- thiopyran-4-yl)-6-[2- (trifluoromethyl)-1H- pyrrolo[2,3-b]pyridin-4-yl]-3- pyridinesulfonamide trifluoroacetate | 476 | 2.9 |
| 38 | 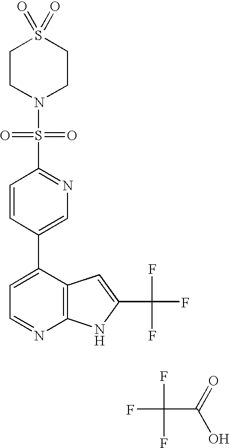 | 4-{6-[(1,1-dioxido-4- thiomorpholinyl)sulfonyl]-3- pyridinyl}-2-(trifluoromethyl)- 1H-pyrrolo[2,3-b]pyridine trifluoroacetate | 461 | 2.92 |
| 39 | 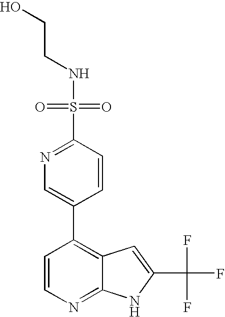 | N-(2-hydroxyethyl)-5-[2- (trifluoromethyl)-1H- pyrrolo[2,3-b]pyridin-4-yl]-2- pyridinesulfonamide | 387 | 2.62 |
| 40 | 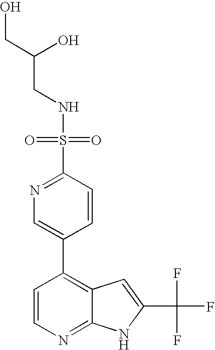 | N-(2,3-dihydroxypropyl)-5-[2- (trifluoromethyl)-1H- pyrrolo[2,3-b]pyridin-4-yl]-2- pyridinesulfonamide | 417 | 2.51 |
| 41 | 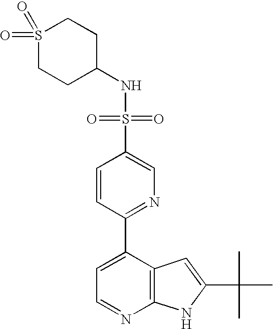 | 6-[2-(1,1-dimethylethyl)-1H- pyrrolo[2,3-b]pyridin-4-yl]-N- (1,1-dioxidotetrahydro-2H- thiopyran-4-yl)-3- pyridinesulfonamide | 464 | 2.93 |
| 42 | 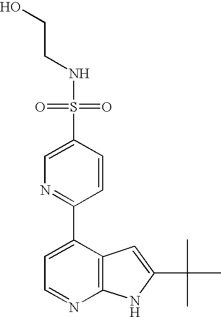 | 6-[2-(1,1-dimethylethyl)-1H- pyrrolo[2,3-b]pyridin-4-yl]-N- (2-hydroxyethyl)-3- pyridinesulfonamide | 376 | 2.78 |
| 43 | 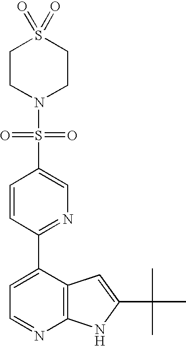 | 2-(1,1-dimethylethyl)-4-{5- [(1,1-dioxido-4- thiomorpholinyl)sulfonyl]-2- pyridinyl}-1H-pyrrolo[2,3- b]pyridine | 450 | 3.04 |
| 44 | 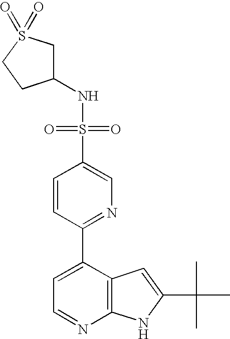 | 6-[2-(1,1-dimethylethyl)-1H- pyrrolo[2,3-b]pyridin-4-yl]-N- (1,1-dioxidotetrahydro-3- thienyl)-3- pyridinesulfonamide | 450 | 2.94 |
| 45 | 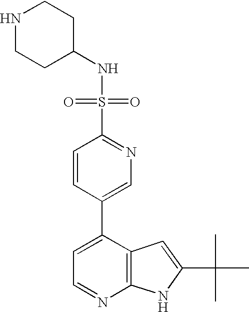 | 5-[2-(1,1-dimethylethyl)-1H- pyrrolo[2,3-b]pyridin-4-yl]-N- 4-piperidinyl-2- pyridinesulfonamide | 415 | 2.36 |
| 46 | 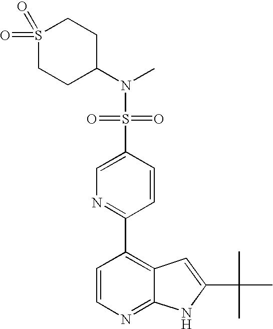 | 6-[2-(1,1-dimethylethyl)-1H- pyrrolo[2,3-b]pyridin-4-yl]-N- (1,1-dioxidotetrahydro-2H- thiopyran-4-yl)-N-methyl-3- pyridinesulfonamide | 478 | 3.07 |
| 47 | 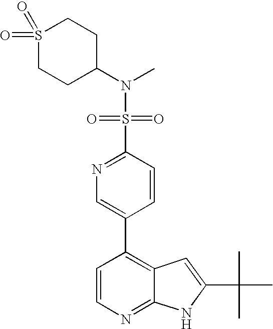 | 5-[2-(1,1-dimethylethyl)-1H- pyrrolo[2,3-b]pyridin-4-yl]-N- (1,1-dioxidotetrahydro-2H- thiopyran-4-yl)-N-methyl-2- pyridinesulfonamide | 478 | 3.04 |
| 48 | 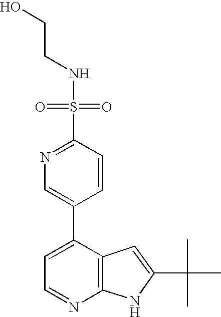 | 5-[2-(1,1-dimethylethyl)-1H- pyrrolo[2,3-b]pyridin-4-yl]-N- (2-hydroxyethyl)-2- pyridinesulfonamide | 376 | 2.73 |
| 49 | 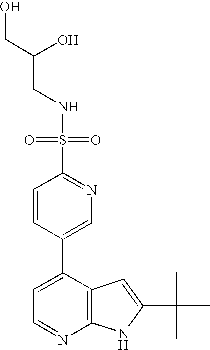 | N-(2,3-dihydroxypropyl)-5-[2- (1,1-dimethylethyl)-1H- pyrrolo[2,3-b]pyridin-4-yl]-2- pyridinesulfonamide | 406 | 2.61 |
| 50 | 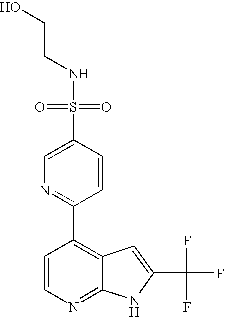 | N-(2-hydroxyethyl)-6-[2- (trifluoromethyl)-1H- pyrrolo[2,3-b]pyridin-4-yl]-3- pyridinesulfonamide | 387 | 2.74 |
| 51 | 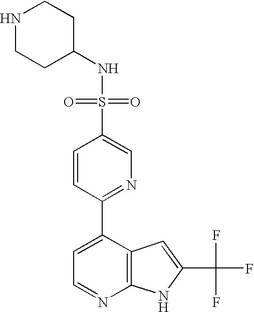 | N-4-piperidinyl-6-[2- (trifluoromethyl)-1H- pyrrolo[2,3-b]pyridin-4-yl]-3- pyridinesulfonamide | 426 | 2.37 |
| 52 | 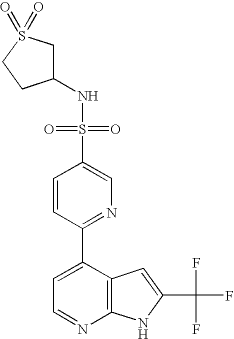 | N-(1,1-dioxidotetrahydro-3- thienyl)-6-[2-(trifluoromethyl)- 1H-pyrrolo[2,3-b]pyridin-4-yl]- 3-pyridinesulfonamide | 461 | 2.9 |
| 53 | 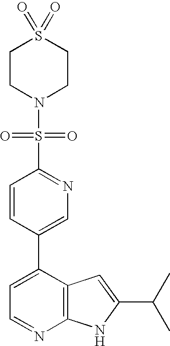 | 4-{6-[(1,1-dioxido-4- thiomorpholinyl)sulfonyl]-3- pyridinyl}-2-(trifluoromethyl)- 1H-pyrrolo[2,3-b]pyridine | 461 | 2.94 |
| 54 | 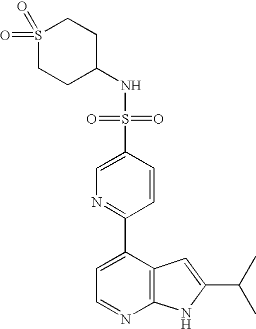 | N-(1,1-dioxidotetrahydro-2H- thiopyran-4-yl)-6-[2-(1- methylethyl)-1H-pyrrolo[2,3- b]pyridin-4-yl]-3- pyridinesulfonamide | 450 | 0.92 |
| 55 | 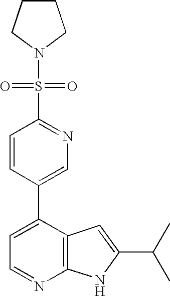 | 2-(1-methylethyl)-4-[6-(1- pyrrolidinylsulfonyl)-3- pyridinyl]-1H-pyrrolo[2,3- b]pyridine | 372 | 1.12 |
| 56 | 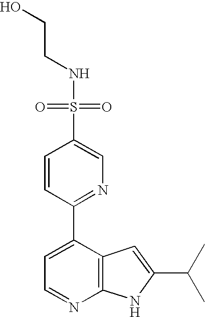 | N-(2-hydroxyethyl)-6-[2-(1- methylethyl)-1H-pyrrolo[2,3- b]pyridin-4-yl]-3- pyridinesulfonamide | 361 | 0.86 |
| 57 | 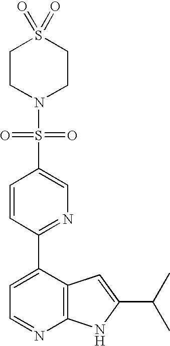 | 4-{5-[(1,1-dioxido-4- thiomorpholinyl)sulfonyl]-2- pyridinyl}-2-(1-methylethyl)- 1H-pyrrolo[2,3-b]pyridine | 436 | 0.98 |
| 58 | 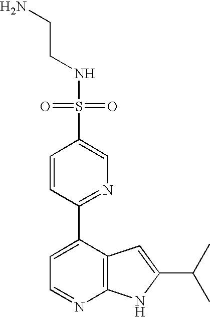 | N-(2-aminoethyl)-6-[2-(1- methylethyl)-1H-pyrrolo[2,3- b]pyridin-4-yl]-3- pyridinesulfonamide | 360 | 0.73 |
| 59 | 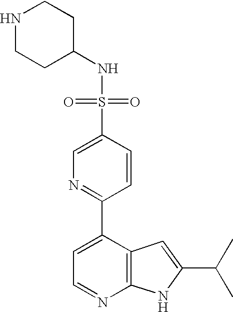 | 6-[2-(1-methylethyl)-1H- pyrrolo[2,3-b]pyridin-4-yl]-N- 4-piperidinyl-3- pyridinesulfonamide | 401 | 0.77 |
| 60 | 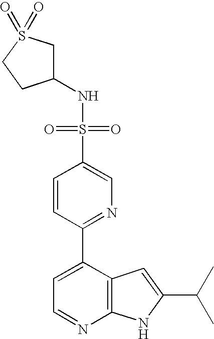 | N-(1,1-dioxidotetrahydro-3- thienyl)-6-[2-(1-methylethyl)- 1H-pyrrolo[2,3-b]pyridin-4-yl]- 3-pyridinesulfonamide | 436 | 0.93 |
| 61 | 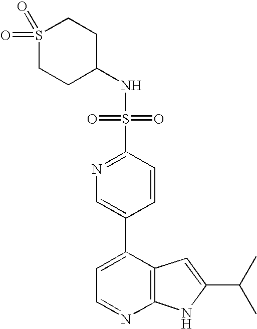 | N-(1,1-dioxidotetrahydro-2H- thiopyran-4-yl)-5-[2-(1- methylethyl)-1H-pyrrolo[2,3- b]pyridin-4-yl]-2- pyridinesulfonamide | 450 | 0.91 |
| 62 | 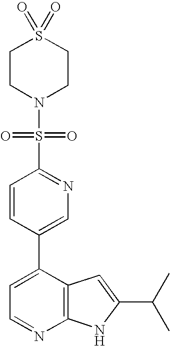 | 4-{6-[(1,1-dioxido-4- thiomorpholinyl)sulfonyl]-3- pyridinyl}-2-(1-methylethyl)- 1H-pyrrolo[2,3-b]pyridine | 436 | 0.99 |
| 63 | 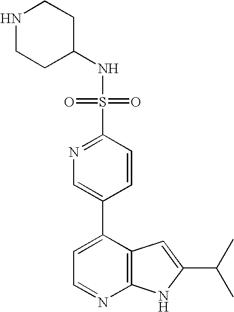 | 5-[2-(1-methylethyl)-1H- pyrrolo[2,3-b]pyridin-4-yl]-N- 4-piperidinyl-2- pyridinesulfonamide | 401 | 0.76 |
| 64 | 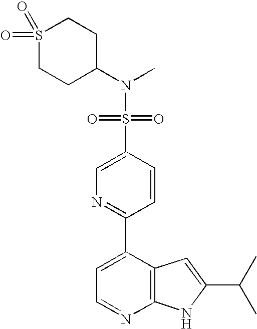 | N-(1,1-dioxidotetrahydro-2H- thiopyran-4-yl)-N-methyl-6-[2- (1-methylethyl)-1H- pyrrolo[2,3-b]pyridin-4-yl]-3- pyridinesulfonamide | 464 | 1 |
| 65 | 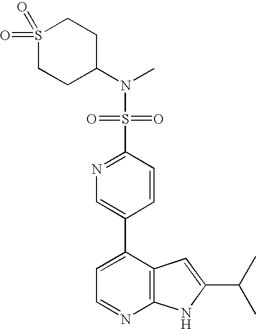 | N-(1,1-dioxidotetrahydro-2H- thiopyran-4-yl)-N-methyl-5-[2- (1-methylethyl)-1H- pyrrolo[2,3-b]pyridin-4-yl]-2- pyridinesulfonamide | 464 | 0.99 |
| 66 | 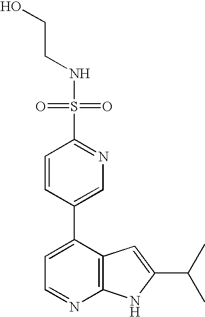 | N-(2-hydroxyethyl)-5-[2-(1- methylethyl)-1H-pyrrolo[2,3- b]pyridin-4-yl]-2- pyridinesulfonamide | 361 | 0.84 |
| 67 | 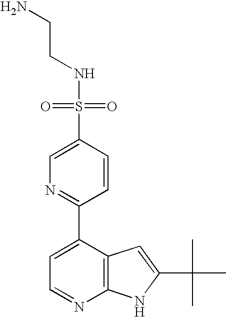 | N-(2-aminoethyl)-6-[2-(1,1- dimethylethyl)-1H-pyrrolo[2,3- b]pyridin-4-yl]-3- pyridinesulfonamide | 375 | 0.78 |
| 68 | 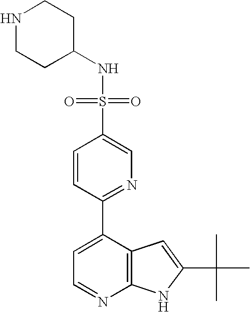 | 6-[2-(1,1-dimethylethyl)-1H- pyrrolo[2,3-b]pyridin-4-yl]-N- 4-piperidinyl-3- pyridinesulfonamide | 415 | 0.82 |
| 69 | 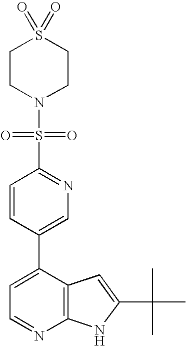 | 2-(1,1-dimethylethyl)-4-{6- [(1,1-dioxido-4- thiomorpholinyl)sulfonyl]-3- pyridinyl}-1H-pyrrolo[2,3- b]pyridine | 450 | 1.04 |

### Example 70

### 2-(1,1-Dimethylethyl)-4-[6-(1-pyrrolidinylsulfonyl)-3-pyridinyl]-1H-pyrrolo[2,3-b]pyridine

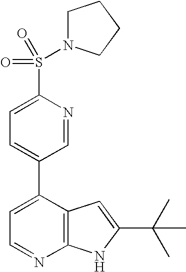

### Method A

To a degassed solution of [2-(1,1-dimethylethyl)-1H-pyrrolo[2,3-b]pyridin-4-yl]boronic acid (150 mg, 0.69 mmol), 5-bromo-2-(1-pyrrolidinylsulfonyl)pyridine (240 mg, 0.826 mmol) and potassium phosphate (146 mg, 0.69 mmol) in 5:1 Dioxane:water (10.8 mL) was added 2′-(dimethylamino)-2-biphenyl-palladium(II) chloride dinorbonylphosphine complex (39 mg, 0.07 mmol) and the reaction heated to 120° C. for 30 min (Biotage Initiator microwave). The reaction was poured onto water (15 mL) was extracted with 1:1 Chloroform/Ethylacetate (2×20 mL). The combined extracts were dried (hydrophobic frit) and concentrated in vacuo to a yellow solid. Purification by chromatography (silica 20 g, 0-100% ethylacetate in cyclohexane over 40 min) gave the title compound as a white solid (65 mg).

LCMS MH+=385, rt=1.17 min

### Method B

The title compound was also prepared similarly to Example 35.

MH+=386, rt=3.3 min

### Example 71

### 2-(1,1-Dimethylethyl)-4-[6-(1-pyrrolidinylsulfonyl)-3-pyridinyl]-1H-pyrrolo[2,3-b]pyridine hydrochloride

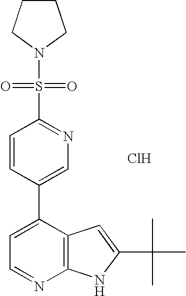

To a solution of 2-(1,1-dimethylethyl)-4-[6-(1-pyrrolidinylsulfonyl)-3-pyridinyl]-1H-pyrrolo[2,3-b]pyridine (20 mg, 0.052 mmol) in methanol (2 mL) was added 4M hydrochloric acid in dioxane (13.24). Concentration by nitrogen blowdown gave the title compound (21 mg).

LCMS MH+=385, rt=3.33 min

### Example 72

### 2-(1,1-Dimethylethyl)-4-[6-(1-pyrrolidinylsulfonyl)-3-pyridinyl]-1H-pyrrolo[2,3-b]pyridine sulfate

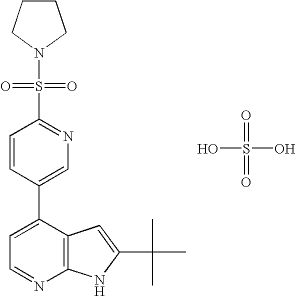

To a solution of 2-(1,1-dimethylethyl)-4-[6-(1-pyrrolidinylsulfonyl)-3-pyridinyl]-1H-pyrrolo[2,3-b]pyridine (16 mg, 0.052 mmol) in methanol (2 mL) was added 0.19M sulphuric acid in methanol (0.25 mL). Concentration by nitrogen blowdown gave the title compound (16 mg).

LCMS MH+=385, rt=3.33 min

### Example 73

### N-(2-Aminoethyl)-5-[2-(difluoromethyl)-1H-pyrrolo[2,3-b]pyridin-4-yl]-2-thiophenesulfonamide

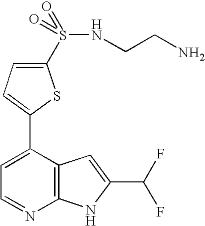

To a stirred solution of N-(2-aminoethyl)-5-[2-(difluoromethyl)-1-(phenylsulfonyl)-1H-pyrrolo[2,3-b]pyridin-4-yl]-2-thiophenesulfonamide (23 mg, 0.04 mmol) in tetrahydrofuran (2 mL) and D6 Dimethylsulfoxide (0.5 mL) was added tetrabutylammoniumfluoride 1M in tetrahydrofuran (0.1 mL) and stirred at ambient temperature for 1.5 h. The reaction was loaded onto a SCX-II cartridge (2 g, washed with methanol, eluted with 2N ammonia in methanol) the eluent was concentrated (nitrogen blowdown) and triturated with dichloromethane, the supernatant decanted and the solid dried to give the title compound (7.5 mg).

LCMS MH+=373, rt=0.74 min

### Example 74

### 5-[2-(Difluoromethyl)-1H-pyrrolo[2,3-b]pyridin-4-yl]-N-(1,1-dioxidotetrahydro-3-thienyl)-2-thiophenesulfonamide

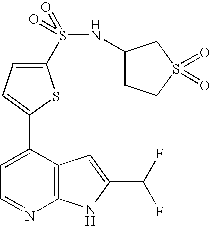

To a stirred solution of 5-[2-(difluoromethyl)-1-(phenylsulfonyl)-1H-pyrrolo[2,3-b]pyridin-4-yl]-N-(1,1-dioxidotetrahydro-3-thienyl)-2-thiophenesulfonamide (17 mg, 0.03 mmol) in tetrahydrofuran (2 mL) and D6 Dimethylsulfoxide (0.5 mL) was added tetrabutylammoniumfluoride 1M in tetrahydrofuran (0.06 mL) and stirred at ambient temperature for 1.5 h. The reaction was loaded onto a SCX-II cartridge (2 g, washed with methanol, eluted with 2N ammonia in methanol) the eluent was concentrated (nitrogen blowdown) to give the title compound (12 mg).

LCMS MH+=448, rt=0.92 min

### Example 75

### 5-[2-(Difluoromethyl)-1H-pyrrolo[2,3-b]pyridin-4-yl]-N-(1,1-dioxidotetrahydro-2H-thiopyran-4-yl)-2-thiophenesulfonamide

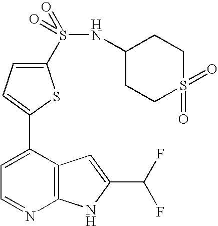

To a stirred solution of 5-[2-(difluoromethyl)-1-(phenylsulfonyl)-1H-pyrrolo[2,3-b]pyridin-4-yl]-N-(1,1-dioxidotetrahydro-2H-thiopyran-4-yl)-2-thiophenesulfonamide (40 mg, 0.07 mmol) in tetrahydrofuran (2 mL) and D6 Dimethylsulfoxide (0.5 mL) was added tetrabutylammoniumfluoride 1M in tetrahydrofuran (0.14 mL) and stirred at ambient temperature for 1.5 h. The reaction was loaded onto a SCX-II cartridge (2 g, washed with methanol, eluted with 2N ammonia in methanol) the eluent was concentrated (nitrogen blowdown) and triturated with methanol, the supernatant decanted and the solid dried to give the title compound (18 mg).

LCMS MH+=462, rt=0.92 min

### Example 76

### N-(2-Hydroxy-2-methylpropyl)-5-[2-(trifluoromethyl)-1H-pyrrolo[2,3-b]pyridin-4-yl]-2-thiophenesulfonamide trifluoroacetate (salt)

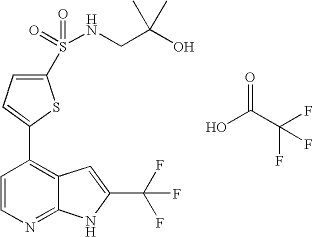

To a stirred suspension of [2-(trifluoromethyl)-1H-pyrrolo[2,3-b]pyridin-4-yl]boronic acid (50 mg, 0.217 mmol), and 5-bromo-N-(2-hydroxy-2-methylpropyl)-2-thiophenesulfonamide (82 mg, 0.26 mmol) in isopropanol (1.95 mL) and 1M aqueous sodium bicarbonate (0.65 mL) was added Bis(diphenylphosphino)ferrocenedichloro palladium (II) (9 mg, 0.011 mmol) before heating to 120° C. for 30 min (Biotage initiator Microwave). The reaction was diluted with water (10 mL) and extracted with 1:1 ethylacetate:chloroform (20 mL). The extract was concentrated (nitrogen blowdown) and purified by MDAP to give the title compound (14 mg, 28%).

LCMS MH+=419, rt=3.03 min

Examples 77 and 78 were similarly prepared:

### Example 77

### N-(2-Hydroxyethyl)-5-[2-(trifluoromethyl)-1H-pyrrolo[2,3-b]pyridin-4-yl]-2-thiophenesulfonamide trifluoroacetate (salt)

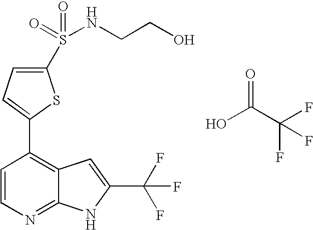

(41 mg) LCMS MH+=392, rt=2.87 min

### Example 78

### N-(Tetrahydro-2H-pyran-4-yl)-5-[2-(trifluoromethyl)-1H-pyrrolo[2,3-b]pyridin-4-yl]-2-thiophenesulfonamide trifluoroacetate

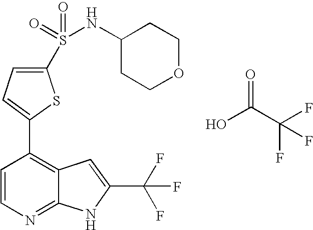

(16 mg) LCMS MH+=432, rt=3.11 min

### Example 79

### N-(2,3-Dihydroxypropyl)-5-[2-(trifluoromethyl)-1H-pyrrolo[2,3-b]pyridin-4-yl]-2-5 thiophenesulfonamide trifluoroacetate (salt)

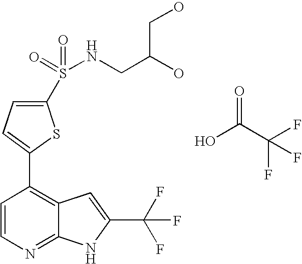

To a stirred suspension of [2-(trifluoromethyl)-1H-pyrrolo[2,3-b]pyridin-4-yl]boronic acid (50 mg, 0.217 mmol), and 5-bromo-N-[(2,2-dimethyl-1,3-dioxolan-4-yl)methyl]-2-thiophenesulfonamide (93 mg, 0.26 mmol) in isopropanol (1.95 mL) and 1M aqueous sodium bicarbonate (0.65 mL) was added Bis(diphenylphosphino)ferrocenedichloro palladium (II) (9 mg, 0.011 mmol) before heating to 120° C. for 30 min (Biotage initiator Microwave). The reaction was diluted with water (10 mL) and extracted with 1:1 ethylacetate:chloroform (20 mL). The extract was concentrated (nitrogen blowdown) and the residue suspended in methanol and treated with 2M aqueous hydrochloric acid for 16 h and purified by MDAP to give the title compound (51 mg).

LCMS MH+=422, rt=2.70 min

### Example 80

### N-(2-Aminoethyl)-5-[2-(trifluoromethyl)-1H-pyrrolo[2,3-b]pyridin-4-yl]-2-thiophenesulfonamide trifluoroacetate

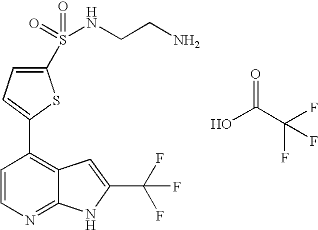

To a stirred suspension of [2-(trifluoromethyl)-1H-pyrrolo[2,3-b]pyridin-4-yl]boronic acid (50 mg, 0.217 mmol), and 1,1-dimethylethyl (2-{[(5-bromo-2-thienyl)sulfonyl]amino}ethyl)carbamate (100 mg, 0.26 mmol) in isopropanol (1.95 mL) and 1M aqueous sodium bicarbonate (0.65 mL) was added Bis(diphenylphosphino)ferrocenedichloro palladium (II) (9 mg, 0.011 mmol) before heating to 120° C. for 30 min (Biotage initiator). The reaction was diluted with water (10 mL) and extracted with 1:1 ethylacetate:chloroform (20 mL). The extract was concentrated (nitrogen blowdown) and the residue suspended in treated with trifluoroacetic acid for 16 h and purified by MDAP to give the title compound (52 mg). LCMS MH+=391, rt=2.60 min

Example 81 was similarly prepared:

### Example 81

### N-4-Piperidinyl-5-[2-(trifluoromethyl)-1H-pyrrolo[2,3-b]pyridin-4-yl]-2-thiophenesulfonamide trifluoroacetate

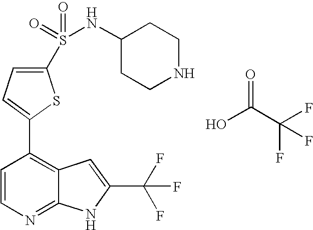

(55 mg) LCMS MH+=431, rt=2.72 min

### Example 82

### Formic acid—N-(tetrahydro-2H-thiopyran-4-yl)-6-[2-(trifluoromethyl)-1H-pyrrolo[2,3-b]pyridin-4-yl]-3-pyridinesulfonamide (1:1)

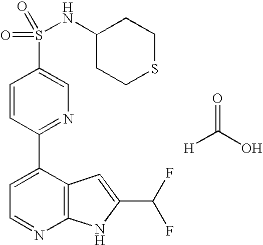

To solution of 6-chloro-N-(tetrahydro-2H-thiopyran-4-yl)-3-pyridinesulfonamide (61 mg, 0.21 mmol), [2-(trifluoromethyl)-1H-pyrrolo[2,3-b]pyridin-4-yl]boronic acid (40 mg, 0.174 mmol) and potassium phosphate (37 mg, 0.174 mmol) in 5:1 Dioxane:water (2.4 mL) was added 2′-(dimethylamino)-2-biphenyl-palladium(II) chloride dinorbonylphosphine complex (14 mg, 0.02 mmol) and the reaction heated to 120° C. for 30 min (Biotage Initiator microwave). The reaction was diluted with water (10 mL) was extracted with 1:1 Chloroform/Ethylacetate (2×20 mL). The combined extracts were dried (hydrophobic frit) and concentrated in vacuo. Purification by MDAP twice gave the title compound as a white solid (4 mg).

LCMS MH+=443, rt=3.29 min

### Example 83

### Formic acid—N-(2-aminoethyl)-6-[2-(trifluoromethyl)-1H-pyrrolo[2,3-b]pyridin-4-yl]-3-pyridinesulfonamide

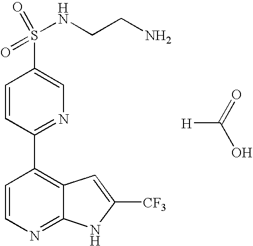

A solution of 1,1-dimethylethyl {2-[({6-[2-(trifluoromethyl)-1H-pyrrolo[2,3-b]pyridin-4-yl]-3-pyridinyl}sulfonyl)amino]ethyl}carbamate (600 mg, 1.2 mmol) was dissolved in DCM (5 ml) and trifluoroacetic acid (3 ml) was added dropwise. After 3 hours the reaction mixture was evaporated to dryness and triturated with ether (20 ml). The resulting solid was purified by mass directed autoprep to give the title compound (68 mg) as a cream solid.

MH⁺386, rt=2.43 min.

Further compounds which were prepared are:

| Example | Structure | Name |
|---|---|---|
| 84 | 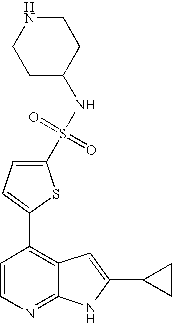 | 5-(2-cyclopropyl-1H-pyrrolo[2,3- b]pyridin-4-yl)-N-4-piperidinyl-2- thiophenesulfonamide |
| 85 | 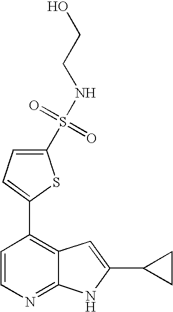 | 5-(2-cyclopropyl-1H-pyrrolo[2,3- b]pyridin-4-yl)-N-(2-hydroxyethyl)- 2-thiophenesulfonamide |
| 86 | 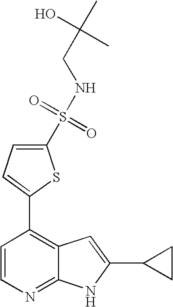 | 5-(2-cyclopropyl-1H-pyrrolo[2,3- b]pyridin-4-yl)-N-(2-hydroxy-2- methylpropyl)-2- thiophenesulfonamide |
| 87 | 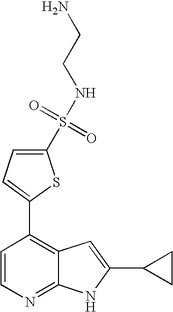 | N-(2-aminoethyl)-5-(2-cyclopropyl- 1H-pyrrolo[2,3-b]pyridin-4-yl)-2- thiophenesulfonamide |
| 88 | 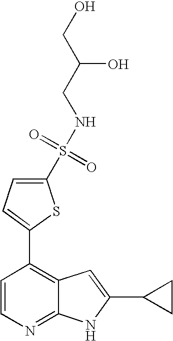 | 5-(2-cyclopropyl-1H-pyrrolo[2,3- b]pyridin-4-yl)-N-(2,3- dihydroxypropyl)-2- thiophenesulfonamide |
| 89 | 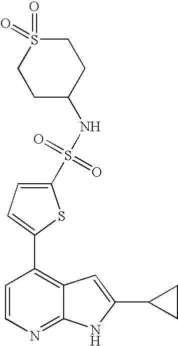 | 5-(2-cyclopropyl-1H-pyrrolo[2,3- b]pyridin-4-yl)-N-(1,1- dioxidotetrahydro-2H-thiopyran-4- yl)-2-thiophenesulfonamide |
| 90 | 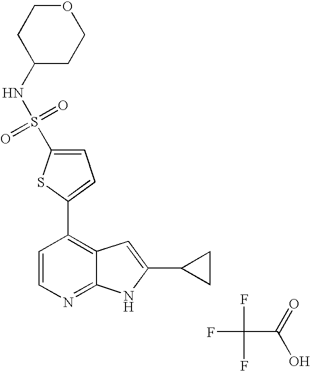 | 5-(2-cyclopropyl-1H-pyrrolo[2,3- b]pyridin-4-yl)-N-(tetrahydro-2H- pyran-4-yl)-2- thiophenesulfonamide trifluoroacetate |
| 91 | 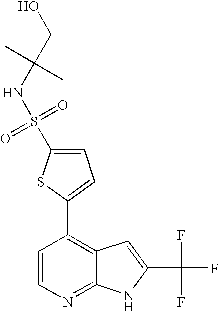 | N-(2-hydroxy-1,1-dimethylethyl)-5- [2-(trifluoromethyl)-1H-pyrrolo[2,3- b]pyridin-4-yl]-2- thiophenesulfonamide |
| 92 | 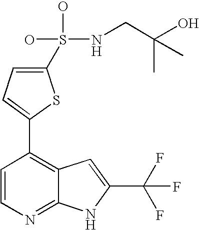 | N-(2-hydroxy-2-methylpropyl)-5-[2- (trifluoromethyl)-1H-pyrrolo[2,3- b]pyridin-4-yl]-2- thiophenesulfonamide |
| 93 | 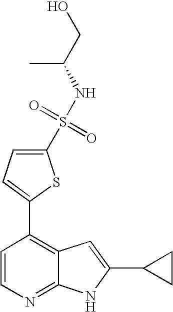 | 5-(2-cyclopropyl-1H-pyrrolo[2,3- b]pyridin-4-yl)-N-[(1R)-2-hydroxy-1- methylethyl]-2- thiophenesulfonamide |
| 94 | 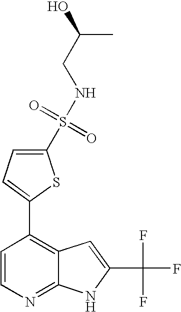 | N-[(2S)-2-hydroxypropyl]-5-[2- (trifluoromethyl)-1H-pyrrolo[2,3- b]pyridin-4-yl]-2- thiophenesulfonamide |
| 95 | 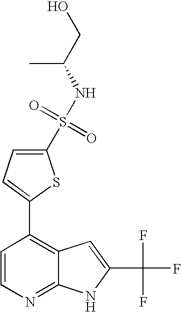 | N-[(1R)-2-hydroxy-1-methylethyl]- 5-[2-(trifluoromethyl)-1H- pyrrolo[2,3-b]pyridin-4-yl]-2- thiophenesulfonamide |
| 96 | 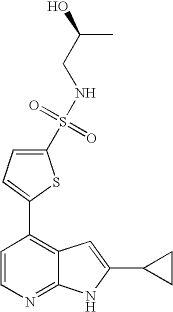 | 5-(2-cyclopropyl-1H-pyrrolo[2,3- b]pyridin-4-yl)-N-[(2S)-2- hydroxypropyl]-2- thiophenesulfonamide |
| 97 | 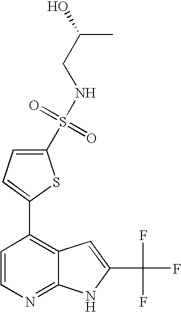 | N-[(2R)-2-hydroxypropyl]-5-[2- (trifluoromethyl)-1H-pyrrolo[2,3- b]pyridin-4-yl]-2- thiophenesulfonamide |
| 98 | 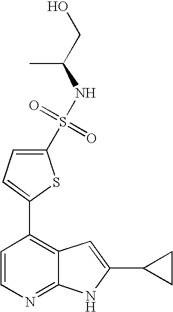 | 5-(2-cyclopropyl-1H-pyrrolo[2,3- b]pyridin-4-yl)-N-[(1S)-2-hydroxy-1- methylethyl]-2- thiophenesulfonamide |
| 99 | 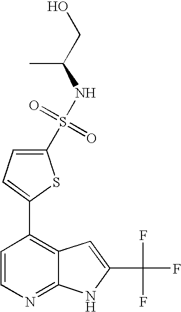 | N-[(1S)-2-hydroxy-1-methylethyl]-5- [2-(trifluoromethyl)-1H-pyrrolo[2,3- b]pyridin-4-yl]-2- thiophenesulfonamide |
| 100 | 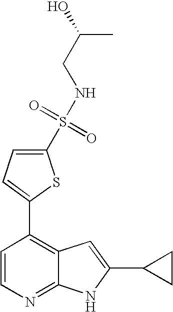 | 5-(2-cyclopropyl-1H-pyrrolo[2,3- b]pyridin-4-yl)-N-[(2R)-2- hydroxypropyl]-2- thiophenesulfonamide |
| 101 | 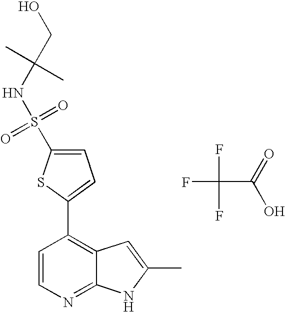 | N-(2-hydroxy-1,1-dimethylethyl)-5- (2-methyl-1H-pyrrolo[2,3-b]pyridin- 4-yl)-2-thiophenesulfonamide trifluoroacetate (salt) |
| 102 | 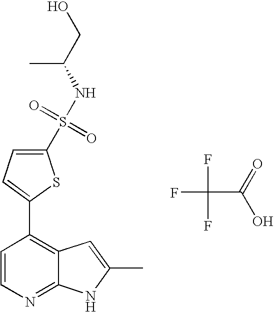 | N-[(1R)-2-hydroxy-1-methylethyl]- 5-(2-methyl-1H-pyrrolo[2,3- b]pyridin-4-yl)-2- thiophenesulfonamide trifluoroacetate (salt) |
| 103 | 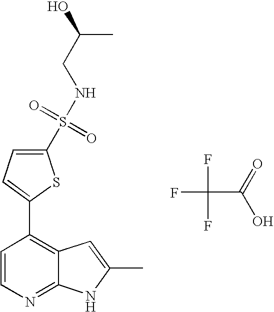 | N-[(2S)-2-hydroxypropyl]-5-(2- methyl-1H-pyrrolo[2,3-b]pyridin-4- yl)-2-thiophenesulfonamide trifluoroacetate (salt) |
| 104 | 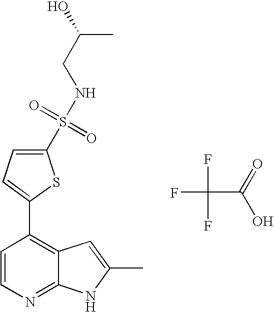 | N-[(2R)-2-hydroxypropyl]-5-(2- methyl-1H-pyrrolo[2,3-b]pyridin-4- yl)-2-thiophenesulfonamide trifluoroacetate (salt) |
| 105 | 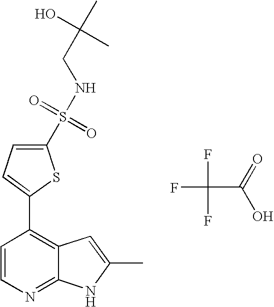 | N-(2-hydroxy-2-methylpropyl)-5-(2- methyl-1H-pyrrolo[2,3-b]pyridin-4- yl)-2-thiophenesulfonamide trifluoroacetate (salt) |
| 106 | 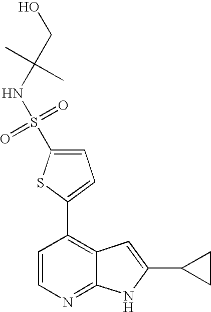 | 5-(2-cyclopropyl-1H-pyrrolo[2,3- b]pyridin-4-yl)-N-(2-hydroxy-1,1- dimethylethyl)-2- thiophenesulfonamide |
| 107 | 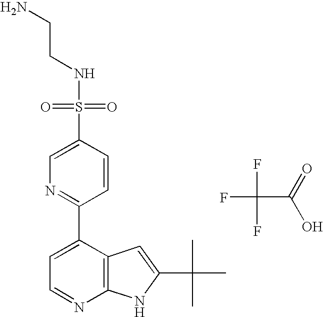 | N-(2-aminoethyl)-6-[2-(1,1- dimethylethyl)-1H-pyrrolo[2,3- b]pyridin-4-yl]-3- pyridinesulfonamide trifluoroacetate |
| 108 | 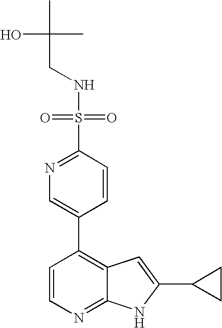 | 5-(2-cyclopropyl-1H-pyrrolo[2,3- b]pyridin-4-yl)-N-(2-hydroxy-2- methylpropyl)-2- pyridinesulfonamide |
| 109 | 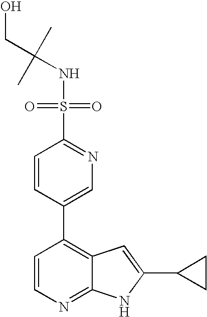 | 5-(2-cyclopropyl-1H-pyrrolo[2,3- b]pyridin-4-yl)-N-(2-hydroxy-1,1- dimethylethyl)-2- pyridinesulfonamide |
| 110 | 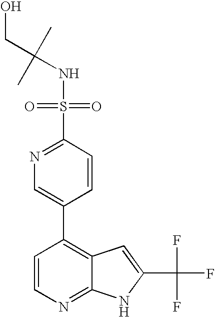 | N-(2-hydroxy-1,1-dimethylethyl)-5- [2-(trifluoromethyl)-1H-pyrrolo[2,3- b]pyridin-4-yl]-2- pyridinesulfonamide |
| 111 | 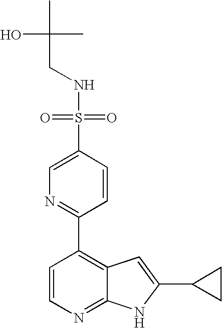 | 6-(2-cyclopropyl-1H-pyrrolo[2,3- b]pyridin-4-yl)-N-(2-hydroxy-2- methylpropyl)-3- pyridinesulfonamide |
| 112 | 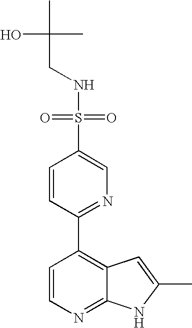 | N-(2-hydroxy-2-methylpropyl)-6-(2- methyl-1H-pyrrolo[2,3-b]pyridin-4- yl)-3-pyridinesulfonamide |
| 113 | 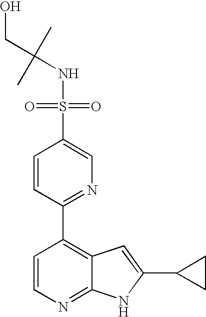 | 6-(2-cyclopropyl-1H-pyrrolo[2,3- b]pyridin-4-yl)-N-(2-hydroxy-1,1- dimethylethyl)-3- pyridinesulfonamide |
| 114 | 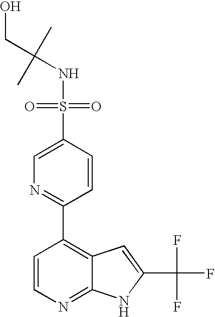 | N-(2-hydroxy-1,1-dimethylethyl)-6- [2-(trifluoromethyl)-1H-pyrrolo[2,3- b]pyridin-4-yl]-3- pyridinesulfonamide |
| 115 | 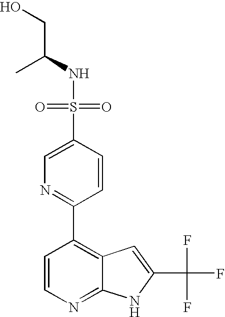 | N-[(1S)-2-hydroxy-1-methylethyl]-6- [2-(trifluoromethyl)-1H-pyrrolo[2,3- b]pyridin-4-yl]-3- pyridinesulfonamide |
| 116 | 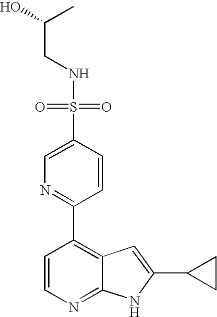 | 6-(2-cyclopropyl-1H-pyrrolo[2,3- b]pyridin-4-yl)-N-[(2R)-2- hydroxypropyl]-3- pyridinesulfonamide |
| 117 | 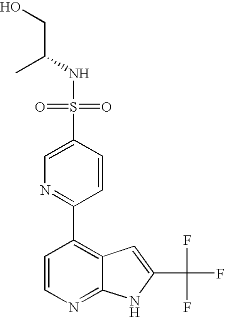 | N-[(1R)-2-hydroxy-1-methylethyl]- 6-[2-(trifluoromethyl)-1H- pyrrolo[2,3-b]pyridin-4-yl]-3- pyridinesulfonamide |
| 118 | 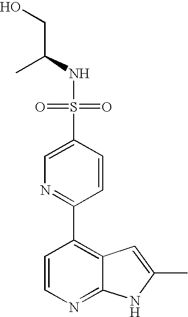 | N-[(1S)-2-hydroxy-1-methylethyl]-6- (2-methyl-1H-pyrrolo[2,3-b]pyridin- 4-yl)-3-pyridinesulfonamide |
| 119 | 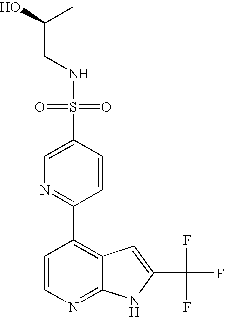 | N-[(2S)-2-hydroxypropyl]-6-[2- (trifluoromethyl)-1H-pyrrolo[2,3- b]pyridin-4-yl]-3- pyridinesulfonamide |
| 120 | 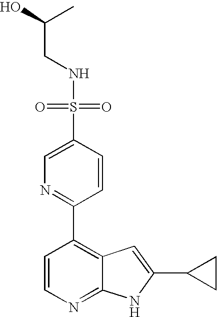 | 6-(2-cyclopropyl-1H-pyrrolo[2,3- b]pyridin-4-yl)-N-[(2S)-2- hydroxypropyl]-3- pyridinesulfonamide |
| 121 | 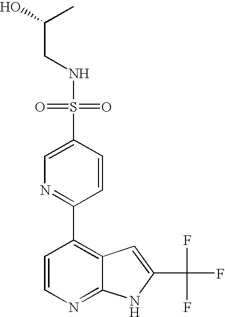 | N-[(2R)-2-hydroxypropyl]-6-[2- (trifluoromethyl)-1H-pyrrolo[2,3- b]pyridin-4-yl]-3- pyridinesulfonamide |
| 122 | 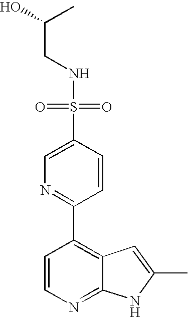 | N-[(2R)-2-hydroxypropyl]-6-(2- methyl-1H-pyrrolo[2,3-b]pyridin-4- yl)-3-pyridinesulfonamide |
| 123 | 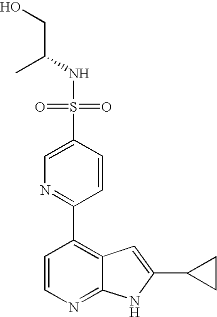 | 6-(2-cyclopropyl-1H-pyrrolo[2,3- b]pyridin-4-yl)-N-[(1R)-2-hydroxy-1- methylethyl]-3-pyridinesulfonamide |
| 124 | 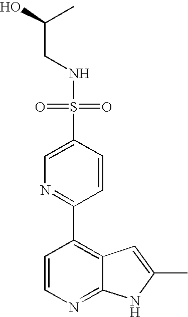 | N-[(2S)-2-hydroxypropyl]-6-(2- methyl-1H-pyrrolo[2,3-b]pyridin-4- yl)-3-pyridinesulfonamide |
| 125 | 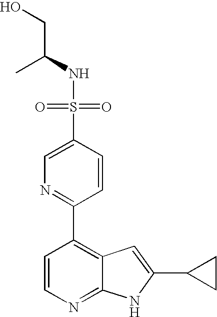 | 6-(2-cyclopropyl-1H-pyrrolo[2,3- b]pyridin-4-yl)-N-[(1S)-2-hydroxy-1- methylethyl]-3-pyridinesulfonamide |
| 126 | 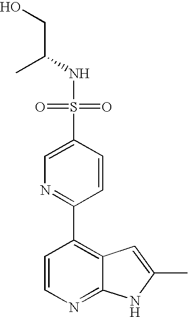 | N-[(1R)-2-hydroxy-1-methylethyl]- 6-(2-methyl-1H-pyrrolo[2,3- b]pyridin-4-yl)-3- pyridinesulfonamide |
| 127 | 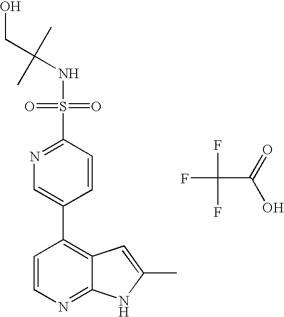 | N-(2-hydroxy-1,1-dimethylethyl)-5- (2-methyl-1H-pyrrolo[2,3-b]pyridin- 4-yl)-2-pyridinesulfonamide trifluoroacetate (salt) |
| 128 | 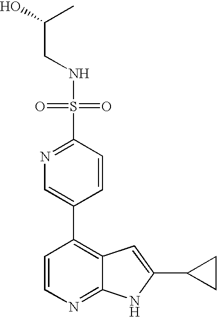 | 5-(2-cyclopropyl-1H-pyrrolo[2,3- b]pyridin-4-yl)-N-[(2R)-2- hydroxypropyl]-2- pyridinesulfonamide |
| 129 | 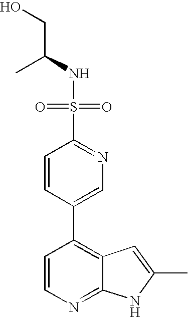 | N-[(1S)-2-hydroxy-1-methylethyl]-5- (2-methyl-1H-pyrrolo[2,3-b]pyridin- 4-yl)-2-pyridinesulfonamide |
| 130 | 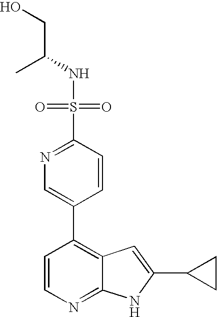 | 5-(2-cyclopropyl-1H-pyrrolo[2,3- b]pyridin-4-yl)-N-[(1R)-2-hydroxy-1- methylethyl]-2-pyridinesulfonamide |
| 131 | 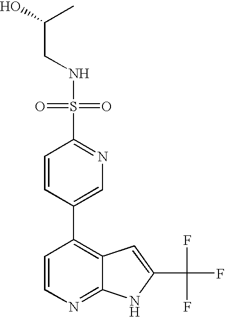 | N-[(2R)-2-hydroxypropyl]-5-[2- (trifluoromethyl)-1H-pyrrolo[2,3- b]pyridin-4-yl]-2- pyridinesulfonamide |
| 132 | 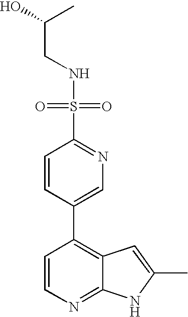 | N-[(2R)-2-hydroxypropyl]-5-(2- methyl-1H-pyrrolo[2,3-b]pyridin-4- yl)-2-pyridinesulfonamide |
| 133 | 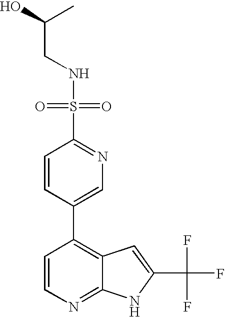 | N-[(2S)-2-hydroxypropyl]-5-[2- (trifluoromethyl)-1H-pyrrolo[2,3- b]pyridin-4-yl]-2- pyridinesulfonamide |
| 134 | 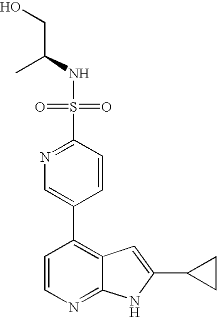 | 5-(2-cyclopropyl-1H-pyrrolo[2,3- b]pyridin-4-yl)-N-[(1S)-2-hydroxy-1- methylethyl]-2-pyridinesulfonamide |
| 135 | 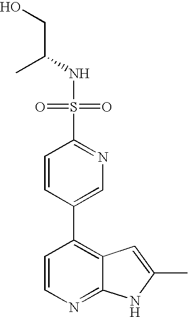 | N-[(1R)-2-hydroxy-1-methylethyl]- 5-(2-methyl-1H-pyrrolo[2,3- b]pyridin-4-yl)-2- pyridinesulfonamide |
| 136 | 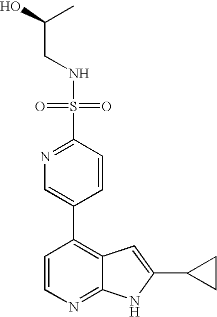 | 5-(2-cyclopropyl-1H-pyrrolo[2,3- b]pyridin-4-yl)-N-[(2S)-2- hydroxypropyl]-2- pyridinesulfonamide |
| 137 | 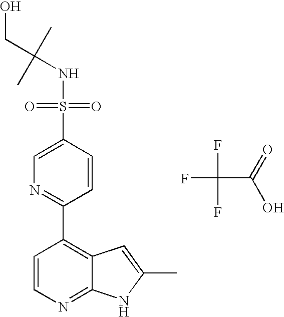 | N-(2-hydroxy-1,1-dimethylethyl)-6- (2-methyl-1H-pyrrolo[2,3-b]pyridin- 4-yl)-3-pyridinesulfonamide trifluoroacetate (salt) |
| 138 | 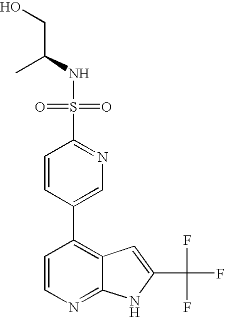 | N-[(1S)-2-hydroxy-1-methylethyl]-5- [2-(trifluoromethyl)-1H-pyrrolo[2,3- b]pyridin-4-yl]-2- pyridinesulfonamide |
| 139 | 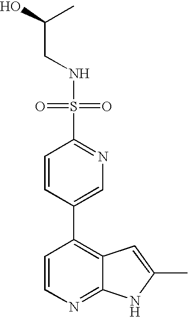 | N-[(2S)-2-hydroxypropyl]-5-(2- methyl-1H-pyrrolo[2,3-b]pyridin-4- yl)-2-pyridinesulfonamide |
| 140 | 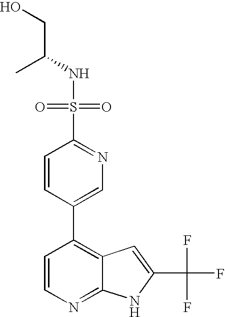 | N-[(1R)-2-hydroxy-1-methylethyl]- 5-[2-(trifluoromethyl)-1H- pyrrolo[2,3-b]pyridin-4-yl]-2- pyridinesulfonamide |

Other compounds which were prepared were:

### Example 141

### 5-(2-Cyclopropyl-1H-pyrrolo[2,3-b]pyridin-4-yl)-N-(2-hydroxy-1,1-dimethylpropyl)-2-pyridinesulfonamide

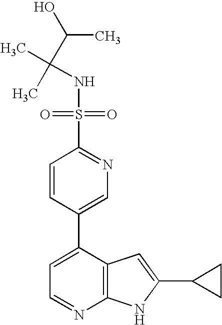

The title compound was obtained as a yellow solid (15.8 mg).

MH+=401.10, rt=2.86 min

### Example 142

### 5-(2-Cyclopropyl-1H-pyrrolo[2,3-b]pyridin-4-yl)-N-(2-hydroxy-1,2-dimethylpropyl)-2-pyridinesulfonamide

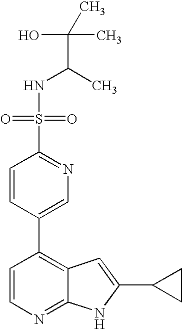

The title compound was obtained as a brown gum which solidified to a brown solid (23 mg).

MH+=401.16, rt=2.79 min

### Example 143

### 5-(2-Cyclopropyl-1H-pyrrolo[2,3-b]pyridin-4-yl)-N-[(1R)-1-(hydroxymethyl)propyl]-2-pyridinesulfonamide

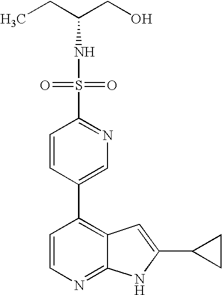

The title compound was obtained as a yellow solid (18 mg).

MH+=387.10, rt=2.74 min

### Example 144

### N-(2-Hydroxy-2-methylpropyl)-5-(2-methyl-1H-pyrrolo[2,3-b]pyridin-4-yl)-2-pyridinesulfonamide trifluoroacetate (salt)

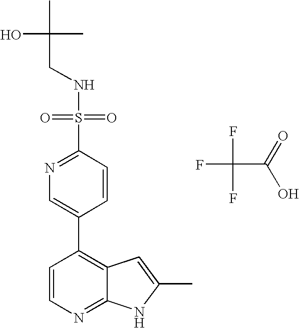

### Biological Data

### IKK2 Assay

Recombinant human IKKβ (residues 1-737) was expressed in baculovirus as a C-terminal GST-tagged fusion protein, and its activity was assessed using a time-resolved fluorescence resonance energy transfer (TR-FRET) assay. Briefly, IKKβ (0.5-4 nM final concentration) diluted in assay buffer (50 mM HEPES, 10 mM MgCl₂, 1 mM CHAPS pH 7.4 with 1 mM DTT and 0.01% w/v BSA) was added to wells containing various concentrations of compound or DMSO vehicle (1.7% v/v final). The reaction was initiated by the addition of GST-IkappaBalpha substrate (25 nM final)/ATP (1 μM final), in a total volume of 6 μl. The reaction was incubated for 15 min at room temperature, then terminated by the addition of 3 μl of 50 mM EDTA in buffer (100 mM HEPES pH 7.4, 150 mM NaCl and 0.1% w/v BSA) containing antiphosphoserine-IkappaBalpha-32/36 monoclonal antibody clone 12C2 (Cell Signalling Technology, Beverly Mass., USA) labelled with W-1024 europium chelate (Wallac OY, Turku, Finland), and an APC-labelled anti-GST antibody (Prozyme, San Leandro, Calif., USA). The reaction was further incubated for 60 min at room temperature and the degree of phosphorylation of GST-IkappaBalpha measured using a Rubystar plate reader (BMG Instruments, Aylesbury, UK) as a ratio of specific 665 nm energy transfer signal to reference europium 620 nm signal.

### Results

The compounds of Examples 1 to 106 and Examples 108 to 144 were tested for activity against IKK2 and the compounds were found to be inhibitors of IKK2 with plC₅₀ potency of 5.0 or greater.

1. A compound of formula (I): 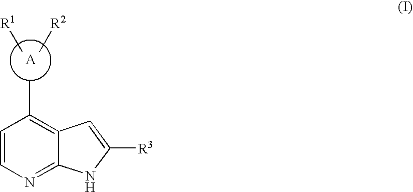
wherein
A is a 5- or 6-membered heteroaryl ring containing one heteroatom selected from nitrogen and sulphur, and optionally one further heteroatom which is nitrogen;
R¹ is hydrogen or methyl;
R² is —SO₂NR⁴R⁵;
R³ is —CR⁶R⁷R⁸, cyclopropyl or piperidinyl;
R⁴ is hydrogen or methyl,
R⁵ is C₁₋₆alkyl optionally substituted by one or two substituents independently selected from —OH and —NH₂, or a 5- or 6-membered heterocyclyl containing one heteroatom selected from oxygen, nitrogen and S(O)ₐ, or
R⁴ and R⁵ are linked to form a 5- or 6-membered heterocyclyl optionally containing one further heteroatom selected from oxygen, nitrogen and S(O)_{b};
R⁶, R⁷ and R⁸ are each independently hydrogen, fluorine or methyl;
a and b are each independently an integer selected from 0 to 2;
or a salt thereof.

2. A compound according to claim 1 wherein A is thienyl, thiazolyl, pyridinyl or pyrimidinyl.

3. A compound according to claim 1 or wherein R¹ is hydrogen.

4. A compound according to claim 1 wherein R³ is cyclopropyl.

5. A compound according to claim 1 wherein R⁴ is hydrogen.

6. A compound according to claim 1 wherein R⁵ is C₁₋₆alkyl optionally substituted by one or two substituents independently selected from —OH and —NH₂.

7. A compound according to claim 1 wherein R⁴ and R⁵ are linked to form a 5- or 6-membered heterocyclyl optionally containing one further heteroatom which is S(O)_{b}.

8. (canceled)

9. A compound which is:
N-(2-hydroxyethyl)-5-(2-methyl-1H-pyrrolo[2,3-b]pyridin-4-yl)-2-thiophenesulfonamide;
N-(1,1-dioxidotetrahydro-2H-thiopyran-4-yl)-5-(2-methyl-1H-pyrrolo[2,3-b]pyridin-4-yl)-2-thiophenesulfonamide;
N-(2-aminoethyl)-5-(2-methyl-1H-pyrrolo[2,3-b]pyridin-4-yl)-2-thiophenesulfonamide;
N-(2-hydroxyethyl)-5-[2-(3-piperidinyl)-1H-pyrrolo[2,3-b]pyridin-4-yl]-2-thiophenesulfonamide;
N-(2-aminoethyl)-5-[2-(3-piperidinyl)-1H-pyrrolo[2,3-b]pyridin-4-yl]-2-thiophenesulfonamide;
N-(1,1-dioxidotetrahydro-3-thienyl)-5-[2-(3-piperidinyl)-1H-pyrrolo[2,3-b]pyridin-4-yl]-2-thiophenesulfonamide;
N-(1,1-dioxidotetrahydro-2H-thiopyran-4-yl)-5-[2-(3-piperidinyl)-1H-pyrrolo[2,3-b]pyridin-4-yl]-2-thiophenesulfonamide;
N-(1,1-dioxidotetrahydro-2H-thiopyran-4-yl)-6-(2-methyl-1H-pyrrolo[2,3-b]pyridin-4-yl)-3-pyridinesulfonamide;
2-methyl-4-[5-(1-pyrrolidinylsulfonyl)-2-pyridinyl]-1H-pyrrolo[2,3-b]pyridine;
N-(1,1-dioxidotetrahydro-2H-thiopyran-4-yl)-5-(2-methyl-1H-pyrrolo[2,3-b]pyridin-4-yl)-2-pyridinesulfonamide;
2-methyl-4-[6-(1-pyrrolidinylsulfonyl)-3-pyridinyl]-1H-pyrrolo[2,3-b]pyridine;
N-(1,1-dioxidotetrahydro-2H-thiopyran-4-yl)-2-methyl-5-[2-(trifluoromethyl)-1H-pyrrolo[2,3-b]pyridin-4-yl]-3-thiophenesulfonamide;
N-(1,1-dioxidotetrahydro-2H-thiopyran-4-yl)-4-methyl-2-[2-(trifluoromethyl)-1H-pyrrolo[2,3-b]pyridin-4-yl]-1,3-thiazole-5-sulfonamide;
N-(1,1-dioxidotetrahydro-2H-thiopyran-4-yl)-2-(2-methyl-1H-pyrrolo[2,3-b]pyridin-4-yl)-5-pyrimidinesulfonamide;
6-[2-(difluoromethyl)-1H-pyrrolo[2,3-b]pyridin-4-yl]-N-(1,1-dioxidotetrahydro-2H-thiopyran-4-yl)-3-pyridinesulfonamide;
6-[2-(difluoromethyl)-1H-pyrrolo[2,3-b]pyridin-4-yl]-N-4-piperidinyl-3-pyridinesulfonamide;
2-(difluoromethyl)-4-{5-[(1,1-dioxido-4-thiomorpholinyl)sulfonyl]-2-pyridinyl}-1H-pyrrolo[2,3-b]pyridine;
2-(difluoromethyl)-4-{6-[(1,1-dioxido-4-thiomorpholinyl)sulfonyl]-3-pyridinyl}-1H-pyrrolo[2,3-b]pyridine;
5-[2-(difluoromethyl)-1H-pyrrolo[2,3-b]pyridin-4-yl]-N-4-piperidinyl-2-pyridinesulfonamide;
6-[2-(difluoromethyl)-1H-pyrrolo[2,3-b]pyridin-4-yl]-N-(1,1-dioxidotetrahydro-2H-thiopyran-4-yl)-N-methyl-3-pyridinesulfonamide;
5-[2-(difluoromethyl)-1H-pyrrolo[2,3-b]pyridin-4-yl]-N-(1,1-dioxidotetrahydro-2H-thiopyran-4-yl)-N-methyl-2-pyridinesulfonamide;
5-[2-(difluoromethyl)-1H-pyrrolo[2,3-b]pyridin-4-yl]-N-(1,1-dioxidotetrahydro-2H-thiopyran-4-yl)-2-pyridinesulfonamide;
N-(1,1-dioxidotetrahydro-2H-thiopyran-4-yl)-6-(2-methyl-1H-pyrrolo[2,3-b]pyridin-4-yl)-3-pyridinesulfonamide;
N-(2-hydroxyethyl)-6-(2-methyl-1H-pyrrolo[2,3-b]pyridin-4-yl)-3-pyridinesulfonamide;
6-(2-methyl-1H-pyrrolo[2,3-b]pyridin-4-yl)-N-4-piperidinyl-3-pyridinesulfonamide;
5-(2-methyl-1H-pyrrolo[2,3-b]pyridin-4-yl)-N-4-piperidinyl-2-pyridinesulfonamide;
N-(1,1-dioxidotetrahydro-2H-thiopyran-4-yl)-N-methyl-6-(2-methyl-1H-pyrrolo[2,3-b]pyridin-4-yl)-3-pyridinesulfonamide;
N-(1,1-dioxidotetrahydro-2H-thiopyran-4-yl)-N-methyl-5-(2-methyl-1H-pyrrolo[2,3-b]pyridin-4-yl)-2-pyridinesulfonamide;
N-(2,3-dihydroxypropyl)-5-(2-methyl-1H-pyrrolo[2,3-b]pyridin-4-yl)-2-pyridinesulfonamide;
N-(2,3-dihydroxypropyl)-5-(2-methyl-1H-pyrrolo[2,3-b]pyridin-4-yl)-2-pyridinesulfonamide;
N-(1,1-dioxidotetrahydro-3-thienyl)-5-(2-methyl-1H-pyrrolo[2,3-b]pyridin-4-yl)-2-thiophenesulfonamide;
2-(1,1-dimethylethyl)-4-[6-(1-pyrrolidinylsulfonyl)-3-pyridinyl]-1H-pyrrolo[2,3-b]pyridine;
N-(2-aminoethyl)-6-[2-(trifluoromethyl)-1H-pyrrolo[2,3-b]pyridin-4-yl]-3-pyridinesulfonamide;
N-(2-aminoethyl)-6-[2-(trifluoromethyl)-1H-pyrrolo[2,3-b]pyridin-4-yl]-3-pyridinesulfonamide trifluoroacetate;
N-(1,1-dioxidotetrahydro-2H-thiopyran-4-yl)-6-[2-(trifluoromethyl)-1H-pyrrolo[2,3-b]pyridin-4-yl]-3-pyridinesulfonamide;
N-4-piperidinyl-5-[2-(trifluoromethyl)-1H-pyrrolo[2,3-b]pyridin-4-yl]-2-pyridinesulfonamide;
N-(1,1-dioxidotetrahydro-2H-thiopyran-4-yl)-6-[2-(trifluoromethyl)-1H-pyrrolo[2,3-b]pyridin-4-yl]-3-pyridinesulfonamide;
4-{6-[(1,1-dioxido-4-thiomorpholinyl)sulfonyl]-3-pyridinyl}-2-(trifluoromethyl)-1H-pyrrolo[2,3-b]pyridine;
N-(2-hydroxyethyl)-5-[2-(trifluoromethyl)-1H-pyrrolo[2,3-b]pyridin-4-yl]-2-pyridinesulfonamide;
N-(2,3-dihydroxypropyl)-5-[2-(trifluoromethyl)-1H-pyrrolo[2,3-b]pyridin-4-yl]-2-pyridinesulfonamide;
6-[2-(1,1-dimethylethyl)-1H-pyrrolo[2,3-b]pyridin-4-yl]-N-(1,1-dioxidotetrahydro-2H-thiopyran-4-yl)-3-pyridinesulfonamide;
6-[2-(1,1-dimethylethyl)-1H-pyrrolo[2,3-b]pyridin-4-yl]-N-(2-hydroxyethyl)-3-pyridinesulfonamide;
2-(1,1-dimethylethyl)-4-{5-[(1,1-dioxido-4-thiomorpholinyl)sulfonyl]-2-pyridinyl}-1H-pyrrolo[2,3-b]pyridine;
6-[2-(1,1-dimethylethyl)-1H-pyrrolo[2,3-b]pyridin-4-yl]-N-(1,1-dioxidotetrahydro-3-thienyl)-3-pyridinesulfonamide;
5-[2-(1,1-dimethylethyl)-1H-pyrrolo[2,3-b]pyridin-4-yl]-N-4-piperidinyl-2-pyridinesulfonamide;
6-[2-(1,1-dimethylethyl)-1H-pyrrolo[2,3-b]pyridin-4-yl]-N-(1,1-dioxidotetrahydro-2H-thiopyran-4-yl)-N-methyl-3-pyridinesulfonamide;
5-[2-(1,1-dimethylethyl)-1H-pyrrolo[2,3-b]pyridin-4-yl]-N-(1,1-dioxidotetrahydro-2H-thiopyran-4-yl)-N-methyl-2-pyridinesulfonamide;
5-[2-(1,1-dimethylethyl)-1H-pyrrolo[2,3-b]pyridin-4-yl]-N-(2-hydroxyethyl)-2-pyridinesulfonamide;
N-(2,3-dihydroxypropyl)-5-[2-(1,1-dimethylethyl)-1H-pyrrolo[2,3-b]pyridin-4-yl]-2-pyridinesulfonamide;
N-(2-hydroxyethyl)-6-[2-(trifluoromethyl)-1H-pyrrolo[2,3-b]pyridin-4-yl]-3-pyridinesulfonamide;
N-4-piperidinyl-6-[2-(trifluoromethyl)-1H-pyrrolo[2,3-b]pyridin-4-yl]-3-pyridinesulfonamide;
N-(1,1-dioxidotetrahydro-3-thienyl)-6-[2-(trifluoromethyl)-1H-pyrrolo[2,3-b]pyridin-4-yl]-3-pyridinesulfonamide;
4-{6-[(1,1-dioxido-4-thiomorpholinyl)sulfonyl]-3-pyridinyl}-2-(trifluoromethyl)-1H-pyrrolo[2,3-b]pyridine;
N-(1,1-dioxidotetrahydro-2H-thiopyran-4-yl)-6-[2-(1-methylethyl)-1H-pyrrolo[2,3-b]pyridin-4-yl]-3-pyridinesulfonamide;
2-(1-methylethyl)-4-[6-(1-pyrrolidinylsulfonyl)-3-pyridinyl]-1H-pyrrolo[2,3-b]pyridine;
N-(2-hydroxyethyl)-6-[2-(1-methylethyl)-1H-pyrrolo[2,3-b]pyridin-4-yl]-3-pyridinesulfonamide;
4-{5-[(1,1-dioxido-4-thiomorpholinyl)sulfonyl]-2-pyridinyl}-2-(1-methylethyl)-1H-pyrrolo[2,3-b]pyridine;
N-(2-aminoethyl)-6-[2-(1-methylethyl)-1H-pyrrolo[2,3-b]pyridin-4-yl]-3-pyridinesulfonamide;
6-[2-(1-methylethyl)-1H-pyrrolo[2,3-b]pyridin-4-yl]-N-4-piperidinyl-3-pyridinesulfonamide;
N-(1,1-dioxidotetrahydro-3-thienyl)-6-[2-(1-methylethyl)-1H-pyrrolo[2,3-b]pyridin-4-yl]-3-pyridinesulfonamide;
N-(1,1-dioxidotetrahydro-2H-thiopyran-4-yl)-5-[2-(1-methylethyl)-1H-pyrrolo[2,3-b]pyridin-4-yl]-2-pyridinesulfonamide;
4-{6-[(1,1-dioxido-4-thiomorpholinyl)sulfonyl]-3-pyridinyl}-2-(1-methylethyl)-1H-pyrrolo[2,3-b]pyridine;
5-[2-(1-methylethyl)-1H-pyrrolo[2,3-b]pyridin-4-yl]-N-4-piperidinyl-2-pyridinesulfonamide;
N-(1,1-dioxidotetrahydro-2H-thiopyran-4-yl)-N-methyl-6-[2-(1-methylethyl)-1H-pyrrolo[2,3-b]pyridin-4-yl]-3-pyridinesulfonamide;
N-(1,1-dioxidotetrahydro-2H-thiopyran-4-yl)-N-methyl-5-[2-(1-methylethyl)-1H-pyrrolo[2,3-b]pyridin-4-yl]-2-pyridinesulfonamide;
N-(2-hydroxyethyl)-5-[2-(1-methylethyl)-1H-pyrrolo[2,3-b]pyridin-4-yl]-2-pyridinesulfonamide;
N-(2-aminoethyl)-6-[2-(1,1-dimethylethyl)-1H-pyrrolo[2,3-b]pyridin-4-yl]-3-pyridinesulfonamide;
6-[2-(1,1-dimethylethyl)-1H-pyrrolo[2,3-b]pyridin-4-yl]-N-4-piperidinyl-3-pyridinesulfonamide;
2-(1,1-dimethylethyl)-4-{6-[(1,1-dioxido-4-thiomorpholinyl)sulfonyl]-3-pyridinyl}-1H-pyrrolo[2,3-b]pyridine;
2-(1,1-dimethylethyl)-4-[6-(1-pyrrolidinylsulfonyl)-3-pyridinyl]-1H-pyrrolo[2,3-b]pyridine;
2-(1,1-dimethylethyl)-4-[6-(1-pyrrolidinylsulfonyl)-3-pyridinyl]-1H-pyrrolo[2,3-b]pyridine hydrochloride;
2-(1,1-dimethylethyl)-4-[6-(1-pyrrolidinylsulfonyl)-3-pyridinyl]-1H-pyrrolo[2,3-b]pyridine sulfate;
N-(2-aminoethyl)-5-[2-(difluoromethyl)-1H-pyrrolo[2,3-b]pyridin-4-yl]-2-thiophenesulfonamide;
5-[2-(difluoromethyl)-1H-pyrrolo[2,3-b]pyridin-4-yl]-N-(1,1-dioxidotetrahydro-3-thienyl)-2-thiophenesulfonamide;
5-[2-(difluoromethyl)-1H-pyrrolo[2,3-b]pyridin-4-yl]-N-(1,1-dioxidotetrahydro-2H-thiopyran-4-yl)-2-thiophenesulfonamide;
N-(2-hydroxy-2-methylpropyl)-5-[2-(trifluoromethyl)-1H-pyrrolo[2,3-b]pyridin-4-yl]-2-thiophenesulfonamide;
N-(2-hydroxyethyl)-5-[2-(trifluoromethyl)-1H-pyrrolo[2,3-b]pyridin-4-yl]-2-thiophenesulfonamide;
N-(tetrahydro-2H-pyran-4-yl)-5-[2-(trifluoromethyl)-1H-pyrrolo[2,3-b]pyridin-4-yl]-2-thiophenesulfonamide;
N-(2,3-dihydroxypropyl)-5-[2-(trifluoromethyl)-1H-pyrrolo[2,3-b]pyridin-4-yl]-2-thiophenesulfonamide;
N-(2-aminoethyl)-5-[2-(trifluoromethyl)-1H-pyrrolo[2,3-b]pyridin-4-yl]-2-thiophenesulfonamide;
N-4-piperidinyl-5-[2-(trifluoromethyl)-1H-pyrrolo[2,3-b]pyridin-4-yl]-2-thiophenesulfonamide;
N-(tetrahydro-2H-thiopyran-4-yl)-6-[2-(trifluoromethyl)-1H-pyrrolo[2,3-b]pyridin-4-yl]-3-pyridinesulfonamide;
N-(2-aminoethyl)-6-[2-(trifluoromethyl)-1H-pyrrolo[2,3-b]pyridin-4-yl]-3-pyridinesulfonamide;
5-(2-cyclopropyl-1H-pyrrolo[2,3-b]pyridin-4-yl)-N-4-piperidinyl-2-thiophenesulfonamide;
5-(2-cyclopropyl-1H-pyrrolo[2,3-b]pyridin-4-yl)-N-(2-hydroxyethyl)-2-thiophenesulfonamide;
5-(2-cyclopropyl-1H-pyrrolo[2,3-b]pyridin-4-yl)-N-(2-hydroxy-2-methylpropyl)-2-thiophenesulfonamide;
N-(2-aminoethyl)-5-(2-cyclopropyl-1H-pyrrolo[2,3-b]pyridin-4-yl)-2-thiophenesulfonamide;
5-(2-cyclopropyl-1H-pyrrolo[2,3-b]pyridin-4-yl)-N-(2,3-dihydroxypropyl)-2-thiophenesulfonamide;
5-(2-cyclopropyl-1H-pyrrolo[2,3-b]pyridin-4-yl)-N-(1,1-dioxidotetrahydro-2H-thiopyran-4-yl)-2-thiophenesulfonamide;
5-(2-cyclopropyl-1H-pyrrolo[2,3-b]pyridin-4-yl)-N-(tetrahydro-2H-pyran-4-yl)-2-thiophenesulfonamide;
N-(2-hydroxy-1,1-dimethylethyl)-5-[2-(trifluoromethyl)-1H-pyrrolo[2,3-b]pyridin-4-yl]-2-thiophenesulfonamide;
N-(2-hydroxy-2-methylpropyl)-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(1R)-2-hydroxy-1-methylethyl]-2-thiophenesulfonamide;
N-[(2S)-2-hydroxypropyl]-5-[2-(trifluoromethyl)-1H-pyrrolo[2,3-b]pyridin-4-yl]-2-thiophenesulfonamide;
N-[(1R)-2-hydroxy-1-methylethyl]-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(2S)-2-hydroxypropyl]-2-thiophenesulfonamide;
N-[(2R)-2-hydroxypropyl]-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(1S)-2-hydroxy-1-methylethyl]-2-thiophenesulfonamide;
N-[(1S)-2-hydroxy-1-methylethyl]-5-[2-(trifluoromethyl)-1H-pyrrolo[2,3-b]pyridin-4-yl]-2-thiophenesulfonamide;
5-(2-cyclopropyl-1H-pyrrolo[2,3-b]pyridin-4-yl)-N-[(2R)-2-hydroxypropyl]-2-thiophenesulfonamide;
N-(2-hydroxy-1,1-dimethylethyl)-5-(2-methyl-1H-pyrrolo[2,3-b]pyridin-4-yl)-2-thiophenesulfonamide;
N-[(1R)-2-hydroxy-1-methylethyl]-5-(2-methyl-1H-pyrrolo[2,3-b]pyridin-4-yl)-2-thiophenesulfonamide;
N-[(2S)-2-hydroxypropyl]-5-(2-methyl-1H-pyrrolo[2,3-b]pyridin-4-yl)-2-thiophenesulfonamide;
N-[(2R)-2-hydroxypropyl]-5-(2-methyl-1H-pyrrolo[2,3-b]pyridin-4-yl)-2-thiophenesulfonamide;
N-(2-hydroxy-2-methylpropyl)-5-(2-methyl-1H-pyrrolo[2,3-b]pyridin-4-yl)-2-thiophenesulfonamide;
5-(2-cyclopropyl-1H-pyrrolo[2,3-b]pyridin-4-yl)-N-(2-hydroxy-1,1-dimethylethyl)-2-thiophenesulfonamide;
N-(2-aminoethyl)-6-[2-(1,1-dimethylethyl)-1H-pyrrolo[2,3-b]pyridin-4-yl]-3-pyridinesulfonamide;
5-(2-cyclopropyl-1H-pyrrolo[2,3-b]pyridin-4-yl)-N-(2-hydroxy-2-methylpropyl)-2-pyridinesulfonamide;
5-(2-cyclopropyl-1H-pyrrolo[2,3-b]pyridin-4-yl)-N-(2-hydroxy-1,1-dimethylethyl)-2-pyridinesulfonamide;
N-(2-hydroxy-1,1-dimethylethyl)-5-[2-(trifluoromethyl)-1H-pyrrolo[2,3-b]pyridin-4-yl]-2-pyridinesulfonamide;
6-(2-cyclopropyl-1H-pyrrolo[2,3-b]pyridin-4-yl)-N-(2-hydroxy-2-methylpropyl)-3-pyridinesulfonamide;
N-(2-hydroxy-2-methylpropyl)-6-(2-methyl-1H-pyrrolo[2,3-b]pyridin-4-yl)-3-pyridinesulfonamide;
6-(2-cyclopropyl-1H-pyrrolo[2,3-b]pyridin-4-yl)-N-(2-hydroxy-1,1-dimethylethyl)-3-pyridinesulfonamide;
N-(2-hydroxy-1,1-dimethylethyl)-6-[2-(trifluoromethyl)-1H-pyrrolo[2,3-b]pyridin-4-yl]-3-pyridinesulfonamide;
N-[(1S)-2-hydroxy-1-methylethyl]-6-[2-(trifluoromethyl)-1H-pyrrolo[2,3-b]pyridin-4-yl]-3-pyridinesulfonamide;
6-(2-cyclopropyl-1H-pyrrolo[2,3-b]pyridin-4-yl)-N-[(2R)-2-hydroxypropyl]-3-pyridinesulfonamide;
N-[(1R)-2-hydroxy-1-methylethyl]-6-[2-(trifluoromethyl)-1H-pyrrolo[2,3-b]pyridin-4-yl]-3-pyridinesulfonamide;
N-[(1S)-2-hydroxy-1-methylethyl]-6-(2-methyl-1H-pyrrolo[2,3-b]pyridin-4-yl)-3-pyridinesulfonamide;
N-[(2S)-2-hydroxypropyl]-6-[2-(trifluoromethyl)-1H-pyrrolo[2,3-b]pyridin-4-yl]-3-pyridinesulfonamide;
6-(2-cyclopropyl-1H-pyrrolo[2,3-b]pyridin-4-yl)-N-[(2S)-2-hydroxypropyl]-3-pyridinesulfonamide;
N-[(2R)-2-hydroxypropyl]-6-[2-(trifluoromethyl)-1H-pyrrolo[2,3-b]pyridin-4-yl]-3-pyridinesulfonamide;
N-[(2R)-2-hydroxypropyl]-6-(2-methyl-1H-pyrrolo[2,3-b]pyridin-4-yl)-3-pyridinesulfonamide;
6-(2-cyclopropyl-1H-pyrrolo[2,3-b]pyridin-4-yl)-N-[(1R)-2-hydroxy-1-methylethyl]-3-pyridinesulfonamide;
N-[(2S)-2-hydroxypropyl]-6-(2-methyl-1H-pyrrolo[2,3-b]pyridin-4-yl)-3-pyridinesulfonamide;
6-(2-cyclopropyl-1H-pyrrolo[2,3-b]pyridin-4-yl)-N-[(1S)-2-hydroxy-1-methylethyl]-3-pyridinesulfonamide;
N-[(1R)-2-hydroxy-1-methylethyl]-6-(2-methyl-1H-pyrrolo[2,3-b]pyridin-4-yl)-3-pyridinesulfonamide;
N-(2-hydroxy-1,1-dimethylethyl)-5-(2-methyl-1H-pyrrolo[2,3-b]pyridin-4-yl)-2-pyridinesulfonamide;
5-(2-cyclopropyl-1H-pyrrolo[2,3-b]pyridin-4-yl)-N-[(2R)-2-hydroxypropyl]-2-pyridinesulfonamide;
N-[(1S)-2-hydroxy-1-methylethyl]-5-(2-methyl-1H-pyrrolo[2,3-b]pyridin-4-yl)-2-pyridinesulfonamide;
5-(2-cyclopropyl-1H-pyrrolo[2,3-b]pyridin-4-yl)-N-[(1R)-2-hydroxy-1-methylethyl]-2-pyridinesulfonamide;
N-[(2R)-2-hydroxypropyl]-5-[2-(trifluoromethyl)-1H-pyrrolo[2,3-b]pyridin-4-yl]-2-pyridinesulfonamide;
N-[(2R)-2-hydroxypropyl]-5-(2-methyl-1H-pyrrolo[2,3-b]pyridin-4-yl)-2-pyridinesulfonamide;
N-[(2S)-2-hydroxypropyl]-5-[2-(trifluoromethyl)-1H-pyrrolo[2,3-b]pyridin-4-yl]-2-pyridinesulfonamide;
5-(2-cyclopropyl-1H-pyrrolo[2,3-b]pyridin-4-yl)-N-[(1S)-2-hydroxy-1-methylethyl]-2-pyridinesulfonamide;
N-[(1R)-2-hydroxy-1-methylethyl]-5-(2-methyl-1H-pyrrolo[2,3-b]pyridin-4-yl)-2-pyridinesulfonamide;
5-(2-cyclopropyl-1H-pyrrolo[2,3-b]pyridin-4-yl)-N-[(2S)-2-hydroxypropyl]-2-pyridinesulfonamide;
N-(2-hydroxy-1,1-dimethylethyl)-6-(2-methyl-1H-pyrrolo[2,3-b]pyridin-4-yl)-3-pyridinesulfonamide;
N-[(1S)-2-hydroxy-1-methylethyl]-5-[2-(trifluoromethyl)-1H-pyrrolo[2,3-b]pyridin-4-yl]-2-pyridinesulfonamide;
N-[(2S)-2-hydroxypropyl]-5-(2-methyl-1H-pyrrolo[2,3-b]pyridin-4-yl)-2-pyridinesulfonamide;
N-[(1R)-2-hydroxy-1-methylethyl]-5-[2-(trifluoromethyl)-1H-pyrrolo[2,3-b]pyridin-4-yl]-2-pyridinesulfonamide;
or a salt thereof.

10. A pharmaceutical composition comprising a compound as claimed in any claim 1, or a pharmaceutically acceptable salt thereof, and one or more pharmaceutically acceptable excipients.

11-13. (canceled)

14. A method of treating a disorder mediated by inappropriate IKK2 activity comprising administering a safe and effective amount of a compound as claimed in claim 1, or a pharmaceutically acceptable salt thereof, to a patient in need thereof.

15. A method according to claim 14 wherein the disorder mediated by inappropriate IKK2 activity is cancer or cachexia.

16. A method according to claim 14 wherein the disorder mediated by inappropriate IKK2 activity is an inflammatory or tissue repair disorder.

17. A method according to claim 14 wherein the disorder mediated by inappropriate IKK2 activity is an autoimmune disease.

18. A method according to claim 17 wherein the autoimmune disease is systemic lupus eythematosus, multiple sclerosis, psoriatic arthritis, or alkylosing spondylitis.

19. A method according to claim 14 wherein the disorder mediated by inappropriate IKK2 activity is selected from the group consisting of: rheumatoid arthritis, inflammatory bowel disease, COPD (chronic obstructive pulmonary disease), asthma, rhinitis, osteoarthritis, osteoporosis, psoriasis, atopic dermatitis, ultraviolet radiation (UV)-induced skin damage, systemic lupus eythematosus, multiple sclerosis, psoriatic arthritis, alkylosing spondylitis, tissue rejection, organ rejection, Alzheimer's disease, stroke, atherosclerosis, restonosis, diabetes, glomerulonephritis, Hodgkin's disease, cachexia, inflammation associated with infection and certain viral infections, including acquired immune deficiency syndrome (AIDS), adult respiratory distress syndrome, and Ataxia Telangiestasia.

20. A method according to claim 19 wherein the disorder mediated by inappropriate IKK2 activity is selected from the group consisting of: Alzheimer's disease, stroke atherosclerosis, restenosis, diabetes, glomerulonephritis, osteoarthritis, osteoporosis, and Ataxia Telangiestasia.

21. A method according to claim 19 wherein the disorder mediated by inappropriate IKK2 activity is rheumatoid arthritis, COPD, asthma or rhinitis.

22. A method according to claim 21 wherein the disorder mediated by inappropriate IKK2 activity is rheumatoid arthritis.

23. A method according to claim 21 wherein the disorder mediated by inappropriate IKK2 activity is COPD.

24. A method according to claim 21 wherein the disorder mediated by inappropriate IKK2 activity is asthma.

25. A method according to claim 21 wherein the disorder mediated by inappropriate IKK2 activity is rhinitis.

26. A process for preparing a compound as claimed in claim 1, or a salt thereof, comprising reacting a compound of formula (II) 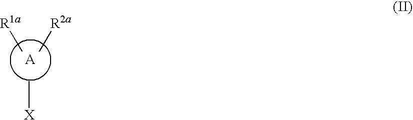
wherein R^{1a} and R^{2a} are R¹ and R² as defined in claim 1 or groups convertible to R¹ and R², and X is a leaving group, with a compound of formula (IIIA) or (IIIB) 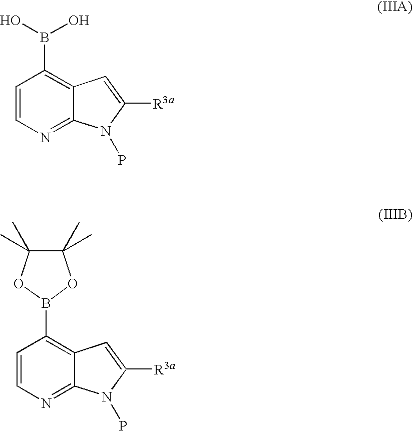
wherein P is hydrogen or a protecting group and R^{3a} is R³ as defined in claim 1 or a group convertible to R³, in the presence of a catalyst.
